(19) United States
(12) Patent Application Publication
(10) Pub. No.: US 2022/0348563 A1
(43) Pub. Date: Nov. 3, 2022

(54) KDM INHIBITORS AND USES THEREOF

(71) Applicants: Xiaofeng Zhang, Boston, MA (US); DANA-FARBER CANCER INSTITUTE, INC., Boston, MA (US)

(72) Inventors: Jun Qi, Sharon, MA (US); Paul M. Park, Waltham, MA (US); Chengkui Pei, Brighton, MA (US); Xiaofeng Zhang, Boston, MA (US)

(73) Assignee: DANA-FARBER CANCER INSTITUTE, INC., Boston, MA (US)

(21) Appl. No.: 17/642,495

(22) PCT Filed: Sep. 10, 2020
(86) PCT No.: PCT/US2020/050165

§ 371 Date: Mar. 11, 2022

Related U.S. Application Data
(60) Provisional application No. 62/900,314, filed on Sep. 13, 2019.

(51) Int. Cl. C07D 405/14; C07D 401/14; C07D 401/12; C07D 295/192; C07D 233/70; C07D 333/38; C07D 213/81; C07C 237/34; C07D 413/14; C07D 493/04

(52) U.S. Cl. CPC C07D 405/14; C07D 401/14; C07D 401/12; C07D 295/192; C07D 233/70; C07D 333/38; C07D 213/81; C07C 237/34; C07D 413/14; C07D 493/04

(57) ABSTRACT

Provided herein are compounds that inhibit histone lysine demethylase (KDM) and pharmaceutically acceptable salts, hydrates, solvates, polymorphs, co-crystals, tautomers, stereoisomers, isotopically labeled derivatives, and prodrugs thereof, pharmaceutical compositions thereof, and methods of treating or preventing diseases (e.g., proliferative diseases, e.g., cancer). In certain embodiments, the compounds described herein are represented by formulas (I) and (II).

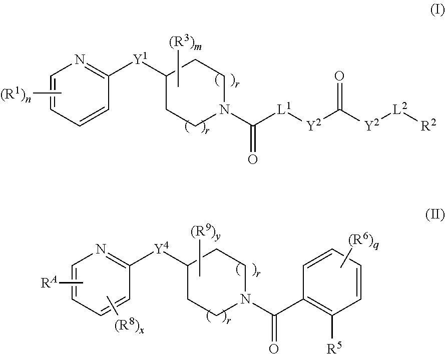

### RELATED APPLICATIONS

This application claims the benefit of priority under 35 U.S.C. § 119(e) to U.S. Provisional Application No. 62/900,314, filed on Sep. 13, 2019, which is incorporated herein by reference in its entirety.

### BACKGROUND OF THE INVENTION

The overexpression of certain histone lysine demethylases (KDMs) (e.g., KDMs in the Jumonji C (JmjC) KDM2-7 subfamily) has been linked to cancer and other diseases. KDMs have also been linked to cell cycle control. In view of the significant role of KDM enzymes (e.g., KDM3) in cell cycle control and in various diseases (e.g., proliferative diseases such as cancer, and cardiovascular diseases), there has been significant interest in developing modulators of the activity of these enzymes, including selective modulators (e.g., selective inhibitors) of particular KDMs (e.g., KDM3). Such inhibitors are useful as research tools as well as therapeutic agents in the treatment of various diseases, including proliferative diseases, such as cancer, and cardiovascular diseases.

### SUMMARY OF THE INVENTION

Provided herein are compounds, and pharmaceutically acceptable salts, hydrates, solvates, polymorphs, co-crystals, tautomers, stereoisomers, isotopically labeled derivatives, and prodrugs thereof, and pharmaceutical compositions thereof, and kits thereof. The compounds provided herein are histone lysine demethylase (KDM) inhibitors (e.g., KDM3 inhibitors), and are therefore useful for the treatment and/or prevention of diseases (e.g., proliferative diseases, e.g., cancer, cardiovascular diseases, inflammatory diseases, and autoimmune diseases). In certain embodiments, the compounds described herein are selective inhibitors of a KDM (e.g., KDM3).

In one aspect, provided herein are compounds of Formula (I) or (II):

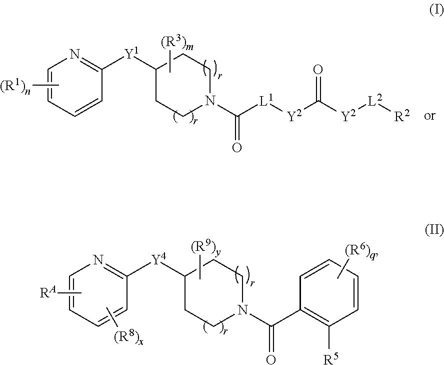

and pharmaceutically acceptable salts, hydrates, solvates, polymorphs, co-crystals, tautomers, stereoisomers, isotopically labeled derivatives, and prodrugs thereof; wherein R^{A}, R¹, R², R³, R⁵, R⁶, R⁸, R⁹, r, n, m, x, y, q, Y¹, Y², Y⁴, L¹, and L² are as defined herein.

In another aspect, provided herein are compounds having the following structures (1-22):

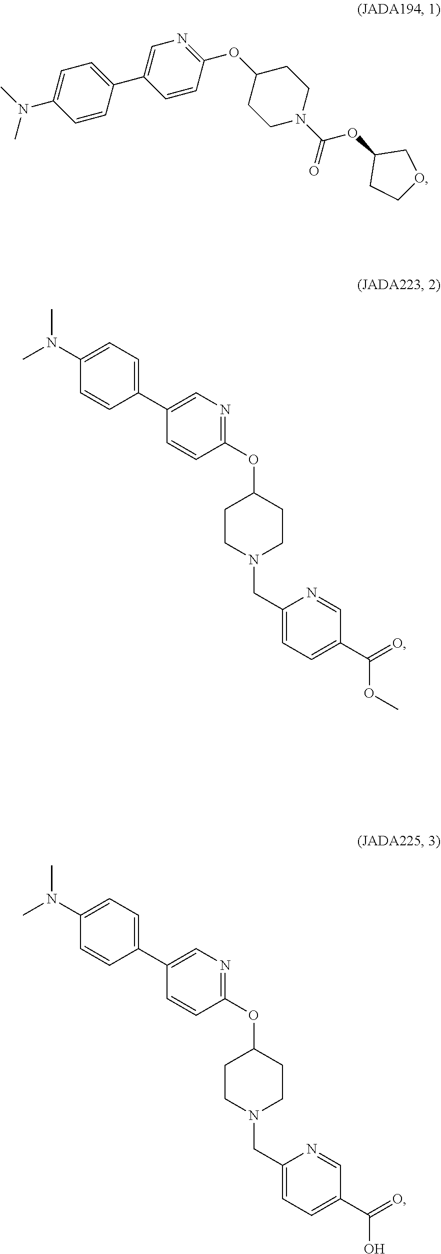

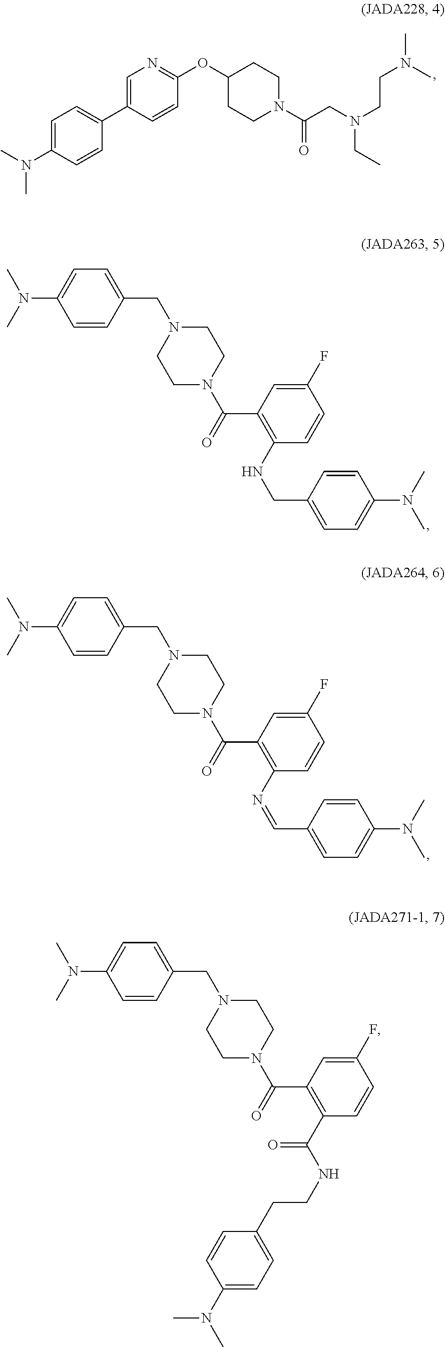

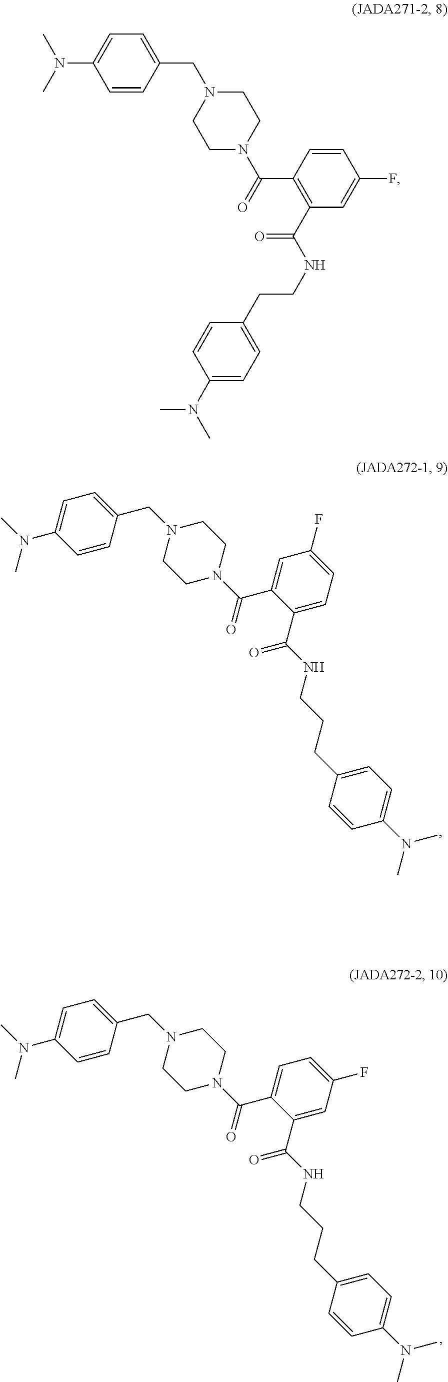

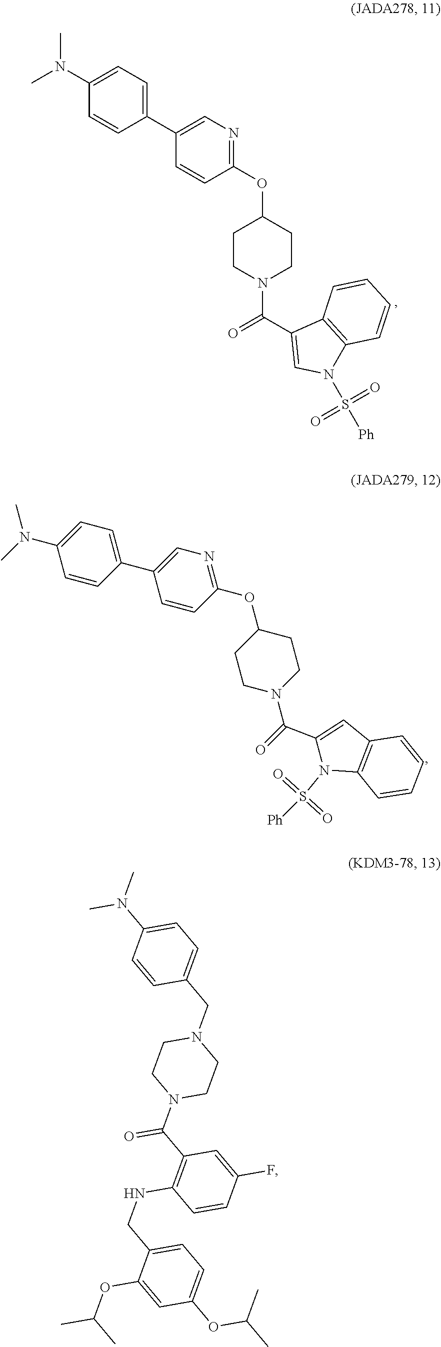 [SMILES: CN(C)c1ccc(-c2ccc(OC3CCN(C(=O)c4cn(S(=O)(=O)c5ccccc5)c5ccccc45)CC3)nc2)cc1 ; CN(C)c1ccc(-c2ccc(OC3CCN(C(=O)c4cc5ccccc5n4S(=O)(=O)c4ccccc4)CC3)nc2)cc1 ; CC(C)Oc1ccc(CNc2ccc(F)cc2C(=O)N2CCN(Cc3ccc(N(C)C)cc3)CC2)c(OC(C)C)c1]

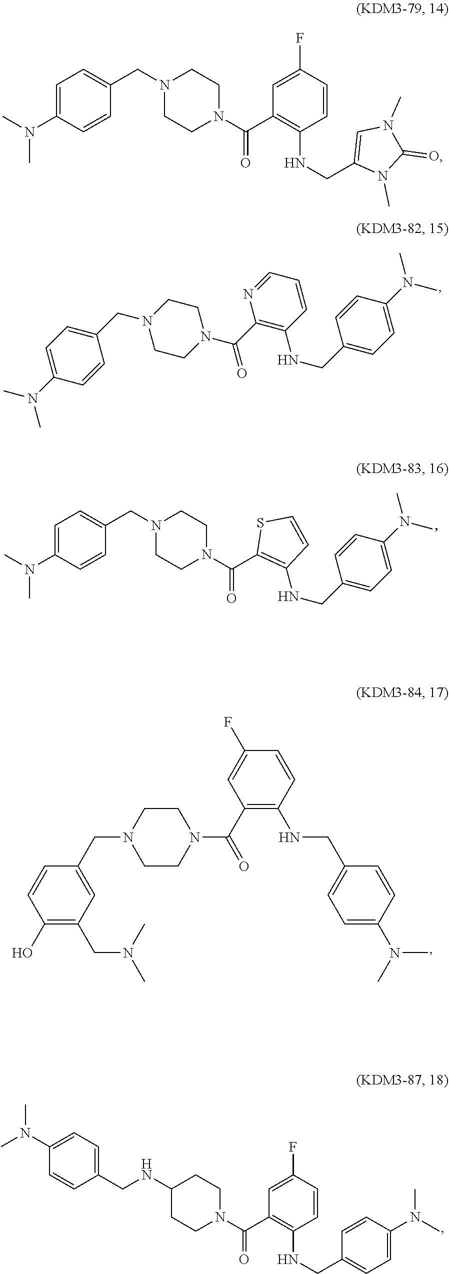

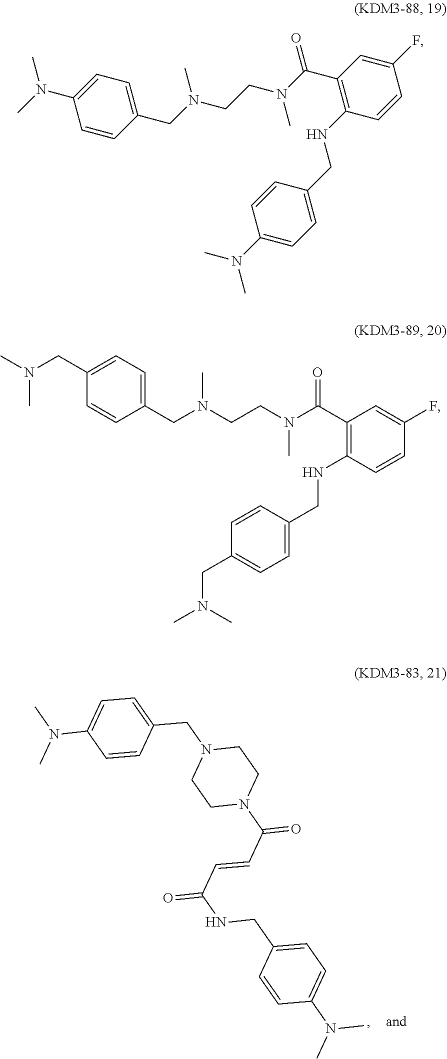

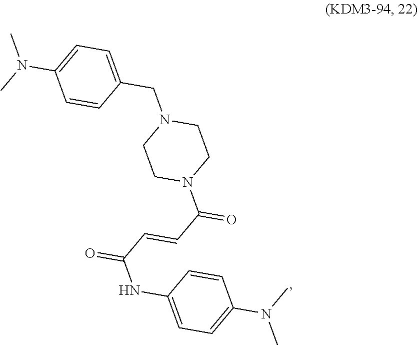

and pharmaceutically acceptable salts, hydrates, solvates, polymorphs, co-crystals, tautomers, stereoisomers, isotopically labeled derivatives, and prodrugs thereof.

In another aspect, provided herein are pharmaceutical compositions comprising a compound of Formula (I) or (II), or any one of compounds 1-22 (collectively “the inventive compounds”), or a pharmaceutically acceptable salt, hydrate, solvate, polymorph, co-crystal, tautomer, stereoisomer, isotopically labeled derivative, or prodrug thereof; and optionally a pharmaceutically acceptable excipient. In certain embodiments, the pharmaceutical compositions described herein include a therapeutically effective amount of a compound of Formula (I) or (II), or any one of compounds 1-22, or a pharmaceutically acceptable salt, solvate, hydrate, polymorph, co-crystal, tautomer, stereoisomer, isotopically labeled derivative, or prodrug thereof. The pharmaceutical compositions described herein may be useful for treating and/or preventing a disease (e.g., a proliferative disease, e.g., cancer) in a subject.

In another aspect, provided herein are methods for:
i. treating diseases or conditions (e.g., proliferative diseases (e.g., cancer), cardiovascular diseases, inflammatory diseases, autoimmune diseases) in a subject;
ii. inhibiting the activity of a KDM (e.g., KDM3) in a subject, cell, or biological sample; and/or
iii. inducing apoptosis in a cell of a subject or biological sample;
   by administering to the subject or contacting the biological sample with a compound of Formula (I) or (II), or any one of compounds 1-22, or a pharmaceutically acceptable salt, solvate, hydrate, polymorph, co-crystal, tautomer, stereoisomer, isotopically labeled derivative, or prodrug thereof, or a pharmaceutical composition thereof.

In another aspect, provided herein are kits comprising an inventive compound, or a salt, hydrate, solvate, polymorph, co-crystal, tautomer, stereoisomer, isotopically labeled derivative, or prodrug thereof, or pharmaceutical composition as described herein. The kits described herein may include a single dose or multiple doses of the inventive compound or pharmaceutical composition thereof. The provided kits may be useful in a method disclosed herein (e.g., a method of treating and/or preventing a disease in a subject, e.g., a proliferative disease, e.g., cancer). A kit of the disclosure may further include instructions for using the kit and its component(s) (e.g., instructions for using the inventive compound or pharmaceutical composition included in the kit).

The details of certain embodiments of the present disclosure are set forth herein. Other features, objects, and advantages of the present disclosure will be apparent from the Detailed Description, Figures, Examples, and Claims.

### Definitions

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, N.Y., 1962); and Wilen, S. H., Tables of Resolving Agents and Optical Resolutions p. 268 (E. L. Eliel, Ed., Univ. of Notre Dame Press, Notre Dame, Ind. 1972). The present disclosure additionally encompasses compounds as individual isomers substantially free of other isomers, and alternatively, as mixtures of various isomers.

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, or the replacement of ¹²C with ¹³C or ¹⁴C are within the scope of the disclosure. Such compounds are useful, for example, as analytical tools or probes in biological assays.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Non-limiting examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g., n-hexyl). Additional non-limiting examples of alkyl groups include n-heptyl (C₇), n-octyl (C₈), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu), unsubstituted iso-butyl (i-Bu)). In certain embodiments, the alkyl group is a substituted C₁₋₁₀ alkyl (such as substituted C₁₋₆ alkyl, e.g., —CF₃, Bn).

The term “haloalkyl” is a substituted alkyl group, wherein one or more of the hydrogen atoms are independently replaced by a halogen atom, e.g., fluoro, bromo, chloro, or iodo. In some embodiments, the haloalkyl moiety has 1 to 8 carbon atoms (“C₁₋₈ haloalkyl”). In some embodiments, the haloalkyl moiety has 1 to 6 carbon atoms (“C₁₋₆ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). Non-limiting examples of haloalkyl groups include —CHF₂, —CH₂F, —CF₃, —CH₂CF₃, —CF₂CF₃, —CF₂CF₂CF₃, —CCl₃, —CFCl₂, —CF₂C₁, and the like.

The term “heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkyl group refers to a saturated group having from 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁₋₃ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀ alkyl. In certain embodiments, the heteroalkyl group is a substituted heteroC₁₋₁₀ alkyl.

The term “alkenyl” refers to a radical of a straight-chain or branched hydrocarbon group having from 2 to 10 carbon atoms and one or more carbon-carbon double bonds (e.g., 1, 2, 3, or 4 double bonds). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Non-limiting examples of C₂₋₆ alkenyl groups include the aforementioned C₂₋₄ alkenyl groups as well as pentenyl (C₅), pentadienyl (C₅), hexenyl (C₆), and the like. Additional non-limiting examples of alkenyl include heptenyl (C₇), octenyl (C₈), octatrienyl (C₈), and the like.

Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₁₀ alkenyl. In certain embodiments, the alkenyl group is a substituted C₂₋₁₀ alkenyl. In an alkenyl group, a C═C double bond for which the stereochemistry is not specified (e.g., —CH═CHCH₃ or

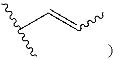

may be an (E)- or (Z)-double bond.

The term “heteroalkenyl” refers to an alkenyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkenyl group refers to a group having from 2 to 10 carbon atoms, at least one double bond, and 1 or more heteroatoms within the parent chain (“heteroC₂₋₁₀ alkenyl”). In some embodiments, a heteroalkenyl group has 2 to 9 carbon atoms at least one double bond, and 1 or more heteroatoms within the parent chain (“heteroC₂₋₉ alkenyl”). In some embodiments, a heteroalkenyl group has 2 to 8 carbon atoms, at least one double bond, and 1 or more heteroatoms within the parent chain (“heteroC₂₋₈ alkenyl”). In some embodiments, a heteroalkenyl group has 2 to 7 carbon atoms, at least one double bond, and 1 or more heteroatoms within the parent chain (“heteroC₂₋₇ alkenyl”). In some embodiments, a heteroalkenyl group has 2 to 6 carbon atoms, at least one double bond, and 1 or more heteroatoms within the parent chain (“heteroC₂₋₆ alkenyl”). In some embodiments, a heteroalkenyl group has 2 to 5 carbon atoms, at least one double bond, and 1 or 2 heteroatoms within the parent chain (“heteroC₂₋₅ alkenyl”). In some embodiments, a heteroalkenyl group has 2 to 4 carbon atoms, at least one double bond, and 1 or 2 heteroatoms within the parent chain (“heteroC₂₋₄ alkenyl”). In some embodiments, a heteroalkenyl group has 2 to 3 carbon atoms, at least one double bond, and 1 heteroatom within the parent chain (“heteroC₂₋₃ alkenyl”). In some embodiments, a heteroalkenyl group has 2 to 6 carbon atoms, at least one double bond, and 1 or 2 heteroatoms within the parent chain (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

The term “alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₁₀ alkynyl. In certain embodiments, the alkynyl group is a substituted C₂₋₁₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkynyl group refers to a group having from 2 to 10 carbon atoms, at least one triple bond, and 1 or more heteroatoms within the parent chain (“heteroC₂₋₁₀ alkynyl”). In some embodiments, a heteroalkynyl group has 2 to 9 carbon atoms, at least one triple bond, and 1 or more heteroatoms within the parent chain (“heteroC₂₋₉ alkynyl”). In some embodiments, a heteroalkynyl group has 2 to 8 carbon atoms, at least one triple bond, and 1 or more heteroatoms within the parent chain (“heteroC₂₋₈ alkynyl”). In some embodiments, a heteroalkynyl group has 2 to 7 carbon atoms, at least one triple bond, and 1 or more heteroatoms within the parent chain (“heteroC₂₋₇ alkynyl”). In some embodiments, a heteroalkynyl group has 2 to 6 carbon atoms, at least one triple bond, and 1 or more heteroatoms within the parent chain (“heteroC₂₋₆ alkynyl”). In some embodiments, a heteroalkynyl group has 2 to 5 carbon atoms, at least one triple bond, and 1 or 2 heteroatoms within the parent chain (“heteroC₂₋₅ alkynyl”). In some embodiments, a heteroalkynyl group has 2 to 4 carbon atoms, at least one triple bond, and for 2 heteroatoms within the parent chain (“heteroC₂₋₄ alkynyl”). In some embodiments, a heteroalkynyl group has 2 to 3 carbon atoms, at least one triple bond, and 1 heteroatom within the parent chain (“heteroC₂₋₃ alkynyl”). In some embodiments, a heteroalkynyl group has 2 to 6 carbon atoms, at least one triple bond, and 1 or 2 heteroatoms within the parent chain (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Non-limiting examples of C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Non-limiting examples of C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Non-limiting examples of C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing non-limiting examples illustrate, in certain embodiments, the carbocyclyl group can be a monocyclic (“monocyclic carbocyclyl”) or polycyclic (e.g., containing a fused, bridged or spiro ring system such as a bicyclic system (“bicyclic carbocyclyl”), a tricyclic system (“tricyclic carbocyclyl”)), or a ring system including four or more rings, and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Non-limiting examples of C₅₋₆ cycloalkyl groups include cyclopentyl (C₅) and cyclohexyl (C₅). Non-limiting examples of C₃₋₆ cycloalkyl groups include the aforementioned C₅₋₆ cycloalkyl groups as well as cyclopropyl (C₃) and cyclobutyl (C₄). Non-limiting examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is an unsubstituted monocyclic C₃₋₁₄ cycloalkyl. In certain embodiments, the cycloalkyl group is a substituted C₃₋₁₄ cycloalkyl. In certain embodiments, the cycloalkyl group is a substituted monocyclic C₃₋₁₄ cycloalkyl.

“Carbocyclylalkyl” is a subset of “alkyl” and refers to an alkyl group substituted by an carbocyclyl group, wherein the point of attachment is on the alkyl moiety.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, sulfur, boron, and phosphorus (“3-14 membered heterocyclyl”). In certain embodiments, each heteroatom is independently selected from nitrogen, oxygen, and sulfur. In heterocyclyl groups that contain one or more nitrogen, sulfur, boron, or phosphorus atoms, the point of attachment can be a carbon, nitrogen, sulfur, boron, or phosphorus atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”), a tricyclic system (“tricyclic heterocyclyl”), or a ring system), or a ring system including four or more rings, and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, and phosphorus (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, sulfur, boron, and phosphorus (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, sulfur, boron, and phosphorus (“5-6 membered heterocyclyl”). In some embodiments, the 5-6 membered heterocyclyl has 1-3 ring heteroatoms selected from nitrogen, oxygen, sulfur, boron, and phosphorus. In some embodiments, the 5-6 membered heterocyclyl has 1-2 ring heteroatoms selected from nitrogen, oxygen, sulfur, boron, and phosphorus. In some embodiments, the 5-6 membered heterocyclyl has 1 ring heteroatom selected from nitrogen, oxygen, sulfur, boron, and phosphorus. In certain embodiments, each heteroatom is independently selected from nitrogen, oxygen, and sulfur.

Non-limiting examples of 3-membered heterocyclyl groups containing 1 heteroatom include azirdinyl, oxiranyl, and thiiranyl. Non-limiting examples of 4-membered heterocyclyl groups containing 1 heteroatom include azetidinyl, oxetanyl, and thietanyl. Non-limiting examples of 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Non-limiting examples of 5-membered heterocyclyl groups containing 2 heteroatoms include dioxolanyl, oxathiolanyl and dithiolanyl. Non-limiting examples of 5-membered heterocyclyl groups containing 3 heteroatoms include triazolinyl, oxadiazolinyl, and thiadiazolinyl. Non-limiting examples of 6-membered heterocyclyl groups containing 1 heteroatom include piperidinyl, tetrahydropyranyl, dihydropyridinyl, and thianyl. Non-limiting examples of 6-membered heterocyclyl groups containing 2 heteroatoms include piperazinyl, morpholinyl, dithianyl, and dioxanyl. Non-limiting examples of 6-membered heterocyclyl groups containing 3 heteroatoms include triazinyl. Non-limiting examples of 7-membered heterocyclyl groups containing 1 heteroatom include azepanyl, oxepanyl and thiepanyl. Non-limiting examples of 8-membered heterocyclyl groups containing 1 heteroatom include azocanyl, oxecanyl and thiocanyl. Non-limiting examples of bicyclic heterocyclyl groups include indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

“Heterocyclylalkyl” is a subset of “alkyl” and refers to an alkyl group substituted by an heterocyclyl group, wherein the point of attachment is on the alkyl moiety.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Arylalkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Non-limiting examples of 5-membered heteroaryl groups containing 1 heteroatom include pyrrolyl, furanyl, and thiophenyl. Non-limiting examples of 5-membered heteroaryl groups containing 2 heteroatoms include imidazolyl, pyrazolyl, oxazolyl, isoxazolyl, thiazolyl, and isothiazolyl. Non-limiting examples of 5-membered heteroaryl groups containing 3 heteroatoms include triazolyl, oxadiazolyl, and thiadiazolyl. Non-limiting examples of 5-membered heteroaryl groups containing 4 heteroatoms include tetrazolyl. Non-limiting examples of 6-membered heteroaryl groups containing 1 heteroatom include pyridinyl. Non-limiting examples of 6-membered heteroaryl groups containing 2 heteroatoms include pyridazinyl, pyrimidinyl, and pyrazinyl. Non-limiting examples of 6-membered heteroaryl groups containing 3 or 4 heteroatoms include triazinyl and tetrazinyl, respectively. Non-limiting examples of 7-membered heteroaryl groups containing 1 heteroatom include azepinyl, oxepinyl, and thiepinyl. Non-limiting examples of 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Non-limiting examples of 6,6-bicyclic heteroaryl groups include naphthyridinyl, pteridinyl, quinolinyl, isoquinolinyl, cinnolinyl, quinoxalinyl, phthalazinyl, and quinazolinyl. Non-limiting examples of tricyclic heteroaryl groups include phenanthridinyl, dibenzofuranyl, carbazolyl, acridinyl, phenothiazinyl, phenoxazinyl, and phenazinyl.

“Heteroaralkyl” is a subset of “alkyl” and refers to an alkyl group substituted by a heteroaryl group, wherein the point of attachment is on the alkyl moiety.

The term “unsaturated bond” refers to a double or triple bond. The term “unsaturated” or “partially unsaturated” refers to a moiety that includes at least one double or triple bond. The term “saturated” refers to a moiety that does not contain a double or triple bond, i.e., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety. The present disclosure is not intended to be limited in any manner by the exemplary substituents described herein.

Non-limiting exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, N(OR^{cc})R^{bb}, —SH, —SR^{aa}, —SSR^{cc}, —C(═O)R^{aa}, —CO₂H, —CHO, —C(OR^{cc})₃, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;

or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};

each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —OR^{aa}, —N(R^{cc})₂, —CN, —C(═O)R^{aa}, C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;

each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂ OR^{ee}, —OSO₂R^{ee}, S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;

each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;

each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, 3-10 membered heterocyclyl, C₆₋₁₀ aryl and 5-10 membered heteroaryl, or two R groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and

each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(═NH)NH(C₁₋₆ alkyl), —OC(═NH)NH₂, —NHC(═NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂(C₁₋₆ alkyl), —SO₂O(C₁₋₆ alkyl), —OSO₂(C₁₋₆ alkyl), —SO(C₁₋₆ alkyl), —Si(C₁₋₆ alkyl)₃, —OSi(C₁₋₆ alkyl)₃—C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb})₂)₂, wherein X⁻, R^{aa}, R^{bb}, and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R_{bb})₂, —NR^{bb}C(═O)R^{aa}, —NR^{bb}CO₂R^{aa}, —R^{bb}C(═O)N(R^{bb})₂, —R^{bb}C(—NR^{bb})N(R_{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “sulfonyl” refers to a group selected from —SO₂N(R^{bb})₂, —SO₂R^{aa}, and —SO₂OR^{aa}, wherein R^{aa} and R^{bb} are as defined herein.

The term “sulfinyl” refers to the group —S(═O)R^{aa}, wherein R^{aa} is as defined herein.

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, —C(═S)O(R^{X1}), —C(═S)S(R^{X1}), —C(—NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Non-limiting examples of acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, acylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “carbonyl” refers to a group wherein the carbon directly attached to the parent molecule is sp² hybridized, and is substituted with an oxygen, nitrogen or sulfur atom, e.g., a group selected from ketones (e.g., —C(═O)R^{aa}), carboxylic acids (e.g., —CO₂H), aldehydes (—CHO), esters (e.g., —CO₂R^{aa}, —C(═O)SR^{aa}, —C(═S)SR^{aa}), amides (e.g., —C(═O)N(R^{bb})₂, —C(═O)NR^{bb}SO₂R^{aa}, —C(═S)N(R^{bb})₂), and imines (e.g., —C(═NR^{bb})R^{aa}, —C(═NR^{bb})OR^{aa}), —C(═NR^{bb})N(R^{bb})₂), wherein R^{aa} and R^{bb} are as defined herein. In certain embodiments, “carbonyl” refers to a group wherein the carbon directly attached to the parent molecule is sp² hybridized, and is substituted with an oxygen atom.

The term “silyl” refers to the group —Si(R^{aa})₃, wherein R^{aa} is as defined herein.

The term “oxo” refers to the group ═O, and the term “thiooxo” refers to the group ═S.

Nitrogen atoms can be substituted or unsubstituted as valency permits, and include primary, secondary, tertiary, and quaternary nitrogen atoms. Non-limiting examples of nitrogen atom substituents include hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{aa}, —SO₂OR^{aa}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on the nitrogen atom is a nitrogen protecting group (also referred to herein as an “amino protecting group”). Non-limiting examples of nitrogen protecting groups include —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, the contents of which are incorporated herein by reference in their entirety.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, and o-(benzoyloxymethyl)benzamide.

Nitrogen protecting groups such as carbamate groups (e.g., —C(═O)OR^{aa}) include, but are not limited to, methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), (3-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-12-(trimethylsilyl)ethoxylmethylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In certain embodiments, a nitrogen protecting group is benzyl (Bn), tert-butyloxycarbonyl (BOC), carbobenzyloxy (Cbz), 9-flurenylmethyloxycarbonyl (Fmoc), trifluoroacetyl, triphenylmethyl, acetyl (Ac), benzoyl (Bz), p-methoxybenzyl (PMB), 3,4-dimethoxybenzyl (DMPM), p-methoxyphenyl (PMP), 2,2,2-trichloroethyloxycarbonyl (Troc), triphenylmethyl (Tr), tosyl (Ts), brosyl (Bs), nosyl (Ns), mesyl (Ms), triflyl (TO, or dansyl (Ds).

In certain embodiments, the substituent present on an oxygen atom is an oxygen protecting group (also referred to herein as an “hydroxyl protecting group”). Non-limiting exemplary oxygen protecting groups include —R^{aa}, —N(R^{bb})₂, —C(═O)SR^{aa}, —C(═O)R^{aa}, —CO₂R^{aa}, —C(═O)N(R^{bb})₂, —C(—NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3rd edition, John Wiley & Sons, 1999, the contents of which are incorporated herein by reference in their entirety.

Non-limiting exemplary oxygen protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxy acetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis (1,1-dimethylpropyl)phenoxyacetate, chlorodiphenylacetate, isobutyrate, monosuccinoate, (E)-2-methyl-2-butenoate, o-(methoxyacyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). In certain embodiments, an oxygen protecting group is silyl. In certain embodiments, an oxygen protecting group is t-butyldiphenylsilyl (TBDPS), t-butyldimethylsilyl (TBDMS), triisoproylsilyl (TIPS), triphenylsilyl (TPS), triethylsilyl (TES), trimethylsilyl (TMS), triisopropylsiloxymethyl (TOM), acetyl (Ac), benzoyl (Bz), allyl carbonate, 2,2,2-trichloroethyl carbonate (Troc), 2-trimethylsilylethyl carbonate, methoxymethyl (MOM), 1-ethoxyethyl (EE), 2-methyoxy-2-propyl (MOP), 2,2,2-trichloroethoxyethyl, 2-methoxyethoxymethyl (MEM), 2-trimethylsilylethoxymethyl (SEM), methylthiomethyl (MTM), tetrahydropyranyl (THP), tetrahydrofuranyl (THF), p-methoxyphenyl (PMP), triphenylmethyl (Tr), methoxytrityl (MMT), dimethoxytrityl (DMT), allyl, p-methoxybenzyl (PMB), t-butyl, benzyl (Bn), allyl, or pivaloyl (Piv).

In certain embodiments, the substituent present on a sulfur atom is a sulfur protecting group (also referred to as a “thiol protecting group”). Non-limiting exemplary sulfur protecting groups include —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3rd edition, John Wiley & Sons, 1999, the contents of which are incorporated herein by reference in their entirety. In certain embodiments, a sulfur protecting group is acetamidomethyl, t-Bu, 3-nitro-2-pyridine sulfenyl, 2-pyridine-sulfenyl, or triphenylmethyl.

An “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Non-limiting exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me5Br6)−). Non-limiting exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

As used herein, use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

A “non-hydrogen group” refers to any group that is defined for a particular variable that is not hydrogen.

These and other non-limiting exemplary substituents are described in more detail in the Detailed Description, Examples, and Claims. The present disclosure is not intended to be limited in any manner by the above exemplary listing of substituents.

### Other Definitions

The following definitions are more general terms used throughout the present application.

As used herein, the term “salt” refers to any and all salts, and encompasses pharmaceutically acceptable salts.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, the contents of which are incorporated herein by reference in their entirety. Pharmaceutically acceptable salts of the compounds of this disclosure include those derived from suitable inorganic and organic acids and bases. Non-limiting examples of pharmaceutically acceptable, nontoxic acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid, or by using other methods known in the art such as ion exchange. Other non-limiting exemplary pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Non-limiting representative alkali or alkaline earth metal salts include sodium, lithium, potassium, calcium, magnesium, and the like. Non-limiting examples of further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound or a salt thereof that are associated with a solvent, usually by a solvolysis reaction. This physical association may include hydrogen bonding. Non-limiting examples of conventional solvents capable of forming solvates include water (i.e., hydrates), methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R.x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R.0.5 H₂O)), and polyhydrates (x is a number greater than 1, e.g., dihydrates (R.2 H₂O) and hexahydrates (R.6 H₂O)).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Non-limiting exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it includes a carbon or phosphorus atom bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “polymorph” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “prodrugs” refers to compounds that have cleavable groups and become by solvolysis or under physiological conditions the compounds described herein, which are pharmaceutically active in vivo. Such examples include, but are not limited to, choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, e.g., Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. In certain embodiments, the prodrug is a C₁-C₈ alkyl, C₂-C₈ alkenyl, C₂-C₈ alkynyl, aryl, C₇-C₁₂ substituted aryl, or C₇-C₁₂ arylalkyl esters.

The terms “composition” and “formulation” are used interchangeably herein.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other non-limiting examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, preventing, minimizing one or more symptoms, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence. In certain embodiments, the treatment is reversing the progress of a disease described herein. In certain embodiments, the treatment is alleviating one or more symptoms associated with a disease described herein. In certain embodiments, the treatment is delaying the onset of a disease described herein. In certain embodiments, the treatment is inhibiting the progress of a disease described herein. In certain embodiments, the treatment is preventing the incidence of a disease described herein. In certain embodiments, the treatment is minimizing one or more symptoms associated with a disease described herein.

The terms “condition,” “disease,” and “disorder” are used interchangeably herein.

An “effective amount” of a compound described herein refers to an amount sufficient to elicit the desired biological response. An effective amount of a compound described herein may vary depending on such factors as the desired biological endpoint, the pharmacokinetics of the compound, the condition being treated, the mode of administration, and the age and health of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent.

A “prophylactically effective amount” of a compound described herein is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

As used herein, “inhibition”, “inhibiting”, “inhibit” and “inhibitor”, and the like, refer to the ability of a compound to reduce, slow, halt, or prevent the activity of a biological process (e.g., a biological process in a cell). In some embodiments, the term refers to a reduction of the level of enzyme activity (e.g., KDM activity, e.g., KDM3 activity), to a level that is statistically significantly lower than an initial level, which may, for example, be a baseline level of enzyme activity. In certain embodiments, such inhibition is of about 1% to 99.9%. In certain embodiments, the inhibition is about 1% to about 95%. In certain embodiments, the inhibition is about 5% to 90%. In certain embodiments, the inhibition is about 10% to 85%. In certain embodiments, the inhibition is about 15% to 80%. In certain embodiments, the inhibition is about 20% to 75%. In certain embodiments, the inhibition is about 25% to 70%. In certain embodiments, the inhibition is about 30% to 65%. In certain embodiments, the inhibition is about 35% to 60%. In certain embodiments, the inhibition is about 40% to 55%. In certain embodiments, the inhibition is about 45% to 50%. In certain embodiments, the inhibition is about 5%, 10%, 15%, 20%, 25%, 30%, 35%, 40%, 45%, 50%, 55%, 60%, 65%, 70%, 75%, 80%, 85%, 90%, 95%, or 99.9%.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Non-limiting exemplary proliferative diseases include cancers (i.e., “malignant neoplasms”), benign neoplasms, angiogenesis, inflammatory diseases, and autoimmune diseases.

The term “angiogenesis” refers to the physiological process through which new blood vessels form from pre-existing vessels. Angiogenesis is distinct from vasculogenesis, which is the de novo formation of endothelial cells from mesoderm cell precursors. The first vessels in a developing embryo form through vasculogenesis, after which angiogenesis is responsible for most blood vessel growth during normal or abnormal development. Angiogenesis is a vital process in growth and development, as well as in wound healing and in the formation of granulation tissue. However, angiogenesis is also a fundamental step in the transition of tumors from a benign state to a malignant one, leading to the use of angiogenesis inhibitors in the treatment of cancer. Angiogenesis may be chemically stimulated by angiogenic proteins, such as growth factors (e.g., VEGF). “Pathological angiogenesis” refers to abnormal (e.g., excessive or insufficient) angiogenesis that amounts to and/or is associated with a disease.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Non-limiting exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a class of diseases characterized by the development of abnormal cells that proliferate uncontrollably and have the ability to infiltrate and destroy normal body tissues. See, e.g., Stedman's Medical Dictionary, 25th ed.; Hensyl ed.; Williams & Wilkins: Philadelphia, 1990. Non-limiting exemplary cancers include acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GU-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

The term “cardiovascular disease” or “heart disease” refers to diseases associated the heart and/or blood vessels. Non-limiting exemplary cardiovascular diseases include coronary heart disease, stroke or cerebrovascular disease, congenital heart defects, peripheral artery disease, heart disease associated with atherosclerosis, ischemic heart disease, hypertensive heart disease, rheumatic heart disease, cardiac arrhythmias, heart failure, congenital heart disease, inflammatory heart disease, cardiomyopathy, pericardial disease, and valvular heart disease. In certain embodiments, the cardiac disease is cardiac fibrosis.

The term “demethylase” refers to any enzyme that catalyzes the removal of methyl groups from a substrate (e.g., nucleic acids, proteins (e.g., histones), metabolites, natural products and intermediates thereto, and other compounds). A “histone demethylase” catalyzes the removal of a methyl group from a histone protein. For example, a “histone lysine demethylase,” or “KDM” catalyzes the removal of a methyl group from the N-methyl lysine residue of a histone protein. KDMs are categorized into two subfamilies: the flavin-dependent KDM1 subfamily, and the 2-oxoglutarate-(2OG) dependent Jumonji C (JmjC) subfamily (KDM2-7 subfamily). In certain embodiments, the histone demethylase is a KDM. The overexpression of certain KDMs (e.g., KDM's in the JmjC KDM2-7 subfamily) has been linked to cancer. Overexpression of JmjC KDMs has been observed in multiple types of cancer cells. Non-limiting examples of KDMs include KDM2/7, KDM3, KDM4, KDM5, and KDM6. Non-limiting examples of KDM5 include KDM5A, KDM5B, and KDM5C. Non-limiting examples of KDM3 include KDM3A, KDM3B, and Jumonji domain containing 1C (JMJD1C) JMJD1C.

### BRIEF DESCRIPTION OF THE DRAWINGS

The accompanying drawings, which constitute a part of this specification, illustrate several embodiments of the present disclosure and together with the description, serve to explain the principles of the present disclosure.

FIG. 1 shows a Western Blot, produced from testing histone methylation activity of JADA94 (a mixture of JADA94-1 and JADA94-2) in RPMI8226 cell line after 24 hours, demonstrating selectivity of JADA94 for KDM3.

FIG. 2A shows MTT cell growth inhibition assay results of JADA94 at 72 hours in H929, RPMI8226, MM.1S, and OPM2 cells. FIG. 2B shows MTT cell growth inhibition assay results of JADA94 at 6 days in H929, RPMI8226, and MM.1S cells.

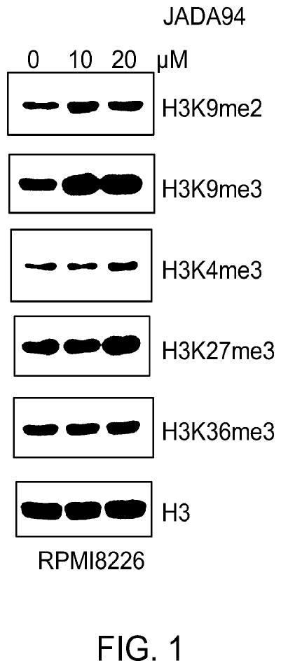

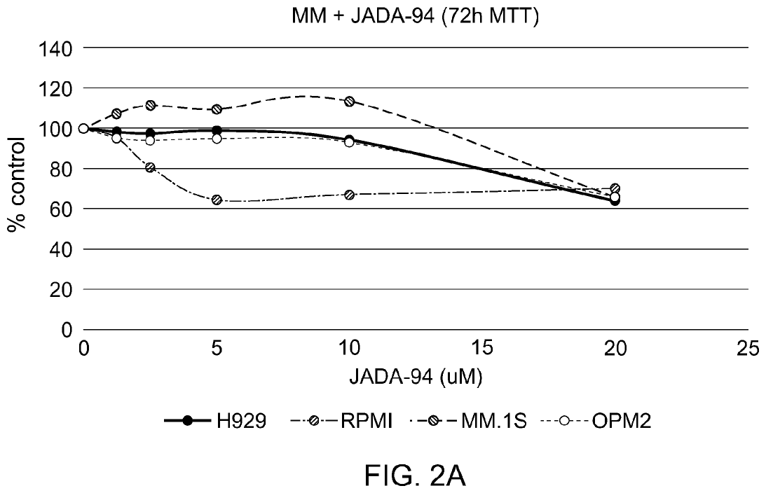

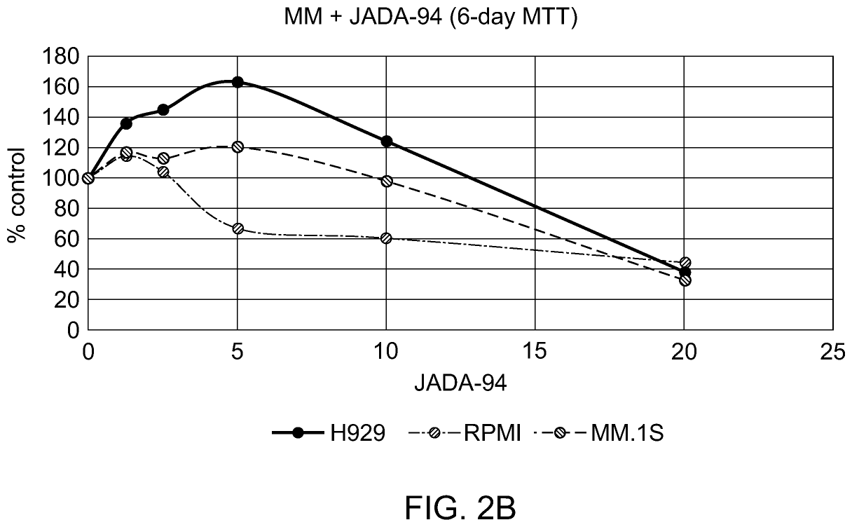

### DETAILED DESCRIPTION OF CERTAIN EMBODIMENTS

Provided herein compounds of Formulas (I), (II), compounds 1-22 and pharmaceutically acceptable salts, hydrates, solvates, polymorphs, co-crystals, tautomers, stereoisomers, isotopically labeled derivatives, and prodrugs thereof, and pharmaceutical compositions thereof. The inventive compounds provided herein are histone lysine demethylase (KDM) inhibitors (e.g., KDM3 inhibitors), and are therefore useful for the treatment and/or prevention of diseases (e.g., proliferative diseases, e.g., cancer). In certain embodiments, the compounds described herein are selective inhibitors of a KDM (e.g., KDM3). Therefore, also provided herein are methods and uses related to treating diseases or conditions (e.g., proliferative diseases (e.g., cancer), cardiovascular diseases) in a subject, inhibiting the activity of a KDM (e.g., KDM3) in a subject or biological sample; inducing apoptosis in the cell of a subject or biological sample, and/or modulating the expression of a gene.

### Compounds of Formula (I)

Provided herein are compounds of Formula (I):

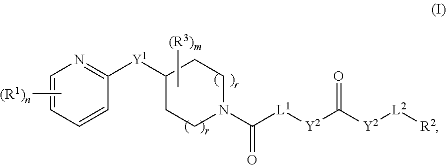

and pharmaceutically acceptable salts, hydrates, solvates, polymorphs, co-crystals, tautomers, stereoisomers, isotopically labeled derivatives, and prodrugs thereof, wherein:

each instance of R¹ is independently hydrogen, halogen, —CN, —N₃, —NO₂, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, optionally substituted sulfonyl, optionally substituted sulfinyl, —OR^{O}, —N(R^{N})₂, or —SR^{S};

n is 0, or an integer from 1 to 4, inclusive;

Y¹ is —C(R^{C})₂—, —O—, —NR^{N}—, or —S—;

each instance of R³ is independently hydrogen, halogen, —CN, —N₃, —NO₂, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, —OR^{O}, —N(R^{N})₂, or —SR^{S};

m is 0, or an integer from 1 to 5, inclusive;

each instance of r is independently 0 or 1;

L¹ is optionally substituted C₁₋₆ alkylene, optionally substituted C₂₋₆ alkenylene, optionally substituted carbocyclyl, optionally substituted heterocyclyl, optionally substituted arylene, or optionally substituted heteroarylene;

each instance of Y² is independently a bond, —O—, or —NR^{N}—;

L² is a bond, optionally substituted C₁₋₆ alkylene, optionally substituted C₁₋₆ heteroalkylene, optionally substituted C₂₋₆ alkenylene, optionally substituted C₂₋₆ alkynylene, optionally substituted C₁₋₆ acylene, or a combination thereof;

R² is —N(R^{N})₂, optionally substituted aryl, optionally substituted heteroaryl, optionally substituted carbocyclyl, or optionally substituted heterocyclyl;

each instance of R^{N} is independently hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, optionally substituted sulfonyl, optionally substituted sulfinyl, or a nitrogen protecting group; or two R^{N} bonded to the same nitrogen atom are taken together with the intervening atoms to form optionally substituted heterocyclyl or optionally substituted heteroaryl;

each instance of R^{C} is independently hydrogen, halogen, —CN, —N₃, —NO₂, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, optionally substituted sulfonyl, optionally substituted sulfinyl, —OR^{O}, —N(R^{N})₂, or —SR^{S}; or two R^{C} bonded to the same carbon are taken together with the intervening atoms to form optionally substituted carbocyclyl, optionally substituted heterocyclyl, or ═O;

each instance of R^{O} is independently hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, or an oxygen protecting group; and

each instance of R^{S} is independently hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, a sulfur protecting group, or R^{S}, taken together with the sulfur atom to which it is attached forms a disulfide group substituted with hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, or optionally substituted acyl, provided that

when L1 is optionally substituted C₁₋₂ alkylene, at least one instance of Y² is —NR^{N}—, R² is optionally substituted aryl or optionally substituted heteroaryl,

when L¹ is optionally substituted C₁₋₂ alkylene, at least one instance of Y² is —NR^{N}—, R² is not optionally substituted carbocyclyl,

when L¹ is optionally substituted C₁₋₄ alkylene, at least one instance of Y² is —NR^{N}—,

when L¹ is optionally substituted C₂ alkylene and at least one instance of Y² is —NR^{N}—, R² is optionally substituted aryl or optionally substituted heteroaryl,

when L¹ is optionally substituted C₂ alkylene and at least one instance of Y² is —NR^{N}—, R² is not optionally substituted carbocyclyl,

when R¹ is optionally substituted C₁ alkylene, at least one instance of Y² is —NR^{N}—,

when R¹ is optionally substituted alkylene, L¹ is optionally substituted C₂ alkylene, each instance of Y2 is a bond, and L2 is a bond, R² is not optionally substituted aryl,

where R¹ is halo, L¹ is optionally substituted C₂ alkylene, one of Y² is —NR^{N}— and the other is a bond, and L² is a bond, R² is not optionally substituted carbocyclyl,

when R¹ is halo, L¹ is optionally substituted C₂ alkylene, one of Y² is —NR^{N}— and the other is a bond, and L² is a bond, R² is not cyclopentyl,

and when L¹ and L² are optionally substituted C₁ alkylene, Y¹ is O, one of Y² is a bond and the other is O, R² is a phenyl, R¹ is not optionally substituted oxazolidinone.

In certain embodiments, the compound is a compound of Formula (I), or a pharmaceutically acceptable salt, hydrate, solvate, polymorph, co-crystal, tautomer, stereoisomer, isotopically labeled derivative, or prodrug thereof; provided that when L¹ is optionally substituted C₁₋₂ alkylene, then at least one instance of Y² is —NR^{N}—; and R² is optionally substituted aryl or optionally substituted heteroaryl. In certain embodiments, when L₁ is optionally substituted C₁₋₂ alkylene, then at least one instance of Y² is —NR^{N}—; and R² is not optionally substituted carbocyclyl. In certain embodiments, when L₁ is optionally substituted C₁₋₂ alkylene, then at least one instance of Y² is —NR^{N}—; and R² is not cyclopentyl.

In certain embodiments, when L¹ is optionally substituted C₁₋₄ alkylene, then at least one instance of Y² is —NR^{N}—. In certain embodiments, when L¹ is optionally substituted C₁ alkylene, then at least one instance of Y² is —NR^{N}—. In certain embodiments, when L₁ is unsubstituted C₄ alkylene, then at least one instance of Y² is —NR^{N}—. In certain embodiments, when L₁ is optionally substituted C₂ alkylene, then at least one instance of Y² is —NR^{N}—; and R² is optionally substituted aryl. In certain embodiments, when L₁ is optionally substituted C₂ alkylene, then at least one instance of Y² is —NR^{N}—; and R² is optionally substituted heteroaryl. In certain embodiments, when L¹ is optionally substituted C₂ alkylene, then at least one instance of Y² is —NR^{N}—; and R² is not optionally substituted carbocyclyl. In certain embodiments, when L¹ is optionally substituted C₂ alkylene, then at least one instance of Y² is —NR^{N}—; and R² is not cyclopentyl.

In certain embodiments, when R¹ is optionally substituted C₁ alkyl, then at least one instance of Y² is —NR^{N}—.

In certain embodiments, when R¹ is optionally substituted alkyl, L¹ is optionally substituted C₂ alkyl, each instance of Y2 is a bond, and L2 is a bond, then R² is not optionally substituted aryl.

In certain embodiments, where R¹ is halo, L₁ is optionally substituted C₂ alkyl, one of Y² is —NR^{N}—, and the other is a bond, and L² is a bond, then R² is not optionally substituted carbocyclyl. In certain embodiments, where R¹ is halo, L¹ is optionally substituted C₂ alkyl, one of Y² is —NR^{N}—, and the other is a bond, and L² is a bond, then R² is not cyclopentyl.

In certain embodiments, the compound is not any one of the following structures:

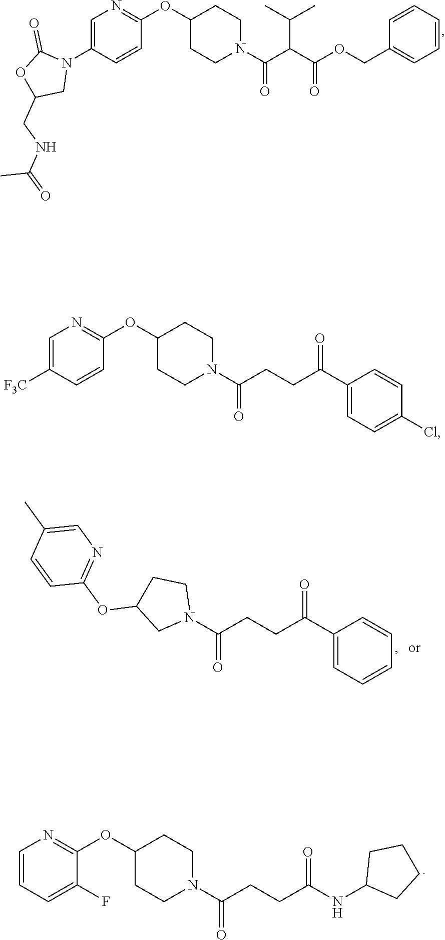 [SMILES: CC(=O)NCC1CN(c2ccc(OC3CCN(C(=O)C(C(=O)OCc4ccccc4)C(C)C)CC3)nc2)C(=O)O1 ; O=C(CCC(=O)N1CCC(Oc2ccc(C(F)(F)F)cn2)CC1)c1ccc(Cl)cc1 ; Cc1ccc(OC2CCN(C(=O)CCC(=O)c3ccccc3)C2)nc1 ; O=C(CCC(=O)N1CCC(Oc2ncccc2F)CC1)NC1CCCC1]

In certain embodiments, R¹ does not include an oxazolidinone.

In certain embodiments, the compound of Formula (I) is of Formula (I-i), (I-ii), or (I-iii):

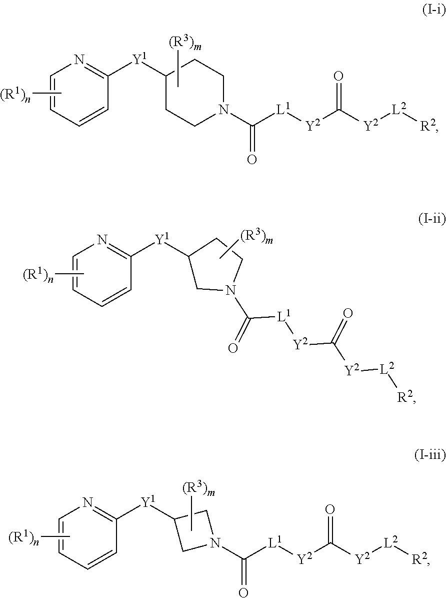

or a pharmaceutically acceptable salt, hydrate, solvate, polymorph, co-crystal, tautomer, stereoisomer, isotopically labeled derivative, or prodrug thereof.

In certain embodiments, the compound of Formula (I) is of Formula (I-a):

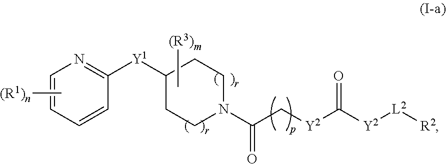

or a pharmaceutically acceptable salt, hydrate, solvate, polymorph, co-crystal, tautomer, stereoisomer, isotopically labeled derivative, or prodrug thereof; wherein:

p is an integer from 1 to 6, inclusive.

In certain embodiments, the compound of Formula (I) is of Formula (I-a-1) or (I-a-2):

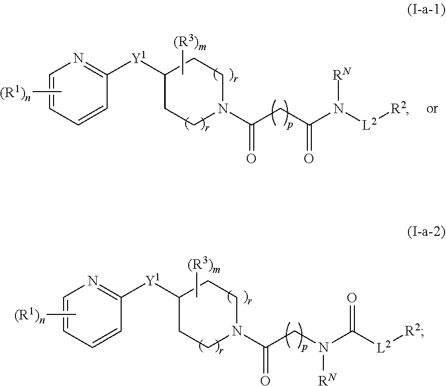

or a pharmaceutically acceptable salt, hydrate, solvate, polymorph, co-crystal, tautomer, stereoisomer, isotopically labeled derivative, or prodrug thereof.

In certain embodiments, the compound of Formula (I) is of Formula (I-a-3) or (I-a-4):

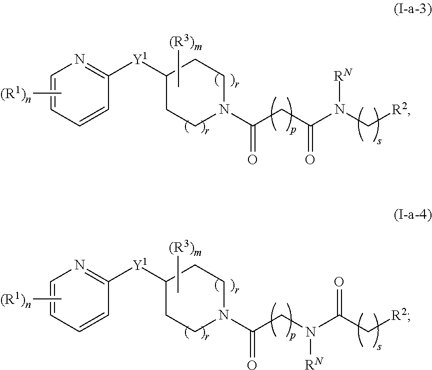

or a pharmaceutically acceptable salt, hydrate, solvate, polymorph, co-crystal, tautomer, stereoisomer, isotopically labeled derivative, or prodrug thereof; wherein:

s is an integer from 1 to 6, inclusive.

In certain embodiments, the compound of Formula (I) is of Formula (I-a-5):

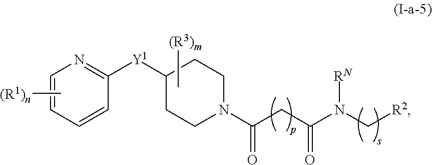

or a pharmaceutically acceptable salt, hydrate, solvate, polymorph, co-crystal, tautomer, stereoisomer, isotopically labeled derivative, or prodrug thereof.

In certain embodiments, the compound of Formula (I) is of Formula (I-b):

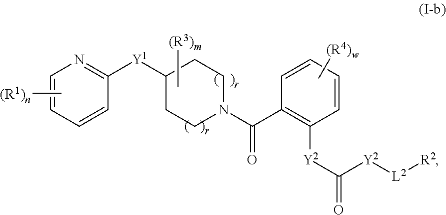

or a pharmaceutically acceptable salt, hydrate, solvate, polymorph, co-crystal, tautomer, stereoisomer, isotopically labeled derivative, or prodrug thereof; wherein:

each instance of R⁴ is independently hydrogen, halogen, —CN, —N₃, —NO₂, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, optionally substituted sulfonyl, optionally substituted sulfinyl, —OR^{O}, —N(R^{N})₂, or —SR^{S}; and

w is 0, or an integer from 1 to 4, inclusive.

In certain embodiments, the compound of Formula (I) is of Formula (I-b-1) or (I-b-2):

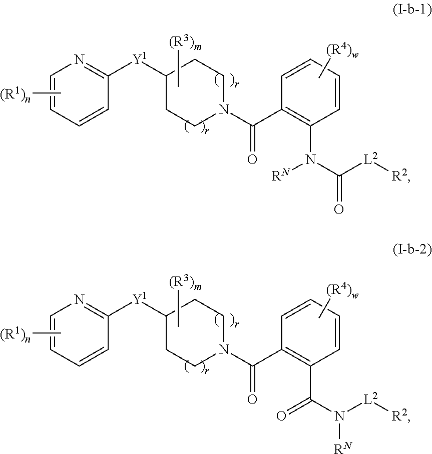

or a pharmaceutically acceptable salt, hydrate, solvate, polymorph, co-crystal, tautomer, stereoisomer, isotopically labeled derivative, or prodrug thereof.

In certain embodiments, the compound of Formula (I) is of Formula (I-b-3) or (I-b-4):

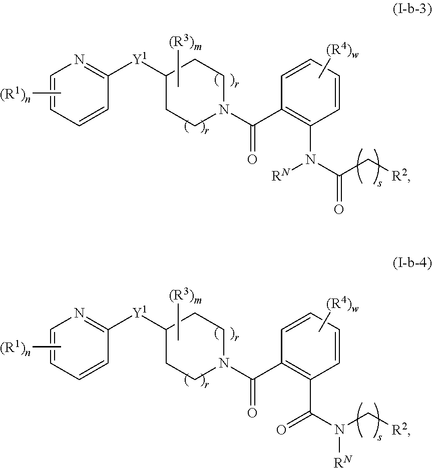

or a pharmaceutically acceptable salt, hydrate, solvate, polymorph, co-crystal, tautomer, stereoisomer, isotopically labeled derivative, or prodrug thereof; wherein:

s is an integer from 1 to 6, inclusive.

In certain embodiments, the compound of Formula (I) is of Formula (I-b-5) or (I-b-6):

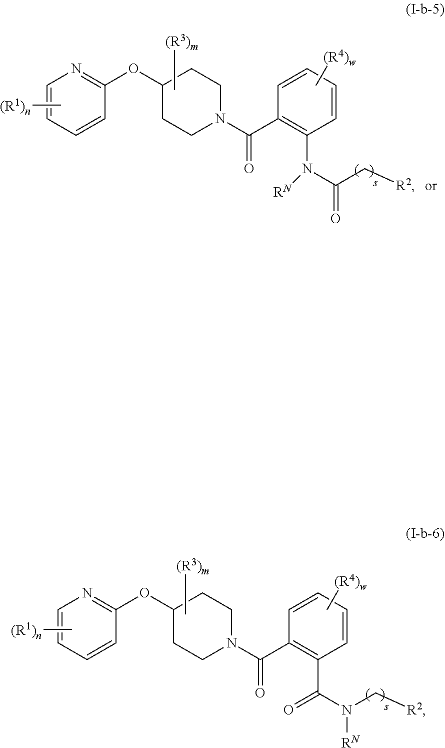

or a pharmaceutically acceptable salt, hydrate, solvate, polymorph, co-crystal, tautomer, stereoisomer, isotopically labeled derivative, or prodrug thereof.

In certain embodiments, the compound of Formula (I) is of Formula (I-c):

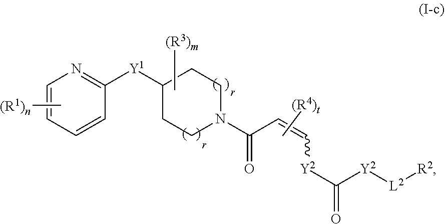

or a pharmaceutically acceptable salt, hydrate, solvate, polymorph, co-crystal, tautomer, stereoisomer, isotopically labeled derivative, or prodrug thereof; wherein:

each instance of R⁴ is independently hydrogen, halogen, —CN, —N₃, —NO₂, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, optionally substituted sulfonyl, optionally substituted sulfinyl, —OR^{O}, —N(R^{N})₂, or —SR^{S}; and

t is 0, or an integer from 1 to 2, inclusive.

In certain embodiments, the compound of Formula (I) is of Formula (I-c-1):

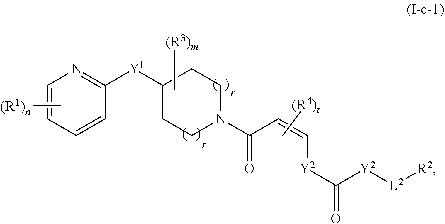

or a pharmaceutically acceptable salt, hydrate, solvate, polymorph, co-crystal, tautomer, stereoisomer, isotopically labeled derivative, or prodrug thereof.

In certain embodiments, the compound of Formula (I) is of Formula (I-c-2):

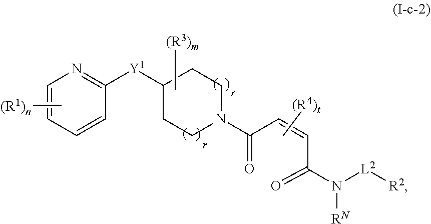

or a pharmaceutically acceptable salt, hydrate, solvate, polymorph, co-crystal, tautomer, stereoisomer, isotopically labeled derivative, or prodrug thereof.

In certain embodiments, the compound of Formula (I) is of Formula (I-c-3):

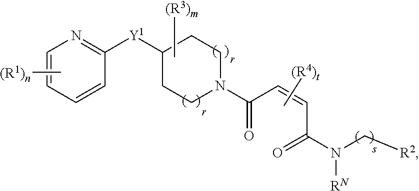

or a pharmaceutically acceptable salt, hydrate, solvate, polymorph, co-crystal, tautomer, stereoisomer, isotopically labeled derivative, or prodrug thereof; wherein:

s is an integer from 1 to 6, inclusive.

In certain embodiments, the compound of Formula (I) is of Formula (I-c-4):

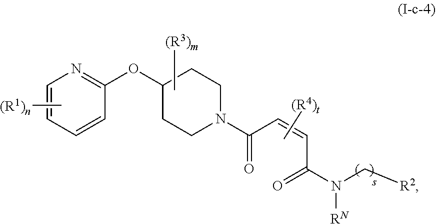

or a pharmaceutically acceptable salt, hydrate, solvate, polymorph, co-crystal, tautomer, stereoisomer, isotopically labeled derivative, or prodrug thereof.

In certain embodiments, the compound of Formula (I) is of Formula (I-c-5):

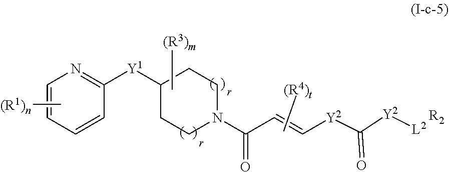

or a pharmaceutically acceptable salt, hydrate, solvate, polymorph, co-crystal, tautomer, stereoisomer, isotopically labeled derivative, or prodrug thereof.

In certain embodiments, the compound of Formula (I) is of Formula (I-c-6):

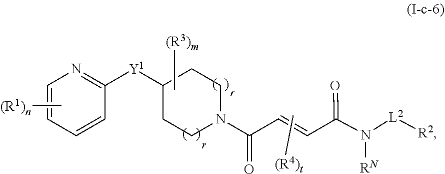

or a pharmaceutically acceptable salt, hydrate, solvate, polymorph, co-crystal, tautomer, stereoisomer, isotopically labeled derivative, or prodrug thereof.

In certain embodiments, the compound of Formula (I) is of Formula (I-c-7):

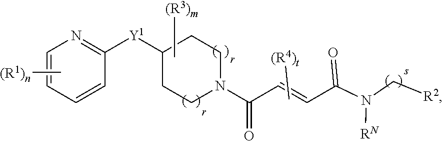

or a pharmaceutically acceptable salt, hydrate, solvate, polymorph, co-crystal, tautomer, stereoisomer, isotopically labeled derivative, or prodrug thereof; wherein:

s is an integer from 1 to 6, inclusive.

In certain embodiments, the compound of Formula (I) is of Formula (I-c-8):

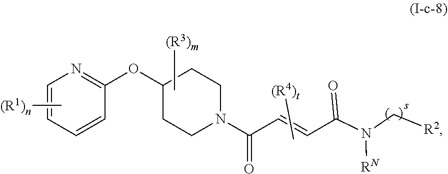

or a pharmaceutically acceptable salt, hydrate, solvate, polymorph, co-crystal, tautomer, stereoisomer, isotopically labeled derivative, or prodrug thereof.

In certain embodiments, the compound of Formula (I) is represented by any one of structures:

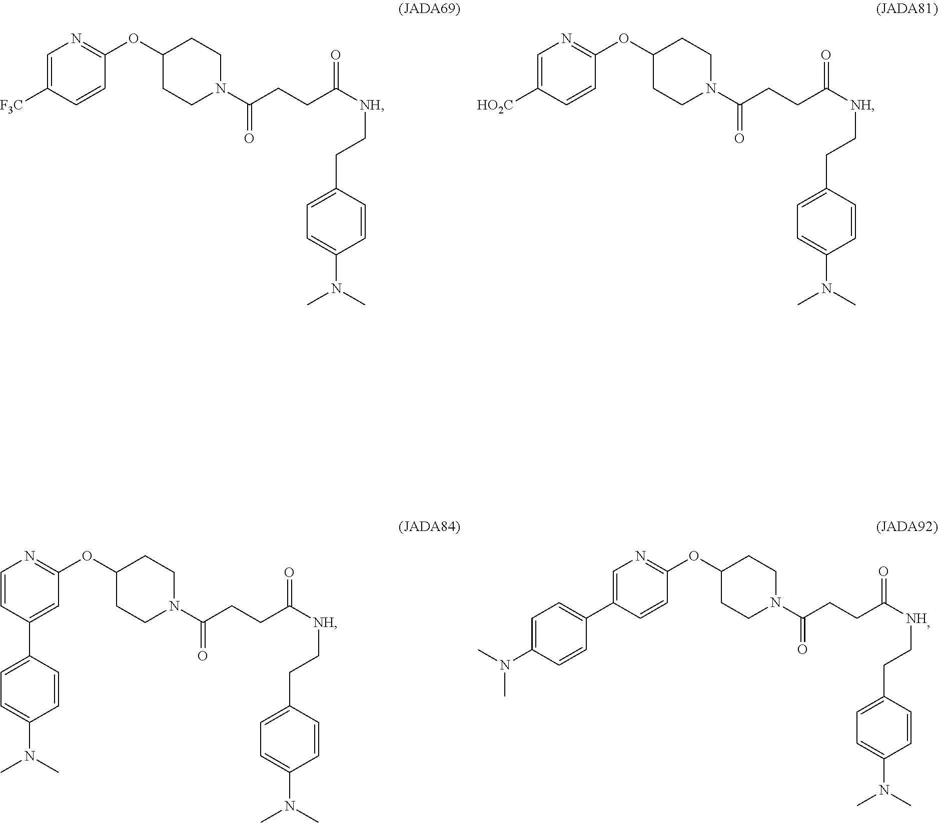

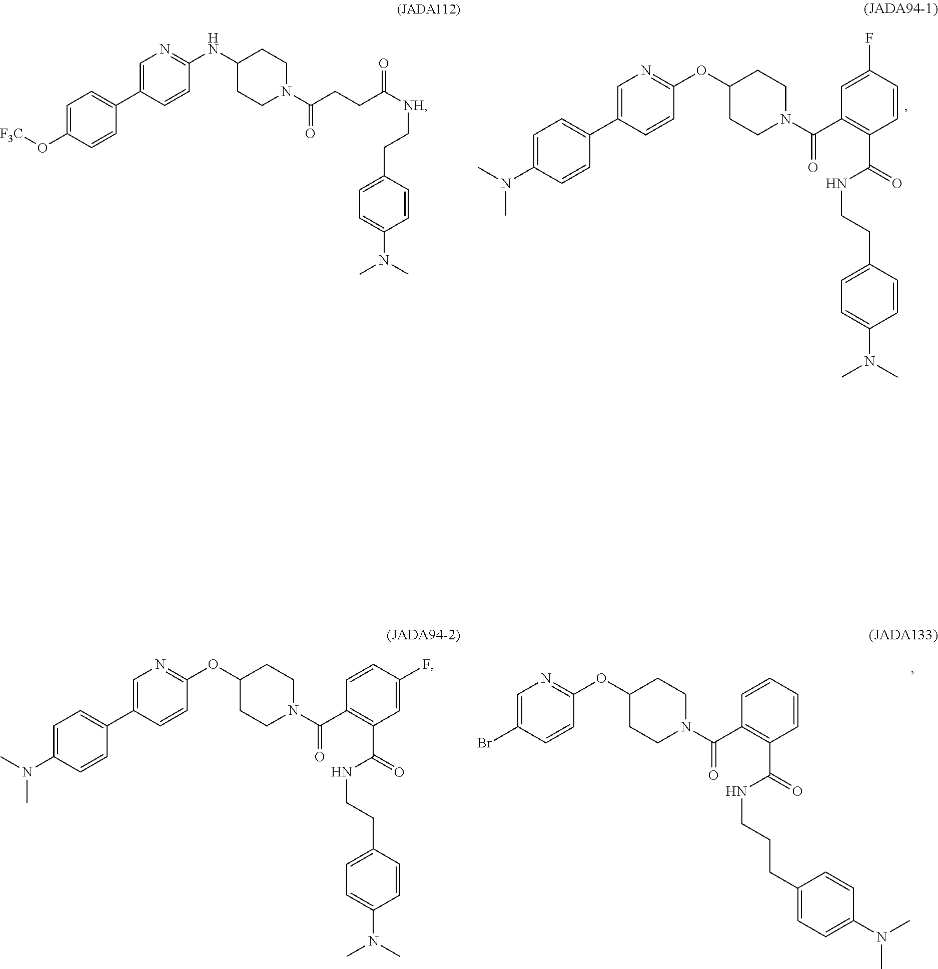 [SMILES: CN(C)c1ccc(CCNC(=O)CCC(=O)N2CCC(Nc3ccc(-c4ccc(OC(F)(F)F)cc4)cn3)CC2)cc1 ; CN(C)c1ccc(CCNC(=O)c2ccc(F)cc2C(=O)N2CCC(Oc3ccc(-c4ccc(N(C)C)cc4)cn3)CC2)cc1 ; CN(C)c1ccc(CCNC(=O)c2cc(F)ccc2C(=O)N2CCC(Oc3ccc(-c4ccc(N(C)C)cc4)cn3)CC2)cc1 ; CN(C)c1ccc(CCCNC(=O)c2ccccc2C(=O)N2CCC(Oc3ccc(Br)cn3)CC2)cc1]

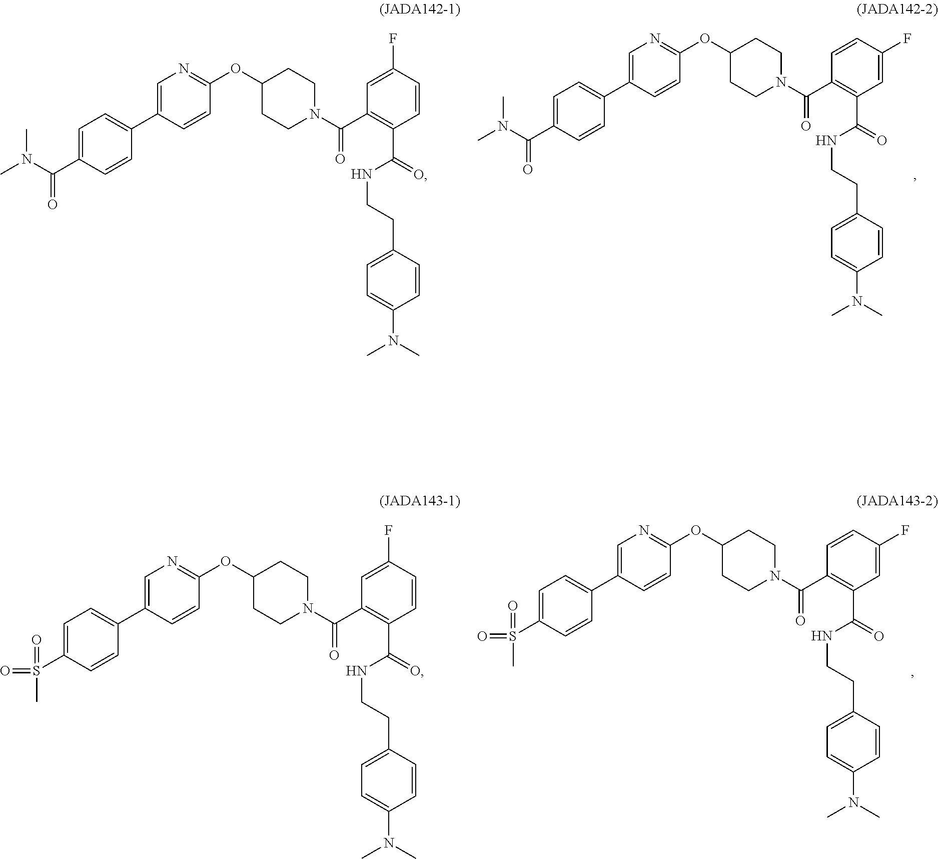 [SMILES: CN(C)C(=O)c1ccc(-c2ccc(OC3CCN(C(=O)c4cc(F)ccc4C(=O)NCCc4ccc(N(C)C)cc4)CC3)nc2)cc1 ; CN(C)C(=O)c1ccc(-c2ccc(OC3CCN(C(=O)c4ccc(F)cc4C(=O)NCCc4ccc(N(C)C)cc4)CC3)nc2)cc1 ; CN(C)c1ccc(CCNC(=O)c2ccc(F)cc2C(=O)N2CCC(Oc3ccc(-c4ccc(S(C)(=O)=O)cc4)cn3)CC2)cc1 ; CN(C)c1ccc(CCNC(=O)c2cc(F)ccc2C(=O)N2CCC(Oc3ccc(-c4ccc(S(C)(=O)=O)cc4)cn3)CC2)cc1]

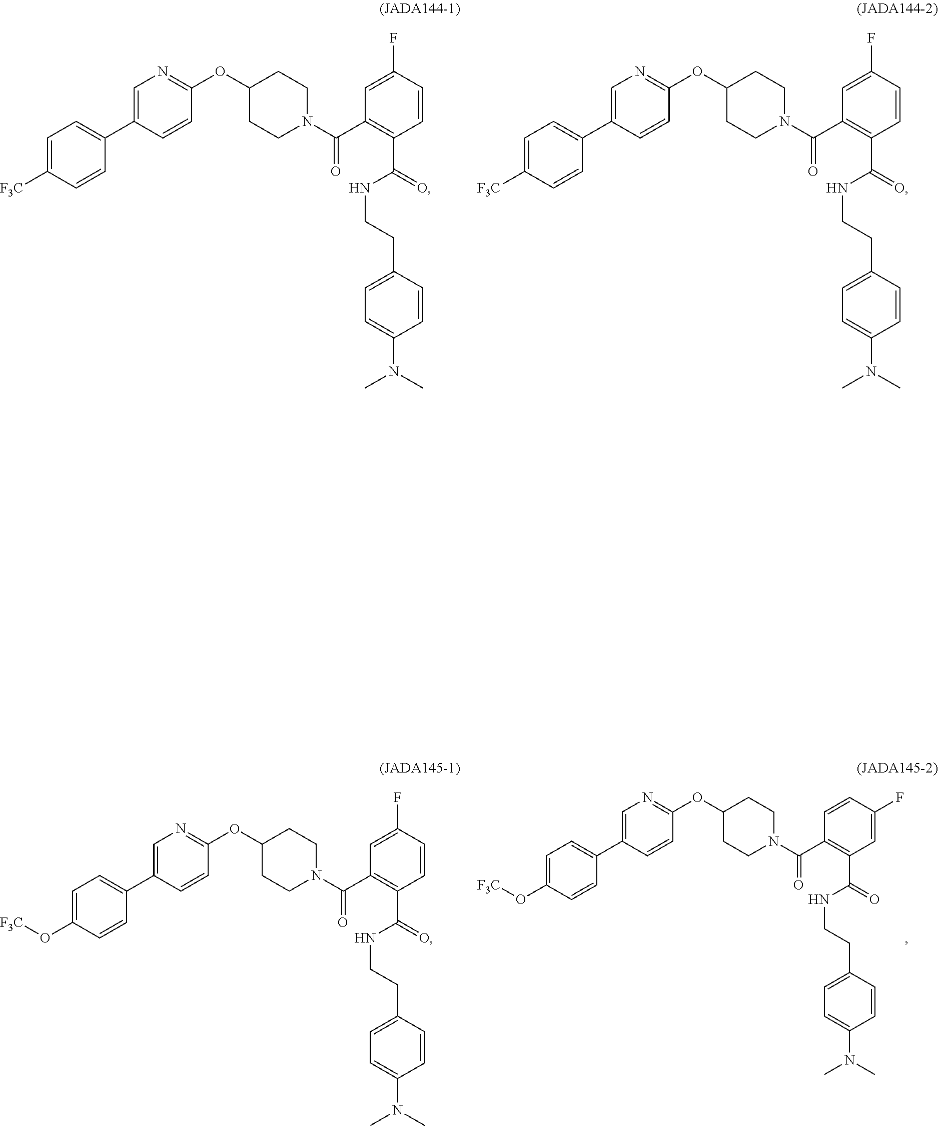 [SMILES: CN(C)c1ccc(CCNC(=O)c2ccc(F)cc2C(=O)N2CCC(Oc3ccc(-c4ccc(C(F)(F)F)cc4)cn3)CC2)cc1 ; CN(C)c1ccc(CCNC(=O)c2ccc(F)cc2C(=O)N2CCC(Oc3ccc(-c4ccc(C(F)(F)F)cc4)cn3)CC2)cc1 ; CN(C)c1ccc(CCNC(=O)c2ccc(F)cc2C(=O)N2CCC(Oc3ccc(-c4ccc(OC(F)(F)F)cc4)cn3)CC2)cc1 ; CN(C)c1ccc(CCNC(=O)c2cc(F)ccc2C(=O)N2CCC(Oc3ccc(-c4ccc(OC(F)(F)F)cc4)cn3)CC2)cc1]

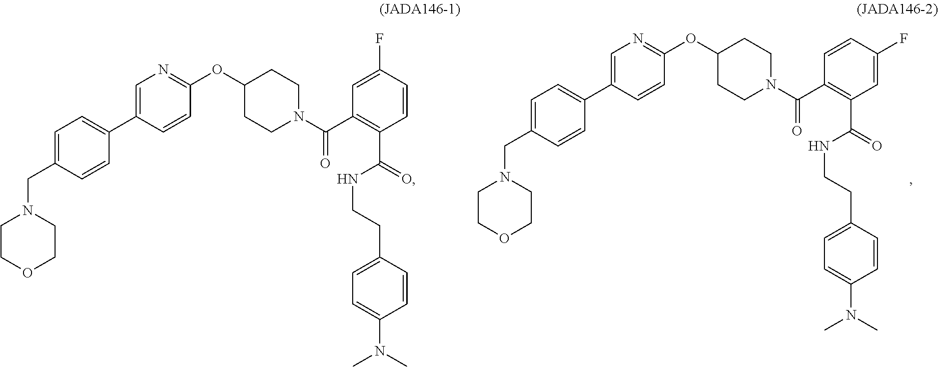 [SMILES: CN(C)c1ccc(CCNC(=O)c2ccc(F)cc2C(=O)N2CCC(Oc3ccc(-c4ccc(CN5CCOCC5)cc4)cn3)CC2)cc1 ; CN(C)c1ccc(CCNC(=O)c2cc(F)ccc2C(=O)N2CCC(Oc3ccc(-c4ccc(CN5CCOCC5)cc4)cn3)CC2)cc1]

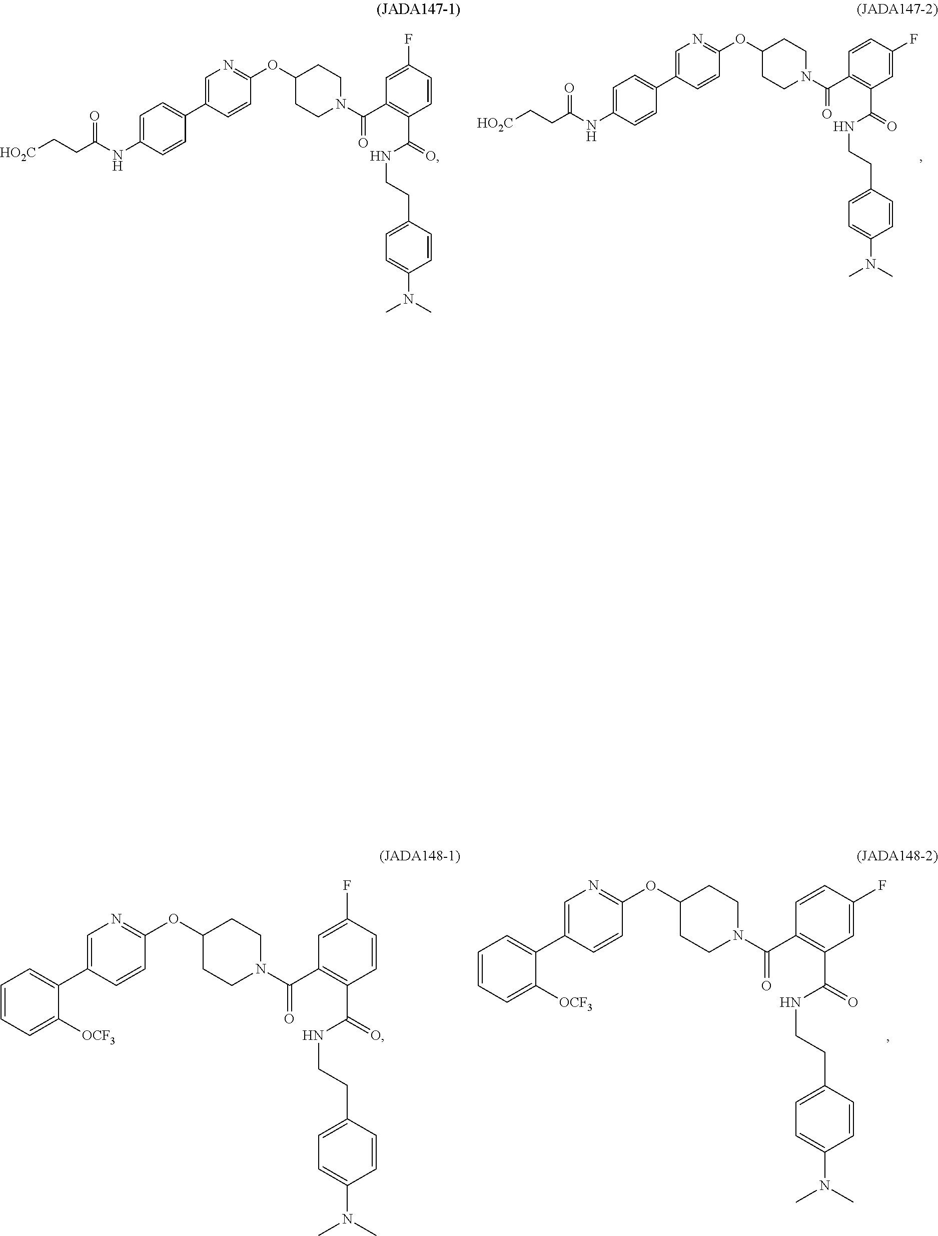 [SMILES: CN(C)c1ccc(CCNC(=O)c2ccc(F)cc2C(=O)N2CCC(Oc3ccc(-c4ccc(NC(=O)CCC(=O)O)cc4)cn3)CC2)cc1 ; CN(C)c1ccc(CCNC(=O)c2cc(F)ccc2C(=O)N2CCC(Oc3ccc(-c4ccc(NC(=O)CCC(=O)O)cc4)cn3)CC2)cc1 ; CN(C)c1ccc(CCNC(=O)c2ccc(F)cc2C(=O)N2CCC(Oc3ccc(-c4ccccc4OC(F)(F)F)cn3)CC2)cc1 ; CN(C)c1ccc(CCNC(=O)c2cc(F)ccc2C(=O)N2CCC(Oc3ccc(-c4ccccc4OC(F)(F)F)cn3)CC2)cc1]

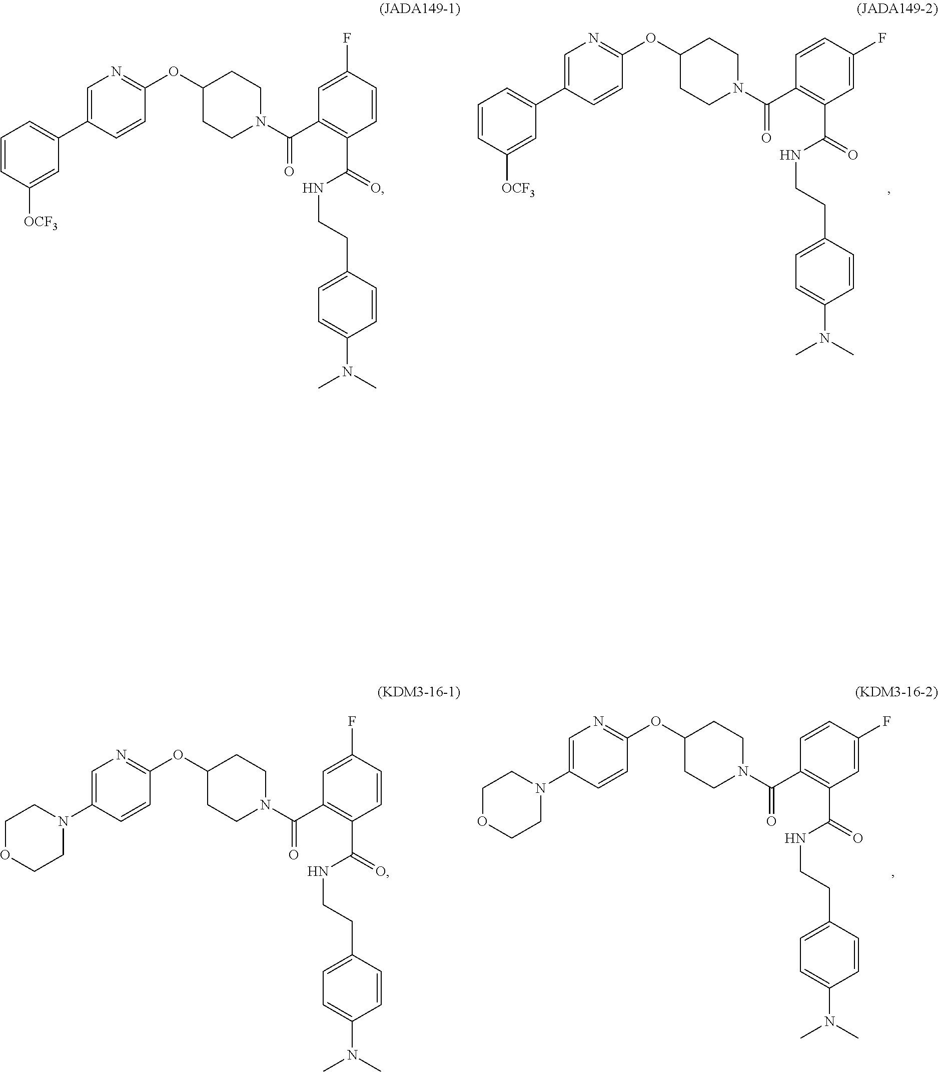 [SMILES: CN(C)c1ccc(CCNC(=O)c2ccc(F)cc2C(=O)N2CCC(Oc3ccc(-c4cccc(OC(F)(F)F)c4)cn3)CC2)cc1 ; CN(C)c1ccc(CCNC(=O)c2cc(F)ccc2C(=O)N2CCC(Oc3ccc(-c4cccc(OC(F)(F)F)c4)cn3)CC2)cc1 ; CN(C)c1ccc(CCNC(=O)c2ccc(F)cc2C(=O)N2CCC(Oc3ccc(N4CCOCC4)cn3)CC2)cc1 ; CN(C)c1ccc(CCNC(=O)c2cc(F)ccc2C(=O)N2CCC(Oc3ccc(N4CCOCC4)cn3)CC2)cc1]

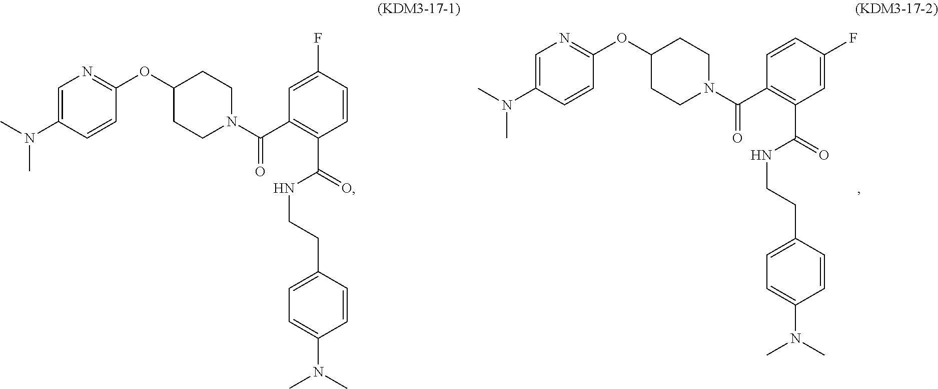

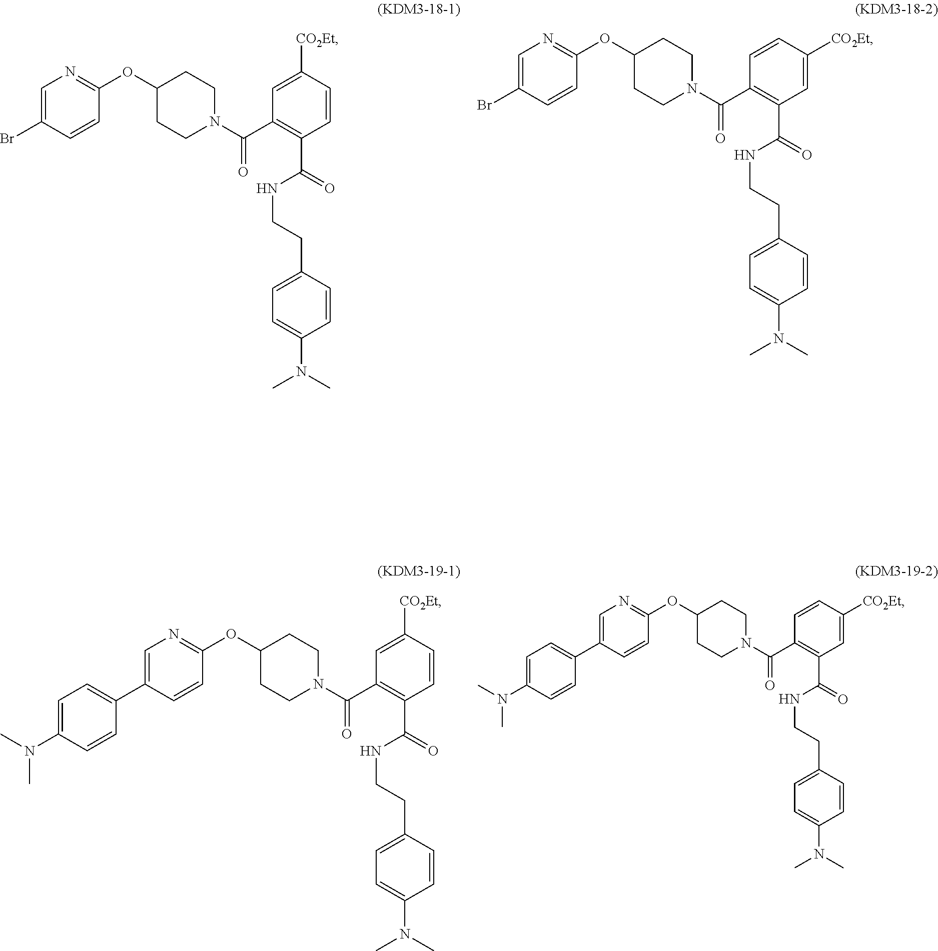

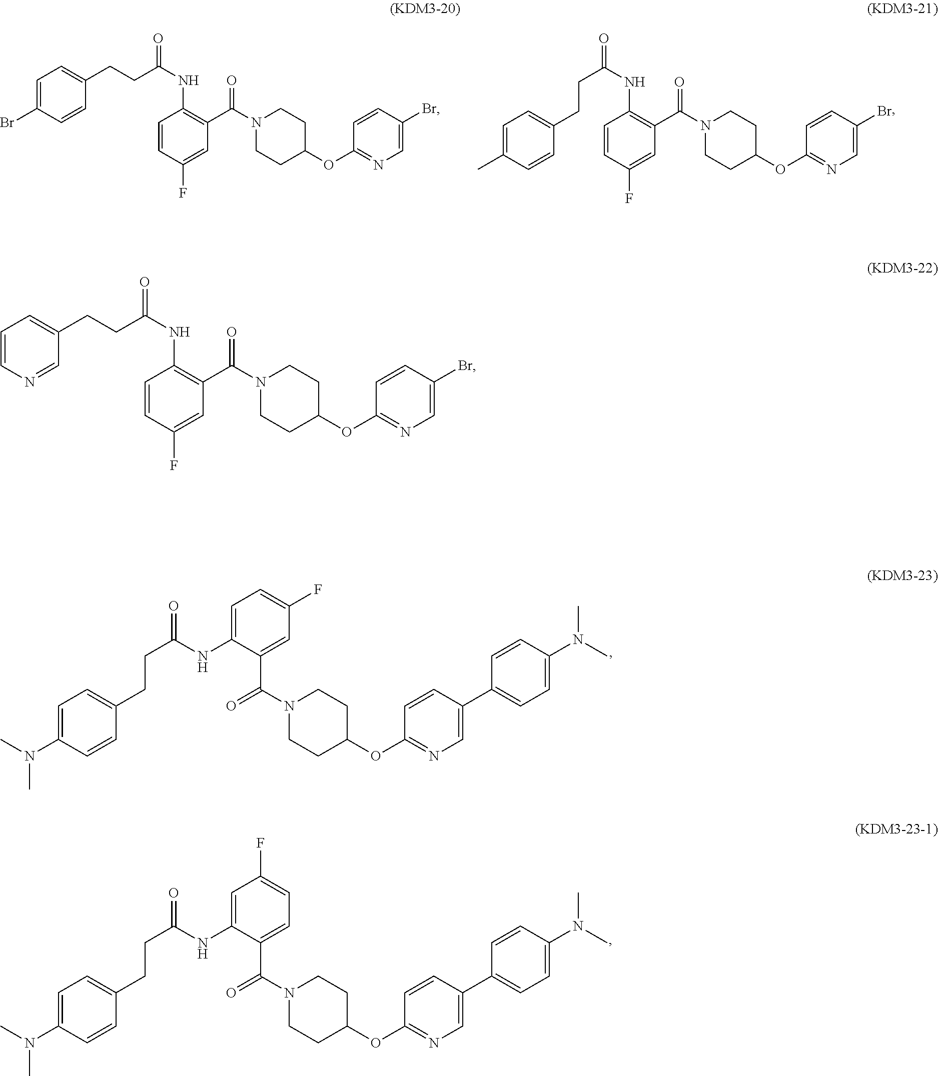

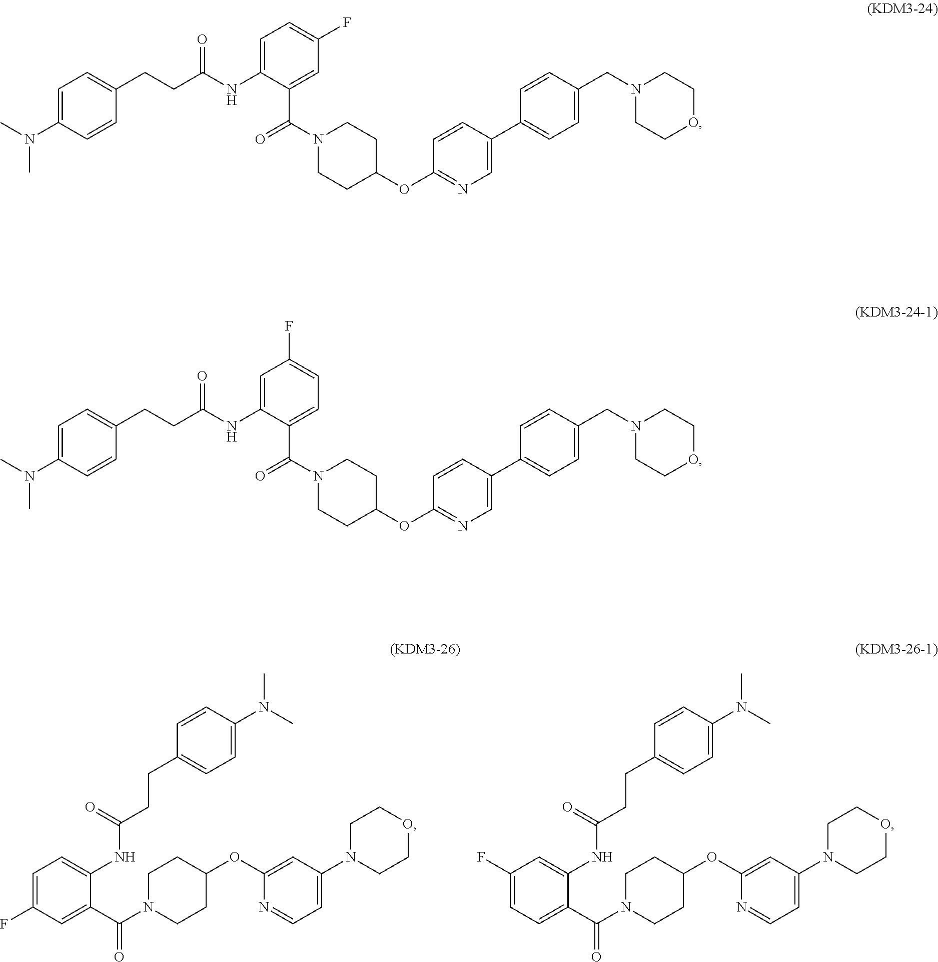 [SMILES: CN(C)c1ccc(CCC(=O)Nc2ccc(F)cc2C(=O)N2CCC(Oc3ccc(-c4ccc(CN5CCOCC5)cc4)cn3)CC2)cc1 ; CN(C)c1ccc(CCC(=O)Nc2cc(F)ccc2C(=O)N2CCC(Oc3ccc(-c4ccc(CN5CCOCC5)cc4)cn3)CC2)cc1 ; CN(C)c1ccc(CCC(=O)Nc2ccc(F)cc2C(=O)N2CCC(Oc3cc(N4CCOCC4)ccn3)CC2)cc1 ; CN(C)c1ccc(CCC(=O)Nc2cc(F)ccc2C(=O)N2CCC(Oc3cc(N4CCOCC4)ccn3)CC2)cc1]

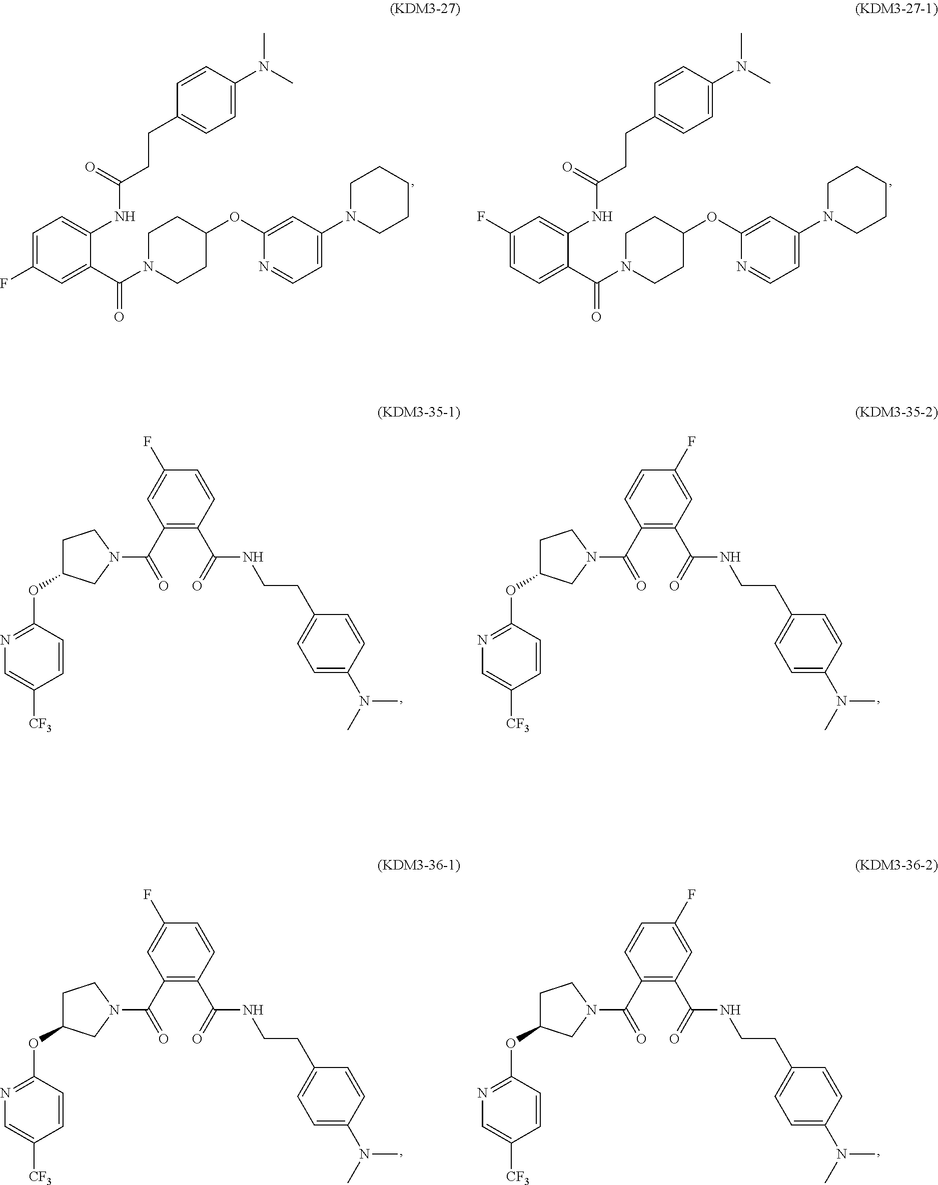 [SMILES: CN(C)c1ccc(CCC(=O)Nc2ccc(F)cc2C(=O)N2CCC(Oc3cc(N4CCCCC4)ccn3)CC2)cc1 ; CN(C)c1ccc(CCC(=O)Nc2cc(F)ccc2C(=O)N2CCC(Oc3cc(N4CCCCC4)ccn3)CC2)cc1 ; CN(C)c1ccc(CCNC(=O)c2ccc(F)cc2C(=O)N2CC[C@@H](Oc3ccc(C(F)(F)F)cn3)C2)cc1 ; CN(C)c1ccc(CCNC(=O)c2cc(F)ccc2C(=O)N2CC[C@@H](Oc3ccc(C(F)(F)F)cn3)C2)cc1 ; CN(C)c1ccc(CCNC(=O)c2ccc(F)cc2C(=O)N2CC[C@H](Oc3ccc(C(F)(F)F)cn3)C2)cc1 ; CN(C)c1ccc(CCNC(=O)c2cc(F)ccc2C(=O)N2CC[C@H](Oc3ccc(C(F)(F)F)cn3)C2)cc1]

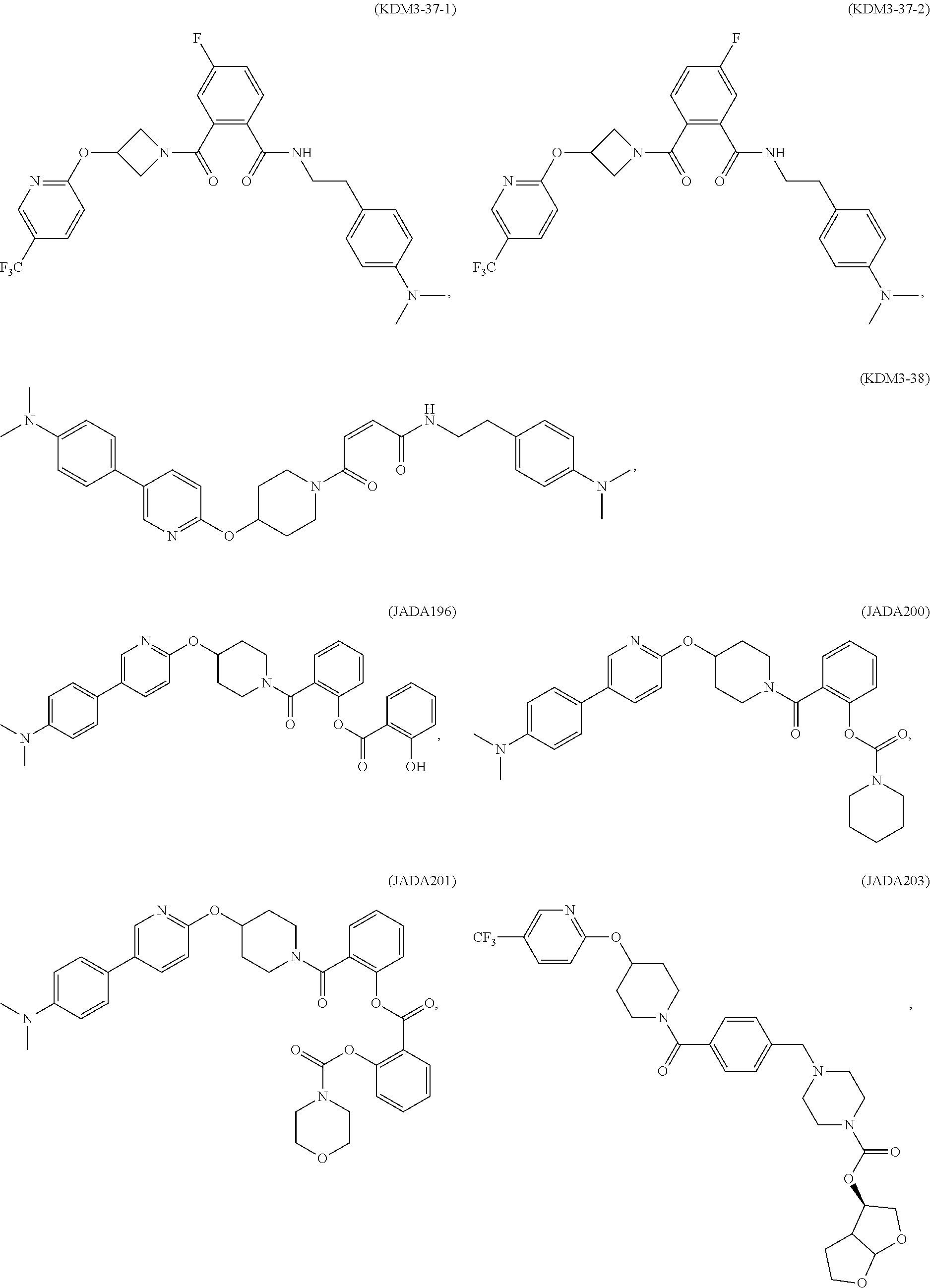

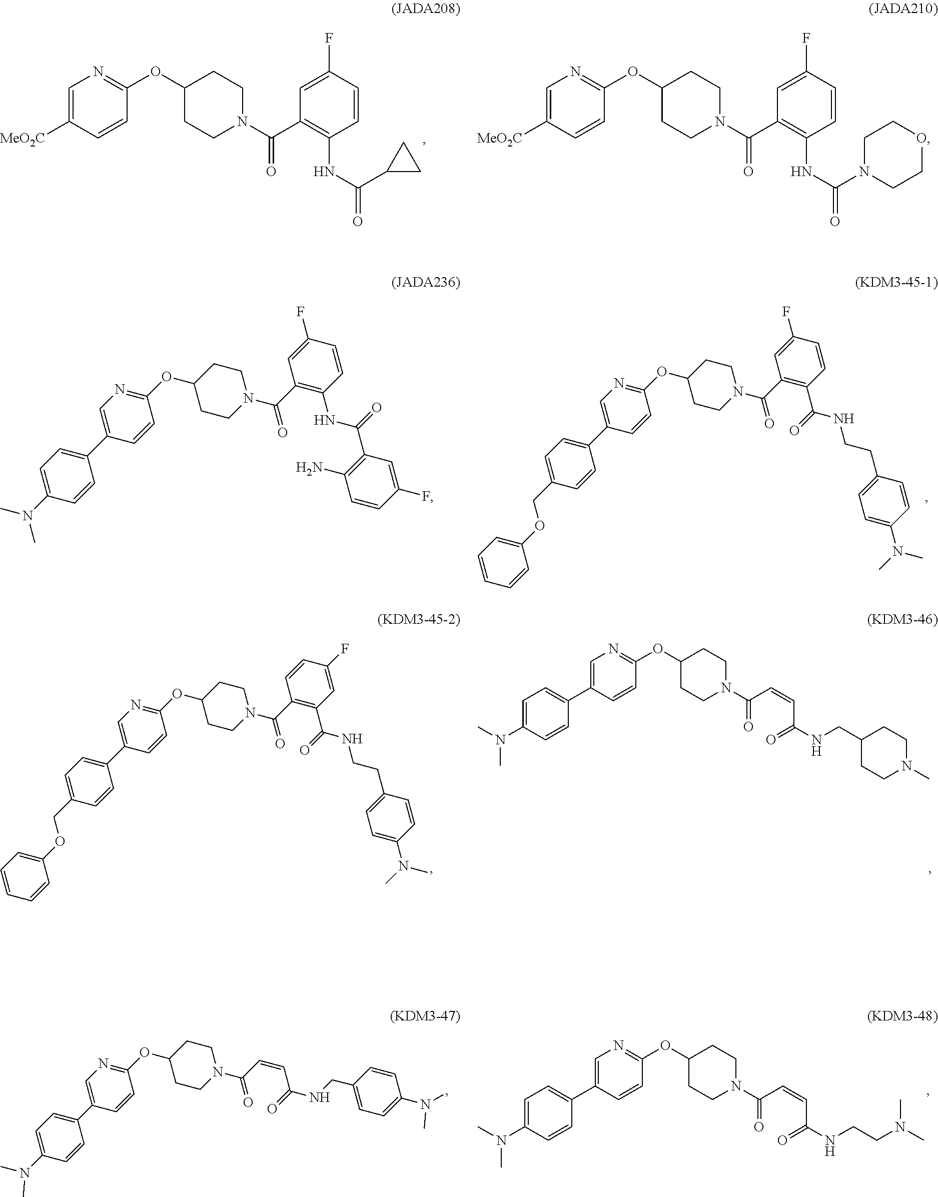

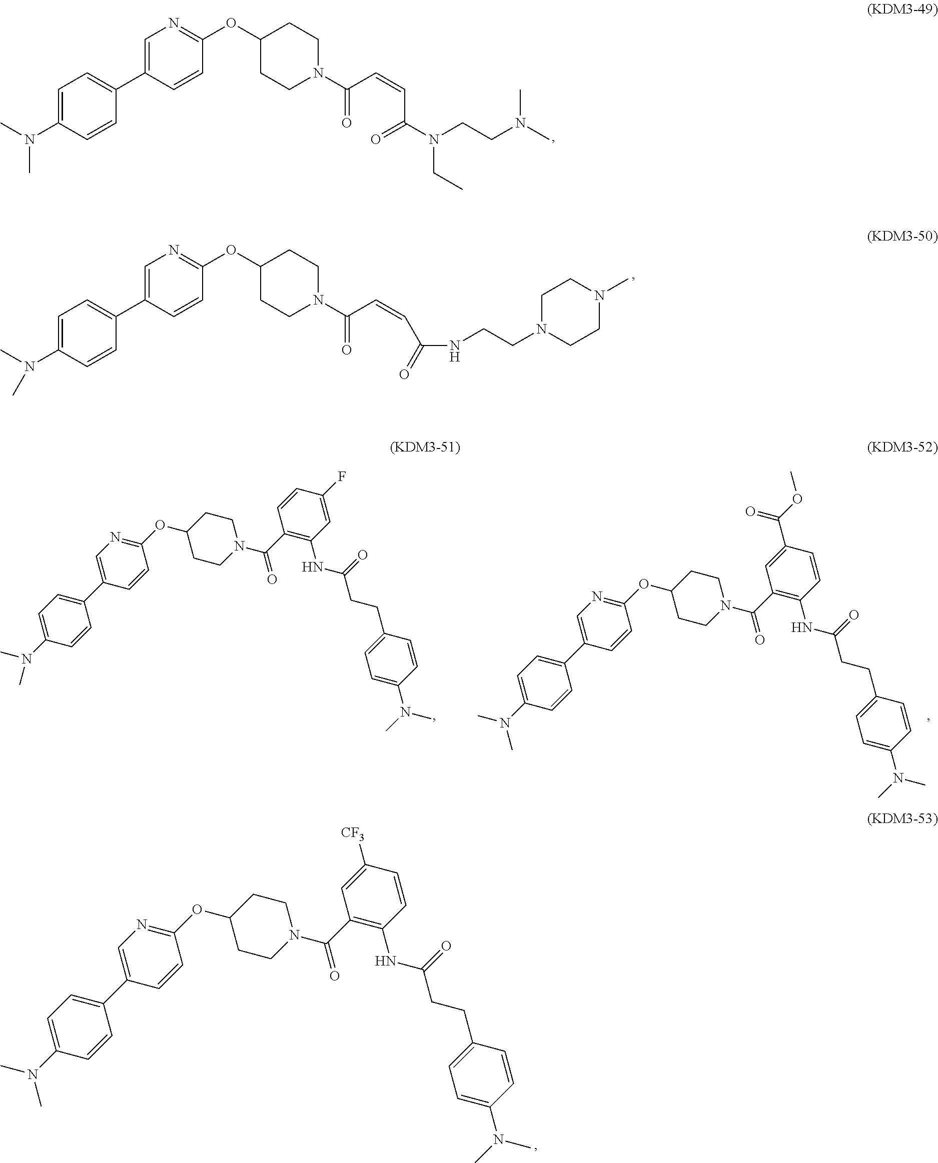

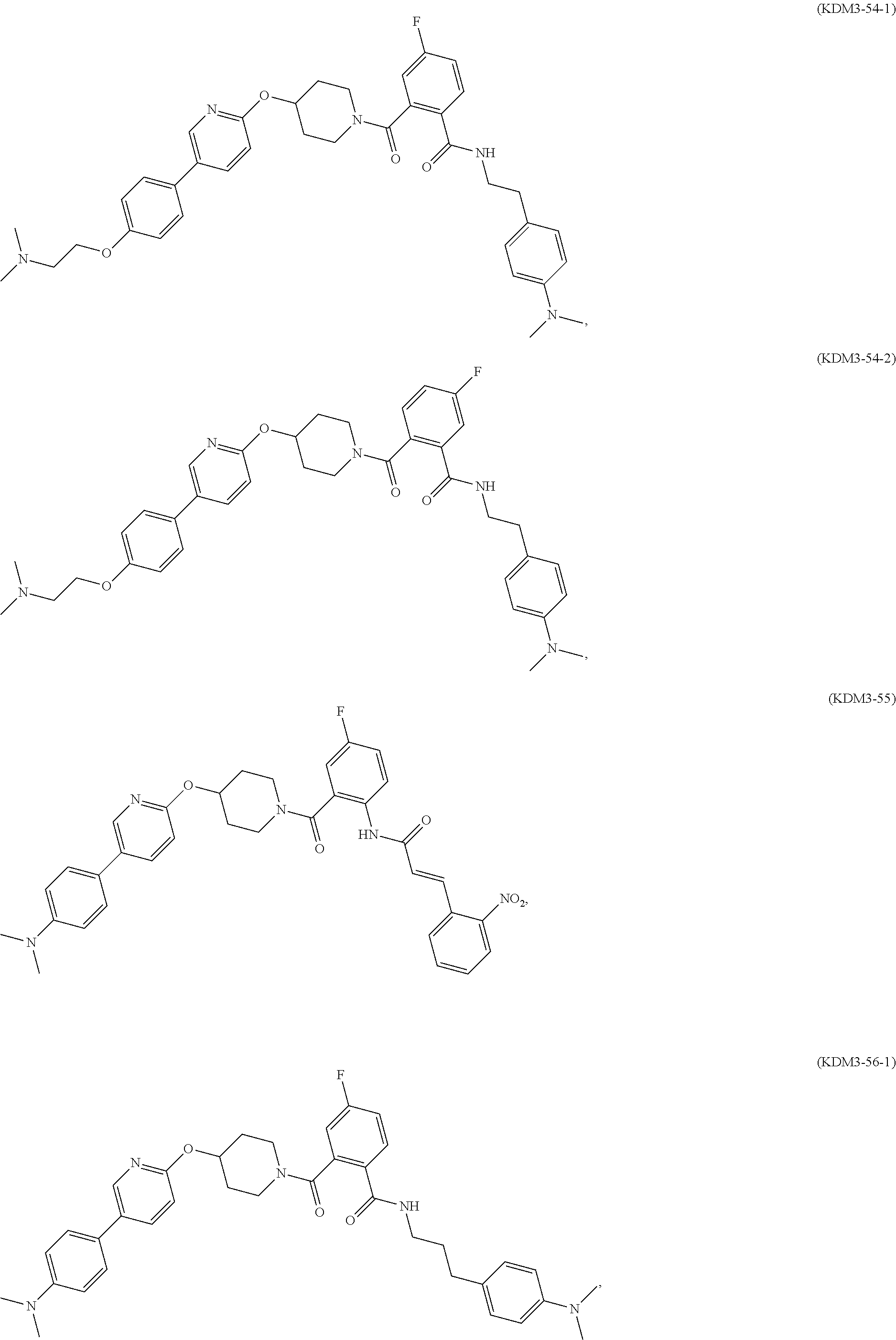

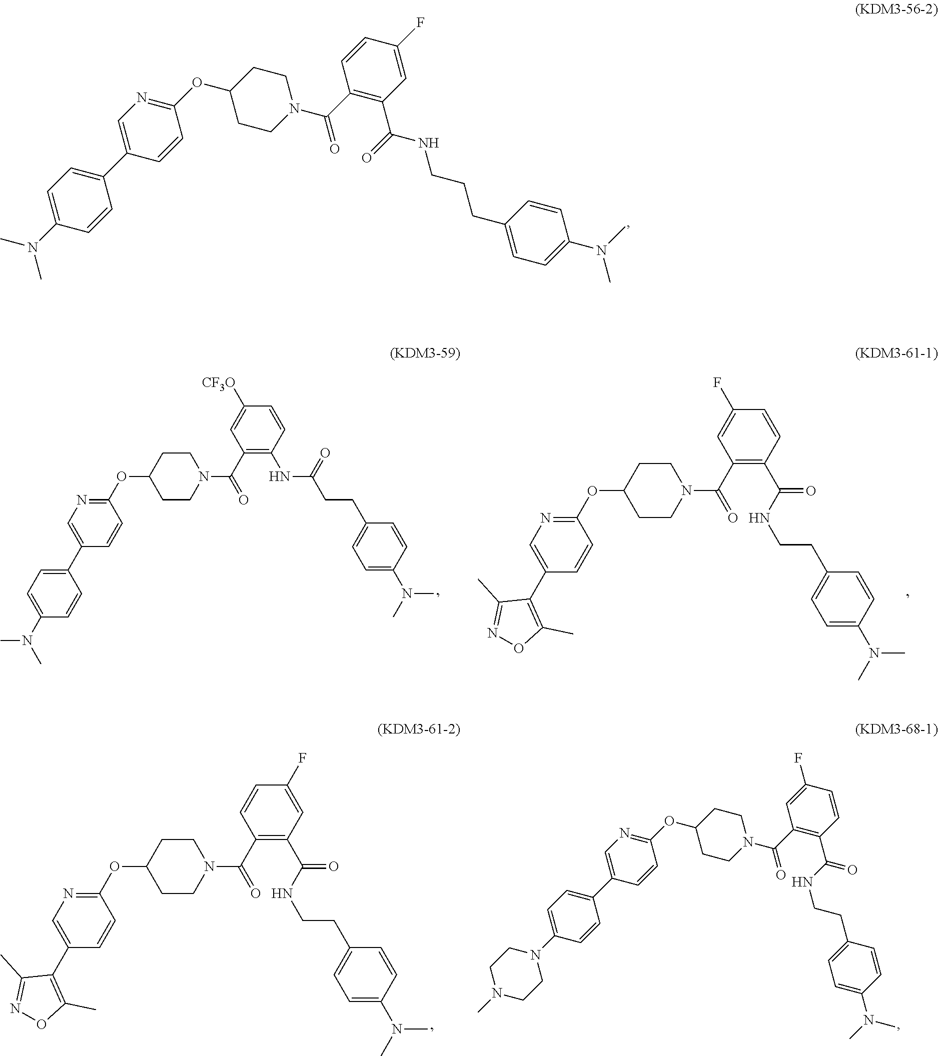

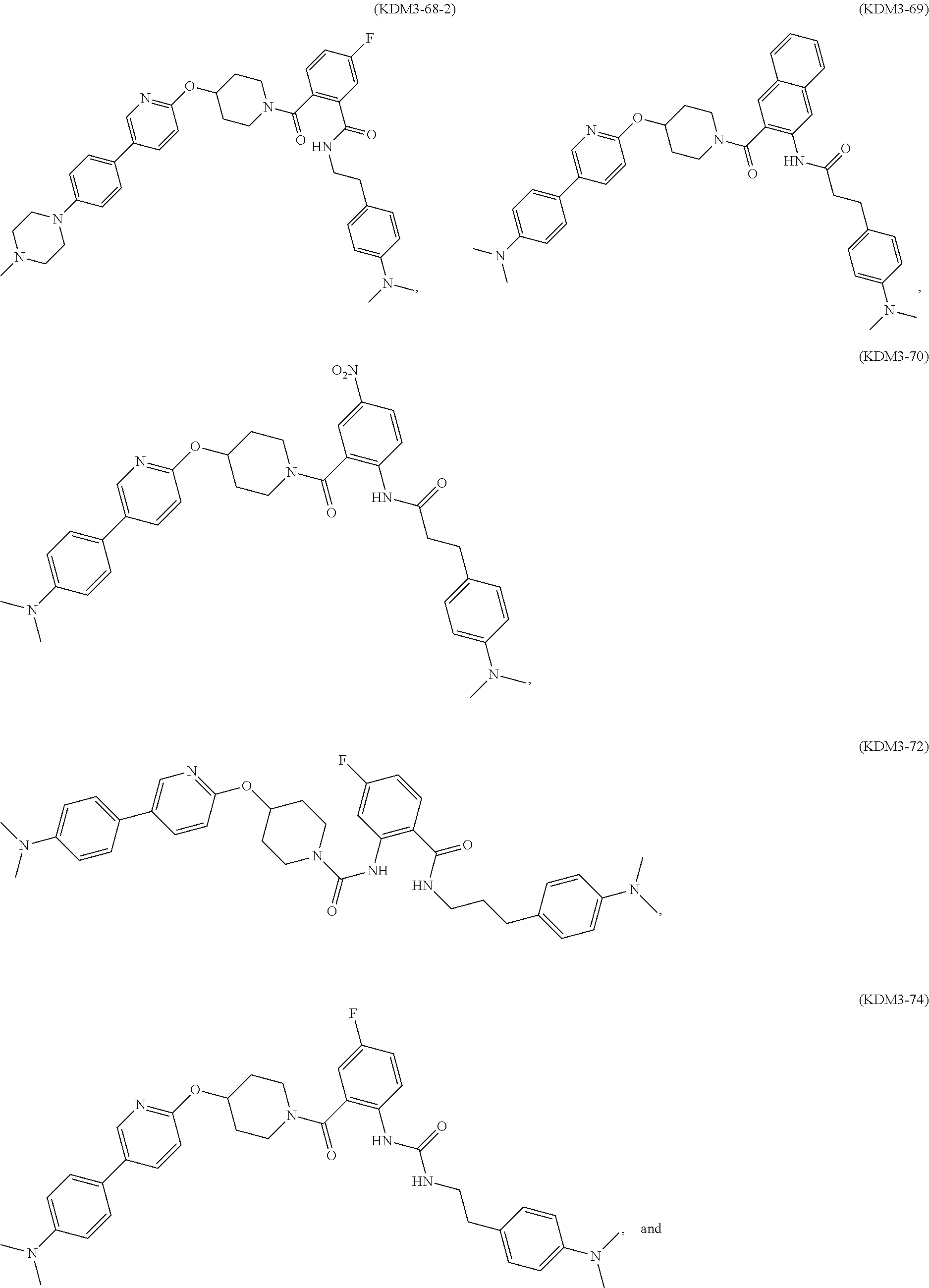

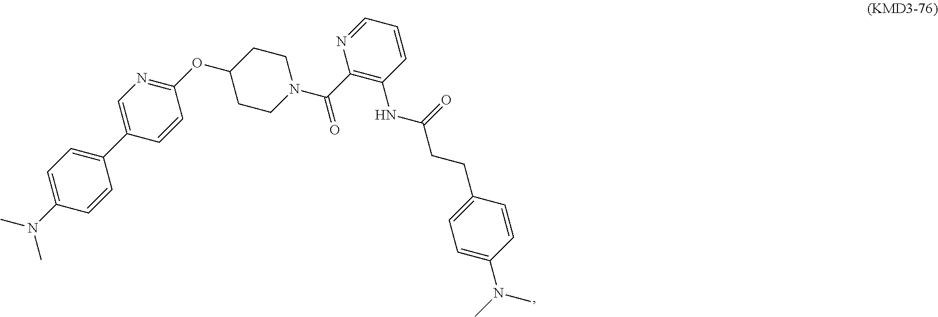

and pharmaceutically acceptable salts, hydrates, solvates, polymorphs, co-crystals, tautomers, stereoisomers, isotopically labeled derivatives, or prodrugs thereof.

### Compounds of Formula (II)

Other inventive compounds are represented by Formula (II):

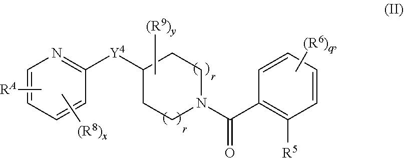

or a pharmaceutically acceptable salt, hydrate, solvate, polymorph, co-crystal, tautomer, stereoisomer, isotopically labeled derivative, or prodrug thereof, wherein:

each instance of R^{A} is independently optionally substituted aryl, optionally substituted heteroaryl, optionally substituted carbocyclyl, optionally substituted heterocyclyl, or —N(R^{N})₂;

each instance of R⁸ is independently hydrogen, halogen, —CN, —N₃, —NO₂, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, optionally substituted sulfonyl, optionally substituted sulfinyl, —OR^{O}, —N(R^{N})₂, or —SR^{S};

x is 0, or an integer from 1-3, inclusive;

Y⁴ is —C(R^{C})₂—, —O—, —NR^{N}—, or —S—;

each instance of R⁹ is independently hydrogen, halogen, —CN, —N₃, —NO₂, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, —OR^{O}, —N(R^{N})₂, or —SR^{S};

y is 0, or an integer from 1 to 9, inclusive;

each instance of r is independently 0 or 1;

R⁵ is hydrogen, —OH, —NO₂, —N(R^{N1})₂, —C(═O)N(R^{N1})₂, —C(═O)OR^{O1}, or —C(═O)R^{C1};

each instance of R^{N1} is independently hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, optionally substituted sulfonyl, optionally substituted sulfinyl, or a nitrogen protecting group; or two R^{N1} bonded to the same nitrogen atom are taken together with the intervening atoms to form optionally substituted heterocyclyl or optionally substituted heteroaryl;

each instance of R^{O1} is independently hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, or an oxygen protecting group;

each instance of R^{C1} is independently hydrogen, halogen, —CN, —N₃, —NO₂, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, optionally substituted sulfonyl, optionally substituted sulfinyl, —OR^{O}, —N(R^{N})₂, or —SR^{S}; or two R^{C} bonded to the same carbon are taken together with the intervening atoms to form optionally substituted carbocyclyl or optionally substituted heterocyclyl;

each instance of R⁶ is independently hydrogen, halogen, —CN, —N₃, —NO₂, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, optionally substituted sulfonyl, optionally substituted sulfinyl, —OR^{O}, —N(R^{N})₂, or —SR^{S};

q is 0, or an integer from 1 to 4, inclusive;

each instance of R^{N} is independently hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, optionally substituted sulfonyl, optionally substituted sulfinyl, or a nitrogen protecting group; or two R^{N} bonded to the same nitrogen atom are taken together with the intervening atoms to form optionally substituted heterocyclyl or optionally substituted heteroaryl;

each instance of R^{C} is independently hydrogen, halogen, —CN, —N₃, —NO₂, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, optionally substituted sulfonyl, optionally substituted sulfinyl, —OR^{O}, —N(R^{N})₂, or —SR^{S}; or two R^{C} bonded to the same carbon are taken together with the intervening atoms to form optionally substituted carbocyclyl or optionally substituted heterocyclyl;

each instance of R^{O} is independently hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, optionally substituted sulfonyl, optionally substituted sulfinyl, or an oxygen protecting group; and

each instance of R^{S} is independently hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, a sulfur protecting group, or R^{S}, taken together with the sulfur atom to which it is attached forms a disulfide group substituted with hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, or optionally substituted acyl, provided that

when Y⁴ is —NH—, each instance of r is 1, and R⁵ is —N(Me)₂, R^{A} is not optionally substituted pyridyl,

when Y⁴ is —NR^{N}—, each instance of r is 1, and R⁵ is —N(R^{N1})₂, R^{A} is not optionally substituted pyridyl,

when Y⁴ is —NH—, each instance of r is 1, and R⁵ is —N(Me)₂, R^{A} is not optionally substituted heteroaryl, and

when Y⁴ is —NR^{N}—, each instance of r is 1, and R⁵ is —N(R^{N1})₂, R^{A} is not optionally substituted heteroaryl.

In certain embodiments, when Y⁴ is —NH—, each instance of r is 1, and R⁵ is —N(Me)₂, then R^{A} is not optionally substituted pyridyl. In certain embodiments, when Y⁴ is —NR^{N}—, each instance of r is 1, and R⁵ is —N(R^{N1})₂, then R^{A} is not optionally substituted pyridyl. In certain embodiments, when Y⁴ is —NH—, each instance of r is 1, and R⁵ is —N(Me)₂, then R^{A} is not optionally substituted heteroaryl. In certain embodiments, when Y⁴ is —NR^{N}—, each instance of r is 1, and R⁵ is —N(R^{N1})₂, then R^{A} is not optionally substituted heteroaryl.

In certain embodiments, when Y⁴ is —NH—, each instance of r is 1, and R⁵ is —N(Me)₂, then R^{A} is optionally substituted aryl. In certain embodiments, when Y⁴ is —NR^{N}—, each instance of r is 1, and R⁵ is —N(R^{N1})₂, R^{A} is optionally substituted aryl. In certain embodiments, when Y⁴ is —NH—, each instance of r is 1, and R⁵ is —N(Me)₂, then R^{A} is optionally substituted phenyl. In certain embodiments, when Y⁴ is —NR^{N}—, each instance of r is 1, and R⁵ is —N(R^{N1})₂, R^{A} is optionally substituted phenyl.

In certain embodiments, the compound is not of the formula:

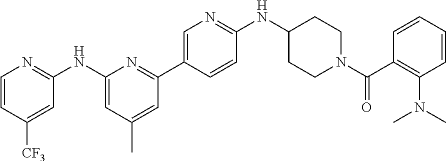 [SMILES: Cc1cc(Nc2cc(C(F)(F)F)ccn2)nc(-c2ccc(NC3CCN(C(=O)c4ccccc4N(C)C)CC3)nc2)c1]

In certain embodiments, the compound of Formula (II) is of Formula (II-i):

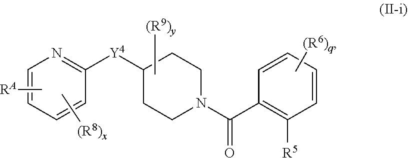

or a pharmaceutically acceptable salt, hydrate, solvate, polymorph, co-crystal, tautomer, stereoisomer, isotopically labeled derivative, or prodrug thereof.

In certain embodiments, the compound of Formula (II) is of Formula (II-ii):

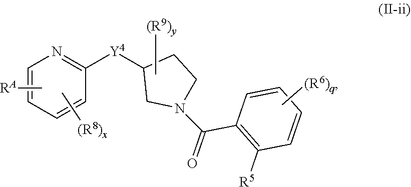

or a pharmaceutically acceptable salt, hydrate, solvate, polymorph, co-crystal, tautomer, stereoisomer, isotopically labeled derivative, or prodrug thereof.

In certain embodiments, the compound of Formula (II) is of Formula (II-iii):

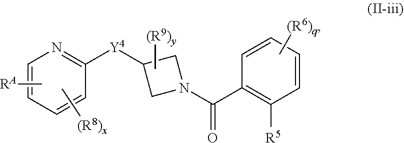

or a pharmaceutically acceptable salt, hydrate, solvate, polymorph, co-crystal, tautomer, stereoisomer, isotopically labeled derivative, or prodrug thereof.

In certain embodiments, the compound of Formula (II) is of Formula (II-a):

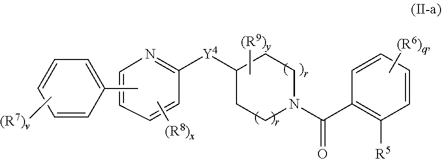

or a pharmaceutically acceptable salt, hydrate, solvate, polymorph, co-crystal, tautomer, stereoisomer, isotopically labeled derivative, or prodrug thereof; wherein:

each instance of R⁷ is independently hydrogen, halogen, —CN, —N₃, —NO₂, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, optionally substituted sulfonyl, optionally substituted sulfinyl, —OR^{O}, —N(R^{N})₂, or —SR^{S}; and

v is 0, or an integer from 1 to 5, inclusive.

In certain embodiments, the compound of Formula (II) is of Formula (II-a-1) or (II-a-2):

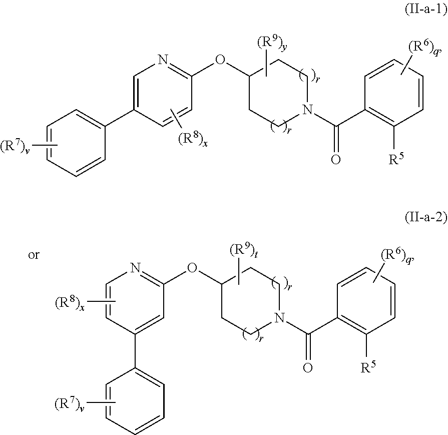

or a pharmaceutically acceptable salt, hydrate, solvate, polymorph, co-crystal, tautomer, stereoisomer, isotopically labeled derivative, or prodrug thereof.

In certain embodiments, the compound of Formula (II) is of Formula (II-a-3) or (II-a-4):

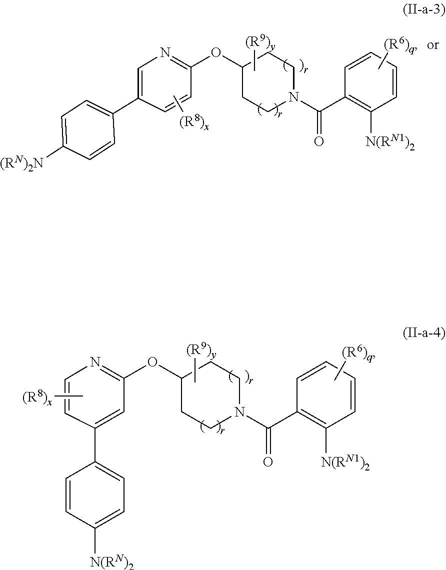

or a pharmaceutically acceptable salt, hydrate, solvate, polymorph, co-crystal, tautomer, stereoisomer, isotopically labeled derivative, or prodrug thereof.

In certain embodiments, the compound of Formula (II) is of Formula (II-a-5) or (II-a-6):

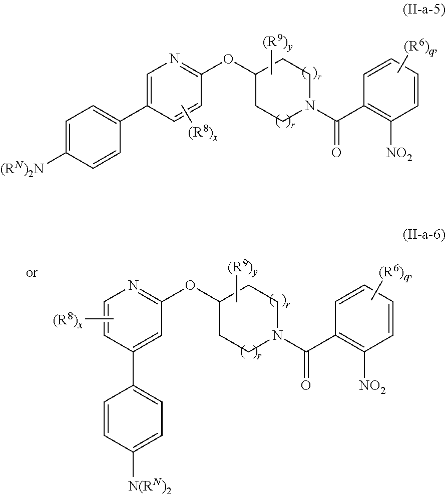

or a pharmaceutically acceptable salt, hydrate, solvate, polymorph, co-crystal, tautomer, stereoisomer, isotopically labeled derivative, or prodrug thereof.

In certain embodiments, the compound of Formula (II) is represented by any one of structures:

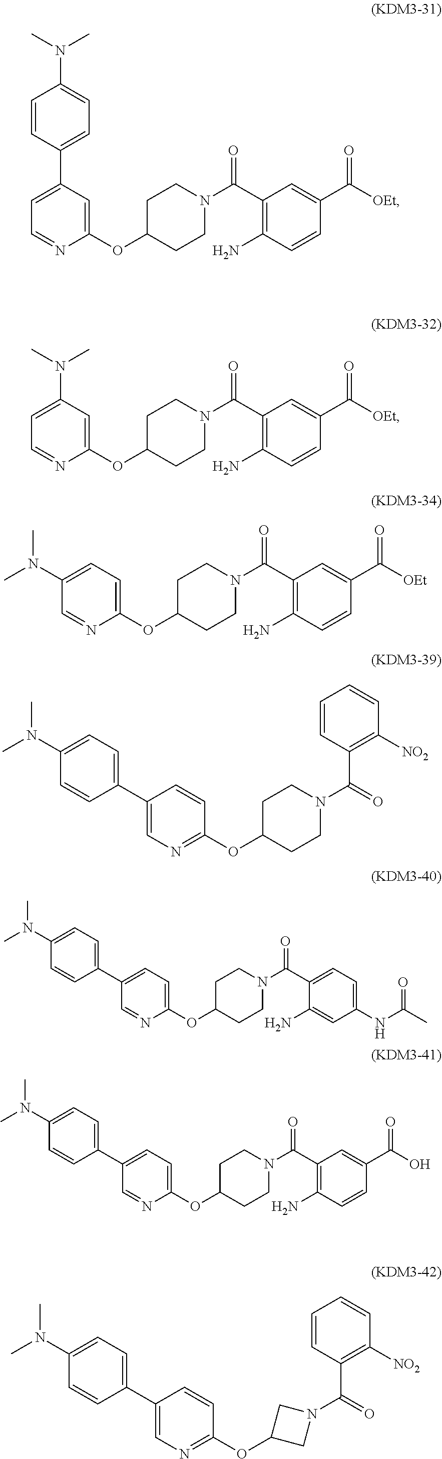

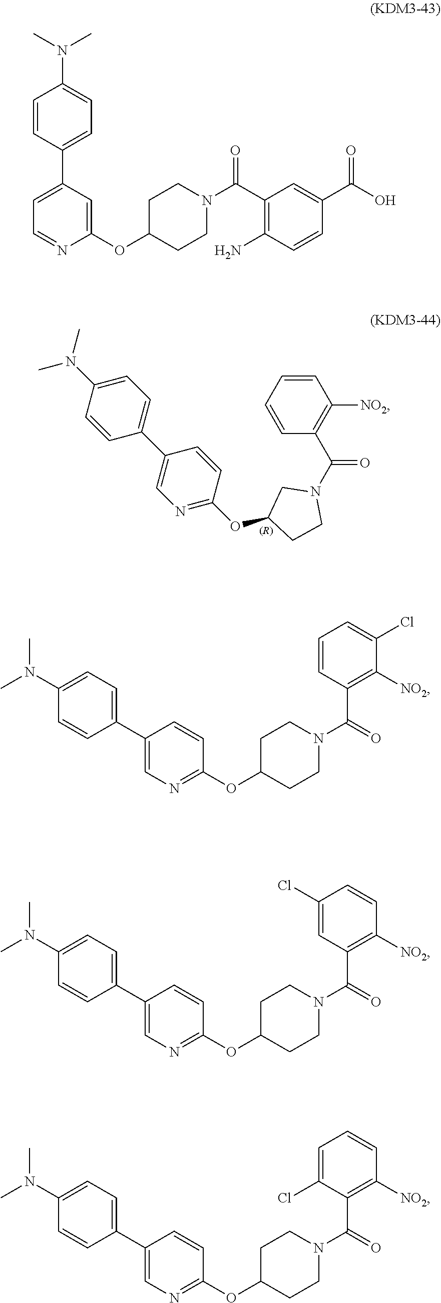

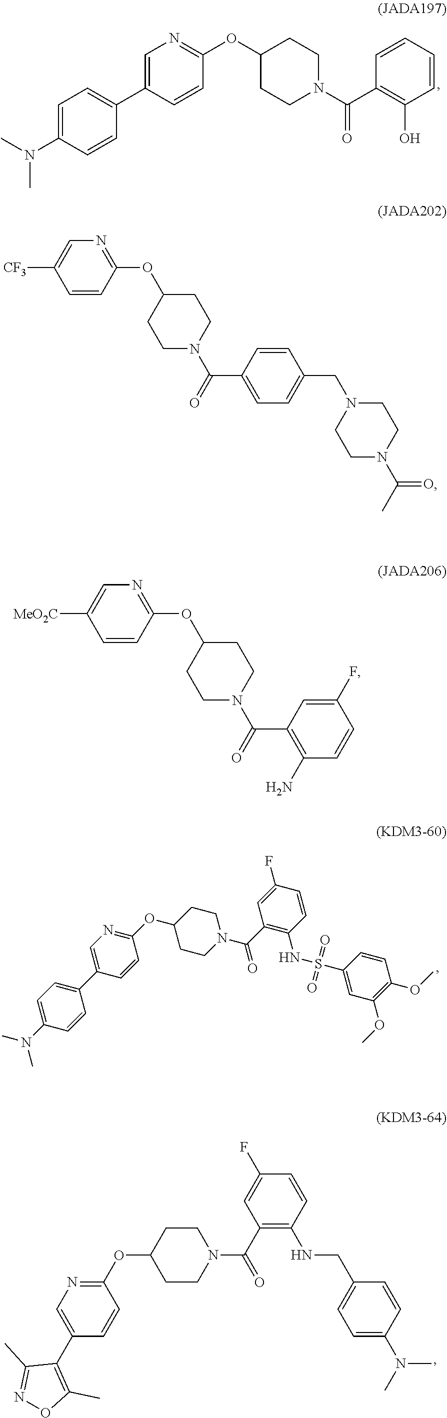

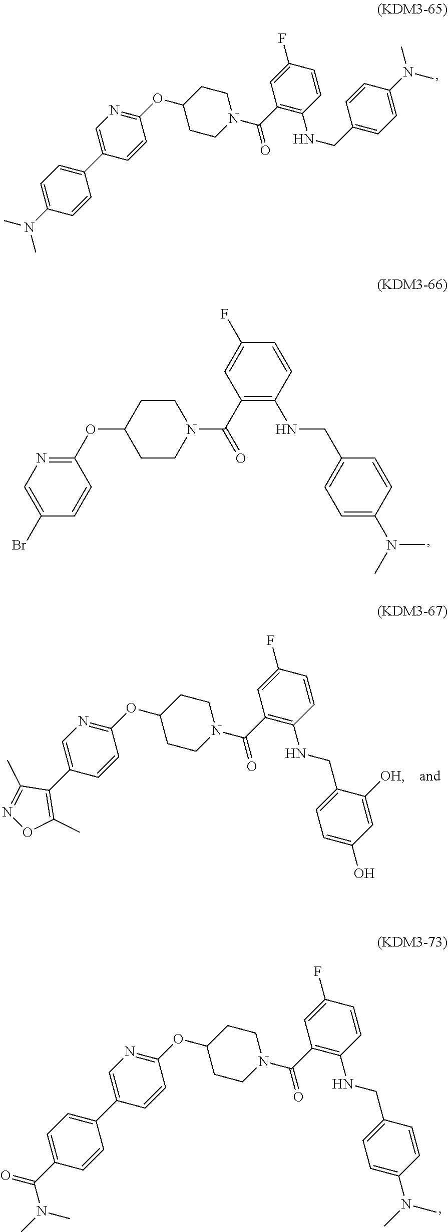

and pharmaceutically acceptable salts, hydrates, solvates, polymorphs, co-crystals, tautomers, stereoisomers, isotopically labeled derivatives, and prodrugs thereof.

### Group Definitions

The following definitions and embodiments apply to all formulas provided herein.

### L¹ and p

As defined herein, L¹ is optionally substituted C₁₋₆ alkylene, optionally substituted C₂₋₆ alkenylene, optionally substituted carbocyclyl, optionally substituted heterocyclyl, optionally substituted arylene, or optionally substituted heteroarylene. In certain embodiments, L¹ is optionally substituted C₁₋₆ alkylene, optionally substituted C₂₋₆ alkenylene, optionally substituted arylene, or optionally substituted heteroarylene. In certain embodiments, L¹ is optionally substituted C₁₋₆ alkylene. In certain embodiments, L¹ is optionally substituted C₂₋₆ alkenylene. In certain embodiments, L¹ is optionally substituted arylene. In certain embodiments, L¹ is optionally substituted heteroarylene. In certain embodiments, L¹ is optionally substituted carbocyclyl. In certain embodiments, L¹ is optionally substituted heterocyclyl,

In certain embodiments, L¹ is optionally substituted C₁₋₆ alkylene. In certain embodiments, L¹ is optionally substituted C₂₋₃ alkylene. In certain embodiments, L¹ is optionally substituted C₂ alkylene. In certain embodiments, L¹ is optionally substituted C₃ alkylene. In certain embodiments, L¹ is of structure:

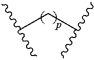

In certain embodiments, L¹ is of the formula —CH₂CH₂—. In certain embodiments, L¹ is of the formula —CH₂CH₂CH₂—.

As defined herein, p is an integer from 1 to 6, inclusive (i.e., 1, 2, 3, 4, 5, or 6). In certain embodiments, p is 1. In certain embodiments, p is 2. In certain embodiments, p is 3. In certain embodiments, p is 4. In certain embodiments, p is 5. In certain embodiments, p is 6.

In certain embodiments, L¹ is optionally substituted arylene. In certain embodiments, L¹ is optionally substituted phenylene. In certain embodiments, L¹ is unsubstituted phenylene. In certain embodiments, L¹ is of structure:

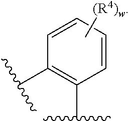

In certain embodiments, L¹ is of one of the following structures:

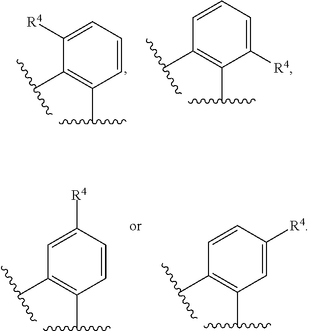

In certain embodiments, L¹ is one of the following structures:

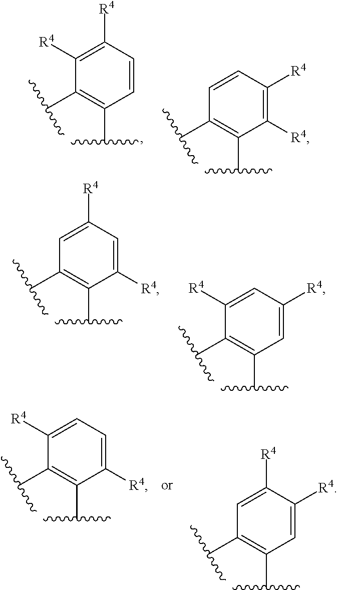

In certain embodiments, L¹ is one of the following structures:

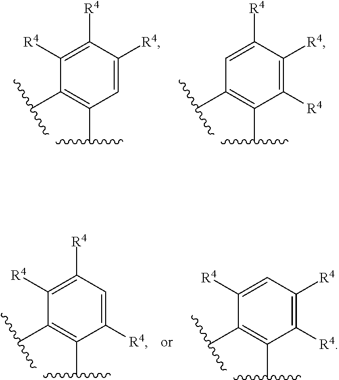

In certain embodiments, L¹ is of the following structure:

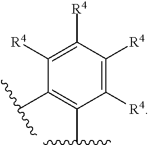

In certain embodiments, L1 is of the following structure:

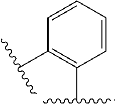 [SMILES: *c1ccccc1*]

In certain embodiments, L¹ is of one of the following structures:

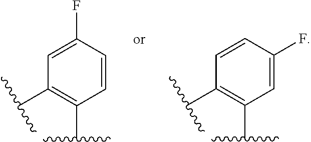 [SMILES: *c1ccc(F)cc1* ; *c1ccc(F)cc1*]

In certain embodiments, L¹ is of one of the following structures:

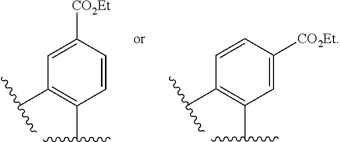 [SMILES: *c1ccc(C(=O)OCC)cc1* ; *c1ccc(C(=O)OCC)cc1*]

In certain embodiments, L¹ is optionally substituted C₂₋₆ alkenylene. In certain embodiments, L¹ is of structure:

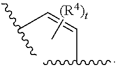

In certain embodiments, L¹ is of the formula:

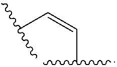 [SMILES: */C=C\*]

In certain embodiments, L¹ is of structure:

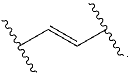 [SMILES: */C=C/*]

### R⁴, w, and t

As defined herein, each instance of R⁴ is independently hydrogen, halogen, —CN, —N₃, —NO₂, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, optionally substituted sulfonyl, optionally substituted sulfinyl, —OR^{O}, —N(R^{N})₂, or —SR^{S}. In certain embodiments, at least one instance of R⁴ is hydrogen. In certain embodiments, at least one instance of R⁴ is halogen. In certain embodiments, at least one instance of R⁴ is —CN. In certain embodiments, at least one instance of R⁴ is —N₃. In certain embodiments, at least one instance of R⁴ is —NO₂. In certain embodiments, at least one instance of R⁴ is optionally substituted alkyl. In certain embodiments, at least one instance of R⁴ is optionally substituted alkenyl. In certain embodiments, at least one instance of R⁴ is optionally substituted alkynyl. In certain embodiments, at least one instance of R⁴ is optionally substituted aryl. In certain embodiments, at least one instance of R⁴ is optionally substituted heteroaryl. In certain embodiments, at least one instance of R⁴ is optionally substituted carbocyclyl. In certain embodiments, at least one instance of R⁴ is optionally substituted heterocyclyl. In certain embodiments, at least one instance of R⁴ is optionally substituted acyl. In certain embodiments, at least one instance of R⁴ is optionally substituted sulfonyl. In certain embodiments, at least one instance of R⁴ is optionally substituted sulfinyl. In certain embodiments, at least one instance of R⁴ is —OR^{O}. In certain embodiments, at least one instance of R⁴ is —N(R^{N})₂. In certain embodiments, at least one instance of R⁴ is —SR^{S}.

In certain embodiments, each instance of R⁴ is hydrogen.

In certain embodiments, at least one instance of R⁴ is halogen. In certain embodiments, at least one instance of R⁴ is —F.

In certain embodiments, at least one instance of R⁴ is optionally substituted C₁₋₆ acyl. In certain embodiments, at least one instance of R⁴ is —CO₂alkyl. In certain embodiments, at least one instance of R⁴ is —CO₂Et.

As defined herein, w is 0, or an integer from 1 to 4, inclusive (i.e., 0, 1, 2, 3, or 4). In certain embodiments, w is 0. In certain embodiments, w is 1. In certain embodiments, w is 2. In certain embodiments, w is 3. In certain embodiments, w is 4.

As defined herein, t is 0, 1, or 2. In certain embodiments, t is 0. In certain embodiments, t is 1. In certain embodiments, t is 2.

Y¹ and Y²

As defined herein, Y¹ is —C(R^{C})₂—, —O—, —NR^{N}—, or —S—. In certain embodiments, Y¹ is —C(R^{C})₂—. In certain embodiments, Y¹ is —CH₂—. In certain embodiments, Y¹ is —S—. In certain embodiments, Y¹ is —O—. In certain embodiments, Y¹ is —NR^{N}—. In certain embodiments, Y¹ is —NH—.

As defined herein, each instance of Y² is independently a bond, —O—, or —NR^{N}—. In certain embodiments, at least one instance of Y² is a bond. In certain embodiments, at least one instance of Y² is —O—. In certain embodiments, at least one instance of Y² is —N(R^{N})—. In certain embodiments, at least one instance of Y² is —NH—.

In certain embodiments, one instance of Y² is —N(R^{N})—; and the other instance is a bond. In certain embodiments, the instance of Y² proximal to R² is —N(R^{N})—; and the other instance is a bond. In certain embodiments, the instance of Y² distal to R² is —N(R^{N})—; and the other instance is a bond. In certain embodiments, one instance of Y² is —NH—; and the other instance is a bond. In certain embodiments, the instance of Y² proximal to R² is —NH—; and the other instance is a bond. In certain embodiments, the instance of Y² distal to R² is —NH—; and the other instance is a bond.

### L² and s

As defined herein, L² is a bond, optionally substituted C₁₋₆ alkylene, optionally substituted C₁₋₆ heteroalkylene, optionally substituted C₂₋₆ alkenylene, optionally substituted C₂₋₆ alkynylene, optionally substituted C₁₋₆ acylene, or a combination thereof. In certain embodiments, L² is a bond. In certain embodiments, L² is optionally substituted C₁₋₆ alkylene. In certain embodiments, L² is optionally substituted C₁₋₆ heteroalkylene. In certain embodiments, L² is optionally substituted C₂₋₆ alkenylene. In certain embodiments, L² is optionally substituted C₂₋₆ alkynylene. In certain embodiments, L² is optionally substituted C₁₋₆ acylene.

In certain embodiments, L² is optionally substituted C₁₋₆ alkylene. In certain embodiments, L² is optionally substituted C₂₋₃ alkylene. In certain embodiments, L² is optionally substituted C₂ alkylene. In certain embodiments, L² is optionally substituted C₃ alkylene. In certain embodiments, L² is of structure:

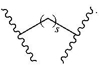

In certain embodiments, L² is of the formula —CH₂CH₂—. In certain embodiments, L² is of the formula —CH₂CH₂CH₂—.

As defined herein, s is an integer from 1 to 6, inclusive (i.e., 1, 2, 3, 4, 5, or 6). In certain embodiments, s is 1. In certain embodiments, s is 2. In certain embodiments, s is 3. In certain embodiments, s is 4. In certain embodiments, s is 5. In certain embodiments, s is 6.

R¹, n, R², and R^{A}

As defined herein, each instance of R¹ is independently hydrogen, halogen, —CN, —N₃, —NO₂, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, optionally substituted sulfonyl, optionally substituted sulfinyl, —OR^{O}, —N(R^{N})₂, or —SR^{S}. In certain embodiments, at least one instance of R¹ is hydrogen. In certain embodiments, at least one instance of R¹ is halogen. In certain embodiments, at least one instance of R¹ is —CN. In certain embodiments, at least one instance of R¹ is —N₃. In certain embodiments, at least one instance of R¹ is —NO₂. In certain embodiments, at least one instance of R¹ is optionally substituted alkyl. In certain embodiments, at least one instance of R¹ is optionally substituted alkenyl. In certain embodiments, at least one instance of R¹ is optionally substituted alkynyl. In certain embodiments, at least one instance of R¹ is optionally substituted aryl. In certain embodiments, at least one instance of R¹ is optionally substituted heteroaryl. In certain embodiments, at least one instance of R¹ is optionally substituted carbocyclyl. In certain embodiments, at least one instance of R¹ is optionally substituted heterocyclyl. In certain embodiments, at least one instance of R¹ is optionally substituted morpholinyl. In certain embodiments, at least one instance of R¹ is optionally substituted piperidinyl. In certain embodiments, at least one instance of R¹ is optionally substituted acyl. In certain embodiments, at least one instance of R¹ is optionally substituted sulfonyl. In certain embodiments, at least one instance of R¹ is optionally substituted sulfinyl. In certain embodiments, at least one instance of R¹ is —OR^{O}. In certain embodiments, at least one instance of R¹ is —N(R^{N})₂. In certain embodiments, at least one instance of R¹ is —SR^{S}.

In certain embodiments, at least one instance of R¹ is optionally substituted aryl. In certain embodiments, at least one instance of R¹ is optionally substituted phenyl. In certain embodiments, at least one instance of R¹ is of structure:

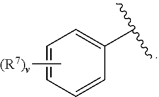

In certain embodiments, at least one instance of R¹ is of structure:

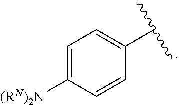

In certain embodiments, at least one instance of R¹ is of the structure:

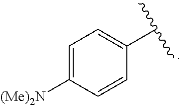

In certain embodiments, at least one instance of R¹ is of the structure:

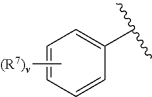

and each instance of R⁷ is independently optionally substituted alkyl, optionally substituted acyl, optionally substituted sulfonyl, optionally substituted sulfinyl, —OR^{O}, or —N(R^{N})₂. In certain embodiments, at least one instance of R¹ is of the structure:

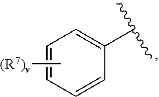

and at least one instance of R⁷ is optionally substituted alkyl. In certain embodiments, at least one instance of R¹ is of the structure:

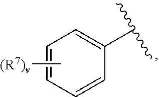

and at least one instance of R⁷ is haloalkyl. In certain embodiments, at least one instance of R¹ is of the structure:

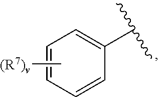

and at least one instance of R⁷ is alkyl-heterocyclyl. In certain embodiments, at least one instance of R¹ is of the structure:

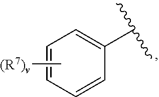

and at least one instance of R⁷ is alkyl-morpholinyl. In certain embodiments, at least one instance of R¹ is of the structure:

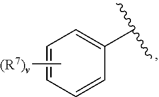

and at least one instance of R⁷ is alkyl-piperidinyl. In certain embodiments, at least one instance of R¹ is of the structure:

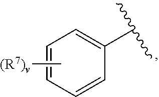

and at least one instance of R⁷ is —NHC(O)-alkyl. In certain embodiments, at least one instance of R¹ is of the structure:

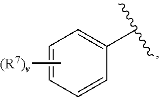

and at least one instance of R⁷ is —NHC(O)-alkyl-CO(O)H. In certain embodiments, at least one instance of R¹ is of the structure:

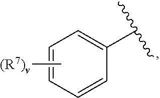

and at least one instance of R⁷ is —NHC(O)-alkyl-CO(O)alkyl.

In certain embodiments, at least one instance of R¹ is hydrogen. In certain embodiments, each instance of R¹ is hydrogen.

In certain embodiments, at least one instance of R¹ is haloalkyl. In certain embodiments, at least one instance of R¹ is —CF₃. In certain embodiments, at least one instance of R¹ is —CH₂F. In certain embodiments, at least one instance of R¹ is —CHF₂. In certain embodiments, at least one instance of R¹ is —OCF₃.

As defined herein, n is 0, or an integer from 1 to 4, inclusive (i.e., 0, 1, 2, 3, or 4). In certain embodiments, n is 0. In certain embodiments, n is 1. In certain embodiments, n is 2. In certain embodiments, n is 3. In certain embodiments, n is 4.

As defined herein, R² is optionally substituted aryl, optionally substituted heteroaryl, optionally substituted carbocyclyl, or optionally substituted heterocyclyl. In certain embodiments, R² is optionally substituted aryl. In certain embodiments, R² is optionally substituted heteroaryl. In certain embodiments, R² is optionally substituted carbocyclyl. In certain embodiments, R² is optionally substituted heterocyclyl.

In certain embodiments, R² is optionally substituted aryl. In certain embodiments, R² is optionally substituted phenyl. In certain embodiments, R² is of the structure:

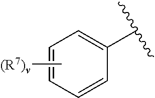

In certain embodiments, R² is of the structure:

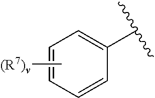

and at least one instance of R⁷ is halo. In certain embodiments, R² is of the structure:

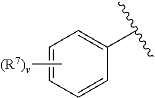

and at least one instance of R⁷ is optionally substituted alkyl. In certain embodiments, R² is of the structure:

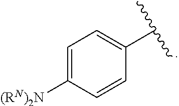

In certain embodiments, R² is of the structure:

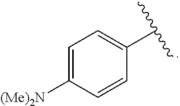

In certain embodiments, R² is of the structure:

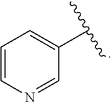 [SMILES: *c1cccnc1]

In certain embodiments, R² is of the structure:

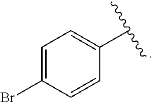 [SMILES: *c1ccc(Br)cc1]

In certain embodiments, R² is of the structure:

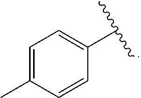 [SMILES: *c1ccc(C)cc1]

As defined herein, R^{A} optionally substituted aryl, optionally substituted heteroaryl, optionally substituted carbocyclyl, optionally substituted heterocyclyl, or —N(R^{N})₂. In certain embodiments, R^{A} is optionally substituted aryl. In certain embodiments, R^{A} is optionally substituted heteroaryl. In certain embodiments, R^{A} is optionally substituted carbocyclyl. In certain embodiments, R^{A} is optionally substituted heterocyclyl. In certain embodiments, R^{A} is —N(R^{N})₂.

In certain embodiments, R^{A} is optionally substituted aryl. In certain embodiments, R^{A} is optionally substituted phenyl. In certain embodiments, R^{A} is of the structure:

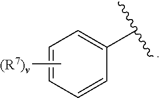

In certain embodiments, R^{A} is of the structure:

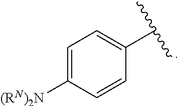

In certain embodiments, R^{A} is of the structure

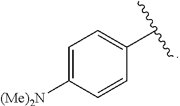

### R⁷ and v

As defined herein, each instance of R⁷ is independently hydrogen, halogen, —CN, —N₃, —NO₂, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, optionally substituted sulfonyl, optionally substituted sulfinyl, —OR^{O}, —N(R^{N})₂, or —SR^{S}. In certain embodiments, at least one instance of R⁷ is hydrogen. In certain embodiments, at least one instance of R⁷ is halogen. In certain embodiments, at least one instance of R⁷ is —CN. In certain embodiments, at least one instance of R⁷ is —N₃. In certain embodiments, at least one instance of R⁷ is —NO₂. In certain embodiments, at least one instance of R⁷ is optionally substituted alkyl. In certain embodiments, at least one instance of R⁷ is optionally substituted alkenyl. In certain embodiments, at least one instance of R⁷ is optionally substituted alkynyl. In certain embodiments, at least one instance of R⁷ is optionally substituted aryl. In certain embodiments, at least one instance of R⁷ is optionally substituted heteroaryl. In certain embodiments, at least one instance of R⁷ is optionally substituted carbocyclyl. In certain embodiments, at least one instance of R⁷ is optionally substituted heterocyclyl. In certain embodiments, at least one instance of R⁷ is optionally substituted acyl. In certain embodiments, at least one instance of R⁷ is optionally substituted sulfonyl. In certain embodiments, at least one instance of R⁷ is optionally substituted sulfinyl. In certain embodiments, at least one instance of R⁷ is —OR^{O}. In certain embodiments, at least one instance of R⁷ is —N(R^{N})₂. In certain embodiments, at least one instance of R⁷ is —SR^{S}.

In certain embodiments, at least one instance of R⁷ is —N(R^{N})₂. In certain embodiments, at least one instance of R⁷ is —N(Me)₂. In certain embodiments, one instance of R⁷ is —N(R^{N})₂. In certain embodiments, one instance of R⁷ is —N(Me)₂. In certain embodiments, one instance of R⁷ is Br. In certain embodiments, one instance of R⁷ is methyl.

As defined herein, v is 0, or an integer from 1 to 5, inclusive (i.e., 0, 1, 2, 3, 4, or 5). In certain embodiments, v is 0. In certain embodiments, v is 1. In certain embodiments, v is 2. In certain embodiments, v is 3. In certain embodiments, v is 4. In certain embodiments, v is 5.

### R³. m, and r

As defined herein, each instance of R³ is independently hydrogen, halogen, —CN, —N₃, —NO₂, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, —OR^{O}, —N(R^{N})₂, or —SR^{S}. In certain embodiments, at least one instance of R³ is hydrogen. In certain embodiments, at least one instance of R³ is halogen. In certain embodiments, at least one instance of R³ is —CN. In certain embodiments, at least one instance of R³ is —N₃. In certain embodiments, at least one instance of R³ is —NO₂. In certain embodiments, at least one instance of R³ is optionally substituted alkyl. In certain embodiments, at least one instance of R³ is optionally substituted alkenyl. In certain embodiments, at least one instance of R³ is optionally substituted alkynyl. In certain embodiments, at least one instance of R³ is optionally substituted aryl. In certain embodiments, at least one instance of R³ is optionally substituted heteroaryl. In certain embodiments, at least one instance of R³ is optionally substituted carbocyclyl. In certain embodiments, at least one instance of R³ is optionally substituted heterocyclyl. In certain embodiments, at least one instance of R³ is optionally substituted acyl. In certain embodiments, at least one instance of R³ is —OR^{O}. In certain embodiments, at least one instance of R³ is —N(R^{N})₂. In certain embodiments, at least one instance of R³ is —SR^{S}.

In certain embodiments, at least one instance of R³ is hydrogen. In certain embodiments, each instance of R³ is hydrogen.

As defined herein, m is 0, or an integer from 1 to 5, inclusive (i.e., 0, 1, 2, 5, 4, or 5). In certain embodiments, m is 0. In certain embodiments, m is 1. In certain embodiments, m is 2. In certain embodiments, m is 3. In certain embodiments, m is 4. In certain embodiments, m is 5.

As defined herein, each instance of r is independently 0 or 1.

In certain embodiments, each instance of r is 1. In certain embodiments, one instance of r is 1; and the other instance is 0. In certain embodiments, each instance of r is 0.

R⁵ and R^{N1}

As defined herein, R⁵ is —NO₂, —N(R^{N1})₂, or —C(═O)N(R^{N1})₂. In certain embodiments, R⁵ is —NO₂. In certain embodiments, R⁵ is —N(R^{N1})₂. In certain embodiments, R⁵ is —NH₂. In certain embodiments, R⁵ is —C(═O)N(R^{N1})₂. In certain embodiments, R⁵ is —C(═O)NH₂. In certain embodiments, R⁵ is —C(═O)OH. In certain embodiments, R⁵ is —C(═O)OEt. In certain embodiments, R⁵ is —NHC(═O)CH₃.

As defined herein, each instance of R^{N1} is independently hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, optionally substituted sulfonyl, optionally substituted sulfinyl, or a nitrogen protecting group; or two R^{N1} bonded to the same nitrogen atom are taken together with the intervening atoms to form optionally substituted heterocyclyl or optionally substituted heteroaryl. In certain embodiments, at least one instance of R^{N1} is hydrogen. In certain embodiments, at least one instance of R^{N1} is optionally substituted alkyl. In certain embodiments, at least one instance of R^{N1} is optionally substituted alkenyl. In certain embodiments, at least one instance of R^{N1} is optionally substituted alkynyl. In certain embodiments, at least one instance of R^{N1} is optionally substituted aryl. In certain embodiments, at least one instance of R^{N1} is optionally substituted heteroaryl. In certain embodiments, at least one instance of R^{N1} is optionally substituted carbocyclyl. In certain embodiments, at least one instance of R^{N1} is optionally substituted heterocyclyl. In certain embodiments, at least one instance of R^{N1} is optionally substituted acyl. In certain embodiments, at least one instance of R^{N1} is optionally substituted sulfonyl. In certain embodiments, at least one instance of R^{N1} is optionally substituted sulfinyl. In certain embodiments, at least one instance of R^{N1} is a nitrogen protecting group. In certain embodiments, two R^{N1} bonded to the same nitrogen atom are taken together with the intervening atoms to form optionally substituted heterocyclyl. In certain embodiments, two R^{N1} bonded to the same nitrogen atom are taken together with the intervening atoms to form optionally substituted heteroaryl.

In certain embodiments, each instance of R^{N1} is hydrogen. In certain embodiments, each instance of R^{N1} is optionally substituted aryl. In certain embodiments, each instance of R^{N1} is optionally substituted phenyl.

### R⁶ and q

As defined herein, each instance of R⁶ is independently hydrogen, halogen, —CN, —N₃, —NO₂, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, optionally substituted sulfonyl, optionally substituted sulfinyl, —OR^{O}, —N(R^{N})₂, or —SR^{S}. In certain embodiments, at least one instance of R⁶ is hydrogen. In certain embodiments, at least one instance of R⁶ is halogen. In certain embodiments, at least one instance of R⁶ is —CN. In certain embodiments, at least one instance of R⁶ is —N₃. In certain embodiments, at least one instance of R⁶ is —NO₂. In certain embodiments, at least one instance of R⁶ is optionally substituted alkyl. In certain embodiments, at least one instance of R⁶ is optionally substituted alkenyl. In certain embodiments, at least one instance of R⁶ is optionally substituted alkynyl. In certain embodiments, at least one instance of R⁶ is optionally substituted aryl. In certain embodiments, at least one instance of R⁶ is optionally substituted heteroaryl. In certain embodiments, at least one instance of R⁶ is optionally substituted carbocyclyl. In certain embodiments, at least one instance of R⁶ is optionally substituted heterocyclyl. In certain embodiments, at least one instance of R⁶ is optionally substituted acyl. In certain embodiments, at least one instance of R⁶ is optionally substituted sulfonyl. In certain embodiments, at least one instance of R⁶ is optionally substituted sulfinyl. In certain embodiments, at least one instance of R⁶ is —OR^{O}. In certain embodiments, at least one instance of R⁶ is —N(R^{N})₂. In certain embodiments, at least one instance of R⁶ is —SR^{S}.

In certain embodiments, at least one instance of R⁶ is optionally substituted C₁₋₆ acyl. In certain embodiments, one instance of R⁶ is optionally substituted acyl. In certain embodiments, at least one instance of R⁶ is —CO₂alkyl. In certain embodiments, at least one instance of R⁶ is —CO₂Et. In certain embodiments, at least one instance of R⁶ is —CO₂H. In certain embodiments, one instance of R⁶ is —CO₂alkyl. In certain embodiments, one instance of R⁶ is —CO₂Et. In certain embodiments, one instance of R⁶ is —CO₂H.

In certain embodiments, at least one instance of R⁶ is —N(R^{N})₂. In certain embodiments, one instance of R⁶ is —N(R^{N})₂. In certain embodiments, at least one instance of R⁶ is —NHC(O)Me. In certain embodiments, one instance of R⁶ is —NHC(O)Me.

In certain embodiments, at least one instance of R⁶ is halogen. In certain embodiments, one instance of R⁶ is halogen. In certain embodiments, at least one instance of R⁶ is —C₁. In certain embodiments, one instance of R⁶ is —C₁.

In certain embodiments, at least one instance of R⁶ is para relative to R⁵. In certain embodiments, at least one instance of R₆ is meta relative to R⁵. In certain embodiments, at least one instance of R₆ is ortho relative to R⁵. In certain embodiments, q is 1 and R⁶ is para relative to R⁵. In certain embodiments, q is 1 and R₆ is meta relative to R⁵. In certain embodiments, q is 1 and R₆ is ortho relative to R⁵.

In certain embodiments, q is 0, or an integer from 1 to 4, inclusive (i.e., 0, 1, 2, 3, or 4). In certain embodiments, q is 0. In certain embodiments, q is 1. In certain embodiments, q is 2. In certain embodiments, q is 3. In certain embodiments, q is 4.

R^{C}, R^{N}, R^{O}, and R^{S}

As defined herein, each instance of R^{C} is independently hydrogen, halogen, —CN, —N₃, —NO₂, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, optionally substituted sulfonyl, optionally substituted sulfinyl, —OR^{O}, —N(R^{N})₂, or —SR^{S}; or two R^{C} bonded to the same carbon are taken together with the intervening atoms to form optionally substituted carbocyclyl, optionally substituted heterocyclyl, or ═O. In certain embodiments, at least one instance of R^{C} is hydrogen. In certain embodiments, at least one instance of R^{C} is halogen. In certain embodiments, at least one instance of R^{C} is —CN. In certain embodiments, at least one instance of R^{C} is —N₃. In certain embodiments, at least one instance of R^{C} is —NO₂. In certain embodiments, at least one instance of R^{C} is optionally substituted alkyl. In certain embodiments, at least one instance of R^{C} is optionally substituted alkenyl. In certain embodiments, at least one instance of R^{C} is optionally substituted alkynyl. In certain embodiments, at least one instance of R^{C} is optionally substituted aryl. In certain embodiments, at least one instance of R^{C} is optionally substituted heteroaryl. In certain embodiments, at least one instance of R^{C} is optionally substituted carbocyclyl. In certain embodiments, at least one instance of R^{C} is optionally substituted heterocyclyl. In certain embodiments, at least one instance of R^{C} is optionally substituted acyl. In certain embodiments, at least one instance of R^{C} is optionally substituted sulfonyl. In certain embodiments, at least one instance of R^{C} is optionally substituted sulfinyl. In certain embodiments, at least one instance of R^{C} is —OR^{O}. In certain embodiments, at least one instance of R^{C} is —N(R^{N})₂. In certain embodiments, at least one instance of R^{C} is or —SR^{S}. In certain embodiments, two R^{C} bonded to the same carbon are taken together with the intervening atoms to form optionally substituted carbocyclyl. In certain embodiments, two R^{C} bonded to the same carbon are taken together with the intervening atoms to form optionally substituted heterocyclyl. In certain embodiments, two R^{C} bonded to the same carbon are joined together to form ═O.

In certain embodiments, each instance of R^{C} is hydrogen.

As defined herein, each instance of R^{N} is independently hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, optionally substituted sulfonyl, optionally substituted sulfinyl, or a nitrogen protecting group; or two R^{N} bonded to the same nitrogen atom are taken together with the intervening atoms to form optionally substituted heterocyclyl or optionally substituted heteroaryl. In certain embodiments, at least one instance of R^{N} is hydrogen. In certain embodiments, at least one instance of R^{N} is optionally substituted alkyl. In certain embodiments, at least one instance of R^{N} is optionally substituted alkenyl. In certain embodiments, at least one instance of R^{N} is optionally substituted alkynyl. In certain embodiments, at least one instance of R^{N} is optionally substituted aryl. In certain embodiments, at least one instance of R^{N} is optionally substituted heteroaryl. In certain embodiments, at least one instance of R^{N} is optionally substituted carbocyclyl. In certain embodiments, at least one instance of R^{N} is optionally substituted heterocyclyl. In certain embodiments, at least one instance of R^{N} is optionally substituted acyl. In certain embodiments, at least one instance of R^{N} is optionally substituted sulfonyl. In certain embodiments, at least one instance of R^{N} is optionally substituted sulfinyl. In certain embodiments, at least one instance of R^{N} is a nitrogen protecting group. In certain embodiments, two R^{N} bonded to the same nitrogen atom are taken together with the intervening atoms to form optionally substituted heterocyclyl. In certain embodiments, two R^{N} bonded to the same nitrogen atom are taken together with the intervening atoms to form optionally substituted heteroaryl.

In certain embodiments, each instance of R^{N} is hydrogen. In certain embodiments, at least one instance of R^{N} is optionally substituted C₁₋₆ alkyl. In certain embodiments, each instance of R^{N} is optionally substituted C₁₋₆ alkyl. In certain embodiments, at least one instance of R^{N} is unsubstituted C₁₋₆ alkyl. In certain embodiments, each instance of R^{N} is unsubstituted C₁₋₆ alkyl. In certain embodiments, at least one instance of R^{N} is unsubstituted C₁₋₃ alkyl. In certain embodiments, each instance of R^{N} is unsubstituted C₁₋₃ alkyl. In certain embodiments, at least one instance of R^{N} is methyl. In certain embodiments, each instance of R^{N} is methyl. In certain embodiments, each instance of R^{N} is acetyl.

As defined herein, each instance of R^{O} is independently hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, or an oxygen protecting group. In certain embodiments, at least one instance of R^{O} is hydrogen. In certain embodiments, at least one instance of R^{O} is optionally substituted alkyl. In certain embodiments, at least one instance of R^{O} is optionally substituted methyl. In certain embodiments, at least one instance of R^{O} is optionally substituted alkenyl. In certain embodiments, at least one instance of R^{O} is optionally substituted alkynyl. In certain embodiments, at least one instance of R^{O} is optionally substituted aryl. In certain embodiments, at least one instance of R^{O} is optionally substituted heteroaryl. In certain embodiments, at least one instance of R^{O} is optionally substituted carbocyclyl. In certain embodiments, at least one instance of R^{O} is optionally substituted heterocyclyl. In certain embodiments, at least one instance of R^{O} is optionally substituted acyl. In certain embodiments, at least one instance of R^{O} is optionally substituted acetyl. In certain embodiments, at least one instance of R^{O} is an oxygen protecting group.

In certain embodiments, each instance of R^{O} is hydrogen.

As defined herein, each instance of R^{S} is independently hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, or a sulfur protecting group. In certain embodiments, at least one instance of R^{S} is hydrogen. In certain embodiments, at least one instance of R^{S} is optionally substituted alkyl. In certain embodiments, at least one instance of R^{S} is optionally substituted alkenyl. In certain embodiments, at least one instance of R^{S} is optionally substituted alkynyl. In certain embodiments, at least one instance of R^{S} is optionally substituted aryl. In certain embodiments, at least one instance of R^{S} is optionally substituted heteroaryl. In certain embodiments, at least one instance of R^{S} is optionally substituted carbocyclyl. In certain embodiments, at least one instance of R^{S} is optionally substituted heterocyclyl. In certain embodiments, at least one instance of R^{S} is optionally substituted acyl. In certain embodiments, at least one instance of R^{S} is an oxygen protecting group.

In certain embodiments, each instance of R^{S} is hydrogen.

### L1, Y2, and L2

In certain embodiments,

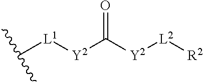

is represented by one of structures:

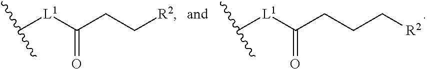

In certain embodiments,

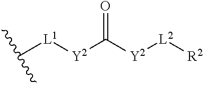

is represented by structure:

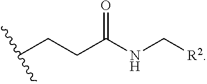

In certain embodiments,

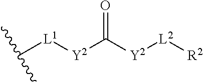

is represented by any one of structures:

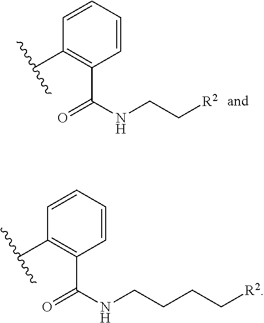

In certain embodiments,

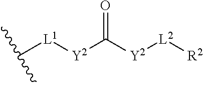

is represented by any one of the structures:

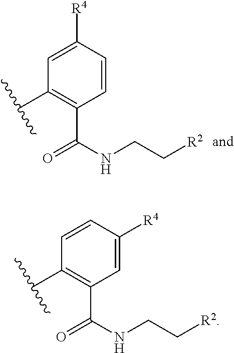

In certain embodiments,

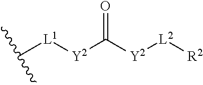

is represented by one of structures:

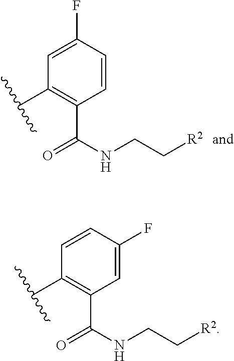

In certain embodiments,

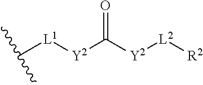

is represented by any one of structures:

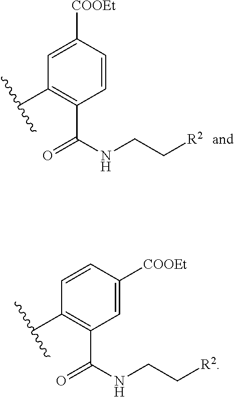

In certain embodiments,

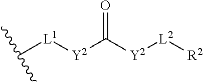

is represented by any one of structures:

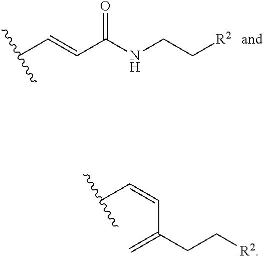

Yet other inventive compounds are represented by the following structures 1-22:

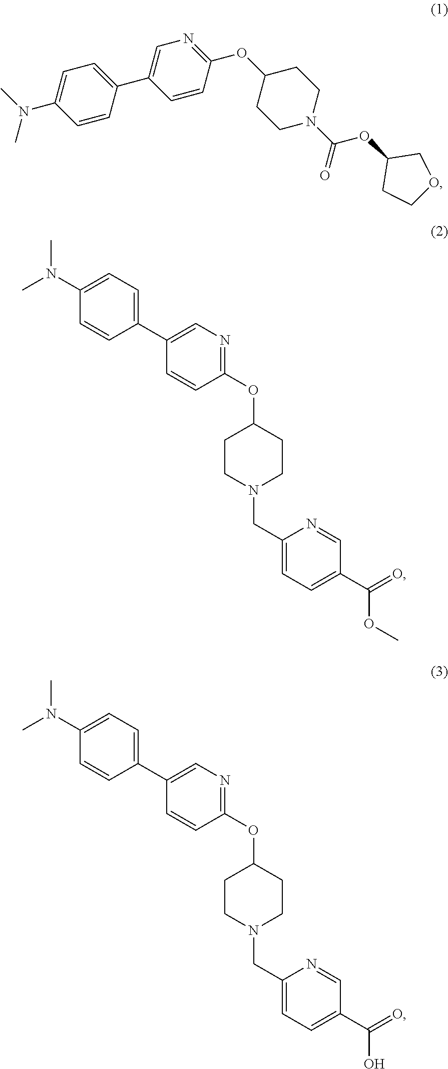

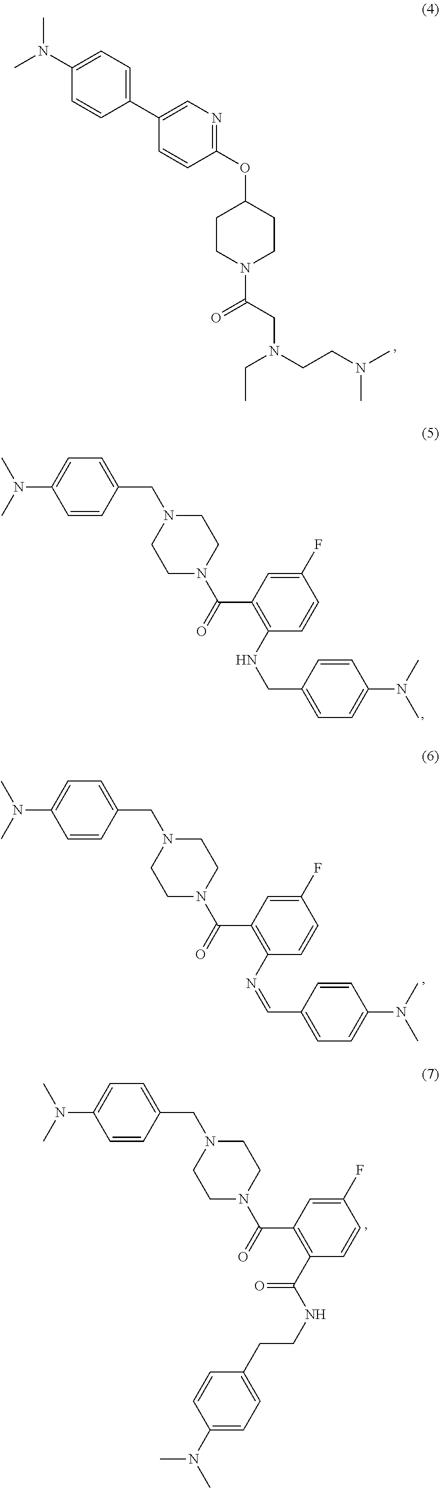

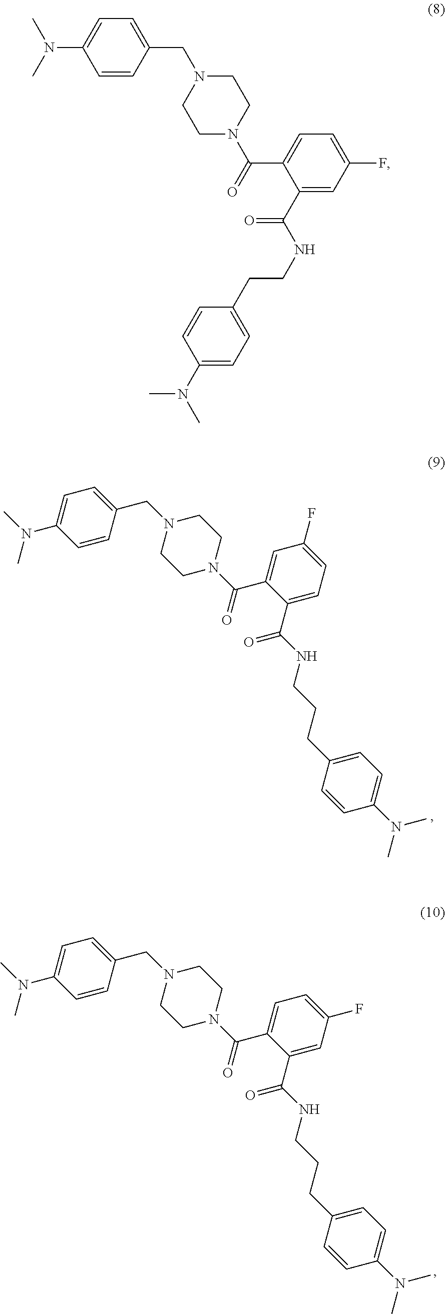

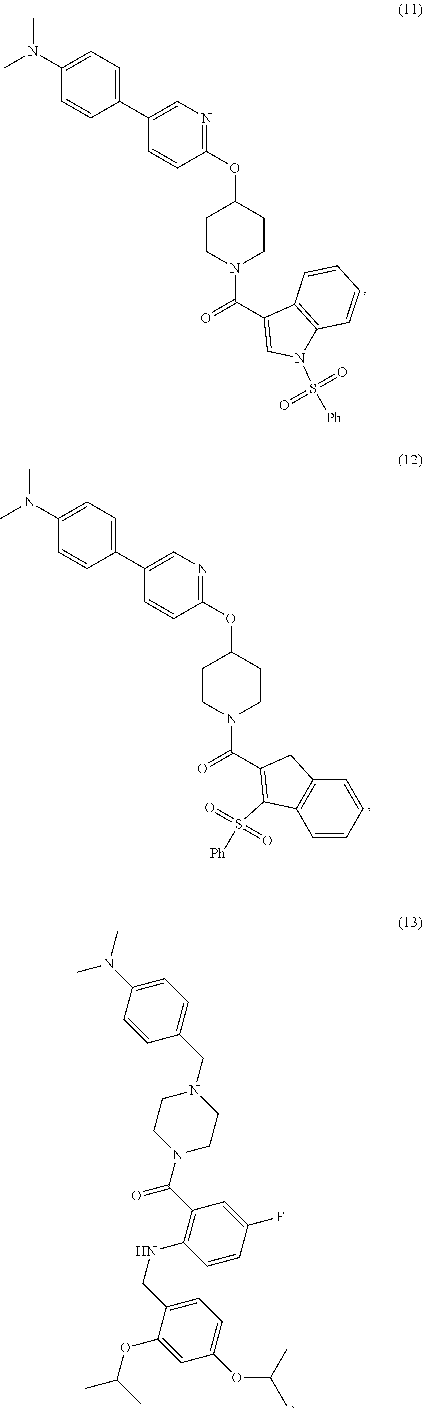 [SMILES: CN(C)c1ccc(-c2ccc(OC3CCN(C(=O)c4cn(S(=O)(=O)c5ccccc5)c5ccccc45)CC3)nc2)cc1 ; CN(C)c1ccc(-c2ccc(OC3CCN(C(=O)C4=C(S(=O)(=O)c5ccccc5)c5ccccc5C4)CC3)nc2)cc1 ; CC(C)Oc1ccc(CNc2ccc(F)cc2C(=O)N2CCN(Cc3ccc(N(C)C)cc3)CC2)c(OC(C)C)c1]

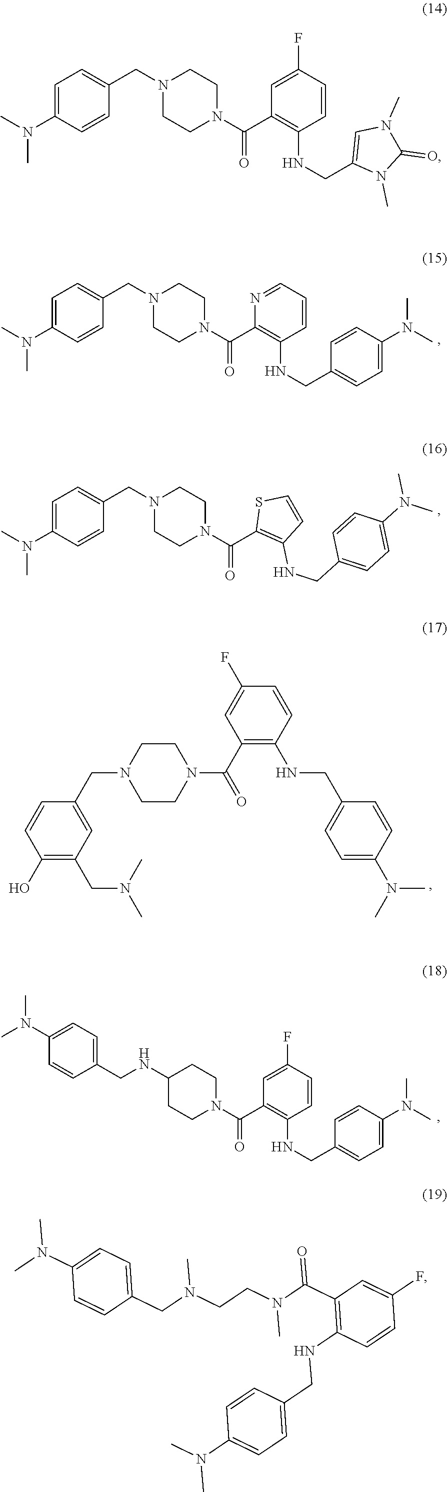

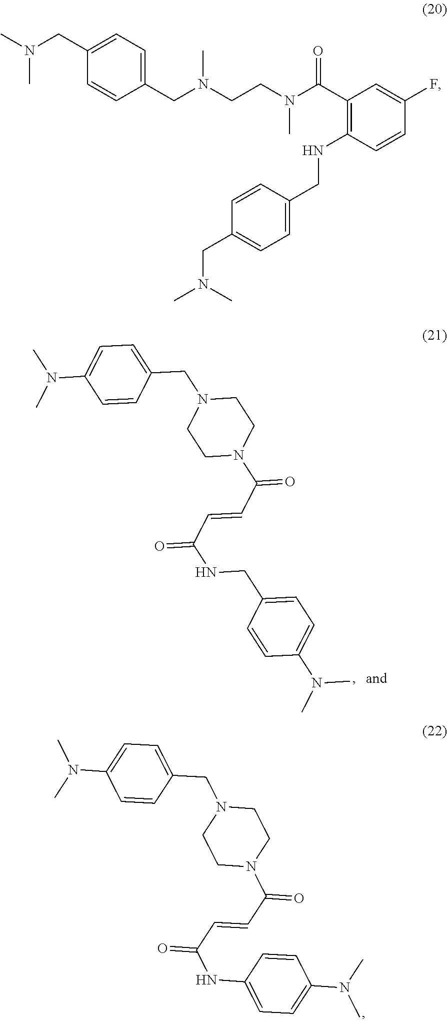

and pharmaceutically acceptable salts, hydrates, solvates, polymorphs, co-crystals, tautomers, stereoisomers, isotopically labeled derivatives, and prodrugs thereof.

### Pharmaceutical Compositions, Kits, and Administration

The present disclosure provides pharmaceutical compositions comprising a compound of Formula (I) or (II), or any one of compounds 1-22, or a pharmaceutically acceptable salt, solvate, hydrate, polymorph, co-crystal, tautomer, stereoisomer, isotopically labeled derivative, or prodrug thereof, and optionally a pharmaceutically acceptable excipient. In certain embodiments, the pharmaceutical composition described herein comprises a compound of Formula (I) or (II), or any one of compounds 1-22, or a pharmaceutically acceptable salt, solvate, hydrate, polymorph, co-crystal, tautomer, stereoisomer, isotopically labeled derivative, or prodrug thereof, and a pharmaceutically acceptable excipient.

In certain embodiments, an inventive compound, or pharmaceutically acceptable salt, solvate, hydrate, polymorph, co-crystal, tautomer, stereoisomer, isotopically labeled derivative, or prodrug thereof, is provided in an effective amount in the pharmaceutical composition. In certain embodiments, the effective amount is a therapeutically effective amount. In certain embodiments, the effective amount is an amount effective for treating a proliferative disease (e.g., cancer) in a subject in need thereof. In certain embodiments, the effective amount is an amount effective for treating a cardiovascular disease in a subject in need thereof. In certain embodiments, the effective amount is a prophylactically effective amount.

Pharmaceutical compositions described herein can be prepared by any method known in the art of pharmacology. In general, such preparatory methods include bringing an inventive compound (also referred to as the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Non-limiting exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Non-limiting exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Non-limiting exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Non-limiting exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Non-limiting exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Non-limiting exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Non-limiting exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Non-limiting exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Non-limiting exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid. Non-limiting exemplary alcohol preservatives include ethanol, polyethylene glycol, phenol, phenolic compounds, bisphenol, chlorobutanol, hydroxybenzoate, and phenylethyl alcohol.

Non-limiting exemplary acidic preservatives include vitamin A, vitamin C, vitamin E, beta-carotene, citric acid, acetic acid, dehydroacetic acid, ascorbic acid, sorbic acid, and phytic acid.

Other non-limiting exemplary preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Non-limiting exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Non-limiting exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Non-limiting exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Non-limiting exemplary synthetic oils include butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Non-limiting exemplary liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, by way of non-limiting example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants, non-limiting examples of which include as wetting agents, emulsifying and suspending agents, sweetening, flavoring, and perfuming agents. In certain embodiments for parenteral administration, the conjugates described herein are mixed with solubilizing agents, non-limiting examples of which include Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (0 absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polyethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of an inventive compound may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form. Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other ophthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Inventive compounds are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The inventive compounds and compositions can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound or pharmaceutical composition described herein is suitable for topical administration to the eye of a subject.

The exact amount of an inventive compound required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of a compound described herein. In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein. In certain embodiments, a dose described herein includes independently between 1 mg and 3 mg, inclusive, of a compound described herein. In certain embodiments, a dose described herein includes independently between 3 mg and 10 mg, inclusive, of a compound described herein. In certain embodiments, a dose described herein includes independently between 10 mg and 30 mg, inclusive, of a compound described herein. In certain embodiments, a dose described herein includes independently between 30 mg and 100 mg, inclusive, of a compound described herein.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

An inventive compound or composition, as described herein, can be administered in combination with one or more additional pharmaceutical agents (e.g., therapeutically and/or prophylactically active agents). The compounds or compositions can be administered in combination with additional pharmaceutical agents that improve their activity (e.g., activity (e.g., potency and/or efficacy) in treating a disease in a subject in need thereof, in preventing a disease in a subject in need thereof, in reducing the risk to develop a disease in a subject in need thereof), improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including an inventive compound and an additional pharmaceutical agent shows a synergistic effect that is absent in a pharmaceutical composition including one of an inventive compound and the additional pharmaceutical agent, but not both.

An inventive compound or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., a proliferative disease, e.g., cancer). Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, anti-proliferative agents, anti-cancer agents, anti-angiogenesis agents, anti-inflammatory agents, immunosuppressants, anti-bacterial agents, anti-viral agents, cardiovascular agents, cholesterol-lowering agents, anti-diabetic agents, anti-allergic agents, contraceptive agents, and pain-relieving agents. In certain embodiments, the additional pharmaceutical agent is an anti-proliferative agent. In certain embodiments, the additional pharmaceutical agent is an anti-cancer agent.

Anti-cancer agents encompass biotherapeutic anti-cancer agents as well as chemotherapeutic agents. Non-limiting exemplary biotherapeutic anti-cancer agents include, but are not limited to, interferons, cytokines (e.g., tumor necrosis factor, interferon α, interferon γ), vaccines, hematopoietic growth factors, monoclonal serotherapy, immunostimulants and/or immunodulatory agents (e.g., IL-1, 2, 4, 6, or 12), immune cell growth factors (e.g., GM-CSF) and antibodies (e.g. HERCEPTIN® (trastuzumab), T-DM1, AVASTIN (bevacizumab), ERBITUX® (cetuximab), VECTIBIX® (panitumumab), RITUXAN® (rituximab), BEXXAR® (tositumomab)).

Non-limiting exemplary chemotherapeutic agents include, but are not limited to, anti-estrogens (e.g. tamoxifen, raloxifene, and megestrol), LHRH agonists (e.g. goscrclin and leuprolide), anti-androgens (e.g. flutamide and bicalutamide), photodynamic therapies (e.g. vertoporfin (BPD-MA), phthalocyanine, photosensitizer Pc4, and demethoxy-hypocrellin A (2BA-2-DMHA)), nitrogen mustards (e.g. cyclophosphamide, ifosfamide, trofosfamide, chlorambucil, estramustine, and melphalan), nitrosoureas (e.g. carmustine (BCNU) and lomustine (CCNU)), alkylsulphonates (e.g. busulfan and treosulfan), triazenes (e.g. dacarbazine, temozolomide), platinum containing compounds (e.g. cisplatin, carboplatin, oxaliplatin), vinca alkaloids (e.g. vincristine, vinblastine, vindesine, and vinorelbine), taxoids (e.g. paclitaxel or a paclitaxel equivalent such as nanoparticle albumin-bound paclitaxel (ABRAXANE), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g. etoposide, etoposide phosphate, teniposide, topotecan, 9-aminocamptothecin, camptoirinotecan, irinotecan, crisnatol, mytomycin C), anti-metabolites, DHFR inhibitors (e.g. methotrexate, dichloromethotrexate, trimetrexate, edatrexate), IMP dehydrogenase inhibitors (e.g. mycophenolic acid, tiazofurin, ribavirin, and EICAR), ribonuclotide reductase inhibitors (e.g. hydroxyurea and deferoxamine), uracil analogs (e.g. 5-fluorouracil (5-FU), floxuridine, doxifluridine, ratitrexed, tegafur-uracil, capecitabine), cytosine analogs (e.g. cytarabine (ara C), cytosine arabinoside, and fludarabine), purine analogs (e.g. mercaptopurine and Thioguanine), Vitamin D3 analogs (e.g. EB 1089, CB 1093, and KH 1060), isoprenylation inhibitors (e.g. lovastatin), dopaminergic neurotoxins (e.g. 1-methyl-4-phenylpyridinium ion), cell cycle inhibitors (e.g. staurosporine), actinomycin (e.g. actinomycin D, dactinomycin), bleomycin (e.g. bleomycin A2, bleomycin B2, peplomycin), anthracycline (e.g. daunorubicin, doxorubicin, pegylated liposomal doxorubicin, idarubicin, epirubicin, pirarubicin, zorubicin, mitoxantrone), MDR inhibitors (e.g. verapamil), Ca²⁺ ATPase inhibitors (e.g. thapsigargin), imatinib, thalidomide, lenalidomide, tyrosine kinase inhibitors (e.g., axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (VELCADE®)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech™), SF1126 (Semafore) and OSI-027 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine.

Also encompassed by the disclosure are kits (e.g., pharmaceutical packs). The kits provided may comprise an inventive compound or a pharmaceutical composition and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form. Thus, in one aspect, provided are kits including a first container comprising a compound or pharmaceutical composition described herein. In certain embodiments, the kits are useful for treating a disease (e.g., proliferative disease, e.g., cancer) in a subject in need thereof.

In certain embodiments, a kit described herein further includes instructions for using the kit and its component(s). A kit described herein may also include information as required by a regulatory agency such as the U.S. Food and Drug Administration (FDA). In certain embodiments, the information included in the kits is prescribing information. In certain embodiments, the kits and instructions provide for treating a disease (e.g., proliferative disease, e.g., cancer) in a subject in need thereof.

### Methods of Treatment and Uses

The present disclosure also provides methods of using the inventive compounds and pharmaceutically acceptable salts, solvates, hydrates, polymorphs, co-crystals, tautomers, stereoisomers, isotopically labeled derivatives, and prodrugs thereof, and pharmaceutical compositions thereof, for the treatment and/or prevention of diseases or conditions. In certain embodiments, the disease or condition is a genetic disease, proliferative disease (e.g., cancer), a disease associated with angiogenesis, a neoplasm, inflammatory disease, autoimmune disease, liver disease, spleen disease, pulmonary disease, hematological disease, neurological disease, painful condition, psychiatric disorder, cardiovascular disease, or a metabolic disorder (e.g., a diabetic condition). In certain embodiments, the disease or condition is associated with overexpression and/or aberrant activity of a KDM (e.g., KDM3). In certain embodiments, the disease or condition is associated with overexpression of a KDM (e.g., KDM3). In certain embodiments, the disease or condition is associated with aberrant activity (e.g., increased activity) of a KDM (e.g., KDM3). In certain embodiments, the KDM is KDM3. In certain embodiments, the KDM is one or more of KDM2/7, KDM3, KDM4, KDM5, or KDM6. In certain embodiments, the KDM is one or more of KDM5A, KDM5B, or KDM5C. In certain embodiments, the KDM is one or more of KDM3A, KDM3B, or Jumonji domain containing 1C (JMJD1C).

Provided herein are methods for treating a proliferative disease (e.g., cancer) in a subject, the methods comprising administering to the subject a compound of Formula (I) or (II), or any one of compounds 1-22, or a pharmaceutically acceptable salt, solvate, hydrate, polymorph, co-crystal, tautomer, stereoisomer, isotopically labeled derivative, or prodrug thereof, or a pharmaceutical composition thereof. Also provided herein are compounds of Formulas (I) and (II), and compounds 1-22, and pharmaceutically acceptable salts, solvates, hydrates, polymorphs, co-crystals, tautomers, stereoisomers, isotopically labeled derivatives, and prodrugs thereof, or a pharmaceutical compositions thereof, for use in treating proliferative diseases (e.g., cancer) in a subject. Furthermore, also provided herein are uses of the compounds of Formulas (I) and (II), and compounds 1-22, and pharmaceutically acceptable salts, solvates, hydrates, polymorphs, co-crystals, tautomers, stereoisomers, isotopically labeled derivatives, and prodrugs thereof, or a pharmaceutical compositions thereof, for the manufacture of medicaments for treating proliferative diseases (e.g., cancer). In certain embodiments, the proliferative disease is a proliferative disease associated with overexpression and/or aberrant activity (e.g., increased activity) of a KDM (e.g., KDM3). In certain embodiments, the proliferative disease is a proliferative disease associated with the overexpression of a KDM (e.g., KDM3). In certain embodiments, the proliferative disease is a proliferative disease associated with aberrant activity (e.g., increased activity) of a KDM (e.g., KDM3).

In certain embodiments, the proliferative disease is cancer. In certain embodiments, the cancer is lung cancer, breast cancer, liver cancer, pancreatic cancer, gastric cancer, ovarian cancer, colon cancer, or colorectal cancer. In certain embodiments, the cancer is a carcinoma. In certain embodiments, the carcinoma is a carcinoma of the breast, liver, lung, pancreas, stomach, colon, or prostate. The cancer may be any other cancer described herein. In certain embodiments, the cancer is associated with overexpression and/or aberrant activity of a KDM (e.g., KDM3). In certain embodiments, the cancer associated with overexpression of a KDM (e.g., KDM3). In certain embodiments, the cancer is associated with aberrant activity (e.g., increased activity) of a KDM (e.g., KDM3 and/or KDM5).

Provided herein are methods for treating a cardiovascular disease in a subject, the methods comprising administering to the subject a compound of Formula (I) or (II), or any one of compounds 1-22, or a pharmaceutically acceptable salt, solvate, hydrate, polymorph, co-crystal, tautomer, stereoisomer, isotopically labeled derivative, or prodrug thereof, or a pharmaceutical composition thereof. Also provided herein are compounds of Formulas (I) and (II) and compounds 1-22, and pharmaceutically acceptable salts, solvates, hydrates, polymorphs, co-crystals, tautomers, stereoisomers, isotopically labeled derivatives, and prodrugs thereof, or a pharmaceutical compositions thereof, for use in treating cardiovascular diseases in a subject. Furthermore, also provided herein are uses of the compounds of Formulas (I) and (II), and compounds 1-22, and pharmaceutically acceptable salts, solvates, hydrates, polymorphs, co-crystals, tautomers, stereoisomers, isotopically labeled derivatives, and prodrugs thereof, or a pharmaceutical compositions thereof, for the manufacture of medicaments for treating cardiovascular diseases. In certain embodiments, the cardiovascular disease is associated with overexpression and/or aberrant activity of a KDM (e.g., KDM3). In certain embodiments, the cardiovascular disease is associated with overexpression of a KDM (e.g., KDM3). In certain embodiments, the cardiovascular disease is associated with aberrant activity (e.g., increased activity) of a KDM (e.g., KDM3).

In certain embodiments, the methods described herein comprise administering to a subject a therapeutically effective amount of a compound of Formula (I) or (II), or any one of compounds 1-22, or a pharmaceutically acceptable salt, solvate, hydrate, polymorph, co-crystal, tautomer, stereoisomer, isotopically labeled derivative, or prodrug thereof, or a pharmaceutical composition thereof. In certain embodiments, a therapeutically effective amount is an amount sufficient for treating a disease (e.g., proliferative disease (e.g., cancer) or cardiovascular disease). In certain embodiments, a therapeutically effective amount is an amount sufficient for treating a proliferative disease (e.g., cancer). In certain embodiments, a therapeutically effective amount is an amount sufficient for inhibiting the activity of a KDM (e.g., KDM3).

In certain embodiments, the methods described herein comprise administering to a subject a prophylactically effective amount compound of Formula (I) or (II), or any one of compounds 1-22, or a pharmaceutically acceptable salt, solvate, hydrate, polymorph, co-crystal, tautomer, stereoisomer, isotopically labeled derivative, or prodrug thereof, or a pharmaceutical composition thereof. In certain embodiments, a prophylactically effective amount is an amount sufficient for preventing a disease (e.g., proliferative disease or cardiovascular disease). In certain embodiments, a prophylactically effective amount is an amount sufficient for preventing the recurrence of cancer in a subject. In certain embodiments, a prophylactically effective amount is an amount sufficient for inhibiting the activity of KDM (e.g., KDM3).

Also provided herein are methods of inhibiting the activity of a KDM (e.g., KDM3) comprising contacting the KDM enzyme with a compound of Formula (I) or (II), or any one of compounds 1-22, or a pharmaceutically acceptable salt, solvate, hydrate, polymorph, co-crystal, tautomer, stereoisomer, isotopically labeled derivative, or prodrug thereof, or a pharmaceutical composition thereof. Also provided herein are compounds of Formulas (I) and (II), and compounds 1-22, and pharmaceutically acceptable salts, solvates, hydrates, polymorphs, co-crystals, tautomers, stereoisomers, isotopically labeled derivatives, and prodrugs thereof, and pharmaceutical compositions thereof, for use in inhibiting the activity of a KDM (e.g., KDM3). Furthermore, also provided herein compounds of Formulas (I) and (II), and compounds 1-22, and pharmaceutically acceptable salts, solvates, hydrates, polymorphs, co-crystals, tautomers, stereoisomers, isotopically labeled derivatives, and prodrugs thereof, and pharmaceutical compositions thereof, for use in the manufacture of medicaments for inhibiting the activity of a KDM (e.g., KDM3). In certain embodiments, the inhibiting is in a subject, and the method or use comprises administering to the subject a compound of Formula (I) or (II), or any one of compounds 1-22, or a pharmaceutically acceptable salt, solvate, hydrate, polymorph, co-crystal, tautomer, stereoisomer, isotopically labeled derivative, or prodrug thereof, or a pharmaceutical composition thereof. In certain embodiments, the inhibiting is in a biological sample, and the method or use comprises contacting the biological sample with a compound of Formula (I) or (II), or any one of compounds 1-22, or a pharmaceutically acceptable salt, solvate, hydrate, polymorph, co-crystal, tautomer, stereoisomer, isotopically labeled derivative, or prodrug thereof, or a pharmaceutical composition thereof.

In certain embodiments, the KDM is a KDM1, KDM2, KDM3, KDM4, KDM5, or KDM6. In certain embodiments, the KDM is a KDM3. In certain embodiments, the KDM3 is KDM3A, KDM3B, or JMJD1C. In certain embodiments, the KDM3 is KDM3A. In certain embodiments, the KDM3 is KDM3B. In certain embodiments, the KDM3 is JMJD1C.

In certain embodiments, an inventive compound or pharmaceutical composition selectively inhibits one KDM over others. In certain embodiments, a compound or pharmaceutical composition described herein selectively inhibits KDM3. The selectivity of an inventive compound or pharmaceutical composition in inhibiting the activity of a KDM over a different histone demethylase (e.g., a different KDM) may be measured by the quotient of the IC₅₀ value of the compound or pharmaceutical composition in inhibiting the activity of the different histone demethylase over the IC₅₀ value of the compound or pharmaceutical composition in inhibiting the activity of the histone demethylase. The selectivity of a compound or pharmaceutical composition described herein for a histone demethylase (e.g., KDM (e.g., KDM3)) over a different histone demethylase may also be measured by the quotient of the K_{d} value of an adduct of the inventive compound or pharmaceutical composition and the different protein over the K_{d} value of an adduct of the compound or pharmaceutical composition and the histone demethylase. In certain embodiments, the selectivity is at least 2-fold, at least 3-fold, at least 5-fold, at least 10-fold, at least 30-fold, at least 50-fold, at least 100-fold, at least 300-fold, at least 500-fold, at least 1,000-fold, at least 3,000-fold, at least 5,000-fold, at least 10,000-fold, at least 30,000-fold, at least 50,000-fold, or at least 100,000-fold. In certain embodiments, the selectivity is not more than 100,000-fold, not more than 10,000-fold, not more than 1,000-fold, not more than 100-fold, not more than 10-fold, or not more than 2-fold. Combinations of the above-referenced ranges (e.g., at least 2-fold and not more than 10,000-fold) are also within the scope of the disclosure.

Also provided herein are methods of inducing apoptosis in a cell, the methods comprising contacting the cell with a compound of Formula (I) or (II), or any one of compounds 1-22, or a pharmaceutically acceptable salt, solvate, hydrate, polymorph, co-crystal, tautomer, stereoisomer, isotopically labeled derivative, or prodrug thereof, or a pharmaceutical composition thereof. Also provided herein are compounds of Formulas (I) and (II), and compounds 1-22, and pharmaceutically acceptable salts, solvates, hydrates, polymorphs, co-crystals, tautomers, stereoisomers, isotopically labeled derivatives, and prodrugs thereof, and pharmaceutical compositions thereof, for use in inducing apoptosis in a cell. Furthermore, also provided herein are uses of compounds of Formulas (I) and (II), and compounds 1-22, and pharmaceutically acceptable salts, solvates, hydrates, polymorphs, co-crystals, tautomers, stereoisomers, isotopically labeled derivatives, and prodrugs thereof, and pharmaceutical compositions thereof, for the manufacture of medicaments for inducing apoptosis in a cell. In certain embodiments, the cell is in a subject, and the method or use comprises administering to the subject a compound of Formula (I) or (II), or any one of compounds 1-22, or a pharmaceutically acceptable salt, solvate, hydrate, polymorph, co-crystal, tautomer, stereoisomer, isotopically labeled derivative, or prodrug thereof, or a pharmaceutical composition thereof. In certain embodiments, the cell is in a biological sample, and the method or use comprises contacting the biological sample with a compound of Formula (I) or (II), or any one of compounds 1-22, or a pharmaceutically acceptable salt, solvate, hydrate, polymorph, co-crystal, tautomer, stereoisomer, isotopically labeled derivative, or prodrug thereof, or a pharmaceutical composition thereof.

In certain embodiments, the provided methods and uses comprise contacting a cell with an effective amount of an inventive compound, or salt, solvate, hydrate, polymorph, co-crystal, tautomer, stereoisomer, isotopically labeled derivative, or prodrug thereof, or a pharmaceutical composition, as described herein. The cell may be contacted in vitro or in vivo. In certain embodiments, the contacting is in vivo. In certain embodiments, the contacting is in vitro. In certain embodiments, the cell is a cancer cell.

### EXAMPLES

These and other aspects of the present disclosure will be further appreciated upon consideration of the following Examples, which are intended to illustrate certain particular embodiments of the present disclosure but are not intended to limit its scope, as defined by the claims.

### Example 1: KDM3 Alphascreen Assays

AlphaScreen assays were performed in 384-well plate format using white AlphaPlate™ (PerkinElmer®, USA), and transfer of pre-diluted compound (100 nL) was performed using a Janus® Workstation (PerkinElmer®, USA). All subsequent steps were carried out in assay buffer (50 mM 4-(2-hydroxyethyl)-1-piperazineethanesulfonic acid (HEPES), pH 7.5, 0.1% (wt/vol) bovine serum albumin (BSA) and 0.01% (vol/vol) Tween®20). In brief, 10 μL of assay buffer containing demethylase enzyme (2 nM Final) was pre-incubated for 15 min with dilutions of compound. The enzyme reaction was initiated by the addition of substrate (5 μL) consisting of L-ascorbic acid (100 μM Final), 2-oleoylglycerol (OG) (5 μM Final), FAS (10 μM Final) and histone H3(1-21)K9Me3-GGK Biotin (100 nM Final). The enzyme reaction was allowed to proceed for 30 minutes and was stopped by the addition of 5 μL of assay buffer containing ethylenediaminetetraacetic acid (EDTA) (40 mM) and NaCl (1,200 mM). The final concentration of DMSO was 1%. Streptavidin donor beads (0.08 mg/ml) and protein-A-conjugated acceptor beads (0.08 mg/ml) were preincubated for 1 h with antibody to methyl mark (300 ng/mL Final), and the presence of histone H3 product methyl mark was detected using the preincubated AlphaScreen™ beads (5 μL). Detection was allowed to proceed for 2 h at room temperature, and the assay plates were read on the EnVision® 2104 plate reader. Data were normalized to the (no-enzyme) control, and the IC₅₀ values were determined via nonlinear regression curve fit using GraphPad Prism 7.

Results from KDM3B AlphaScreen™ assays are shown in Table 1 below. Data are expressed as IC₅₀ (μM). 2,4-pyridinedicarboxylic acid (PDCA), shown below, was used as a standard:

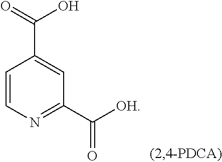 [SMILES: O=C(O)c1ccnc(C(=O)O)c1]

TABLE 1
KDM3B AlphaScreen ™ Assays

| | |
|---|---|
| Compound | IC₅₀ (μM) |

ASSAY 1

| | |
|---|---|
| 2,4 PDCA | 6.02 |
| JADA69 | 27.93 |
| JADA84 | 52.99 |
| JADA92 | 8.78 |
| JADA94 (mixture of JADA94-1 and JADA94-2) | 3.54 |

ASSAY 2

| | |
|---|---|
| 2,4 PDCA | 8.22 |
| JADA 92 | 13.69 |
| JADA94 (mixture of JADA94-1 and JADA94-2) | 8.03 |

ASSAY 3

| | |
|---|---|
| JADA133 | 18.56 |
| JADA142 (mixture of JADA142-1 and JADA142-2) | 40.39 |
| JADA143 (mixture of JADA143-1 and JADA143-2) | 38.23 |
| JADA144 (mixture of JADA144-1 and JADA144-2) | 10 |
| JADA146 (mixture of JADA146-1 and JADA146-2) | 12.23 |
| JADA147 (mixture of JADA147-1 and JADA 147-2) | 113.1 |
| JADA148 (mixture of JADA148-1 and JADA148-2) | 8.87 |
| JADA149 (mixture of JADA149-1 and JADA 149-2) | 8.77 |
| 2,4 PDCA | 4.54 |

ASSAY 4

| | |
|---|---|
| KDM3-16 (mixture of KDM3-16-1 and KDM3-16-2) | 30.08 |
| KDM3-17 (mixture of KDM3-17-1 and KDM3-17-2) | 39.2 |
| 2,4 PDCA | 4.45 |

ASSAY 5

| | |
|---|---|
| KDM3-18 (mixture of KDM3-18-1 and KDM3-18-2) | 15.75 |
| KDM3-19 (mixture of KDM3-19-1 and KDM3-19-2) | 3.4 |
| KDM3-23 | 4.78 |
| KDM3-24 | 10.86 |
| JADA 94 (mixture of JADA94-1 and JADA94-2) | 3.53 |
| 2,4 PDCA | 6.23 |

ASSAY 6

| | |
|---|---|
| KDM3-26 | 74.32 |
| KDM3-27 | 11.31 |
| KDM3-31 | 9.43 |
| 2,4 PDCA | 1.87 |

ASSAY 7

| | |
|---|---|
| KDM3-31 | 8.26 |
| KDM3-38 | 5.65 |
| KDM3-39 | 10.92 |
| JADA92 | 9.79 |
| JADA94 (mixture of JADA94-1 and JADA94-2) | 5.52 |
| JADA144 (mixture of JADA144-1 and JADA144-2) | 8.57 |
| 2,4 PDCA | 3.2 |

ASSAY 8

| | |
|---|---|
| KDM3-31 | 10.81 |
| KDM3-39 | 19.23 |
| KDM3-40 | 22.41 |
| KDM3-41 | 49.39 |
| KDM3-42 | 11.78 |
| KDM3-43 | 61.01 |
| KDM3-44 | 91.31 |
| JADA94 (mixture of JADA94-1 and JADA94-2) | 6.96 |
| 2,4 PDCA | 5.22 |

The data illustrated in Table 1 show that inventive compounds, including JADA94 (mixture of JADA94-1 and JADA94-2), KDM3-19 (mixture of KDM3-19-1 and KDM3-19-2), and KDM3-23, are potent KDM3B inhibitors. Inventive compounds JADA94, KDM3-19 and KDM3-23 showed better IC₅₀ than control compound 2,4, PDCA, indicating that they are strong KDM3 binders. Structure activity relationship (SAR) analysis results are also illustrated in Table 1.

TABLE 2
KDM3 AlphaScreen ™ Assays
ASSAY 9

| Compound | IC₅₀ (μM) |
|---|---|
| JADA196 | 15.97 |
| JADA201 | 24.34 |
| 5 | 7.65 |
| 11 | 33.59 |
| KDM3-47 | 12.98 |
| KDM3-51 | 13.99 |
| KDM3-53 | 20.02 |
| KDM3-56 | 8.83 |
| KDM3-59 | 23.63 |
| KDM3-60 | 34.96 |
| JADA94 (mixture of | 21.34 |
| JADA94-1 and JADA94-2) | |
| 2,4 PDCA | 13.82 |

The data illustrated in Table 2 show that inventive compounds, including compounds 5, 11, and KDM3-56, are potent KDM3A inhibitors. Inventive compounds 5, 11, and KDM3-56 showed better IC₅₀ than control compound 2,4, PDCA, indicating that they are strong KDM3 binders. Structure activity relationship (SAR) analysis results are also illustrated in Table 2.

### Example 2: Synthesis of Compounds

The compounds provided herein can be prepared from readily available starting materials using the following general methods and procedures or methods known in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by those skilled in the art by routine optimization procedures.

Non-limiting exemplary synthetic routes to compounds described herein are shown, below.

### Example 2-1: Synthesis of 3-(4-(dimethylamino)phenyl)-N-(2-(4-((5-(4-(dimethylamino)phenyl)pyridin-2-yl)oxy)piperidine-1-carbonyl)-4-fluorophenyl)propanamide (KDM3-23)

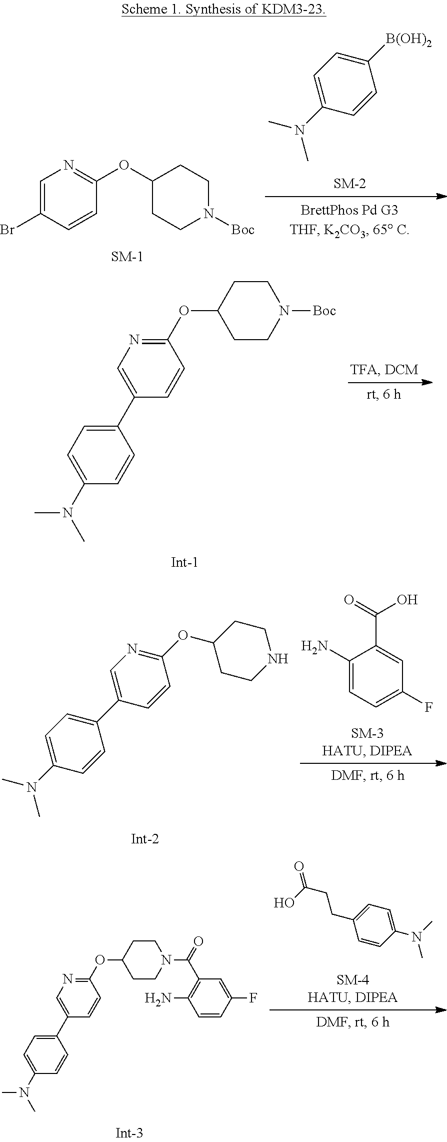

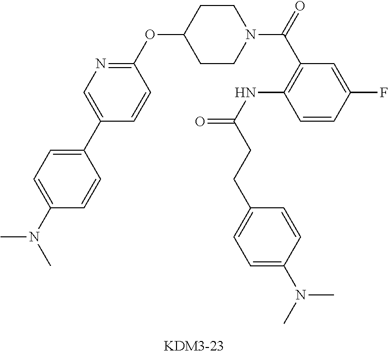 [SMILES: CN(C)c1ccc(CCC(=O)Nc2ccc(F)cc2C(=O)N2CCC(Oc3ccc(-c4ccc(N(C)C)cc4)cn3)CC2)cc1]

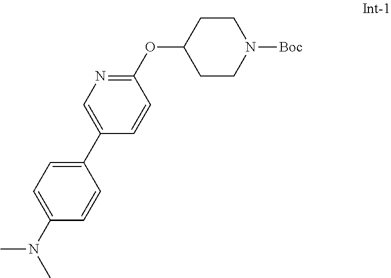 [SMILES: CN(C)c1ccc(-c2ccc(OC3CCN(C(=O)OC(C)(C)C)CC3)nc2)cc1]

A mixture of SM-1 (356 mg, 1.00 mmol), SM-2 (181 mg, 1.1 mmol), BrettPhos Pd G3 catalyst (93 mg, 0.1 mmol), and K₂CO₃ (345 mg, 2.5 mmol) in THF (3 mL) was stirred in a 10-mL flask under N₂ atmosphere at 65° C. for 16 hours (h). After completion of the reaction, the mixture was filtered, and the filtrate was concentrated under reduced pressure. The resulting residue was purified via ISCO chromatography (DCM/MeOH=20/1) to give the Int-1 (210 mg, 53% yield).

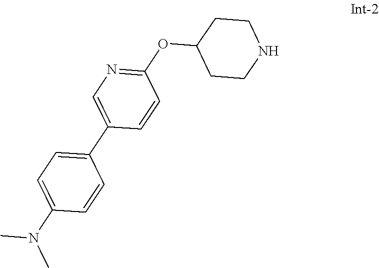 [SMILES: CN(C)c1ccc(-c2ccc(OC3CCNCC3)nc2)cc1]

A solution of Int-1 (210 mg, 0.52 mmol) in DCM (5 mL) and TFA (1.5 mL) in a 10-mL flask was stirred at 25° C. for 6 h. After completion of the reaction, the mixture was concentrated under reduced pressure. The resulting residue was purified via ISCO chromatography (regular 12 g column, DCM/MeOH=10/1) to give the Int-2 (135 mg, 87% yield).

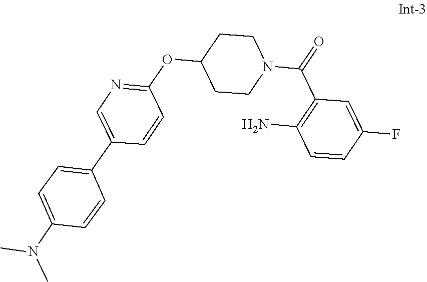 [SMILES: CN(C)c1ccc(-c2ccc(OC3CCN(C(=O)c4cc(F)ccc4N)CC3)nc2)cc1]

To a solution of Int-2 (29.7 mg, 0.1 mmol) and SM-3 (17 mg, 0.11 mmol) in DMF (1 mL), DIPEA (64.5 mg, 0.5 mmol) and HATU (46.6 mg, 0.12 mmol) were added. The reaction was stirred at 25° C. for 6 h prior to dilution with EtOAc (3 mL). The mixture was washed with H₂O (3 mL), and the aqueous phase was extracted with additional EtOAc (2×2 mL). The combined organic phases were concentrated under reduced pressure. The resulting residue was purified by ISCO (DCM/MeOH=100/0-80/20) to give Int-3 (23 mg, 55% yield).

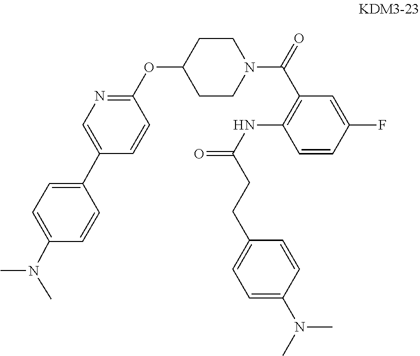 [SMILES: CN(C)c1ccc(CCC(=O)Nc2ccc(F)cc2C(=O)N2CCC(Oc3ccc(-c4ccc(N(C)C)cc4)cn3)CC2)cc1]

To a solution of Int-3 (23 mg, 0.05 mmol) and SM-4 (10.5 mg, 0.06 mmol) in DMF (0.5 mL), N,N-diisopropylethylamine (DIPEA) (27.8 mg, 0.2 mmol) and 1-[bis (dimethylamino)methylene]-1H-1,2,3-triazolo[4,5-b]pyridinium 3-oxid hexafluorophosphate) (HATU) (24 mg, 0.06 mmol) were added. The reaction was stirred at 25° C. for 6 h prior to dilution with EtOAc (2 mL). The mixture was washed with H₂O (2 mL), and the aqueous phase was extracted with additional EtOAc (2×2 mL). The combined organic phases were concentrated under reduced pressure. The resulting residue was purified by ISCO (DCM/MeOH=100/0-90/10) to give KDM3-23 (12 mg, 40% yield).

### Example 2-2: Synthesis of 3-(4-(dimethylamino)phenyl)-N-(2-(4-((5-(4-(dimethylamino)phenyl)pyridin-2-yl)oxy)piperidine-1-carbonyl)-5-fluorophenyl)propanamide (KDM3-51

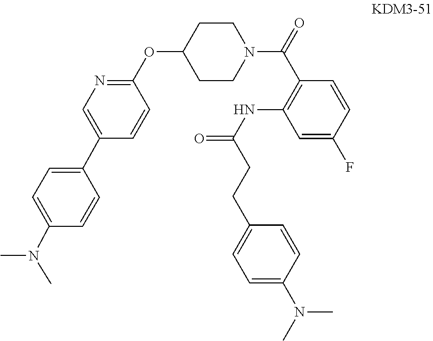 [SMILES: CN(C)c1ccc(CCC(=O)Nc2cc(F)ccc2C(=O)N2CCC(Oc3ccc(-c4ccc(N(C)C)cc4)cn3)CC2)cc1]

KDM3-51 was prepared in an analogous manner to KDM3-23 in Example 2-1 (11.6 mg, 37% yield).

### Example 2-3: Synthesis of methyl 4-(3-(4-(dimethylamino)phenyl)propanamido)-3-(4-((5-(4-(dimethylamino)phenyl)pyridin-2-yl)oxy)piperidine-1-carbonyl)benzoate (KDM3-52)

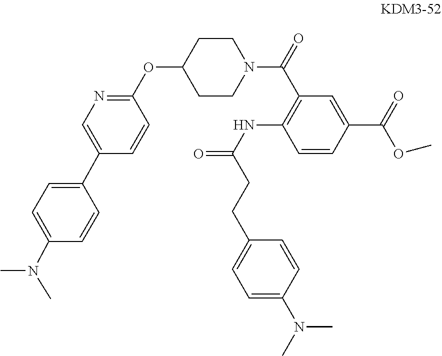

KDM3-52 was prepared in an analogous manner to KDM3-23 in Example 2-1 (11 mg, 35% yield).

### Example 2-4: 3-(4-(dimethylamino)phenyl)-N-(2-(4-((5-(4-(dimethylamino)phenyl)pyridin-2-yl)oxy)piperidine-1-carbonyl)-4-(trifluoromethyl)phenyl)propanamide (KDM3-53)

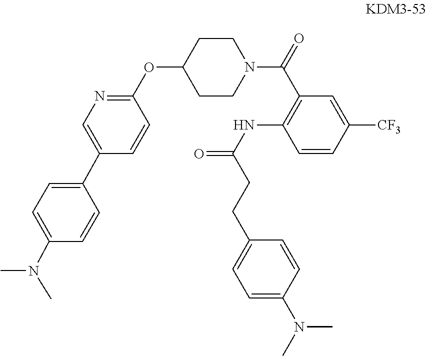 [SMILES: CN(C)c1ccc(CCC(=O)Nc2ccc(C(F)(F)F)cc2C(=O)N2CCC(Oc3ccc(-c4ccc(N(C)C)cc4)cn3)CC2)cc1]

KDM3-53 was prepared in an analogous manner to KDM3-23 in Example 2-1 (10 mg, 30% yield).

### Example 2-5: Synthesis of 3-(4-(dimethylamino)phenyl)-N-(2-(4-((5-(4-(dimethylamino)phenyl)pyridin-2-yl)oxy)piperidine-1-carbonyl)-4-(trifluoromethoxy)phenyl)propanamide (KDM3-59)

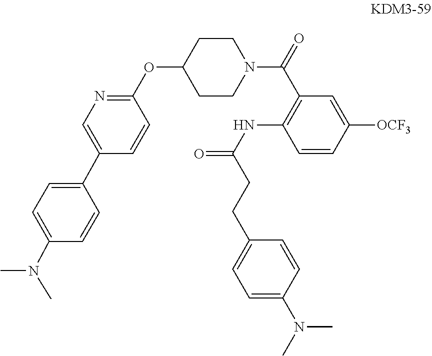 [SMILES: CN(C)c1ccc(CCC(=O)Nc2ccc(OC(F)(F)F)cc2C(=O)N2CCC(Oc3ccc(-c4ccc(N(C)C)cc4)cn3)CC2)cc1]

KDM3-59 was prepared in an analogous manner to KDM3-23 in Example 2-1 (7.3 mg, 18% yield).

### Example 2-6: Synthesis of 3-(4-(dimethylamino)phenyl)-N-(3-(4-((5-(4-(dimethylamino)phenyl)pyridin-2-yl)oxy)piperidine-1-carbonyl)naphthalen-2-yl)propanamide (KDM3-69)

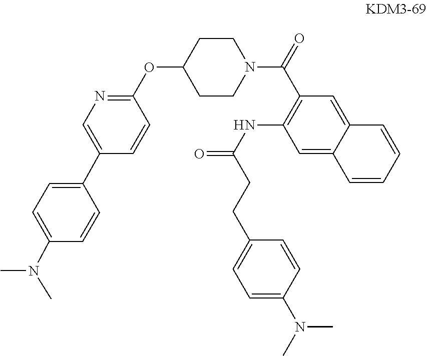 [SMILES: CN(C)c1ccc(CCC(=O)Nc2cc3ccccc3cc2C(=O)N2CCC(Oc3ccc(-c4ccc(N(C)C)cc4)cn3)CC2)cc1]

KDM3-69 was prepared in an analogous manner to KDM3-23 in Example 2-1 (5.3 mg, 11% yield).

### Example 2-7: Synthesis of 3-(4-(dimethylamino)phenyl)-N-(2-(4-((5-(4-(dimethylamino)phenyl)pyridin-2-yl)oxy)piperidine-1-carbonyl)pyridin-3-yl)propanamide (KDM3-76)

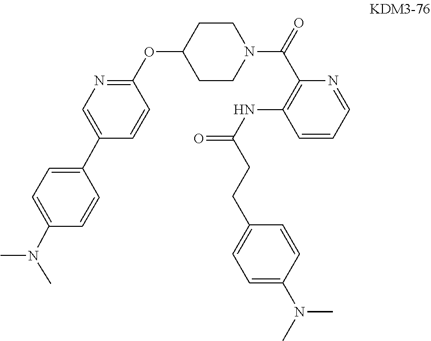 [SMILES: CN(C)c1ccc(CCC(=O)Nc2cccnc2C(=O)N2CCC(Oc3ccc(-c4ccc(N(C)C)cc4)cn3)CC2)cc1]

KDM3-76 was prepared in an analogous manner to KDM3-23 in Example 2-1 (6.0 mg, 10% yield).

### Example 2-8: Synthesis of N-(4-(dimethylamino)phenethyl)-2-(4-((5-(4-(dimethylamino)phenyl)pyridin-2-yl)oxy)piperidine-1-carbonyl)-4-fluorobenzamide (JADA94-1) and N-(4-(dimethylamino)phenethyl)-2-(4-((5-(4-(dimethylamino)phenyl)pyridin-2-yl)oxy)piperidine-1-carbonyl)-5-fluorobenzamide (JADA94-2)

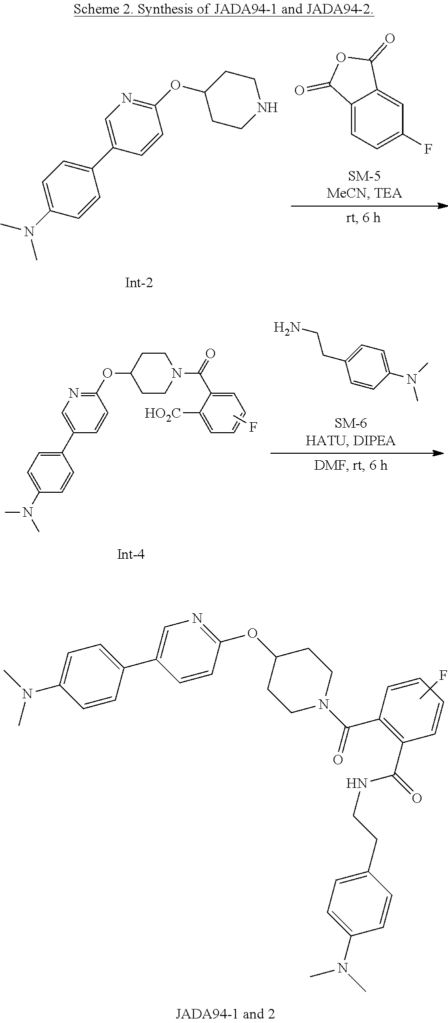

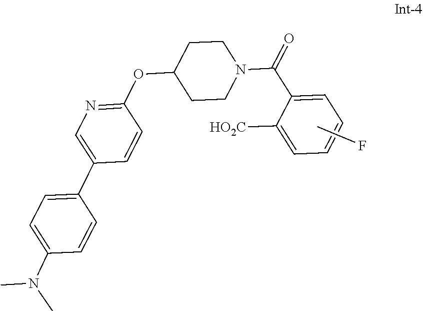

To a solution of Int-2 (29.7 mg, 0.1 mmol) and SM-5 (17 mg, 0.11 mmol) in MeCN (1 mL), TEA (50.5 mg, 0.5 mmol) were added. The reaction was stirred at 25° C. for 6 h prior to concentration of crude mixture under reduced pressure. The resulting residue was purified by ISCO (DCM/MeOH=100/0-80/20) to give Int-4 (35 mg, 75% yield).

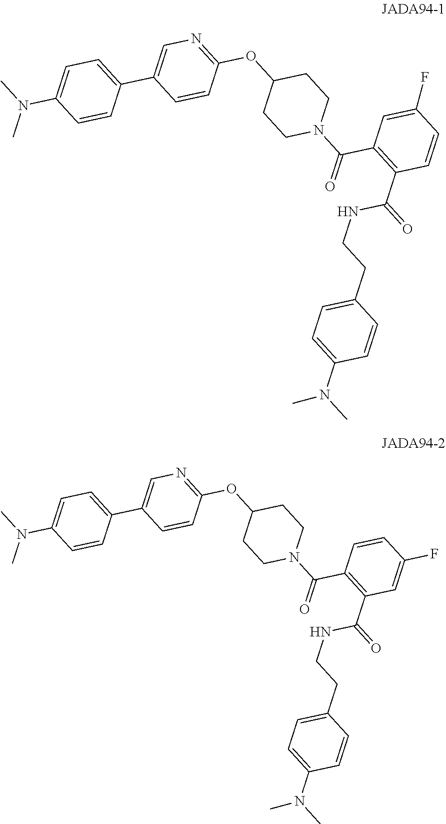 [SMILES: CN(C)c1ccc(CCNC(=O)c2ccc(F)cc2C(=O)N2CCC(Oc3ccc(-c4ccc(N(C)C)cc4)cn3)CC2)cc1 ; CN(C)c1ccc(CCNC(=O)c2cc(F)ccc2C(=O)N2CCC(Oc3ccc(-c4ccc(N(C)C)cc4)cn3)CC2)cc1]

To a solution of Int-4 (23 mg, 0.05 mmol) and SM-6 (9.8 mg, 0.06 mmol) in DMF (0.5 mL), DIPEA (27.8 mg, 0.2 mmol) and HATU (24 mg, 0.06 mmol) were added. The reaction was stirred at 25° C. for 6 h prior to dilution with EtOAc (2 mL). The mixture was washed with H₂O (2 mL), and the aqueous phase was extracted with additional EtOAc (2×2 mL). The combined organic phases were concentrated under reduced pressure. The resulting residue was purified by ISCO (DCM/MeOH=100/0-90/10) to give a mixture of JADA94-1 and JADA94-2 (10.6 mg, 35% yield).

### Example 2-9: Synthesis of N-(4-(dimethylamino)phenethyl)-2-(4-((5-(4-(dimethylcarbamoyl)phenyl)pyridin-2-yl)oxy)piperidine-1-carbonyl)-4-fluorobenzamide (JADA142-1) and N-(4-(dimethylamino)phenethyl)-2-(4-((5-(4-(dimethylcarbamoyl)phenyl)pyridin-2-yl)oxy)piperidine-1-carbonyl)-5-fluorobenzamide (JADA142-2)

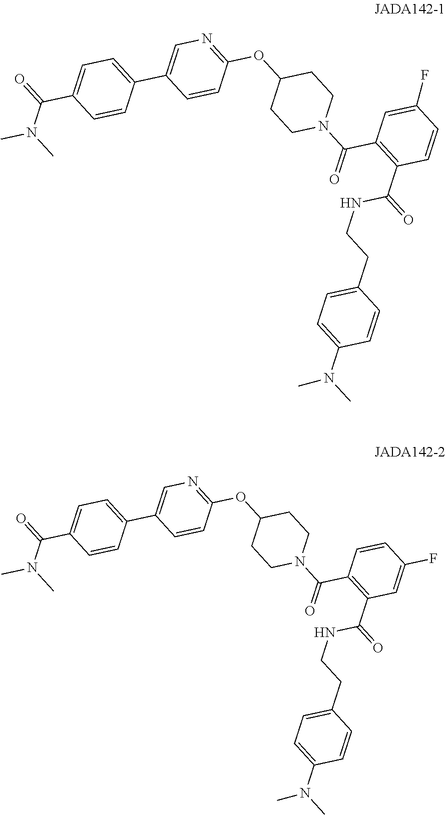 [SMILES: CN(C)C(=O)c1ccc(-c2ccc(OC3CCN(C(=O)c4cc(F)ccc4C(=O)NCCc4ccc(N(C)C)cc4)CC3)nc2)cc1 ; CN(C)C(=O)c1ccc(-c2ccc(OC3CCN(C(=O)c4ccc(F)cc4C(=O)NCCc4ccc(N(C)C)cc4)CC3)nc2)cc1]

JADA142-1 and JADA142-2 were prepared as a mixture in an analogous manner to JADA94-1 and JADA94-2 in Example 2-8 (9.3 mg, 33% yield).

### Example 2-10: Synthesis of N-(4-(dimethylamino)phenethyl)-4-fluoro-2-(4-((5-(4-(methylsulfonyl)phenyl)pyridin-2-yl)oxy)piperidine-1-carbonyl)benzamide (JADA143-1) and N-(4-(dimethylamino)phenethyl)-5-fluoro-2-(4-((5-(4-(methylsulfonyl)phenyl)pyridin-2-yl)oxy)piperidine-1-carbonyl)benzamide (JADA143-2)

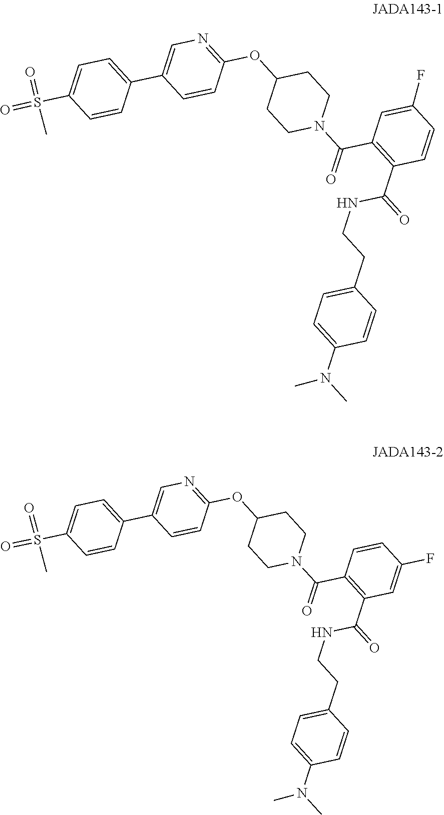 [SMILES: CN(C)c1ccc(CCNC(=O)c2ccc(F)cc2C(=O)N2CCC(Oc3ccc(-c4ccc(S(C)(=O)=O)cc4)cn3)CC2)cc1 ; CN(C)c1ccc(CCNC(=O)c2cc(F)ccc2C(=O)N2CCC(Oc3ccc(-c4ccc(S(C)(=O)=O)cc4)cn3)CC2)cc1]

JADA143-1 and JADA143-2 were prepared as a mixture in an analogous manner to JADA94-1 and JADA94-2 in Example 2-8 (7.3 mg, 21% yield).

### Example 2-11: Synthesis of N-(4-(dimethylamino)phenethyl)-4-fluoro-2-(4-((5-(4-(trifluoromethyl)phenyl)pyridin-2-yl)oxy)piperidine-1-carbonyl)benzamide (JADA144-1) and N-(4-(dimethylamino)phenethyl)-5-fluoro-2-(4-((5-(4-(trifluoromethyl)phenyl)pyridin-2-yl)oxy)piperidine-1-carbonyl)benzamide (JADA144-2)

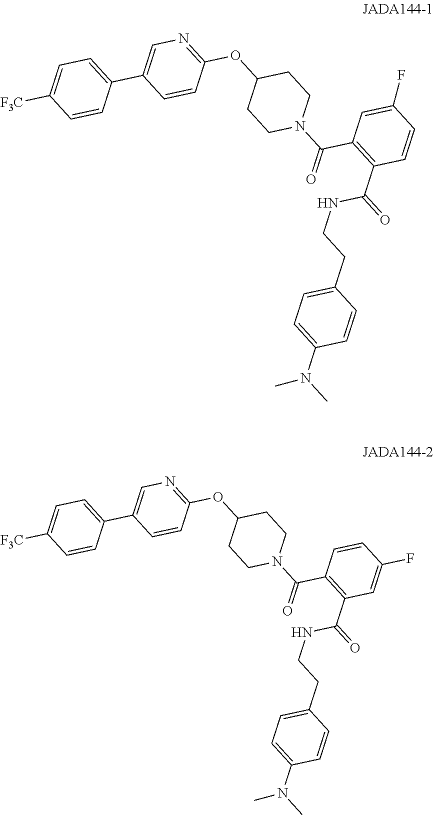 [SMILES: CN(C)c1ccc(CCNC(=O)c2ccc(F)cc2C(=O)N2CCC(Oc3ccc(-c4ccc(C(F)(F)F)cc4)cn3)CC2)cc1 ; CN(C)c1ccc(CCNC(=O)c2cc(F)ccc2C(=O)N2CCC(Oc3ccc(-c4ccc(C(F)(F)F)cc4)cn3)CC2)cc1]

JADA144-1 and JADA144-2 were prepared as a mixture in an analogous manner to JADA94-1 and JADA94-2 in Example 2-8 (9.3 mg, 29% yield).

### Example 2-12: Synthesis of N-(4-(dimethylamino)phenethyl)-4-fluoro-2-(4-((5-(4-(morpholinomethyl)phenyl)pyridin-2-yl)oxy)piperidine-1-carbonyl)benzamide (JADA146-1) and N-(4-(dimethylamino)phenethyl)-5-fluoro-2-(4-((5-(4-(morpholinomethyl)phenyl)pyridin-2-yl)oxy)piperidine-1-carbonyl)benzamide (JADA146-2)

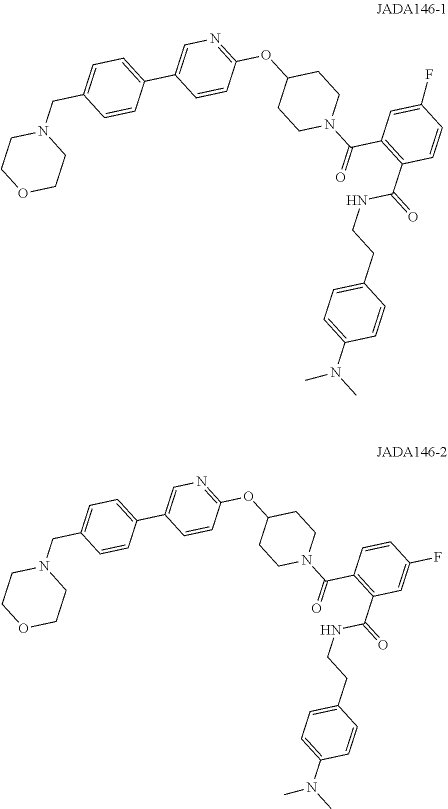 [SMILES: CN(C)c1ccc(CCNC(=O)c2ccc(F)cc2C(=O)N2CCC(Oc3ccc(-c4ccc(CN5CCOCC5)cc4)cn3)CC2)cc1 ; CN(C)c1ccc(CCNC(=O)c2cc(F)ccc2C(=O)N2CCC(Oc3ccc(-c4ccc(CN5CCOCC5)cc4)cn3)CC2)cc1]

JADA146-1 and JADA146-2 were prepared as a mixture in an analogous manner to JADA94-1 and JADA94-2 in Example 2-8 (12.1 mg, 36% yield).

### Example 2-13: Synthesis of N-(4-(dimethylamino)phenethyl)-4-fluoro-2-(4-((5-(2-(trifluoromethoxy)phenyl)pyridin-2-yl)oxy)piperidine-1-carbonyl)benzamide (JADA148-1) and N-(4-(dimethylamino)phenethyl)-5-fluoro-2-(4-((5-(2-(trifluoromethoxy)phenyl)pyridin-2-yl)oxy)piperidine-1-carbonyl)benzamide (JADA148-2)

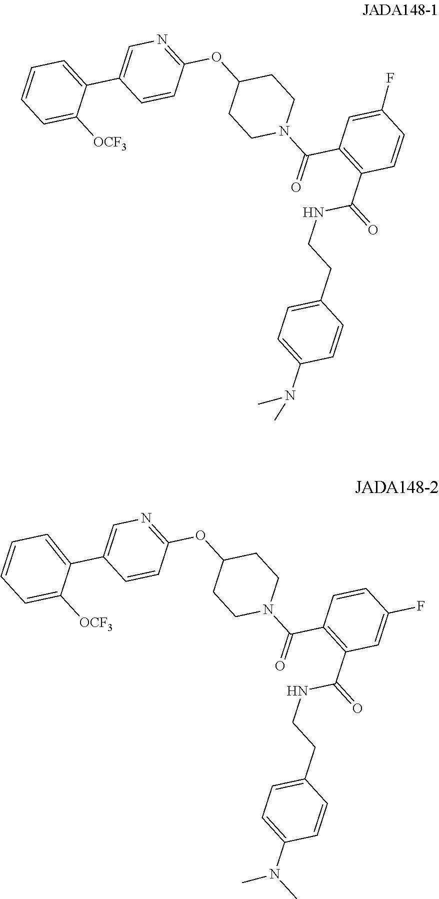 [SMILES: CN(C)c1ccc(CCNC(=O)c2ccc(F)cc2C(=O)N2CCC(Oc3ccc(-c4ccccc4OC(F)(F)F)cn3)CC2)cc1 ; CN(C)c1ccc(CCNC(=O)c2cc(F)ccc2C(=O)N2CCC(Oc3ccc(-c4ccccc4OC(F)(F)F)cn3)CC2)cc1]

JADA148-1 and 2 were prepared as a mixture in an analogous manner to JADA94-1 and JADA94-2 in Example 2-8 (8.1 mg, 26% yield).

### Example 2-14: Synthesis of N-(4-(dimethylamino)phenethyl)-4-fluoro-2-(4-((5-(3-(trifluoromethoxy)phenyl)pyridin-2-yl)oxy)piperidine-1-carbonyl)benzamide (JADA149-1) and N-(4-(dimethylamino)phenethyl)-5-fluoro-2-(4-((5-(3-(trifluoromethoxy)phenyl)pyridin-2-yl)oxy)piperidine-1-carbonyl)benzamide (JADA149-2)

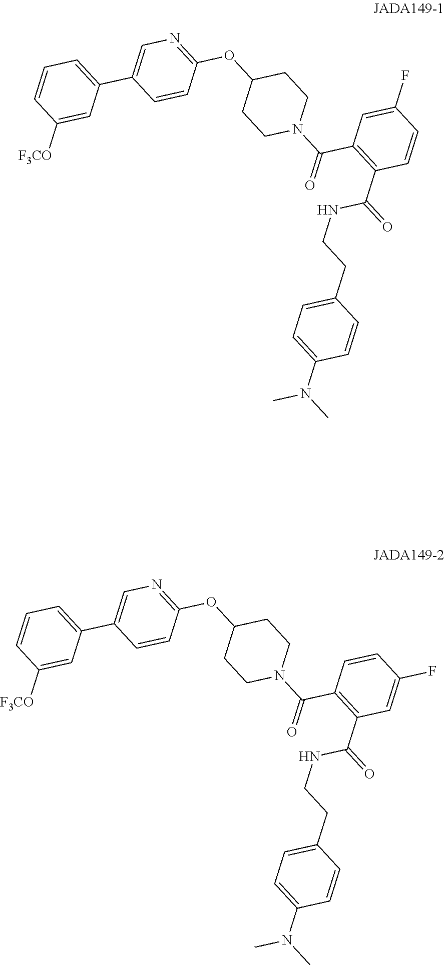 [SMILES: CN(C)c1ccc(CCNC(=O)c2ccc(F)cc2C(=O)N2CCC(Oc3ccc(-c4cccc(OC(F)(F)F)c4)cn3)CC2)cc1 ; CN(C)c1ccc(CCNC(=O)c2cc(F)ccc2C(=O)N2CCC(Oc3ccc(-c4cccc(OC(F)(F)F)c4)cn3)CC2)cc1]

JADA149-1 and JADA149-2 were prepared as a mixture in an analogous manner to JADA94-1 and JADA94-2 in Example 2-8 to (8.3 mg, 28% yield).

### Example 2-15: Synthesis of N-(4-(dimethylamino)phenethyl)-4-fluoro-2-(4-((5-morpholinopyridin-2-yl)oxy)piperidine-1-carbonyl)benzamide(KDM3-16-1) and N-(4-(dimethylamino)phenethyl)-5-fluoro-2-(4-((5-morpholinopyridin-2-yl)oxy)piperidine-1-carbonyl)benzamide (KDM3-16-2)

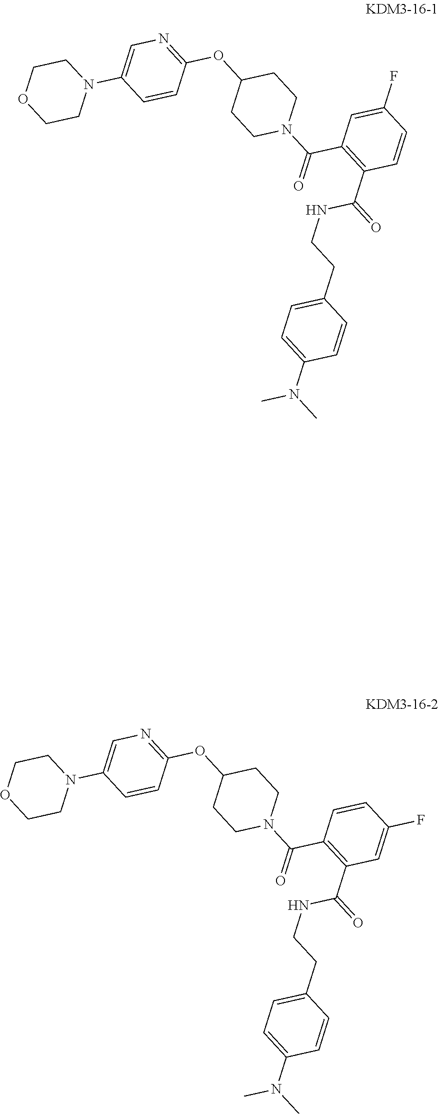 [SMILES: CN(C)c1ccc(CCNC(=O)c2ccc(F)cc2C(=O)N2CCC(Oc3ccc(N4CCOCC4)cn3)CC2)cc1 ; CN(C)c1ccc(CCNC(=O)c2cc(F)ccc2C(=O)N2CCC(Oc3ccc(N4CCOCC4)cn3)CC2)cc1]

KDM3-16-1 and KDM3-16-2 were prepared as a mixture in an analogous manner to JADA94-1 and JADA94-2 in Example 2-8 (11.5 mg, 40% yield).

### Example 2-16: Synthesis of N-(4-(dimethylamino)phenethyl)-2-(4-((5-(dimethylamino)pyridin-2-yl)oxy)piperidine-1-carbonyl)-4-fluorobenzamide (KDM3-17-1) and N-(4-(dimethylamino)phenethyl)-2-(4-((5-(dimethylamino)pyridin-2-yl)oxy)piperidine-1-carbonyl)-5-fluorobenzamide (KDM3-17-2)

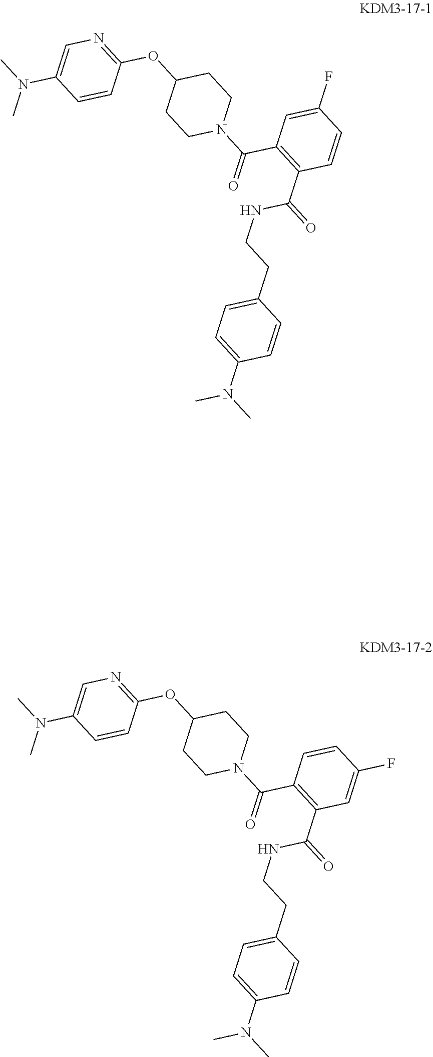 [SMILES: CN(C)c1ccc(CCNC(=O)c2ccc(F)cc2C(=O)N2CCC(Oc3ccc(N(C)C)cn3)CC2)cc1 ; CN(C)c1ccc(CCNC(=O)c2cc(F)ccc2C(=O)N2CCC(Oc3ccc(N(C)C)cn3)CC2)cc1]

KDM3-17-1 and KDM3-17-2 were prepared as a mixture in an analogous manner to JADA94-1 and JADA94-2 in Example 2-8 (13.5 mg, 50% yield).

### Example 2-17: Synthesis of ethyl 4-((4-(dimethylamino)phenethyl)carbamoyl)-3-(4-((5-(4-(dimethylamino)phenyl)pyridin-2-yl)oxy)piperidine-1-carbonyl)benzoate (KDM3-19-1) and ethyl 3-((4-(dimethylamino)phenethyl)carbamoyl)-4-(4-((5-(4-(dimethylamino)phenyl)pyridin-2-yl)oxy)piperidine-1-carbonyl)benzoate (KDM3-19-2)

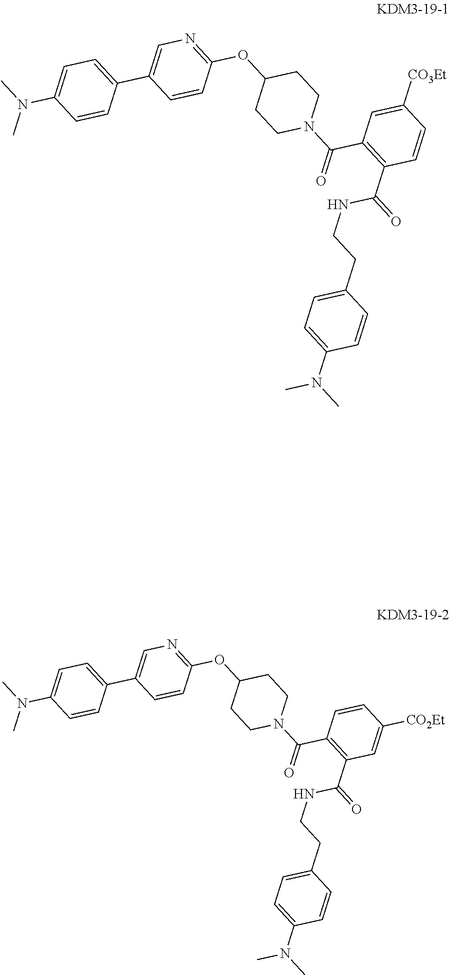

KDM3-19-1 and KDM3-19-2 were prepared as a mixture in an analogous manner to JADA94-1 and JADA94-2 in Example 2-8 (12.5 mg, 38% yield).

### Example 2-18: Synthesis of N-(4-(dimethylamino)phenethyl)-4-fluoro-2-(4-((5-(4-(phenoxymethyl)phenyl)pyridin-2-yl)oxy)piperidine-1-carbonyl)benzamide (KDM3-45-1)N-(4-(dimethylamino)phenethyl)-5-fluoro-2-(4-((5-(4-(phenoxymethyl)phenyl)pyridin-2-yl)oxy)piperidine-1-carbonyl)benzamide (KDM3-45-2)

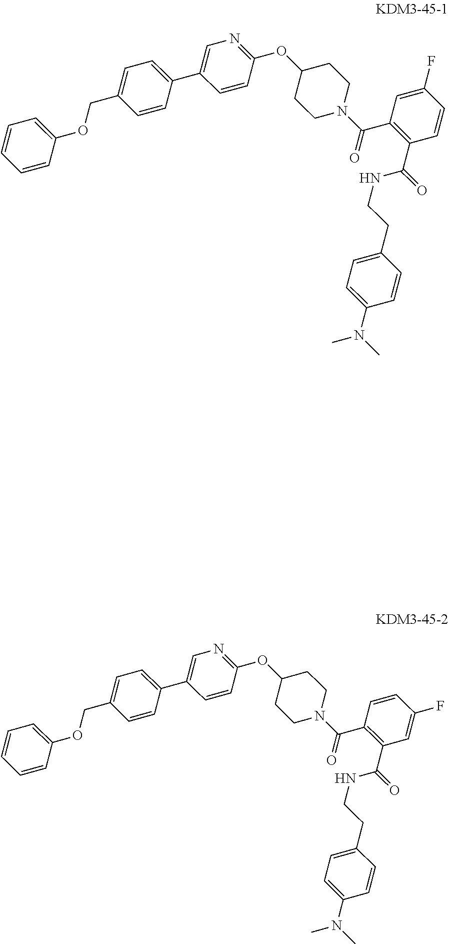 [SMILES: CN(C)c1ccc(CCNC(=O)c2ccc(F)cc2C(=O)N2CCC(Oc3ccc(-c4ccc(COc5ccccc5)cc4)cn3)CC2)cc1 ; CN(C)c1ccc(CCNC(=O)c2cc(F)ccc2C(=O)N2CCC(Oc3ccc(-c4ccc(COc5ccccc5)cc4)cn3)CC2)cc1]

KDM3-45-1 and KDM3-45-2 were prepared as a mixture in an analogous manner to JADA94-1 and JADA94-2 in Example 2-8 (10.1 mg, 30% yield).

### Example 2-19: Synthesis of N-(3-(4-(dimethylamino)phenyl)propyl)-2-(4-((5-(4-(dimethylamino)phenyl)pyridin-2-yl)oxy)piperidine-1-carbonyl)-4-fluorobenzamide (KDM3-56-1) and N-(3-(4-(dimethylamino)phenyl)propyl)-2-(4-((5-(4-(dimethylamino)phenyl)pyridin-2-yl)oxy)piperidine-1-carbonyl)-5-fluorobenzamide (KDM3-56-2)

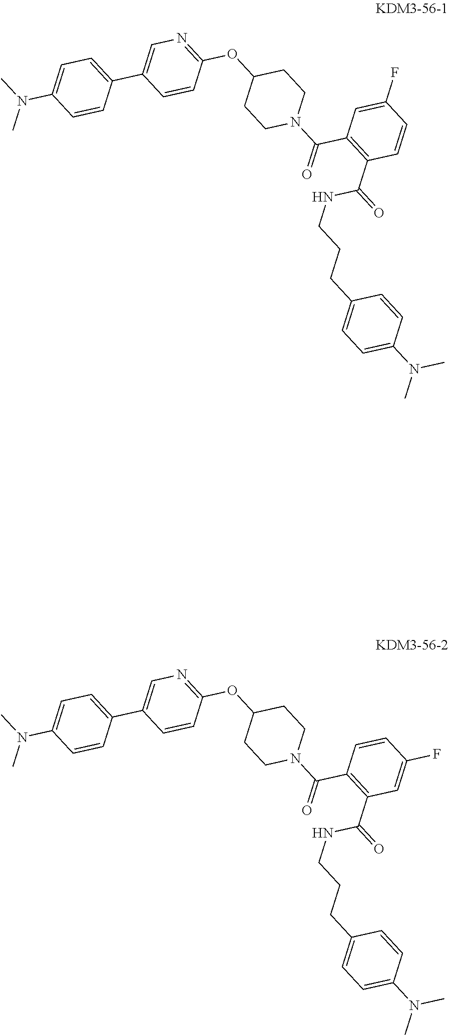

KDM3-56-1 and KDM3-56-2 were prepared as a mixture in an analogous manner to JADA94-1 and JADA94-2 in Example 2-8 (9.6 mg, 31% yield).

### Example 2-20: Synthesis of N-(4-(dimethylamino)phenethyl)-4-fluoro-2-(4-((5-(4-(4-methylpiperazin-1-yl)phenyl)pyridin-2-yl)oxy)piperidine-1-carbonyl)benzamide (KDM3-68-1) and N-(4-(dimethylamino)phenethyl)-5-fluoro-2-(4-((5-(4-(4-methylpiperazin-1-yl)phenyl)pyridin-2-yl)oxy)piperidine-1-carbonyl)benzamide (KDM3-68-2)

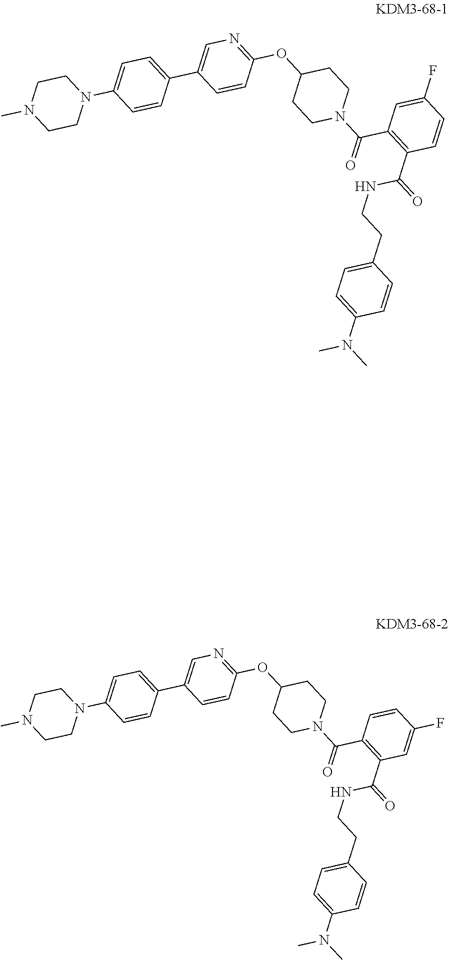 [SMILES: CN1CCN(c2ccc(-c3ccc(OC4CCN(C(=O)c5cc(F)ccc5C(=O)NCCc5ccc(N(C)C)cc5)CC4)nc3)cc2)CC1 ; CN1CCN(c2ccc(-c3ccc(OC4CCN(C(=O)c5ccc(F)cc5C(=O)NCCc5ccc(N(C)C)cc5)CC4)nc3)cc2)CC1]

KDM3-68-1 and KDM3-68-2 were prepared as a mixture in an analogous manner to JADA94-1 and JADA94-2 in Example 2-8 to (12.0 mg, 36% yield).

### Example 2-21: Synthesis of (R)—N-(4-(dimethylamino)phenethyl)-4-fluoro-2-(3-((5-(trifluoromethyl)pyridin-2-yl)oxy)pyrrolidine-1-carbonyl)benzamide (KDM3-35-1) and (R)—N-(4-(dimethylamino)phenethyl)-5-fluoro-2-(3-(trifluoromethyl)pyridin-2-yl)oxy)pyrrolidine-1-carbonyl)benzamide(KDM3-35-2)

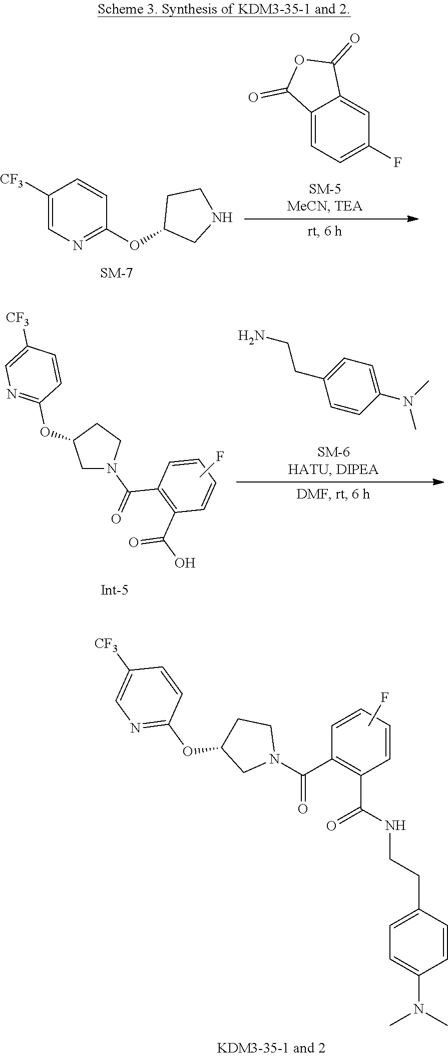

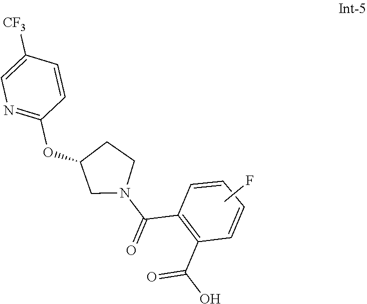

To a solution of SM-7 (23.2 mg, 0.1 mmol) and SM-5 (17 mg, 0.11 mmol) in MeCN (1 mL), TEA (50.5 mg, 0.5 mmol) was added. The reaction was stirred at 25° C. for 6 h prior to concentration of crude mixture under reduced pressure. The resulting residue was purified by ISCO (DCM/MeOH=100/0-70/30) to give Int-5 (31 mg, 79% yield).

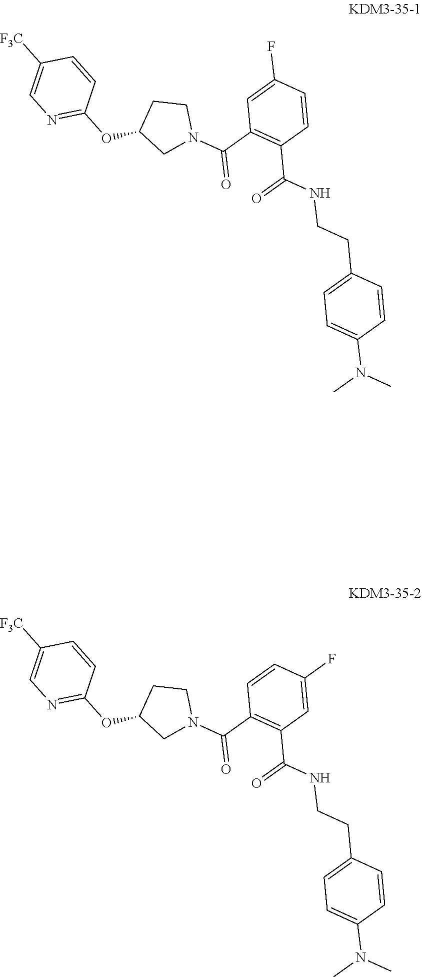 [SMILES: CN(C)c1ccc(CCNC(=O)c2ccc(F)cc2C(=O)N2CC[C@@H](Oc3ccc(C(F)(F)F)cn3)C2)cc1 ; CN(C)c1ccc(CCNC(=O)c2cc(F)ccc2C(=O)N2CC[C@@H](Oc3ccc(C(F)(F)F)cn3)C2)cc1]

To a solution of Int-5 (19 mg, 0.05 mmol) and SM-6 (9.8 mg, 0.06 mmol) in DMF (0.5 mL), DIPEA (27.8 mg, 0.2 mmol) and HATU (24 mg, 0.06 mmol) were added. The reaction was stirred at 25° C. for 6 h prior to dilution with EtOAc (2 mL). The mixture was washed with H₂O (2 mL), and the aqueous phase was extracted with additional EtOAc (2×2 mL). The combined organic phases were concentrated under reduced pressure. The resulting residue was purified by ISCO (DCM/MeOH=100/0-90/10) to give a mixture of KDM35-1 and KDM35-2 (13.6 mg, 51% yield).

### Example 2-22: Synthesis of (S)—N-(4-(dimethylamino)phenethyl)-4-fluoro-2-(3-((5-(trifluoromethyl)pyridin-2-yl)oxy)pyrrolidine-1-carbonyl)benzamide (KDM3-36-1) and (S)—N-(4-(dimethylamino)phenethyl)-5-fluoro-2-(3-(trifluoromethyl)pyridin-2-yl)oxy)pyrrolidine-1-carbonyl)benzamide(KDM3-36-2)

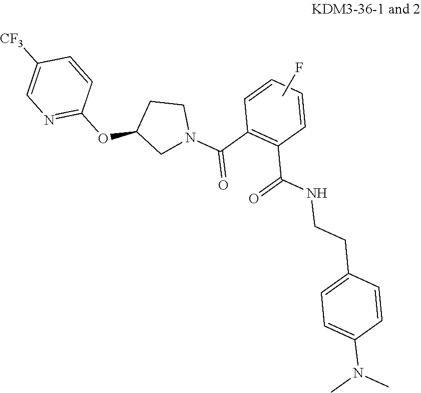

KDM3-36-1 and KDM3-36-2 were prepared as a mixture in an analogous manner to KDM3-35-1 and KDM3-35-2 in Example 2-21 (13.0 mg, 35% yield).

### Example 2-23: Synthesis of N-(4-(dimethylamino)phenethyl)-4-fluoro-2-(3-((5-(trifluoromethyl)pyridin-2-yl)oxy)azetidine-1-carbonyl)benzamide (KDM3-37-1) and N-(4-(dimethylamino)phenethyl)-5-fluoro-2-(3 #5-(trifluoromethyl)pyridin-2-yl)oxy)azetidine-1-carbonyl)benzamide (KDM3-37-2)

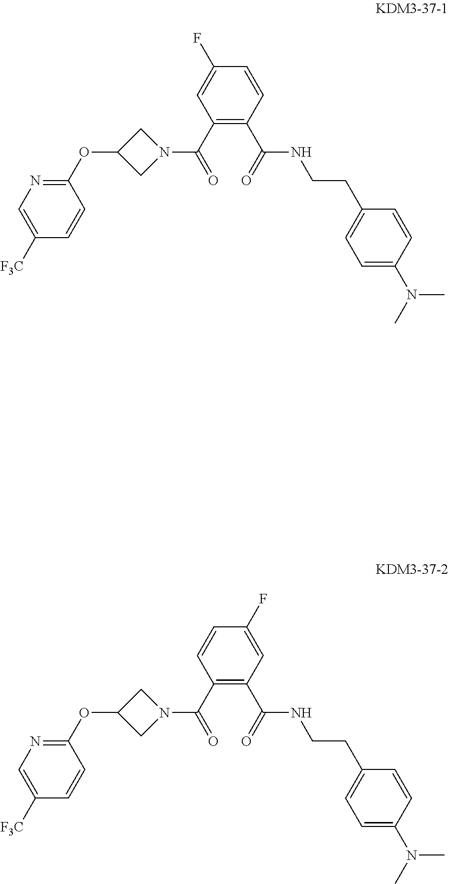 [SMILES: CN(C)c1ccc(CCNC(=O)c2ccc(F)cc2C(=O)N2CC(Oc3ccc(C(F)(F)F)cn3)C2)cc1 ; CN(C)c1ccc(CCNC(=O)c2cc(F)ccc2C(=O)N2CC(Oc3ccc(C(F)(F)F)cn3)C2)cc1]

KDM3-37-1 and KDM3-37-2 were prepared as a mixture in an analogous manner to KDM3-35-1 and KDM3-35-2 in Example 2-21 (10.0 mg, 34% yield).

### Example 2-24: Synthesis of 3-(4-(dimethylamino)phenyl)-N-(4-fluoro-2-(4-((4-morpholinopyridin-2-yl)oxy)piperidine-1-carbonyl)phenyl)propanamide (KDM3-26)

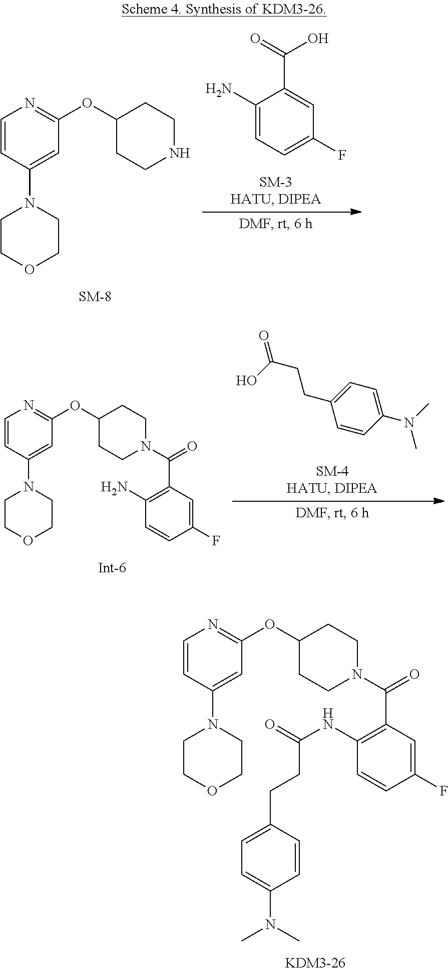 [SMILES: c1cc(N2CCOCC2)cc(OC2CCNCC2)n1 ; Nc1ccc(F)cc1C(=O)O ; Nc1ccc(F)cc1C(=O)N1CCC(Oc2cc(N3CCOCC3)ccn2)CC1 ; CN(C)c1ccc(CCC(=O)O)cc1 ; CN(C)c1ccc(CCC(=O)Nc2ccc(F)cc2C(=O)N2CCC(Oc3cc(N4CCOCC4)ccn3)CC2)cc1]

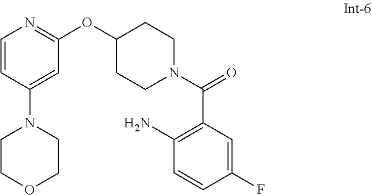 [SMILES: Nc1ccc(F)cc1C(=O)N1CCC(Oc2cc(N3CCOCC3)ccn2)CC1]

To a solution of SM-8 (26.3 mg, 0.1 mmol) and SM-3 (17 mg, 0.11 mmol) in DMF (1 mL), DIPEA (64.5 mg, 0.5 mmol) and HATU (46.6 mg, 0.12 mmol) were added. The reaction was stirred at 25° C. for 6 h prior to dilution with EtOAc (3 mL). The mixture was washed with H₂O (3 mL), and the aqueous phase was extracted with additional EtOAc (2×2 mL). The combined organic phases were concentrated under reduced pressure. The resulting residue was purified by ISCO (DCM/MeOH=100/0-85/15) to give Int-6 (24 mg, 60% yield).

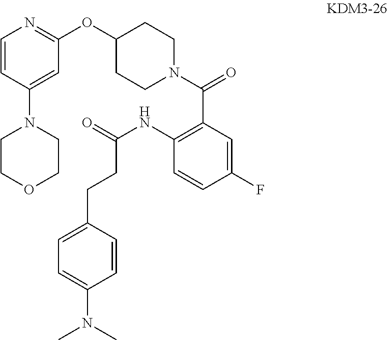 [SMILES: CN(C)c1ccc(CCC(=O)Nc2ccc(F)cc2C(=O)N2CCC(Oc3cc(N4CCOCC4)ccn3)CC2)cc1]

To a solution of Int-6 (20 mg, 0.05 mmol) and SM-4 (10.5 mg, 0.06 mmol) in DMF (0.5 mL), DIPEA (27.8 mg, 0.2 mmol) and HATU (24 mg, 0.06 mmol) were added. The reaction was stirred at 25° C. for 6 h prior to dilution with EtOAc (2 mL). The mixture was washed with H₂O (2 mL), and the aqueous phase was extracted with additional EtOAc (2×2 mL). The combined organic phases were concentrated under reduced pressure. The resulting residue was purified by ISCO (DCM/MeOH=100/0-90/10) to give KDM3-26 (9.5 mg, 36% yield).

### Example 2-25: Synthesis of 3-(4-(dimethylamino)phenyl)-N-(4-fluoro-2-(4-((4-(piperidin-1-yl)pyridin-2-yl)oxy)piperidine-1-carbonyl)phenyl)propanamide (KDM3-27)

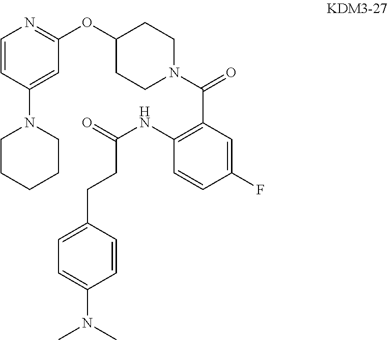 [SMILES: CN(C)c1ccc(CCC(=O)Nc2ccc(F)cc2C(=O)N2CCC(Oc3cc(N4CCCCC4)ccn3)CC2)cc1]

KDM3-27 was prepared in an analogous manner to KDM3-26 in Example 2-24 (12.6 mg, 44% yield).

### Example 2-26: Synthesis of ethyl 4-amino-3-(4-((4-(4-(dimethylamino)phenyl)pyridin-2-yl)oxy)piperidine-1-carbonyl)benzoate (KDM3-31)

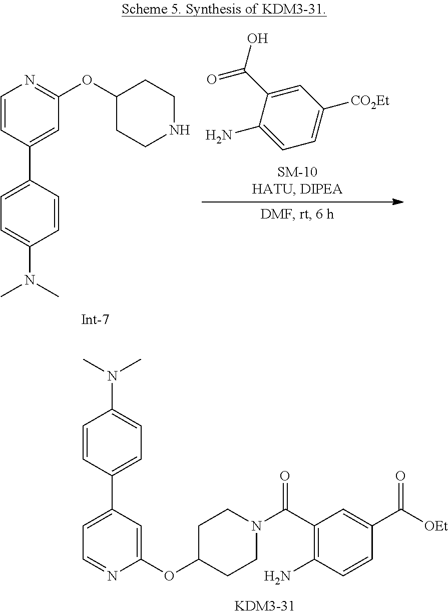

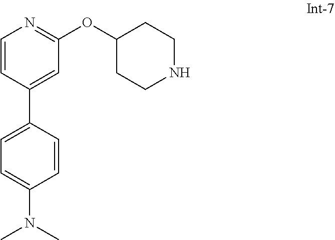 [SMILES: CN(C)c1ccc(-c2ccnc(OC3CCNCC3)c2)cc1]

Int-7 was prepared in an analogous manner to Int-2 in Example 2-1 (131 mg, 85% yield).

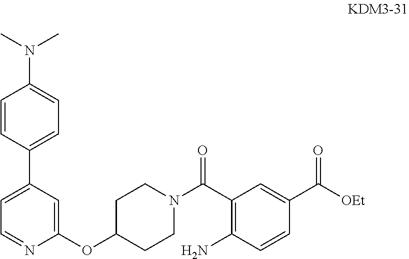 [SMILES: CCOC(=O)c1ccc(N)c(C(=O)N2CCC(Oc3cc(-c4ccc(N(C)C)cc4)ccn3)CC2)c1]

To a solution of Int-7 (20 mg, 0.05 mmol) and SM-10 (12.5 mg, 0.06 mmol) in DMF (0.5 mL), DIPEA (27.8 mg, 0.2 mmol) and HATU (24 mg, 0.06 mmol) were added. The reaction was stirred at 25° C. for 6 h prior to dilution with EtOAc (2 mL). The mixture was washed with H₂O (2 mL), and the aqueous phase was extracted with additional EtOAc (2×2 mL). The combined organic phases were concentrated under reduced pressure. The resulting residue was purified by ISCO (DCM/MeOH=100/0-80/20) to give KDM3-31 (12.5 mg, 50% yield).

### Example 2-27: Synthesis of ethyl 4-amino-3-(4-((4-(dimethylamino)pyridin-2-yl)oxy)piperidine-1-carbonyl)benzoate (KDM3-32)

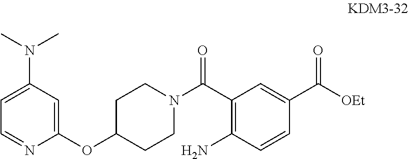 [SMILES: CCOC(=O)c1ccc(N)c(C(=O)N2CCC(Oc3cc(N(C)C)ccn3)CC2)c1]

KDM3-32 was prepared in an analogous manner to KDM3-31 in Example 2-26 (6.3 mg, 33% yield).

### Example 2-28: Synthesis of ethyl 4-amino-3-(4-((5-(dimethylamino)pyridin-2-yl)oxy)piperidine-1-carbonyl)benzoate (KDM3-33)

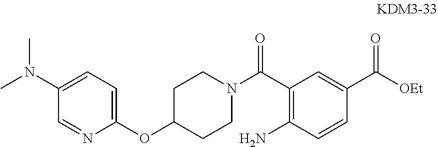 [SMILES: CCOC(=O)c1ccc(N)c(C(=O)N2CCC(Oc3ccc(N(C)C)cn3)CC2)c1]

KDM3-33 was prepared in an analogous manner to KDM3-31 in Example 2-26 (7.3 mg, 35% yield).

### Example 2-29: Synthesis of (4-((5-(4-(dimethylamino)phenyl)pyridin-2-yl)oxy)piperidin-1-yl)(2-nitrophenyl)methanone (KDM3-39)

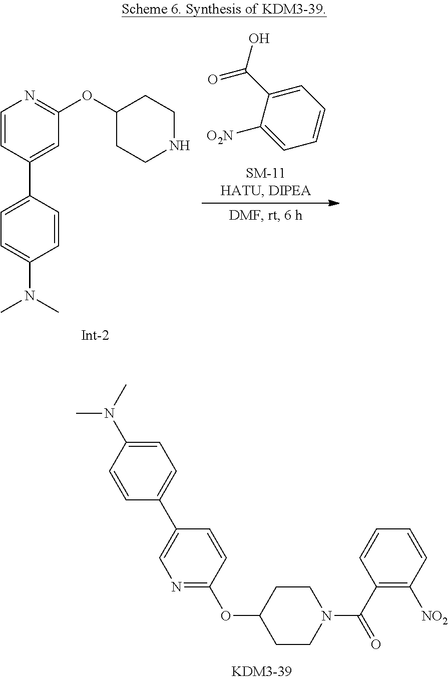 [SMILES: CN(C)c1ccc(-c2ccnc(OC3CCNCC3)c2)cc1 ; O=C(O)c1ccccc1[N+](=O)[O-] ; CN(C)c1ccc(-c2ccc(OC3CCN(C(=O)c4ccccc4[N+](=O)[O-])CC3)nc2)cc1]

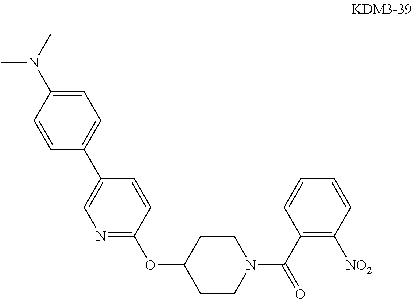 [SMILES: CN(C)c1ccc(-c2ccc(OC3CCN(C(=O)c4ccccc4[N+](=O)[O-])CC3)nc2)cc1]

To a solution of Int-2 (15 mg, 0.05 mmol) and SM-10 (9.1 mg, 0.06 mmol) in DMF (0.5 mL), DIPEA (27.8 mg, 0.2 mmol) and HATU (24 mg, 0.06 mmol) were added. The reaction was stirred at 25° C. for 6 h prior to dilution with EtOAc (2 mL). The mixture was washed with H₂O (2 mL), and the aqueous phase was extracted with additional EtOAc (2×2 mL). The combined organic phases were concentrated under reduced pressure. The resulting residue was purified by ISCO (DCM/MeOH=100/0-80/20) to give KDM3-31 (2.5 mg, 12% yield).

### Example 2-30: Synthesis of N-(3-amino-4-(4-((5-(4-(dimethylamino)phenyl)pyridin-2-yl)oxy)piperidine-1-carbonyl)phenyl)acetamide (KDM3-40)

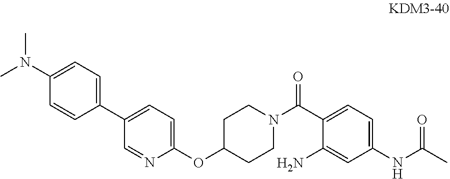 [SMILES: CC(=O)Nc1ccc(C(=O)N2CCC(Oc3ccc(-c4ccc(N(C)C)cc4)cn3)CC2)c(N)c1]

KDM3-40 was prepared in an analogous manner to KDM3-39 in Example 2-29 (5.4 mg, 23% yield).

### Example 2-31: Synthesis of (3-((5-(4-(dimethylamino)phenyl)pyridin-2-yl)oxy)azetidin-1-yl)(2-nitrophenyl)methanone (KDM3-42)

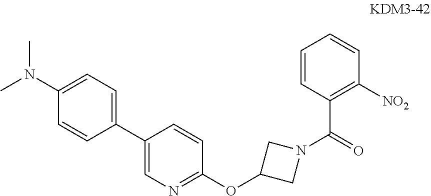 [SMILES: CN(C)c1ccc(-c2ccc(OC3CN(C(=O)c4ccccc4[N+](=O)[O-])C3)nc2)cc1]

KDM3-42 was prepared in an analogous manner to KDM3-39 in Example 2-29 (3.1 mg, 15% yield).

### Example 2-32: Synthesis of (3-((5-(4-(dimethylamino)phenyl)pyridin-2-yl)oxy)azetidin-1-yl)(2-nitrophenyl)methanone (KDM3-44)

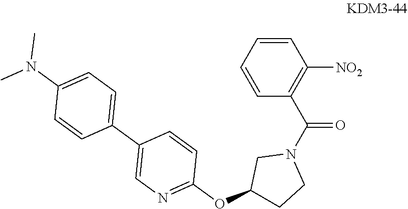 [SMILES: CN(C)c1ccc(-c2ccc(O[C@@H]3CCN(C(=O)c4ccccc4[N+](=O)[O-])C3)nc2)cc1]

KDM3-44 was prepared in an analogous manner to KDM3-39 in Example 2-29 (3.7 mg, 19% yield).

### Example 2-33: Synthesis of 4-amino-3-(4-((5-(4-(dimethylamino)phenyl)pyridin-2-yl)oxy)piperidine-1-carbonyl)benzoic acid (KDM3-41)

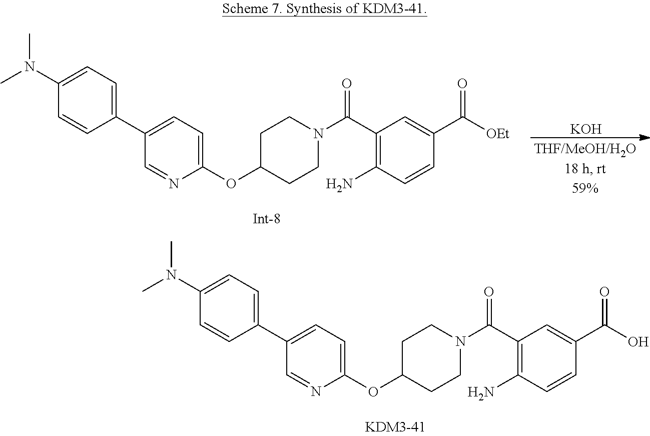 [SMILES: CCOC(=O)c1ccc(N)c(C(=O)N2CCC(Oc3ccc(-c4ccc(N(C)C)cc4)cn3)CC2)c1 ; "KOH" ; CN(C)c1ccc(-c2ccc(OC3CCN(C(=O)c4cc(C(=O)O)ccc4N)CC3)nc2)cc1]

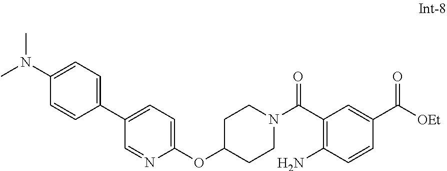 [SMILES: CCOC(=O)c1ccc(N)c(C(=O)N2CCC(Oc3ccc(-c4ccc(N(C)C)cc4)cn3)CC2)c1]

Int-8 was prepared in an analogous manner to KDM3-31 in Example 2-26 (12.6 mg, 28% yield).

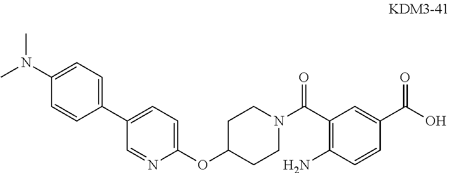 [SMILES: CN(C)c1ccc(-c2ccc(OC3CCN(C(=O)c4cc(C(=O)O)ccc4N)CC3)nc2)cc1]

To a solution of Int-8 (9.5 mg, 0.02 mmol) in a 1:1:1 mixture of THF/MeOH/H2O (1.5 mL), KOH (5.6 mg, 0.1 mmol) was added. The solution was stirred at 25° C. for 24 h. After pH adjustment (pH 4-5) with HCl (1M), the resulting mixture was extracted with EtOAc (1 mL×3) and concentrated under reduced pressure. The crude product was purified by ISCO (CH₂Cl₂/MeOH=100/0˜70/30) to give KDM3-41 (4.6 mg, yield 50%).

### Example 2-34: Synthesis of 4-amino-3-(4-((4-(4-(dimethylamino)phenyl)pyridin-2-yl)oxy)piperidine-1-carbonyl)benzoic acid (KDM3-43)

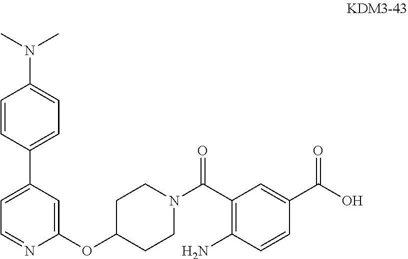 [SMILES: CN(C)c1ccc(-c2ccnc(OC3CCN(C(=O)c4cc(C(=O)O)ccc4N)CC3)c2)cc1]

KDM3-43 was prepared in an analogous manner to KDM3-41 in Example 2-33 (4.2 mg, 40% yield).

### Example 2-35: Synthesis of (Z)-N-(4-(dimethylamino)phenethyl)-4-(4-((5-(4-(dimethylamino)phenyl)pyridin-2-yl)oxy)piperidin-1-yl)-4-oxobut-2-enamide (KDM3-38)

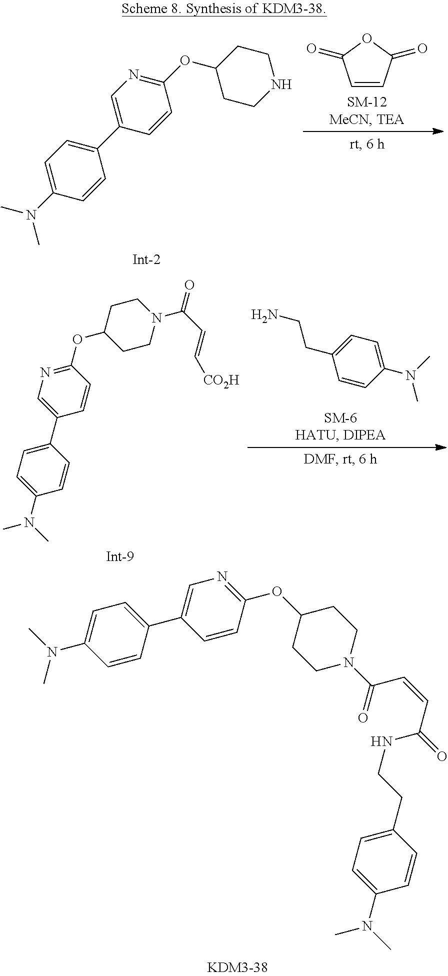 [SMILES: CN(C)c1ccc(-c2ccc(OC3CCNCC3)nc2)cc1 ; O=C1C=CC(=O)O1 ; CN(C)c1ccc(-c2ccc(OC3CCN(C(=O)/C=C/C(=O)O)CC3)nc2)cc1 ; CN(C)c1ccc(CCN)cc1 ; CN(C)c1ccc(CCNC(=O)/C=C\C(=O)N2CCC(Oc3ccc(-c4ccc(N(C)C)cc4)cn3)CC2)cc1]

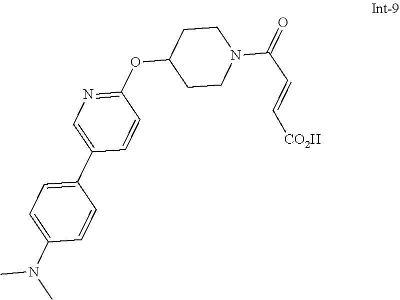 [SMILES: CN(C)c1ccc(-c2ccc(OC3CCN(C(=O)/C=C/C(=O)O)CC3)nc2)cc1]

To a solution of Int-2 (29.7 mg, 0.1 mmol) and SM-12 (10.8 mg, 0.11 mmol) in MeCN (1 mL), TEA (50.5 mg, 0.5 mmol) was added. The reaction was stirred at 25° C. for 6 h prior to concentration of crude mixture under reduced pressure. The resulting residue was purified by ISCO (DCM/MeOH=100/0-70/30) to give Int-9 (31 mg, 80% yield).

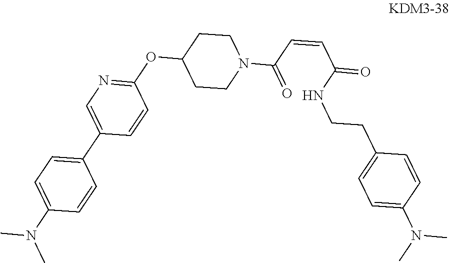

To a solution of Int-9 (19.5 mg, 0.05 mmol) and SM-6 (9.6 mg, 0.06 mmol) in DMF (0.5 mL), DIPEA (27.8 mg, 0.2 mmol) and HATU (24 mg, 0.06 mmol) were added. The reaction was stirred at 25° C. for 6 h prior to dilution with EtOAc (2 mL). The mixture was washed with H₂O (2 mL), and the aqueous phase was extracted with additional EtOAc (2×2 mL). The combined organic phases were concentrated under reduced pressure. The resulting residue was purified by ISCO (DCM/MeOH=100/0-90/10) to give KDM3-38 (9.5 mg, 35% yield).

### Example 2-36: Synthesis of (Z)-4-(4-((5-(4-(dimethylamino)phenyl)pyridin-2-yl)oxy)piperidin-1-yl)-N-((1-methylpiperidin-4-yl)methyl)-4-oxobut-2-enamide (KDM3-46)

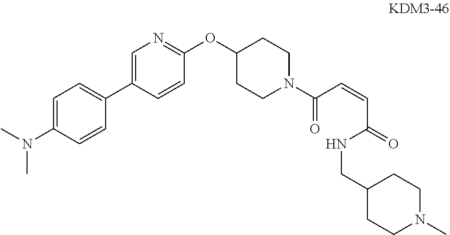

KDM3-46 was prepared in an analogous manner to KDM3-38 in Example 2-35 (10.2 mg, 40% yield).

### Example 2-37: Synthesis of (Z)-N-(4-(dimethylamino)benzyl)-4-(4-((5-(4-(dimethylamino)phenyl)pyridin-2-yl)oxy)piperidin-1-yl)-4-oxobut-2-enamide (KDM3-47)

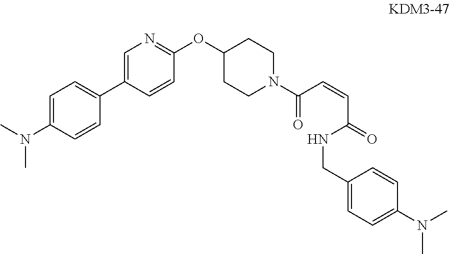

KDM3-47 was prepared in an analogous manner to KDM3-38 in Example 2-35 (8.4 mg, 32% yield).

### Example 2-38: Synthesis of (Z)-N-(2-(dimethylamino)ethyl)-4-(4-((5-(4-(dimethylamino)phenyl)pyridin-2-yl)oxy)piperidin-1-yl)-4-oxobut-2-enamide (KDM3-48)

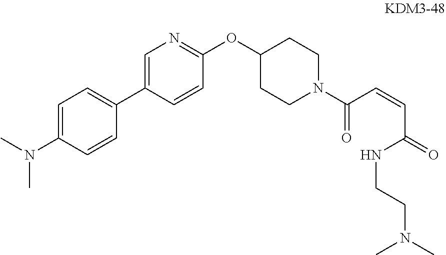

KDM3-48 was prepared in an analogous manner to KDM3-38 in Example 2-35 (6.9 mg, 31% yield).

### Example 2-39: Synthesis of (Z)-N-(2-(dimethylamino)ethyl)-4-(4-((5-(4-(dimethylamino)phenyl)pyridin-2-yl)oxy)piperidin-1-yl)-N-ethyl-4-oxobut-2-enamide (KDM3-49)

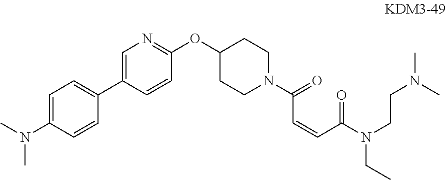

KDM3-49 was prepared in an analogous manner to KDM3-38 in Example 2-35 (7.9 mg, 34% yield).

### Example 2-40: Synthesis of (Z)-4-(4-((5-(4-(dimethylamino)phenyl)pyridin-2-yl)oxy)piperidin-1-yl)-N-(2-(4-methylpiperazin-1-yl)ethyl)-4-oxobut-2-enamide (KDM3-50)

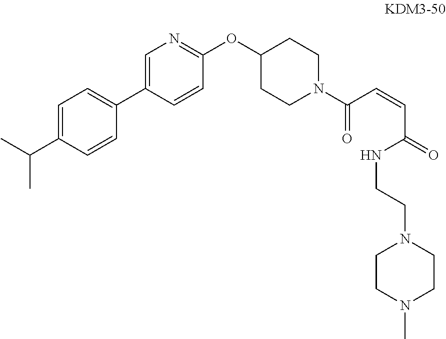 [SMILES: CC(C)c1ccc(-c2ccc(OC3CCN(C(=O)/C=C\C(=O)NCCN4CCN(C)CC4)CC3)nc2)cc1]

KDM3-50 was prepared in an analogous manner to KDM3-38 in Example 2-35 (7.3 mg, 28% yield).

### Example 2-41: Synthesis of N-(4-(dimethylamino)phenethyl)-4-oxo-4-(4-((5-(trifluoromethyl)pyridin-2-yl)oxy)piperidin-1-yl)butanamide (JADA69)

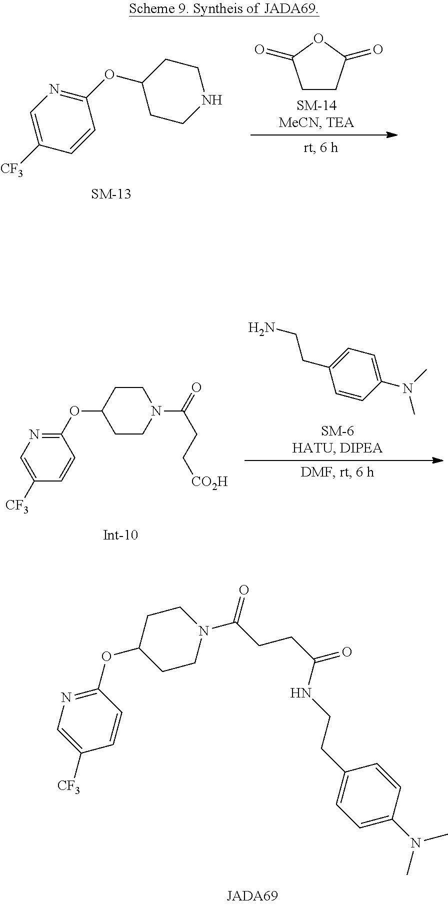 [SMILES: FC(F)(F)c1ccc(OC2CCNCC2)nc1 ; O=C1CCC(=O)O1 ; O=C(O)CCC(=O)N1CCC(Oc2ccc(C(F)(F)F)cn2)CC1 ; CN(C)c1ccc(CCN)cc1 ; CN(C)c1ccc(CCNC(=O)CCC(=O)N2CCC(Oc3ccc(C(F)(F)F)cn3)CC2)cc1]

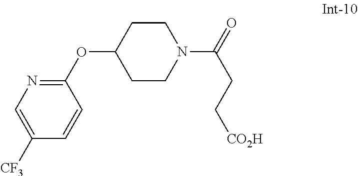 [SMILES: O=C(O)CCC(=O)N1CCC(Oc2ccc(C(F)(F)F)cn2)CC1]

To a solution of SM-13 (24.6 mg, 0.1 mmol) and SM-14 (11.2 mg, 0.11 mmol) in MeCN (1 mL), TEA (50.5 mg, 0.5 mmol) was added. The reaction was stirred at 25° C. for 6 h prior to concentration of crude mixture under reduced pressure. The resulting residue was purified by ISCO (DCM/MeOH=100/0-70/30) to give Int-10 (35 mg, 85% yield).

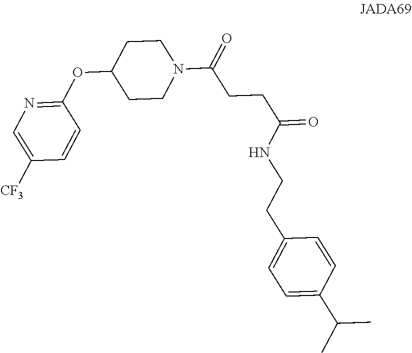 [SMILES: CC(C)c1ccc(CCNC(=O)CCC(=O)N2CCC(Oc3ccc(C(F)(F)F)cn3)CC2)cc1]

To a solution of Int-10 (19.5 mg, 0.05 mmol) and SM-6 (9.6 mg, 0.06 mmol) in DMF (0.5 mL), DIPEA (27.8 mg, 0.2 mmol) and HATU (24 mg, 0.06 mmol) were added. The reaction was stirred at 25° C. for 6 h prior to dilution with EtOAc (2 mL). The mixture was washed with H₂O (2 mL), and the aqueous phase was extracted with additional EtOAc (2×2 mL). The combined organic phases were concentrated under reduced pressure. The resulting residue was purified by ISCO (DCM/MeOH=100/0-85/15) to give JADA69 (9.6 mg, 39% yield).

### Example 2-42: Synthesis of N-(4-(dimethylamino)phenethyl)-4-(4-((4-(4-(dimethylamino)phenyl)pyridin-2-yl)oxy)piperidin-1-yl)-4-oxobutanamide (JADA84)

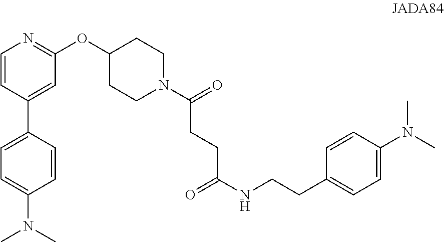 [SMILES: CN(C)c1ccc(CCNC(=O)CCC(=O)N2CCC(Oc3cc(-c4ccc(N(C)C)cc4)ccn3)CC2)cc1]

JADA84 was prepared in an analogous manner to JADA69 in Example 2-41 (11.3 mg, 41% yield).

### Example 2-43: Synthesis of N-(4-(dimethylamino)phenethyl)-4-(4-((5-(4-(dimethylamino)phenyl)pyridin-2-yl)oxy)piperidin-1-yl)-4-oxobutanamide (JADA92)

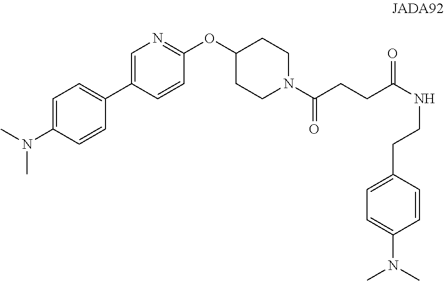

JADA92 was prepared in an analogous manner to JADA69 in Example 2-41 (9.7 mg, 36% yield).

### Example 2-44: Synthesis of N-(4-(dimethylamino)phenethyl)-4-oxo-4-(4-((5-(4-(trifluoromethoxy)phenyl)pyridin-2-yl) amino)piperidin-1-yl)butanamide (JADA112)

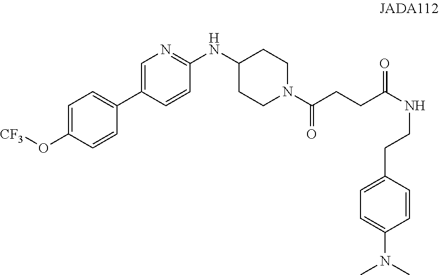 [SMILES: CN(C)c1ccc(CCNC(=O)CCC(=O)N2CCC(Nc3ccc(-c4ccc(OC(F)(F)F)cc4)cn3)CC2)cc1]

JADA112 was prepared in an analogous manner to JADA69 in Example 2-41 (9.0 mg, 31% yield).

### Example 2-45: Synthesis of 3-(4-bromophenyl)-N-(2-(4-((5-bromopyridin-2-yl)oxy)piperidine-1-carbonyl)-4-fluorophenyl)propanamide (KDM3-20)

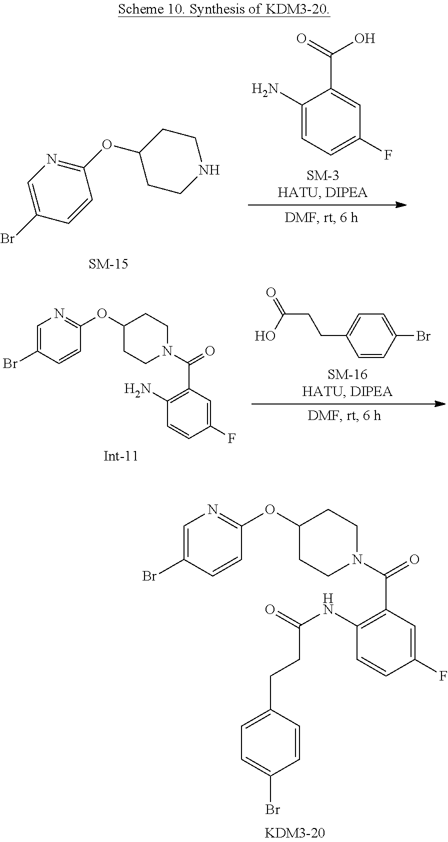 [SMILES: Brc1ccc(OC2CCNCC2)nc1 ; Nc1ccc(F)cc1C(=O)O ; Nc1ccc(F)cc1C(=O)N1CCC(Oc2ccc(Br)cn2)CC1 ; O=C(O)CCc1ccc(Br)cc1 ; O=C(CCc1ccc(Br)cc1)Nc1ccc(F)cc1C(=O)N1CCC(Oc2ccc(Br)cn2)CC1]

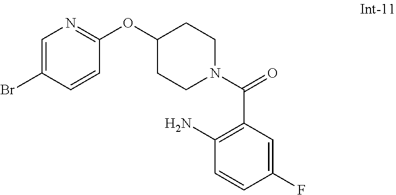 [SMILES: Nc1ccc(F)cc1C(=O)N1CCC(Oc2ccc(Br)cn2)CC1]

To a solution of SM-15 (25.6 mg, 0.1 mmol) and SM-3 (17 mg, 0.11 mmol) in DMF (1 mL), DIPEA (64.5 mg, 0.5 mmol) and HATU (46.6 mg, 0.12 mmol) were added. The reaction was stirred at 25° C. for 6 h prior to dilution with EtOAc (3 mL). The mixture was washed with H₂O (3 mL), and the aqueous phase was extracted with additional EtOAc (2×2 mL). The combined organic phases were concentrated under reduced pressure. The resulting residue was purified by ISCO (DCM/MeOH=100/0-90/10) to give Int-11 (24 mg, 61% yield).

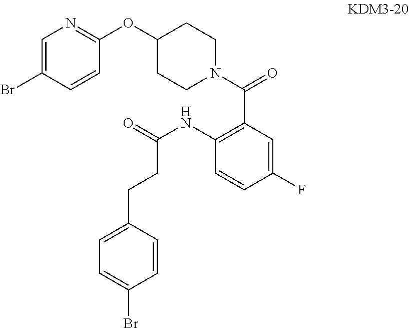 [SMILES: O=C(CCc1ccc(Br)cc1)Nc1ccc(F)cc1C(=O)N1CCC(Oc2ccc(Br)cn2)CC1]

To a solution of Int-11 (20 mg, 0.05 mmol) and SM-16 (13.5 mg, 0.06 mmol) in DMF (0.5 mL), DIPEA (27.8 mg, 0.2 mmol) and HATU (24 mg, 0.06 mmol) were added. The reaction was stirred at 25° C. for 6 h prior to dilution with EtOAc (2 mL). The mixture was washed with H₂O (2 mL), and the aqueous phase was extracted with additional EtOAc (2×2 mL). The combined organic phases were concentrated under vacuum. The residue was purified by ISCO (DCM/MeOH=100/0-90/10) to give KDM3-20 (9.6 mg, 32% yield).

### Example 2-46: Synthesis of N-(2-(4-((5-bromopyridin-2-yl)oxy)piperidine-1-carbonyl)-4-fluorophenyl)-3-(p-tolyl)propanamide (KDM3-21)

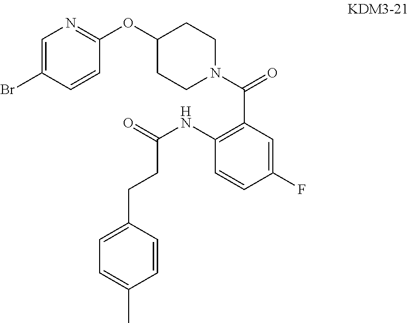 [SMILES: Cc1ccc(CCC(=O)Nc2ccc(F)cc2C(=O)N2CCC(Oc3ccc(Br)cn3)CC2)cc1]

KDM3-21 was prepared in an analogous manner to KDM3-20 in Example 2-45 (10.2 mg, 38% yield).

### Example 2-47: Synthesis of N-(2-(4-((5-bromopyridin-2-yl)oxy)piperidine-1-carbonyl)-4-fluorophenyl)-3-(pyridin-3-yl)propanamide (KDM3-22)

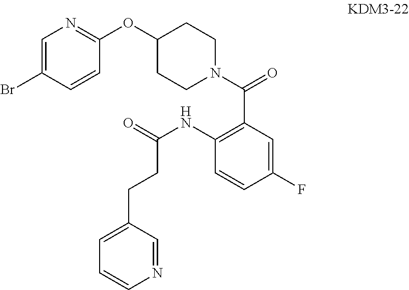 [SMILES: O=C(CCc1cccnc1)Nc1ccc(F)cc1C(=O)N1CCC(Oc2ccc(Br)cn2)CC1]

KDM3-22 was prepared in an analogous manner to KDM3-20 in Example 2-45 (10.2 mg, 38% yield).

### Example 2-48: Synthesis of (2-((4-(dimethylamino)benzyl)amino)-5-fluorophenyl)(4-((5-(3,5-dimethylisoxazol-4-yl)pyridin-2-yl)oxy)piperidin-1-yl)methanone (KDM3-64)

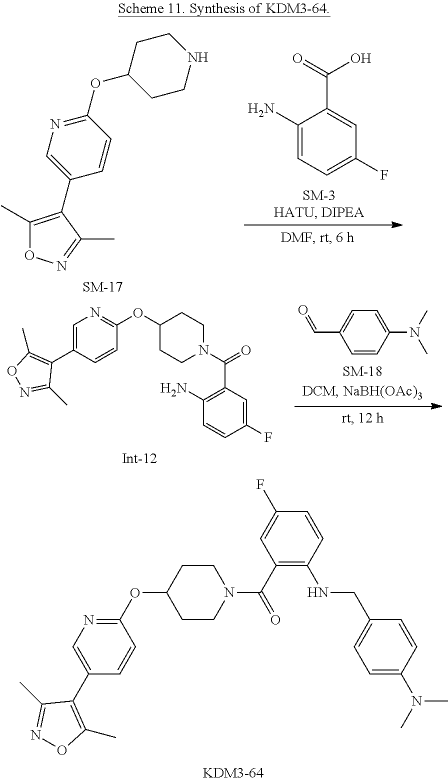

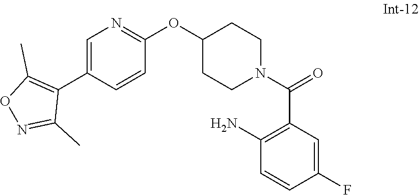 [SMILES: Cc1noc(C)c1-c1ccc(OC2CCN(C(=O)c3cc(F)ccc3N)CC2)nc1]

To a solution of SM-17 (25.6 mg, 0.1 mmol) and SM-3 (17 mg, 0.11 mmol) in DMF (1 mL), DIPEA (64.5 mg, 0.5 mmol) and HATU (46.6 mg, 0.12 mmol) were added. The reaction was stirred at 25° C. for 6 h prior to dilution with EtOAc (3 mL). The mixture was washed with H₂O (3 mL), and the aqueous phase was extracted with additional EtOAc (2×2 mL). The combined organic phases were concentrated under reduced pressure. The resulting residue was purified by ISCO (DCM/MeOH=100/0-90/10) to give Int-12 (26.6 mg, 65% yield).

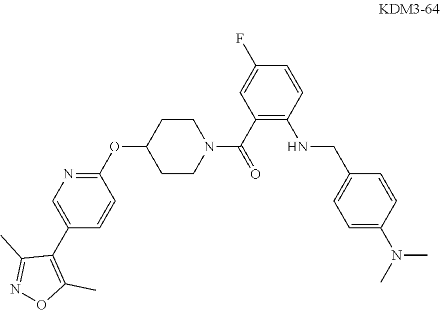

A solution of Int-12 (8.2 mg, 0.02 mmol) and SM-18 (2.9 mg, 0.025 mmol) in CH₂Cl₂ (1 mL) was stirred at 25° C. for 3 h prior to addition of NaBH(AcO)₃ (6.3 mg, 0.03 mmol) in portions. The reaction was stirred at 25° C. for 9 h. The crude mixture was concentrated under reduced pressure, and the resulting residue was purified by ISCO (CH₂Cl₂/MeOH=100/0˜80/20) to give KDM3-64 (8.1 mg, 71% yield).

### Example 2-49: Synthesis of (2-((4-(dimethylamino)benzyl)amino)-5-fluorophenyl)(4-((5-(4-(dimethylamino)phenyl)pyridin-2-yl)oxy)piperidin-1-yl)methanone (KDM3-65)

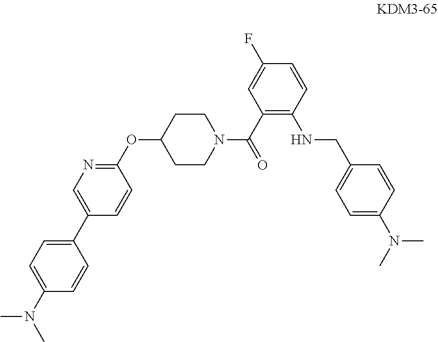

KDM3-65 was prepared in an analogous manner to KDM3-64 in Example 2-48 (7.8 mg, 69% yield).

### Example 2-50: Synthesis of (4-((5-bromopyridin-2-yl)oxy)piperidin-1-yl)(2-((4-(dimethylamino)benzyl)amino)-5-fluorophenyl)methanone (KDM3-66)

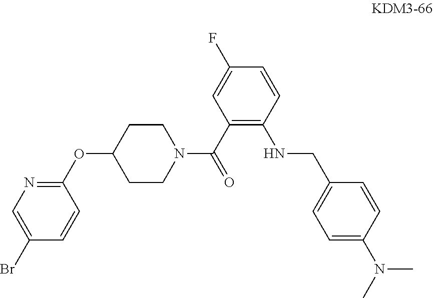 [SMILES: CN(C)c1ccc(CNc2ccc(F)cc2C(=O)N2CCC(Oc3ccc(Br)cn3)CC2)cc1]

KDM3-66 was prepared in an analogous manner to KDM3-64 in Example 2-48 (8.1 mg, 77% yield).

### Example 2-51: Synthesis of (2-((2,4-dihydroxybenzyl)amino)-5-fluorophenyl)(4-((5-(3,5-dimethylisoxazol-4-yl)pyridin-2-yl)oxy)piperidin-1-yl)methanone (KDM3-67)

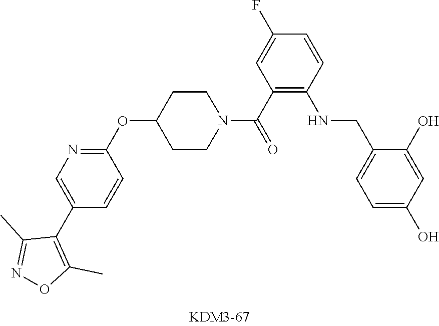 [SMILES: Cc1noc(C)c1-c1ccc(OC2CCN(C(=O)c3cc(F)ccc3NCc3ccc(O)cc3O)CC2)nc1]

KDM3-67 was prepared in an analogous manner to KDM3-64 in Example 2-48 (2.1 mg, 21% yield).

### Example 2-52: Synthesis of 4-(6-((1-(2-((4-(dimethylamino)benzyl)amino)-5-fluorobenzoyl)piperidin-4-yl)oxy)pyridin-3-yl)-N,N-dimethylbenzamide (KDM3-68)

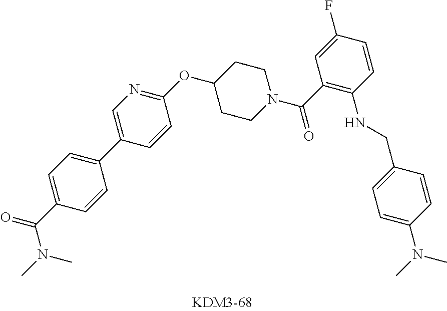 [SMILES: CN(C)C(=O)c1ccc(-c2ccc(OC3CCN(C(=O)c4cc(F)ccc4NCc4ccc(N(C)C)cc4)CC3)nc2)cc1]

KDM3-68 was prepared in an analogous manner to KDM3-64 in Example 2-48 (4.9 mg, 46% yield).

### Example 2-53: Synthesis of 2-(4-((5-(4-(dimethylamino)phenyl)pyridin-2-yl)oxy)piperidine-1-carbonyl)phenyl 2-hydroxybenzoate (JADA196)

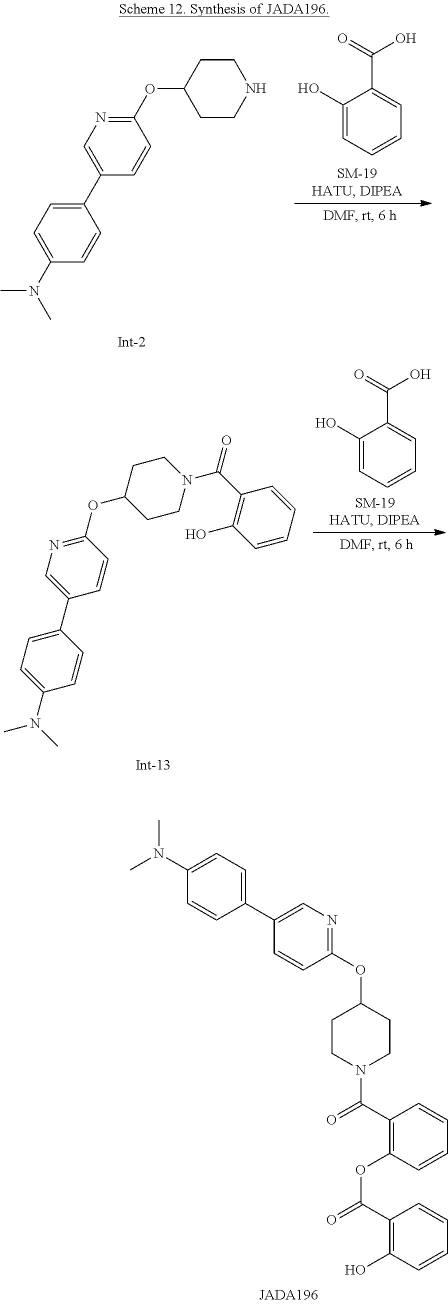 [SMILES: CN(C)c1ccc(-c2ccc(OC3CCNCC3)nc2)cc1 ; O=C(O)c1ccccc1O ; CN(C)c1ccc(-c2ccc(OC3CCN(C(=O)c4ccccc4O)CC3)nc2)cc1 ; O=C(O)c1ccccc1O ; CN(C)c1ccc(-c2ccc(OC3CCN(C(=O)c4ccccc4OC(=O)c4ccccc4O)CC3)nc2)cc1]

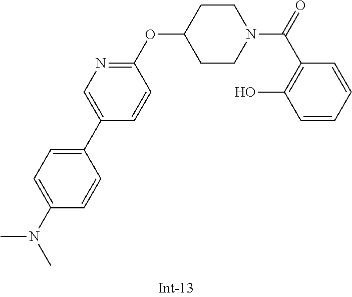 [SMILES: CN(C)c1ccc(-c2ccc(OC3CCN(C(=O)c4ccccc4O)CC3)nc2)cc1]

To a solution of Int-2 (29.7 mg, 0.1 mmol) and SM-19 (14.5 mg, 0.11 mmol) in DMF (1 mL), DIPEA (64.5 mg, 0.5 mmol) and HATU (46.6 mg, 0.12 mmol) were added. The reaction was stirred at 25° C. for 6 h prior to dilution with EtOAc (3 mL). The mixture was washed with H₂O (3 mL), and the aqueous phase was extracted with additional EtOAc (2×2 mL). The combined organic phases were concentrated under reduced pressure. The resulting residue was purified by ISCO (DCM/MeOH=100/0-80/20) to give Int-13 (21.6 mg, 51% yield).

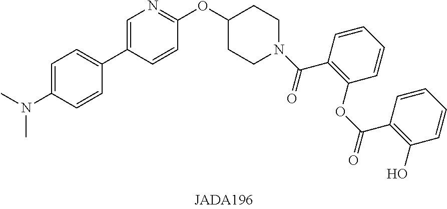 [SMILES: CN(C)c1ccc(-c2ccc(OC3CCN(C(=O)c4ccccc4OC(=O)c4ccccc4O)CC3)nc2)cc1]

To a solution of Int-13 (20.8 mg, 0.05 mmol) and SM-19 (7.9 mg, 0.06 mmol) in DMF (0.5 mL), DIPEA (27.8 mg, 0.2 mmol) and HATU (24 mg, 0.06 mmol) were added. The reaction was stirred at 25° C. for 6 h prior to dilution with EtOAc (2 mL). The mixture was washed with H₂O (2 mL), and the aqueous phase was extracted with additional EtOAc (2×2 mL). The combined organic phases were concentrated under reduced pressure. The resulting residue was purified by ISCO (DCM/MeOH=100/0-90/10) to give JADA196 (8.8 mg, 33% yield).

### Example 2-54: Synthesis of 2-(4-((5-(4-(dimethylamino)phenyl)pyridin-2-yl)oxy)piperidine-1-carbonyl)phenyl morpholine-4-carboxylate (JADA200)

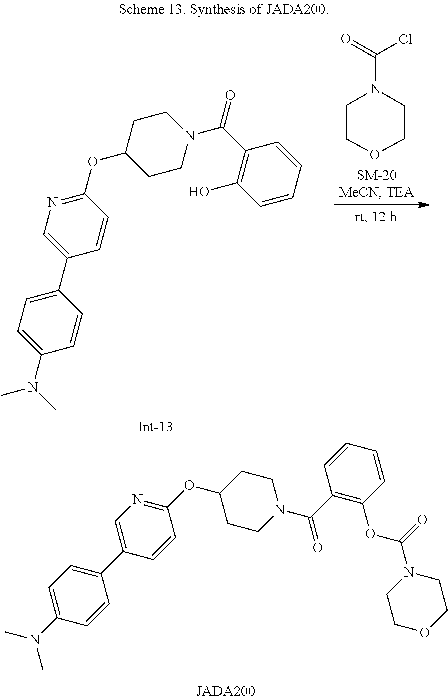 [SMILES: CN(C)c1ccc(-c2ccc(OC3CCN(C(=O)c4ccccc4O)CC3)nc2)cc1 ; O=C(Cl)N1CCOCC1 ; CN(C)c1ccc(-c2ccc(OC3CCN(C(=O)c4ccccc4OC(=O)N4CCOCC4)CC3)nc2)cc1]

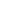

Int-13 was prepared in an analogous manner to JADA196 in Example 2-53 (21.6 mg, 51% yield).

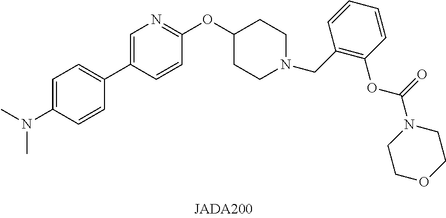 [SMILES: CN(C)c1ccc(-c2ccc(OC3CCN(Cc4ccccc4OC(=O)N4CCOCC4)CC3)nc2)cc1]

To a solution of Int-13 (20.9 mg, 0.05 mmol) and SM-20 (8.1 mg, 0.06 mmol) in MeCN (1 mL), TEA (50.5 mg, 0.5 mmol) was added. The reaction was stirred at 25° C. for 12 h prior to concentration of crude mixture under vacuum. The resulting residue was purified by ISCO (DCM/MeOH=100/0-80/20) to give JADA200 (12.2 mg, yield 46%).

### Example 2-55: Synthesis of 2-((2-(4-((5-(4-(dimethylamino)phenyl)pyridin-2-yl)oxy)piperidine-1-carbonyl)phenoxy)carbonyl)phenyl morpholine-4-carboxylate (JADA201)

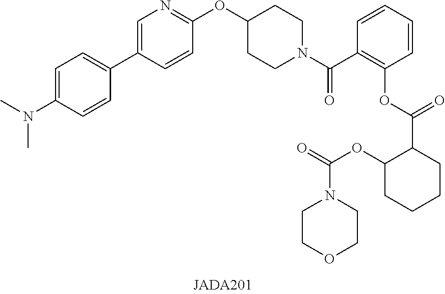 [SMILES: CN(C)c1ccc(-c2ccc(OC3CCN(C(=O)c4ccccc4OC(=O)C4CCCCC4OC(=O)N4CCOCC4)CC3)nc2)cc1]

Int-13 was prepared in an analogous manner to JADA200 in Example 2-54 (2.14 mg, 33% yield).

### Example 2-56: Synthesis of methyl 6-((1-(5-fluoro-2-(morpholine-4-carboxamido)benzoyl)piperidin-4-yl)oxy)nicotinate (JADA210)

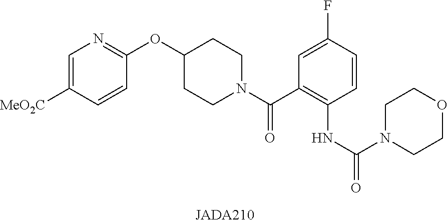 [SMILES: COC(=O)c1ccc(OC2CCN(C(=O)c3cc(F)ccc3NC(=O)N3CCOCC3)CC2)nc1]

JADA210 was prepared in an analogous manner to JADA200 in Example 2-54 (1.94 mg, 40% yield).

### Example 2-57: Synthesis of methyl 6-((1-(5-fluoro-2-(morpholine-4-carboxamido)benzoyl)piperidin-4-yl)oxy)nicotinate (JADA236)

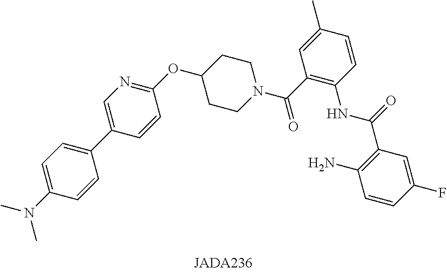 [SMILES: Cc1ccc(NC(=O)c2cc(F)ccc2N)c(C(=O)N2CCC(Oc3ccc(-c4ccc(N(C)C)cc4)cn3)CC2)c1]

JADA236 was prepared in an analogous manner to JADA196 in Example 2-53 (1.19 mg, 21% yield).

### Example 2-58: Synthesis of (2-((4-(dimethylamino)benzyl)amino)-5-fluorophenyl)(4-(4-(dimethylamino)benzyl)piperazin-1-yl)methanone (5)

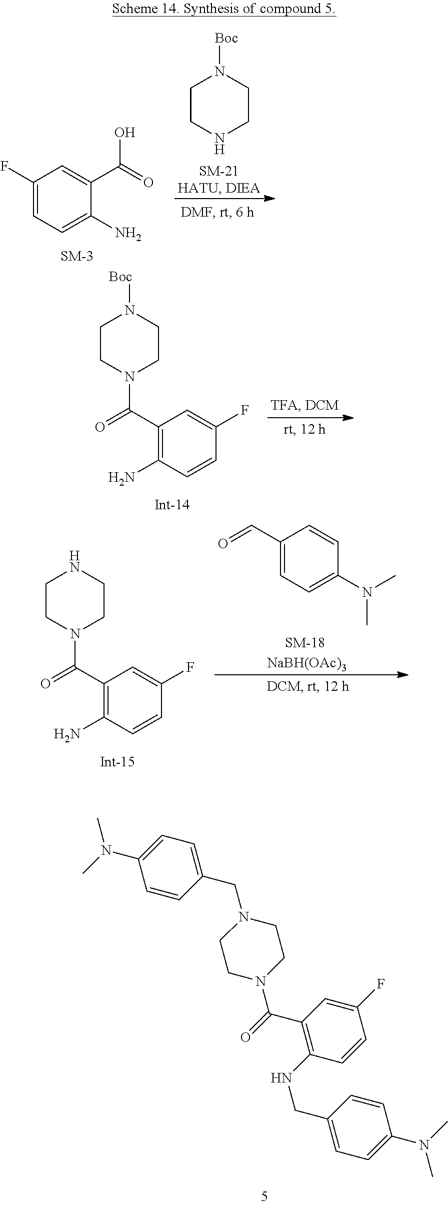

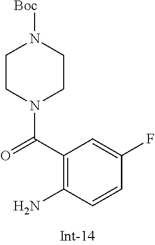 [SMILES: CC(C)(C)OC(=O)N1CCN(C(=O)c2cc(F)ccc2N)CC1]

To a solution of SM-3 (155.0 mg, 1 mmol) and SM-21 (204.5 mg, 1.1 mmol) in DMF (1.5 mL), DIPEA (645 mg, 5 mmol) and HATU (466 mg, 1.2 mmol) were added. The reaction was stirred at 25° C. for 6 h prior to dilution with EtOAc (5 mL). The mixture was washed with H₂O (5 mL), and the aqueous phase was extracted with additional EtOAc (2×3 mL). The combined organic phases were concentrated under reduced pressure. The residue was purified by ISCO (DCM/MeOH=100/0-80/20) to give Int-14 (171.6 mg, 53% yield).

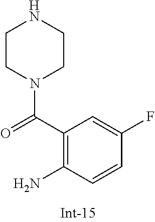 [SMILES: Nc1ccc(F)cc1C(=O)N1CCNCC1]

A solution of Int-14 (161 mg, 0.5 mmol) in DCM (5 mL) and TFA (1.5 mL) in a 10-mL flask was stirred at 25° C. for 12 h. After completion of the reaction, the mixture was concentrated under reduced pressure. The resulting residue was purified via ISCO chromatography (DCM/MeOH=100/0˜60/40) to give the Int-15 (92.5 mg, 83% yield).

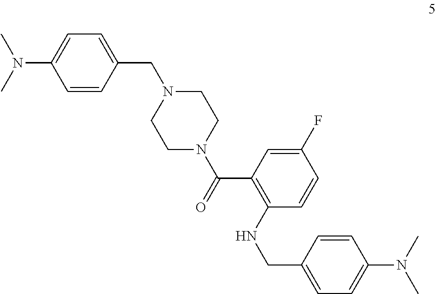

A solution of Int-15 (11.2 mg, 0.05 mmol) and SM-18 (22.4 mg, 0.15 mmol) in CH₂Cl₂ (2 mL) was stirred at 25° C. for 3 h prior to addition of NaBH(AcO)₃ (42.2 mg, 0.2 mmol) in portions. The solution was stirred at 25° C. for 9 h. The crude mixture was concentrated under reduced pressure, and the resulting residue was purified by ISCO (CH₂Cl₂/MeOH=100/0˜80/20) to give compound 5 (5.6 mg, 23% yield).

### Example 2-59: Synthesis of (3-((4-(dimethylamino)benzyl)amino)pyridin-2-yl)(4-(4-(dimethylamino)benzyl)piperazin-1-yl)methanone (15)

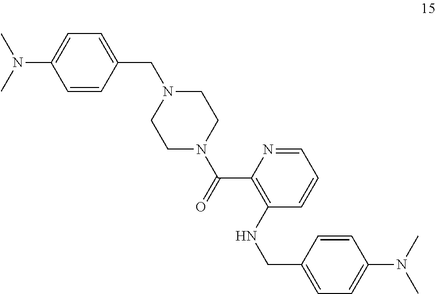

Compound 15 was prepared in an analogous manner to compound 5 in Example 2-58 (3.7 mg, yield 16%).

### Example 2-60: Synthesis of (3-((4-(dimethylamino)benzyl)amino)thiophen-2-yl)(4-(4-(dimethylamino)benzyl)piperazin-1-yl)methanone (16)

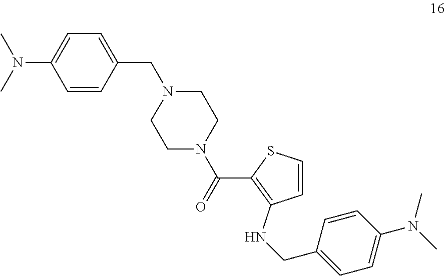

Compound 16 was prepared in an analogous manner to compound 5 in Example 2-58 (2.6 mg, yield 11% yield).

### Example 2-61: Synthesis of 2-(4-(4-(dimethylamino)benzyl)piperazine-1-carbonyl)-N-(4-(dimethylamino)phenethyl)-4-fluorobenzamide (7) and 2-(4-(4-(dimethylamino)benzyl)piperazine-1-carbonyl)-N-(4-(dimethylamino)phenethyl)-5-fluorobenzamide (8)

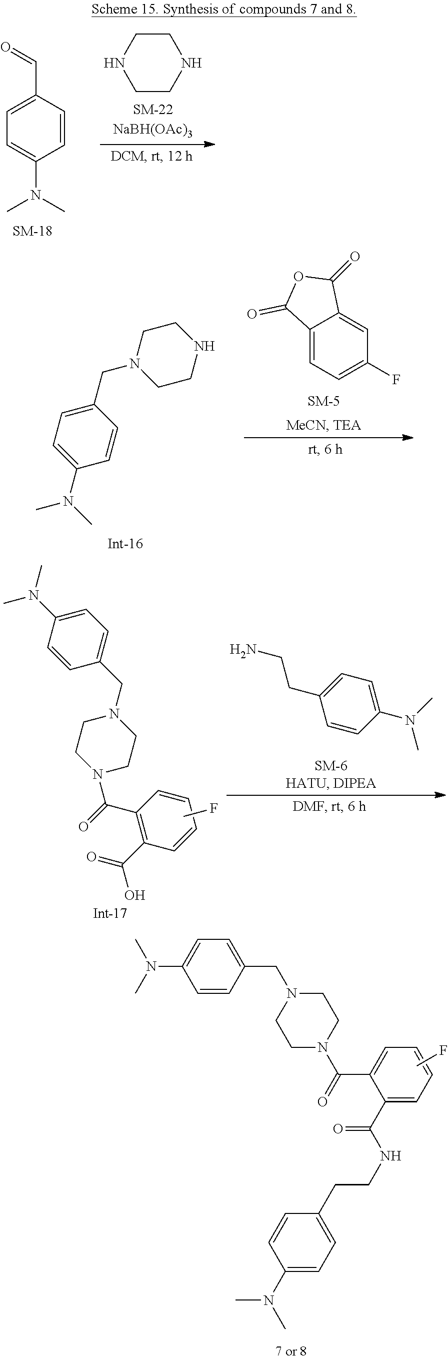

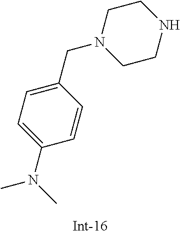 [SMILES: CN(C)c1ccc(CN2CCNCC2)cc1]

A solution of SM-18 (1.49 g, 10 mmol) and SM-22 (2.58 g, 30 mmol) in CH₂Cl₂ (50 mL) was stirred at 25° C. for 3 h prior to addition of NaBH(AcO)₃ (4.22 g, 20 mmol) in portions. The reaction was stirred at 25° C. for 9 h. The crude mixture was concentrated under reduced pressure, and the resulting residue was purified by ISCO (CH₂Cl₂/MeOH=100/0˜60/40) to give Int-16 (1.42 g, 65% yield).

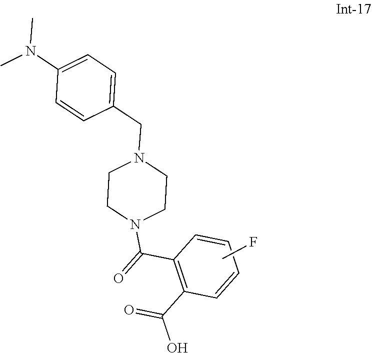

To a solution of Int-16 (24.6 mg, 0.1 mmol) and SM-5 (18.2 mg, 0.11 mmol) in MeCN (1 mL), TEA (50.5 mg, 0.5 mmol) was added. The reaction was stirred at 25° C. for 6 h. The crude mixture was concentrated under reduced pressure, and the resulting residue was purified by ISCO (DCM/MeOH=100/0˜70/30) to give Int-17 (36.1 mg, 86% yield).

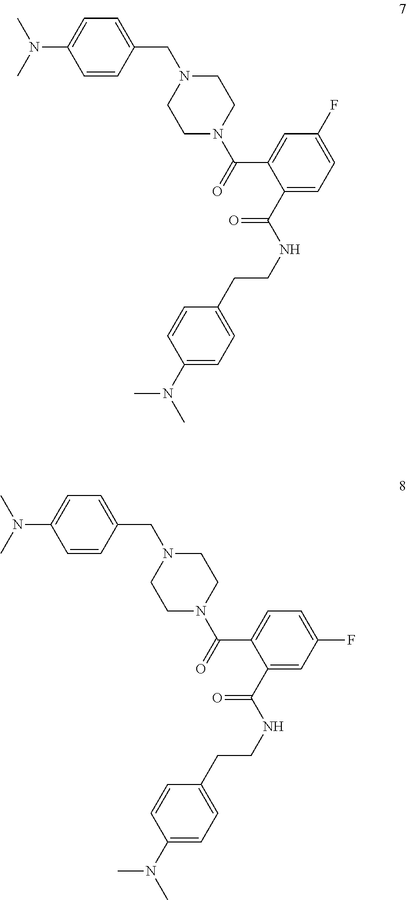 [SMILES: CN(C)c1ccc(CCNC(=O)c2ccc(F)cc2C(=O)N2CCN(Cc3ccc(N(C)C)cc3)CC2)cc1 ; CN(C)c1ccc(CCNC(=O)c2cc(F)ccc2C(=O)N2CCN(Cc3ccc(N(C)C)cc3)CC2)cc1]

To a solution of Int-17 (21.0 mg, 0.05 mmol) and SM-6 (18.0 mg, 0.06 mmol) in DMF (0.5 mL), DIPEA (64.5 mg, 0.5 mmol) and HATU (46.6 mg, 0.12 mmol) were added. The reaction was stirred at 25° C. for 6 h prior to dilution with EtOAc (2 mL). The mixture was washed with H₂O (2 mL), and the aqueous phase was extracted with additional EtOAc (2×2 mL). The combined organic phases were concentrated under reduced pressure. The resulting residue was purified by ISCO (DCM/MeOH=100/0-80/20) to give a mixture of compounds 7 and 8 (7.2 mg, 29% yield).

### Example 2-62: Synthesis of 2-(4-(4-(dimethylamino)benzyl)piperazine-1-carbonyl)-N-(3-(4-(dimethylamino)phenyl)propyl)-4-fluorobenzamide (9) and 2-(4-(4-(dimethylamino)benzyl)piperazine-1-carbonyl)-N-(3-(4-(dimethylamino)phenyl)propyl)-5-fluorobenzamide (10)

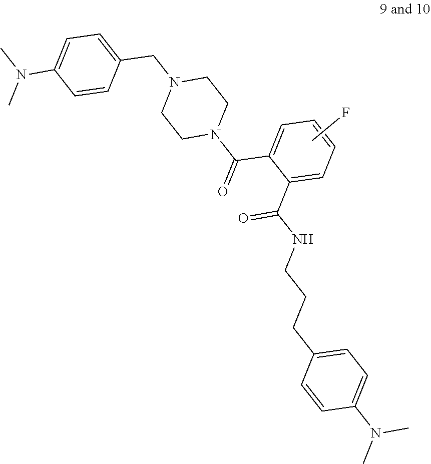

Compounds 9 and 10 were prepared in an analogous manner to compounds 7 and 8 in Example 2-61 (6.2 mg, 25% yield).

### Example 2-63: Synthesis of (2-((2,4-diisopropoxybenzyl)amino)-5-fluorophenyl)(4-(4-(dimethylamino)benzyl)piperazin-1-yl)methanone (13)

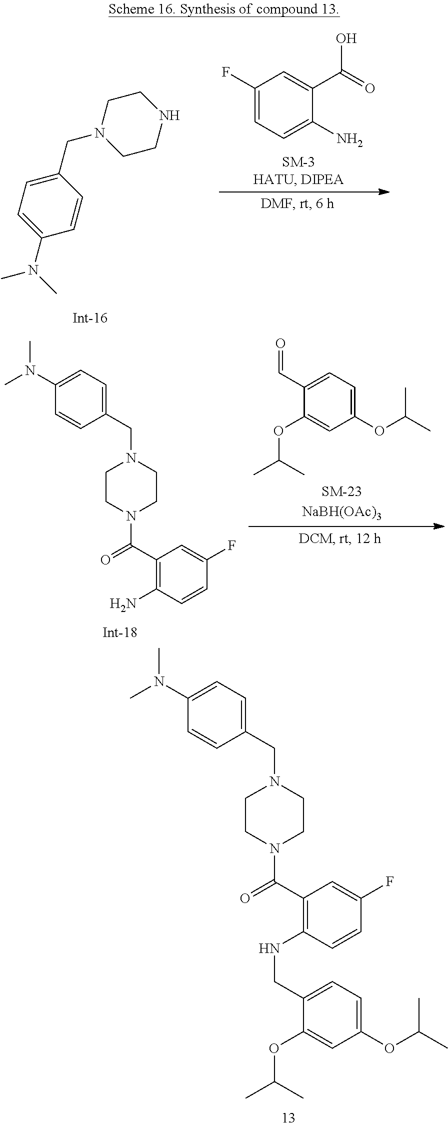 [SMILES: CN(C)c1ccc(CN2CCNCC2)cc1 ; Nc1ccc(F)cc1C(=O)O ; CN(C)c1ccc(CN2CCN(C(=O)c3cc(F)ccc3N)CC2)cc1 ; CC(C)Oc1ccc(C=O)c(OC(C)C)c1 ; CC(C)Oc1ccc(CNc2ccc(F)cc2C(=O)N2CCN(Cc3ccc(N(C)C)cc3)CC2)c(OC(C)C)c1]

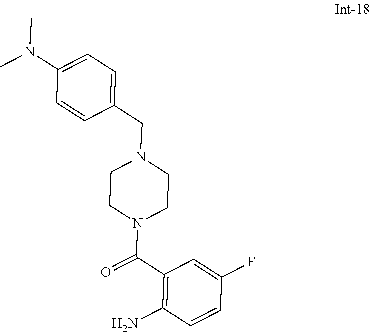 [SMILES: CN(C)c1ccc(CN2CCN(C(=O)c3cc(F)ccc3N)CC2)cc1]

To a solution of Int-16 (21.3 mg, 0.1 mmol) and SM-3 (17.5 mg, 0.11 mmol) in DMF (0.5 mL), DIPEA (64.5 mg, 0.5 mmol) and HATU (46.6 mg, 0.12 mmol) were added. The reaction was stirred at 25° C. for 6 h prior to dilution with EtOAc (3 mL). The mixture was washed with H₂O (2 mL), and the aqueous phase was extracted with additional EtOAc (2×2 mL). The combined organic phases were concentrated under reduced pressure. The resulting residue was purified by ISCO (DCM/MeOH=100/0-70/30) to give Int-18 (16.3 mg, 46% yield).

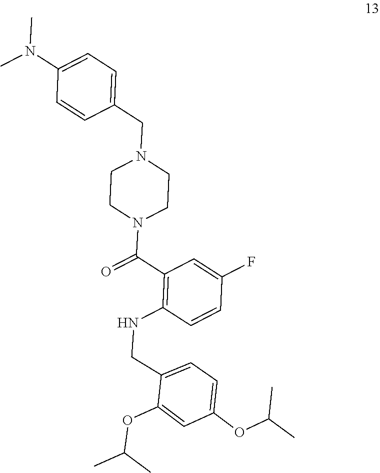 [SMILES: CC(C)Oc1ccc(CNc2ccc(F)cc2C(=O)N2CCN(Cc3ccc(N(C)C)cc3)CC2)c(OC(C)C)c1]

A solution of Int-18 (10.6 mg, 0.03 mmol) and SM-23 (8.9 mg, 0.04 mmol) in CH₂Cl₂ (1 mL) was stirred at 25° C. for 3 h prior addition of NaBH(AcO)₃ (10.55 mg, 0.05 mmol) in portions. The reaction was stirred at 25° C. for 9 h. The crude mixture was concentrated under reduced pressure, and the resulting residue was purified by ISCO (CH₂Cl₂/MeOH=100/0˜90/10) to give compound 13 (3.9 mg, 21% yield).

### Example 2-64: Synthesis of 4-(((2-(4-(4-(dimethylamino)benzyl)piperazine-1-carbonyl)-4-fluorophenyl)amino)methyl)-1,3-dimethyl-1,3-dihydro-2H-imidazol-2-one (14)

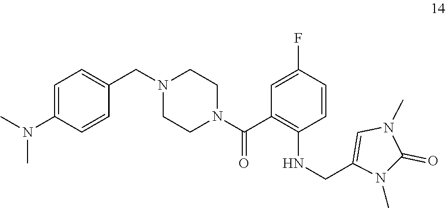 [SMILES: CN(C)c1ccc(CN2CCN(C(=O)c3cc(F)ccc3NCc3cn(C)c(=O)n3C)CC2)cc1]

Compound 14 was prepared in an analogous manner to compound 13 in Example 2-63 (3.7 mg, 23% yield).

### Example 2-65: Synthesis of (2-((4-(dimethylamino)benzyl)amino)-5-fluorophenyl)(4-(3-((dimethylamino)methyl)-4-hydroxy benzyl)piperazin-1-yl)methanone (17)

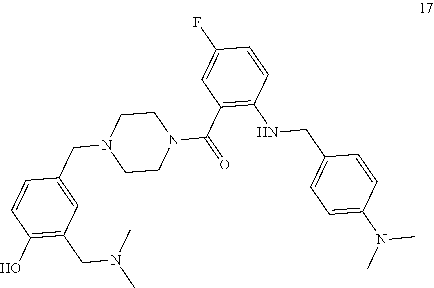 [SMILES: CN(C)Cc1cc(CN2CCN(C(=O)c3cc(F)ccc3NCc3ccc(N(C)C)cc3)CC2)ccc1O]

Compound 17 was prepared in an analogous manner to compound 13 in Example 2-63 (1.73 mg, 11% yield).

### Example 2-66: Synthesis of (2-((4-(dimethylamino)benzyl)amino)-5-fluorophenyl)(4-((4-(dimethylamino)benzyl)amino)piperidin-1-yl)methanone (18)

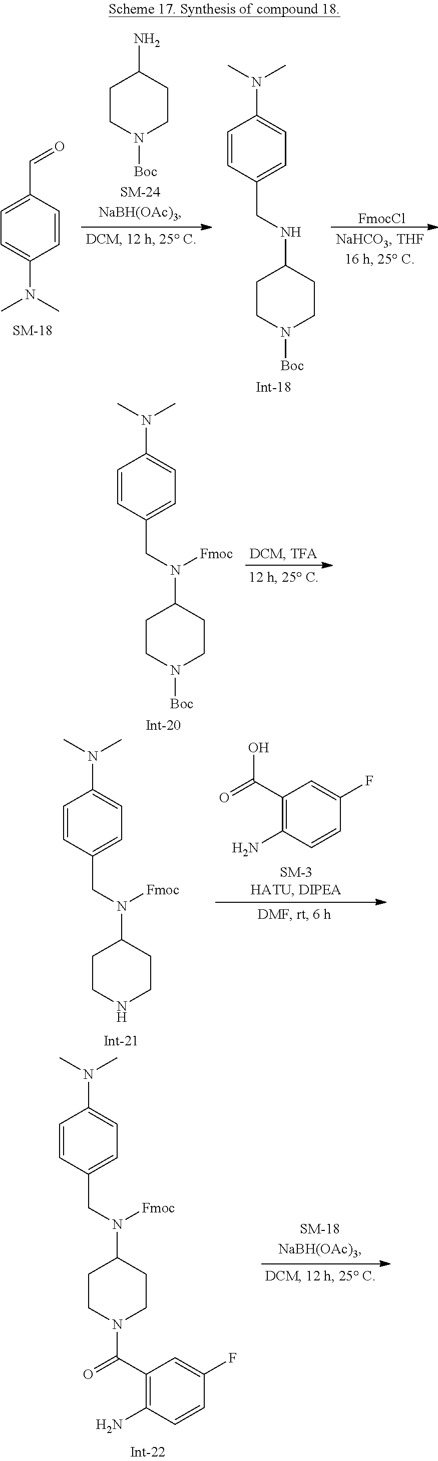

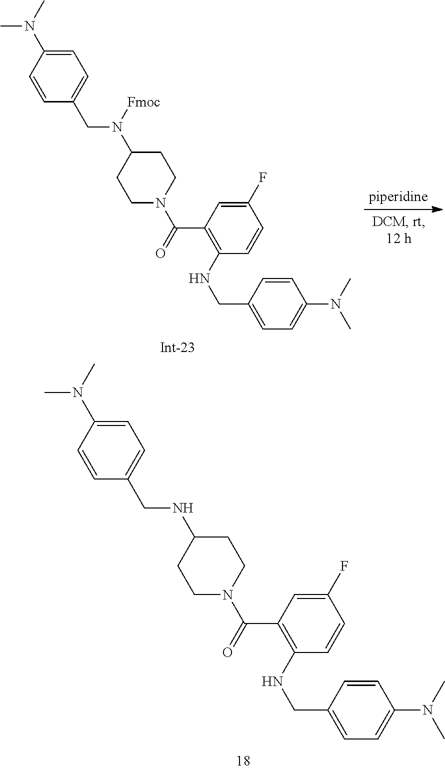

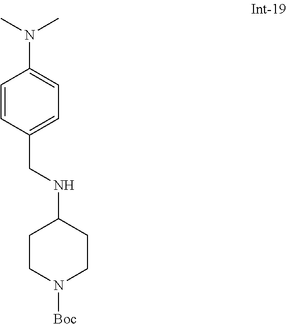 [SMILES: CN(C)c1ccc(CNC2CCN(C(=O)OC(C)(C)C)CC2)cc1]

A solution of SM-18 (1.49 g, 10 mmol) and SM-24 (4.0 g, 20 mmol) in CH₂Cl₂ (30 mL) was stirred at 25° C. for 3 h prior to addition of NaBH(AcO)₃ (4.22 g, 20 mmol) in portions. The reaction was stirred at 25° C. for 9 h. The crude mixture was concentrated under reduced pressure, and the resulting residue was purified by ISCO (CH₂Cl₂/MeOH=100/0˜70/30) to give Int-19 (2.32 g, 70% yield).

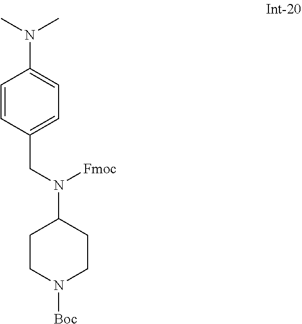

To a solution of Int-19 (1.66 g, 5 mmol) in THF: H₂O (100 mL: 10 mL), NaHCO₃ (0.86 g, 10 mmol) and FmocCl (1.54 g, 6 mmol) were added. The reaction was stirred at 25° C. for 16 h. The reaction mixture was evaporated under reduced pressure and then extracted with EA (50 mL×2). The combined organic phases were concentrated under reduced pressure. The resulting residue was purified by ISCO (CH₂Cl₂/MeOH=100/0˜95/5) to afford Int-20 (1.94 g, 70% yield).

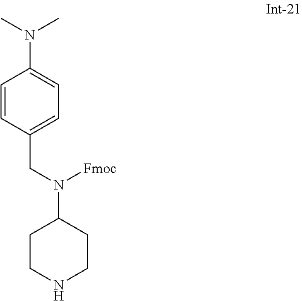

A solution of Int-14 (555 mg, 1 mmol) in DCM (5 mL) and TFA (1.5 mL) in a 10-mL flask was stirred at 25° C. for 12 h. After completion of the reaction, the mixture was concentrated under reduced pressure. The resulting residue was purified via ISCO chromatography (DCM/MeOH=100/0˜80/20) to give Int-21 (364.5 mg, 80% yield).

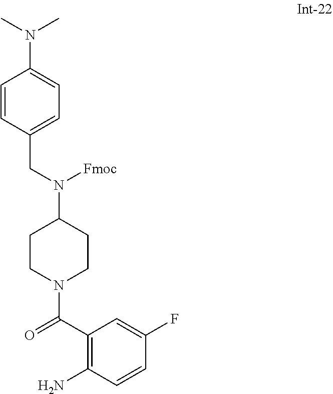

To a solution of Int-21 (91.0 mg, 0.2 mmol) and SM-3 (23.5 mg, 0.15 mmol) in DMF (0.5 mL), DIPEA (64.5 mg, 0.5 mmol) and HATU (76.0 mg, 0.12 mmol) were added. The reaction was stirred at 25° C. for 6 h prior to dilution with EtOAc (3 mL). The mixture was washed with H₂O (2 mL), and the aqueous phase was extracted with additional EtOAc (2×2 mL). The combined organic phases were concentrated under reduced pressure. The resulting residue was purified by ISCO (DCM/MeOH=100/0-70/30) to give Int-22 (58.3 mg, 65% yield).

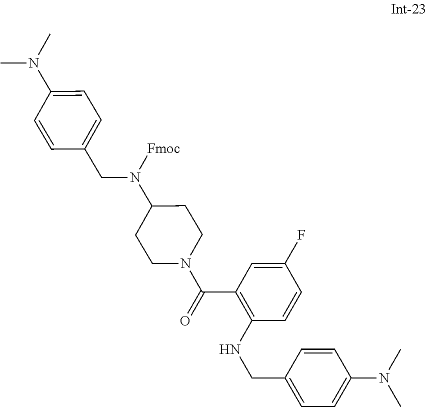

A solution of Int-22 (29.6 mg, 0.05 mmol) and SM-18 (8.9 mg, 0.06 mmol) in CH₂Cl₂ (1 mL) was stirred at 25° C. for 3 h prior to addition of NaBH(AcO)₃ (10.55 mg, 0.05 mmol) in portions. The reaction was stirred at 25° C. for 9 h. The crude mixture was concentrated under reduced pressure, and the resulting residue was purified by ISCO (CH₂Cl₂/MeOH=100/0˜90/10) to give Int-23 (25.7 mg, 71% yield).

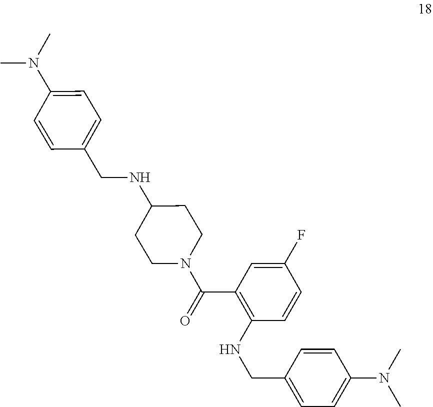

A solution of Int-23 (14.3 mg, 0.002 mmol) and piperidine (8.6 mg, 0.01 mmol) in DCM (2 mL) was stirred at 25° C. for 6 h. After completion of the reaction, the mixture was concentrated under reduced pressure. The resulting residue was purified via ISCO chromatography (DCM/MeOH=100/0˜80/20) to give compound 18 (5.5 mg, 60% yield).

### Example 2-67: Synthesis of N-(2-((4-(dimethylamino)benzyl)(methyl)amino)ethyl)-2-((4-(dimethylamino)benzyl)amino)-5-fluoro-N-methylbenzamide (19)

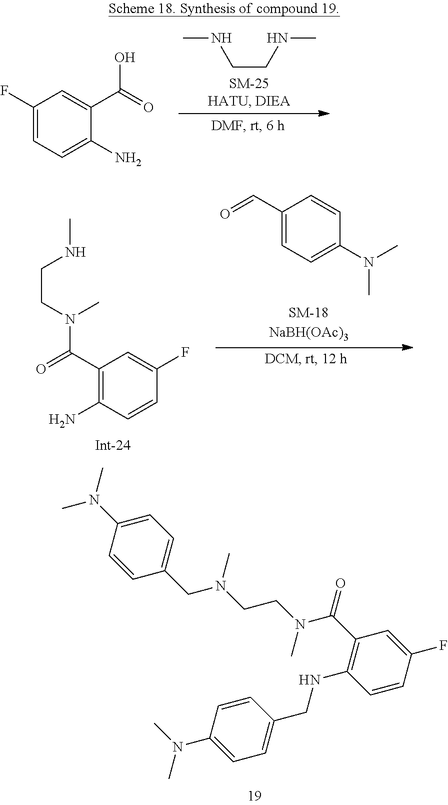

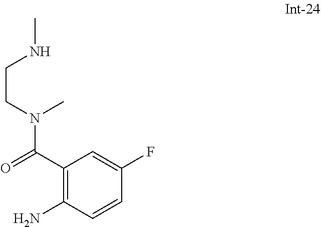 [SMILES: CNCCN(C)C(=O)c1cc(F)ccc1N]

To a solution of Int-3 (235.0 mg, 1.5 mmol) and SM-25 (264.5 mg, 3 mmol) in DMF (1.5 mL), DIPEA (129 mg, 10 mmol) and HATU (760 mg, 2 mmol) were added. The reaction was stirred at 25° C. for 6 h prior to dilution with EtOAc (5 mL). The mixture was washed with H₂O (5 mL), and the aqueous phase was extracted with additional EtOAc (2×3 mL). The combined organic phases were concentrated under reduced. The resulting residue was purified by ISCO (DCM/MeOH=100/0-60/40) to give Int-24 (212.3 mg, 63% yield).

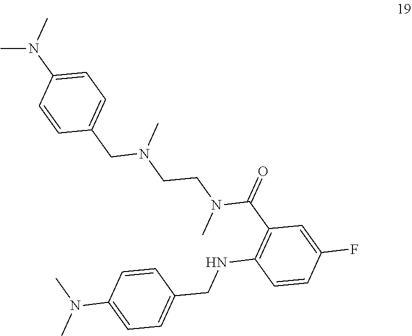 [SMILES: CN(CCN(C)C(=O)c1cc(F)ccc1NCc1ccc(N(C)C)cc1)Cc1ccc(N(C)C)cc1]

A solution of SM-18 (29.8 mg, 0.2 mmol) and Int-24 (11.5 mg, 0.05 mmol) in CH₂Cl₂ (1 mL) was stirred at 25° C. for 3 h prior to addition of NaBH(AcO)₃ (31.6 mg, 0.15 mmol) in portions. The solution was stirred at 25° C. for 9 h. The crude mixture solution was concentrated under reduced pressure, and the resulting residue was purified by ISCO (CH₂Cl₂/MeOH=100/0˜70/30) to give compound 19 (7.3 mg, 30% yield).

### Example 2-68: Synthesis of N-(2-((4-((dimethylamino)methyl)benzyl)(methyl)amino)ethyl)-2-((4-((dimethylamino)methyl)benzyl)amino)-5-fluoro-N-methylbenzamide (20)

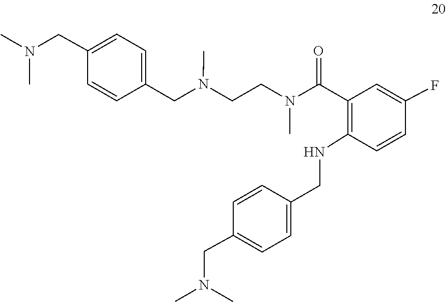 [SMILES: CN(C)Cc1ccc(CNc2ccc(F)cc2C(=O)N(C)CCN(C)Cc2ccc(CN(C)C)cc2)cc1]

Compound 20 was prepared in an analogous manner to compound 19 in Example 2-67 (2.7 mg, 11.5% yield).

### Example 2-69: Synthesis of (E)-N-(4-(dimethylamino)benzyl)-4-(4-(4-(dimethylamino)benzyl)piperazin-1-yl)-4-oxobut-2-enamide (21)

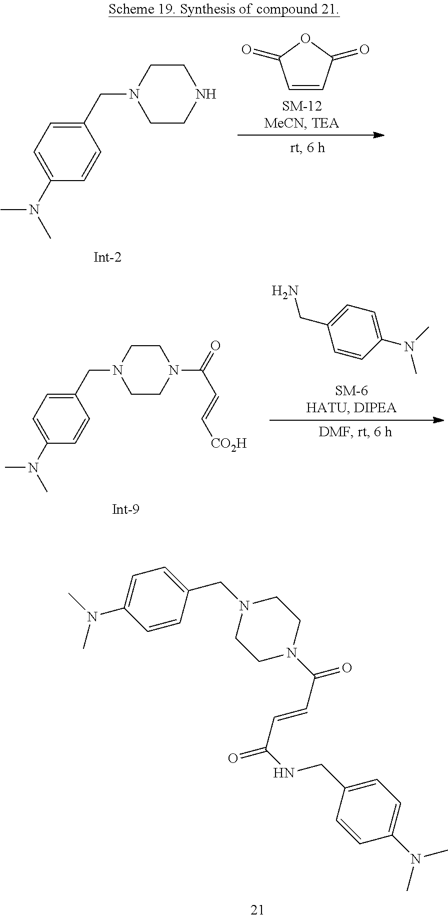

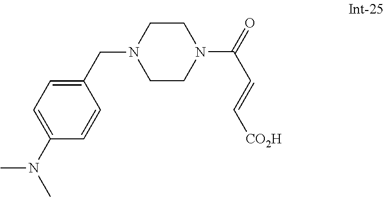 [SMILES: CN(C)c1ccc(CN2CCN(C(=O)/C=C/C(=O)O)CC2)cc1]

To a solution of Int-16 (21.9 mg, 0.1 mmol) and SM-12 (10.8 mg, 0.11 mmol) in MeCN (1 mL), TEA (50.5 mg, 0.5 mmol) was added. The reaction was stirred at 25° C. for 6 h prior to concentration of crude mixture under reduced pressure. The resulting residue was purified by ISCO (DCM/MeOH=100/0-70/30) to give Int-25 (23.1 mg, 73% yield).

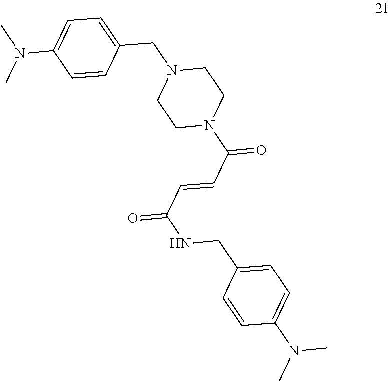

To a solution of Int-25 (15.5 mg, 0.05 mmol) and SM-6 (9.6 mg, 0.06 mmol) in DMF (0.5 mL), DIPEA (27.8 mg, 0.2 mmol) and HATU (24 mg, 0.06 mmol) were added. The reaction was stirred at 25° C. for 6 h prior to dilution with EtOAc (2 mL). The mixture was washed with H₂O (2 mL), and the aqueous phase was extracted with additional EtOAc (2×2 mL). The combined organic phases were concentrated under reduced pressure. The resulting residue was purified by ISCO (DCM/MeOH=100/0-90/10) to give compound 21 (4.2 mg, 19% yield).

### Example 2-70: Synthesis of (E)-4-(4-(4-(dimethylamino)benzyl)piperazin-1-yl)-N-(4-(dimethylamino)phenyl)-4-oxobut-2-enamide (22)

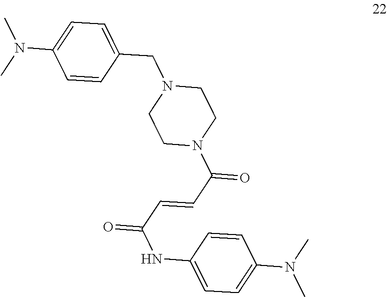

Compound 22 was prepared in an analogous manner to compound 21 in Example 2-69 (3.7 mg, yield 17%).

### Equivalents and Scope

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

Furthermore, the present disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the present disclosure, or aspects of the present disclosure, is/are referred to as comprising particular elements and/or features, certain embodiments of the present disclosure or aspects of the present disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the present disclosure, to the tenth of the unit of the lower limit of the range, unless the context clearly dictates otherwise.

This application refers to various issued patents, published patent applications, journal articles, and other publications, the contents of all of which are incorporated herein by reference in their entireties. If there is a conflict between any of the incorporated publication and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

1. A compound of Formula (I): 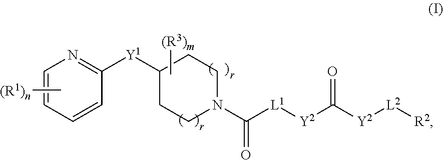
or a pharmaceutically acceptable salt, hydrate, solvate, polymorph, co-crystal, tautomer, stereoisomer, isotopically labeled derivative, or prodrug thereof, wherein:
each instance of R¹ is independently hydrogen, halogen, —CN, —N₃, —NO₂, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, optionally substituted sulfonyl, optionally substituted sulfinyl, —OR^{O}, —N(10)₂, or —SR^{S};
n is an integer from 0-4, inclusive;
Y¹ is —C(R^{C})₂—, —O—, —NR^{N}—, or —S—;
each instance of R³ is independently hydrogen, halogen, —CN, —N₃, —NO₂, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, —OR^{O}, —N(R^{N})₂, or —SR^{S};
m is 0, or an integer from 1 to 5, inclusive;
each instance of r is independently 0 or 1;
L¹ is optionally substituted C₁₋₆ alkylene, optionally substituted C₂₋₆ alkenylene, optionally substituted carbocyclyl, optionally substituted heterocyclyl, optionally substituted arylene, or optionally substituted heteroarylene;
each instance of Y² is independently a bond, —O—, or —NR^{N}—;
L² is a bond, optionally substituted C₁₋₆ alkylene, optionally substituted C₁₋₆ heteroalkylene, optionally substituted C₂₋₆ alkenylene, optionally substituted C₂₋₆ alkynylene, optionally substituted C₁₋₆ acylene, or a combination thereof;
R² is —N(R^{N})₂, optionally substituted aryl, optionally substituted heteroaryl, optionally substituted carbocyclyl, or optionally substituted heterocyclyl;
each instance of R^{N} is independently hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, optionally substituted sulfonyl, optionally substituted sulfinyl, or a nitrogen protecting group; or two R^{N} bonded to the same nitrogen atom are taken together with the intervening atoms to form optionally substituted heterocyclyl or optionally substituted heteroaryl;
each instance of R^{C} is independently hydrogen, halogen, —CN, —N₃, —NO₂, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, optionally substituted sulfonyl, optionally substituted sulfinyl, —OR^{O}, —N(R^{N})₂, or —SR^{S}; or two R^{C} bonded to the same carbon are taken together with the intervening atoms to form optionally substituted carbocyclyl, optionally substituted heterocyclyl, or ═O;
each instance of R^{O} is independently hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, or an oxygen protecting group; and
each instance of R^{S} is independently hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, a sulfur protecting group, or R^{S}, taken together with the sulfur atom to which it is attached forms a disulfide group substituted with hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, or optionally substituted acyl,
provided that the compound is not:

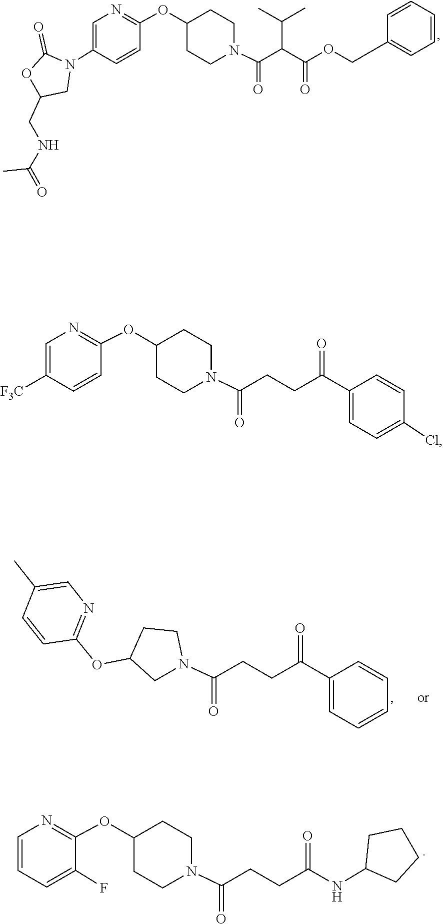 [SMILES: CC(=O)NCC1CN(c2ccc(OC3CCN(C(=O)C(C(=O)OCc4ccccc4)C(C)C)CC3)nc2)C(=O)O1 ; O=C(CCC(=O)N1CCC(Oc2ccc(C(F)(F)F)cn2)CC1)c1ccc(Cl)cc1 ; Cc1ccc(OC2CCN(C(=O)CCC(=O)c3ccccc3)C2)nc1 ; O=C(CCC(=O)N1CCC(Oc2ncccc2F)CC1)NC1CCCC1]

2. The compound of claim 1, wherein the compound is of Formula (I-i), (I-ii), or (I-iii): 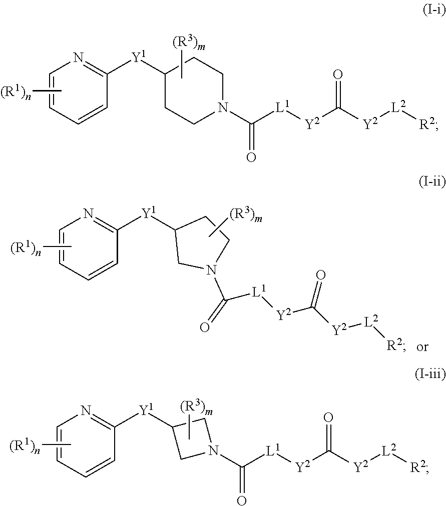
or a pharmaceutically acceptable salt, hydrate, solvate, polymorph, co-crystal, tautomer, stereoisomer, isotopically labeled derivative, or prodrug thereof.

3.-4. (canceled)

5. The compound of claim 1, wherein the compound is of Formula (I-a), (I-b), or (I-c): 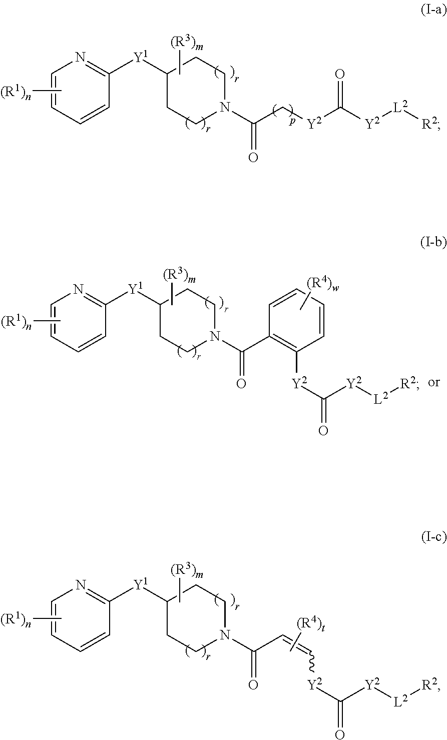
or a pharmaceutically acceptable salt, hydrate, solvate, polymorph, co-crystal, tautomer, stereoisomer, isotopically labeled derivative, or prodrug thereof, wherein:
each instance of R4 is independently hydrogen, halogen, —CN, —N3, —NO₂, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, optionally substituted sulfonyl, optionally substituted sulfinyl, —OR^{O}, —N(R^{N})₂, or —SR^{S},
p is an integer from 1 to 6, inclusive; and
w is 0, or an integer from 1 to 4, inclusive.

6. The compound of claim 5, wherein the compound is of Formula (I-a) and represented by Formula (I-a-1), (I-a-2), (I-a-3), (I-a-4), or (I-a-5): 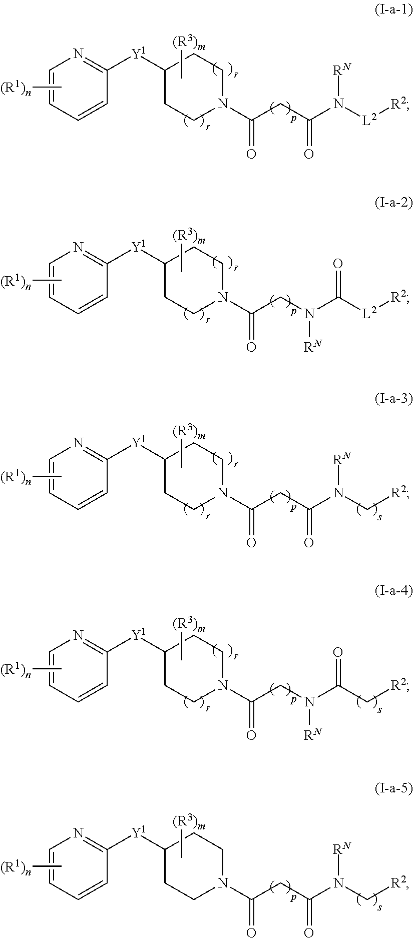
wherein:
s is an integer from 1 to 6, inclusive,
or a pharmaceutically acceptable salt, hydrate, solvate, polymorph, co-crystal, tautomer, stereoisomer, isotopically labeled derivative, or prodrug thereof.

7.-9. (canceled)

10. The compound of claim 5, wherein the compound is of Formula (I-b) and represented by Formula (I-b-1), (I-b-2)), (I-b-3), (I-b-4), (I-b-5), or (I-b-6): 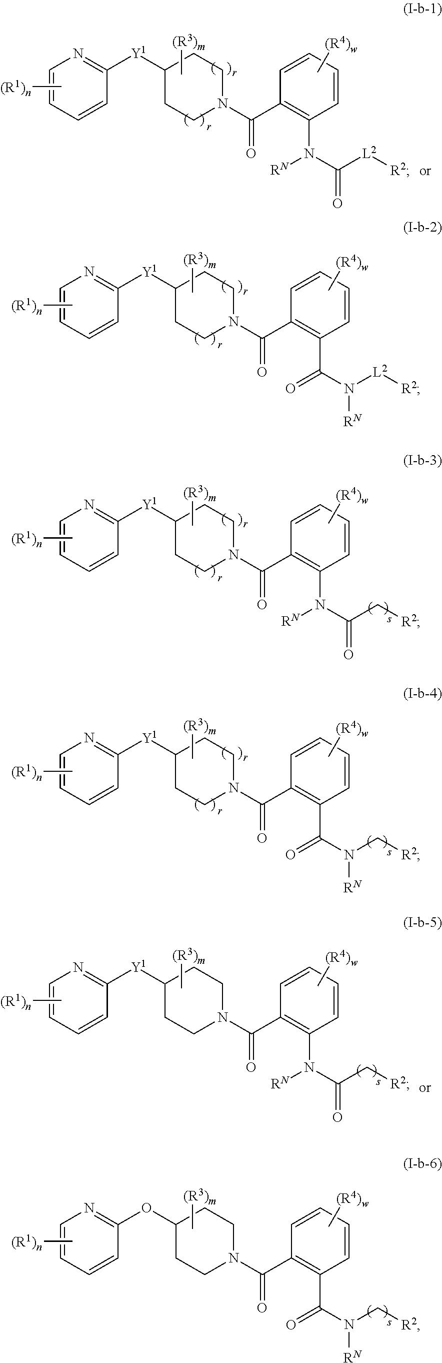
wherein:
s is an integer from 1 to 6, inclusive,
or a pharmaceutically acceptable salt, hydrate, solvate, polymorph, co-crystal, tautomer, stereoisomer, isotopically labeled derivative, or prodrug thereof.

11.-13. (canceled)

14. The compound of claim 5, wherein the compound is of Formula (I-c) and represented by Formula (I-c-1), (I-c-2), (I-c-3), (I-c-4), (I-c-5), (I-c-6), (I-c-7), or (I-c-8): 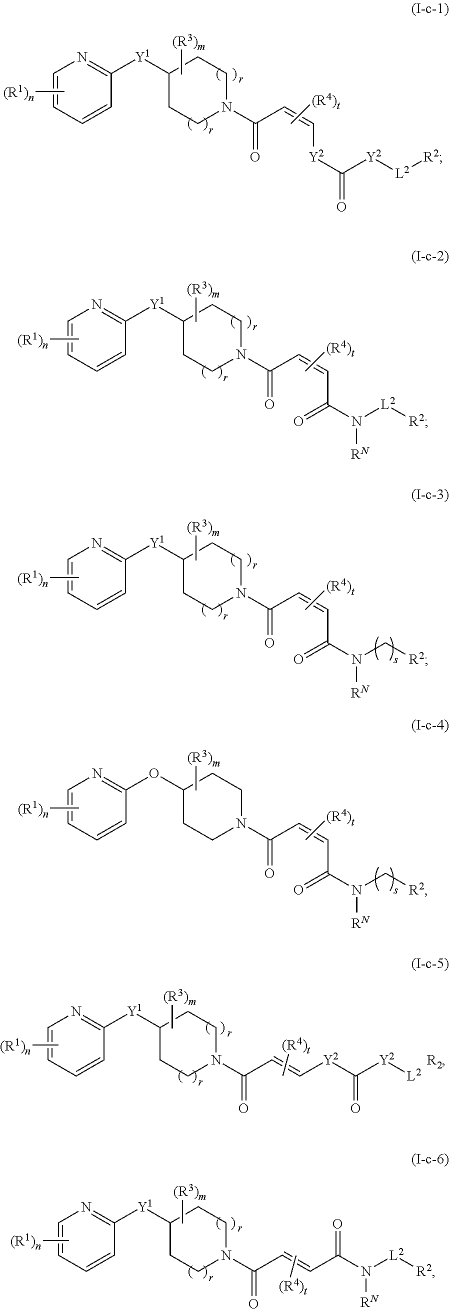 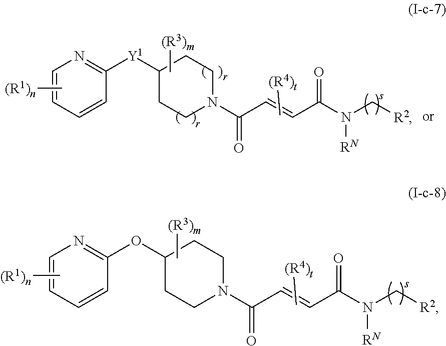
wherein:
s is an integer from 1 to 6, inclusive,
or a pharmaceutically acceptable salt, hydrate, solvate, polymorph, co-crystal, tautomer, stereoisomer, isotopically labeled derivative, or prodrug thereof.

15.-21. (canceled)

22. The compound of claim 1, wherein:
L¹ is optionally substituted C₁₋₆ alkylene, optionally substituted C₂₋₆ alkenylene, optionally substituted arylene, or optionally substituted heteroarylene; or
L¹ is optionally substituted carbocyclyl or optionally substituted heterocyclyl.

23. (canceled)

24. The compound of claim 22, wherein L¹ is optionally substituted C₂₋₃ alkylene.

25.-30. (canceled)

31. The compound of claim 1, wherein L¹ is of the structure: 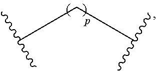
wherein p is an integer from 1 to 6, inclusive.

32. The compound of claim 31, wherein p is 2 or 3.

33.-35. (canceled)

36. The compound of claim 1, wherein L¹ is of structure: 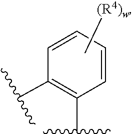
wherein,
each instance of R⁴ is independently hydrogen, halogen, —CN, —N₃, —NO₂, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, optionally substituted sulfonyl, optionally substituted sulfinyl, —OR^{O}, —N(R^{N})₂, or —SR^{S}; and
w is 0, or an integer from 1 to 4, inclusive.

37. The compound of claim 36, wherein L¹ is any one of structures:

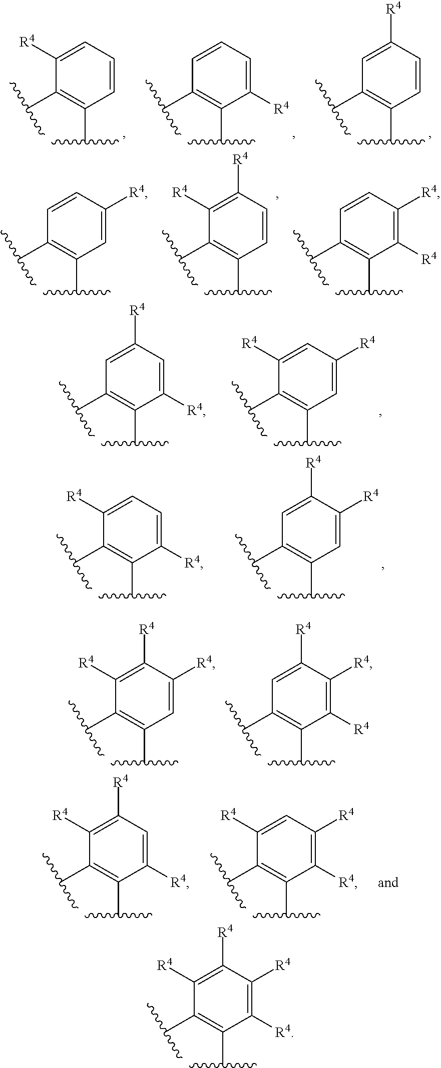

38.-40. (canceled)

41. The compound of claim 36, wherein L¹ is of structure:

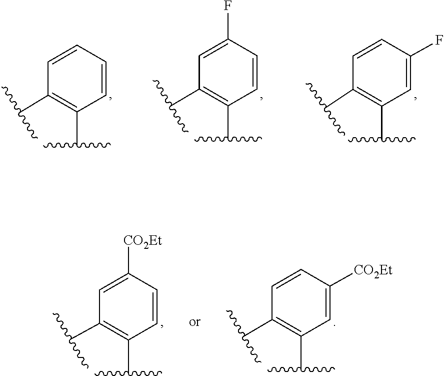 [SMILES: *c1ccccc1* ; *c1ccc(F)cc1* ; *c1ccc(F)cc1* ; *c1ccc(C(=O)OCC)cc1* ; *c1ccc(C(=O)OCC)cc1*]

42.-44. (canceled)

45. The compound of claim 22, wherein L¹ is of structure: 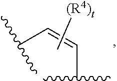
wherein,
each instance of R⁴ is independently hydrogen, halogen, —CN, —N₃, —NO₂, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, optionally substituted sulfonyl, optionally substituted sulfinyl, —OR^{O}, —N(R^{N})₂, or —SR^{S}; and
t is 0, or an integer from 1 to 2, inclusive.

46. The compound of claim 45, wherein L¹ is of structure:

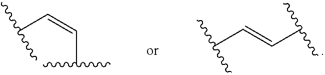

47. (canceled)

48. The compound of claim 45, wherein at least one instance of R⁴ is hydrogen, halogen, or optionally substituted acyl.

49. (canceled)

50. The compound of claim 45, wherein at least one instance of R⁴ is —F, or —CO₂alkyl.

51. (canceled)

52. The compound of claim 45, wherein at least one instance of R⁴ is —CO₂Et.

53. The compound of claim 1, wherein:
w is 0 or 1; or
t is 0; or
Y¹ is —O—, —NR^{N}—, —NH—, or —CH₂—; or
at least one instance of Y² is —NH— or —N(R^{N})—; or
wherein one instance of Y₂ is —NH— or —N(R^{N})—, and the other instance is a bond;
L² is optionally substituted C₁₋₆ alkylene; or
R² is optionally substituted aryl or 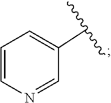 [SMILES: *c1cccnc1]
or
R¹ is optionally substituted alkyl, optionally substituted aryl, or optionally substituted heterocyclyl.

54.-68. (canceled)

69. The compound of claim 53, wherein L² is optionally substituted C₂₋₃ alkylene.

70.-76. (canceled)

77. The compound of claim 53, wherein R² is of structure: 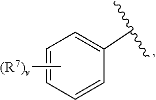
wherein,
each instance of R⁷ is independently hydrogen, halogen, —CN, —N₃, —NO₂, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, optionally substituted sulfonyl, optionally substituted sulfinyl, —OR^{O}, —N(R^{N})₂, or —SR^{S}; and
v is 0, or an integer from 1 to 5, inclusive.

78. The compound of claim 77, wherein R² is of structure:

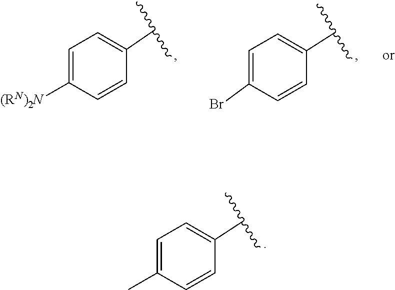

79. The compound of claim 77, wherein R² is of structure:

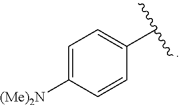

80.-83. (canceled)

84. The compound of claim 53, wherein:
R¹ is haloalkyl; or
R¹ is heterocyclyl; or
R¹ is morpholinyl; or
R¹ is piperidinyl.

85.-89. (canceled)

90. The compound of claim 53, wherein R¹ is of structure: 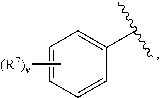
wherein:
each instance of R⁷ is independently Br, optionally substituted alkyl, optionally substituted acyl, optionally substituted sulfonyl, optionally substituted sulfinyl, —OR^{O}, or —N(R^{N})₂.

91. (canceled)

92. The compound of claim 90, wherein R¹ is of structure:

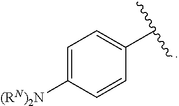

93. The compound of claim 92, wherein R¹ is of structure:

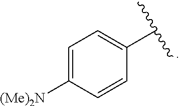

94. The compound of claim 90, wherein at least one instance of R⁷ is:
—N(CH₃)₂; or
methyl; or
haloalkyl; or
alkyl-heterocyclyl; or
alkyl-morpholinyl or alkyl-piperidinyl; or
—NHC(O)-alkyl, —NHC(O)-alkyl-C(O)OH, or —NHC(O)-alkyl-C(O)O-alkyl.

95.-105. (canceled)

106. The compound of claim 1, wherein at least one instance of R³ is hydrogen or wherein each instance of R₃ is hydrogen.

107. (canceled)

108. The compound of claim 1, wherein:
provided that when L¹ is optionally substituted C₁₋₂ alkylene, then at least one instance of Y² is —NR^{N}—; and R² is optionally substituted aryl or optionally substituted heteroaryl; or
provided that when L¹ is optionally substituted C₁₋₂ alkylene, then at least one instance of Y² is —NR^{N}—, and R² is not optionally substituted carbocyclyl; or
provided that when L¹ is optionally substituted C₁₋₂ alkylene, then at least one instance of Y² is —NR^{N}—, and R² is not cyclopentyl; or
provided that when L¹ is optionally substituted C₁₋₄ alkylene, then at least one instance of Y² is —NR^{N}—; or
provided that when L¹ is optionally substituted C₁ alkylene or unsubstituted C₄ alkylene, then at least one instance of Y² is —NR^{N}—; or
provided that when L¹ is optionally substituted C₂ alkylene, then at least one instance of Y² is —NR^{N}—, and R² is optionally substituted aryl or optionally substituted heteroaryl; or
provided that when L¹ is optionally substituted C₂ alkylene, then at least one instance of Y² is —NR^{N}—, and R² is not optionally substituted carbocyclyl; or
provided that when L¹ is optionally substituted C₂ alkylene, then at least one instance of Y² is —NR^{N}—, and R² is not cyclopentyl; or
provided that when R¹ is optionally substituted C₁ alkyl, then at least one instance of Y² is —NR^{N}—; or
provided that when R¹ is optionally substituted alkyl, L¹ is optionally substituted C₂ alkyl, each instance of Y2 is a bond, and L2 is a bond, then R² is not optionally substituted aryl; or
provided that where R¹ is halo, L¹ is optionally substituted C₂ alkyl, one of Y² is —NR^{N}— and the other is a bond, and L² is a bond, then R² is not optionally substituted carbocyclyl; or
provided that where R¹ is halo, L¹ is optionally substituted C₂ alkyl, one of Y² is —NR^{N}— and the other is a bond, and L² is a bond, then R² is not cyclopentyl; or
provided that when R¹ is optionally substituted C₁ alkyl, then at least one instance of Y² is —NR^{N}—; or
provided that when R¹ is optionally substituted alkyl, L¹ is optionally substituted C₂ alkyl, each instance of Y2 is a bond, and L2 is a bond, then R² is not optionally substituted aryl; or
provided that where R¹ is halo, L¹ is optionally substituted C₂ alkyl, one of Y² is —N₁₀— and the other is a bond, and L² is a bond, then R² is not optionally substituted carbocyclyl; or
provided that where R¹ is halo, L¹ is optionally substituted C₂ alkyl, one of Y² is —NR^{N}— and the other is a bond, and L² is a bond, then R² is not cyclopentyl; or
R¹ is not oxazolidinone.

109.-122. (canceled)

123. The compound of claim 1, wherein 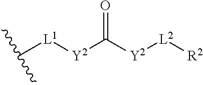
is any one of structures:

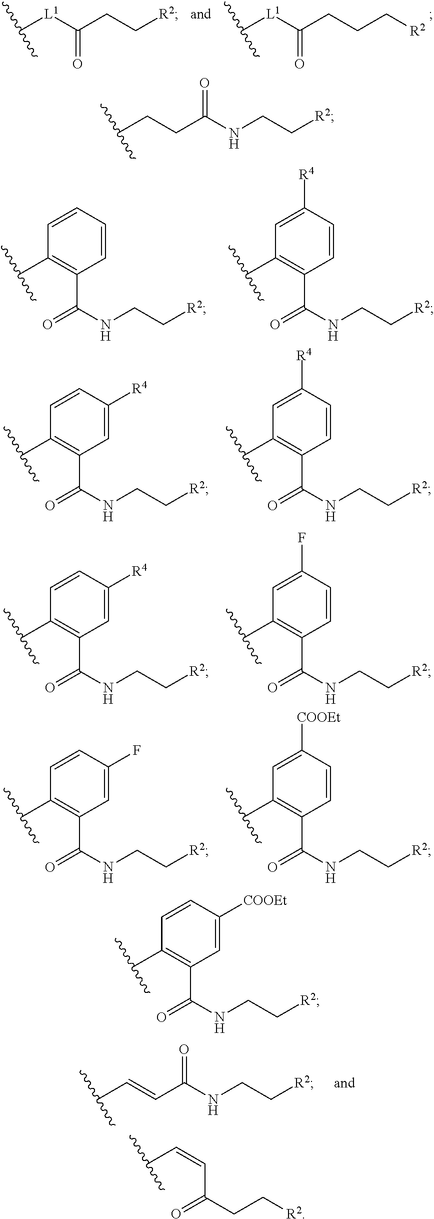

124.-129. (canceled)

130. The compound of claim 1, wherein the compound is any one of structures: 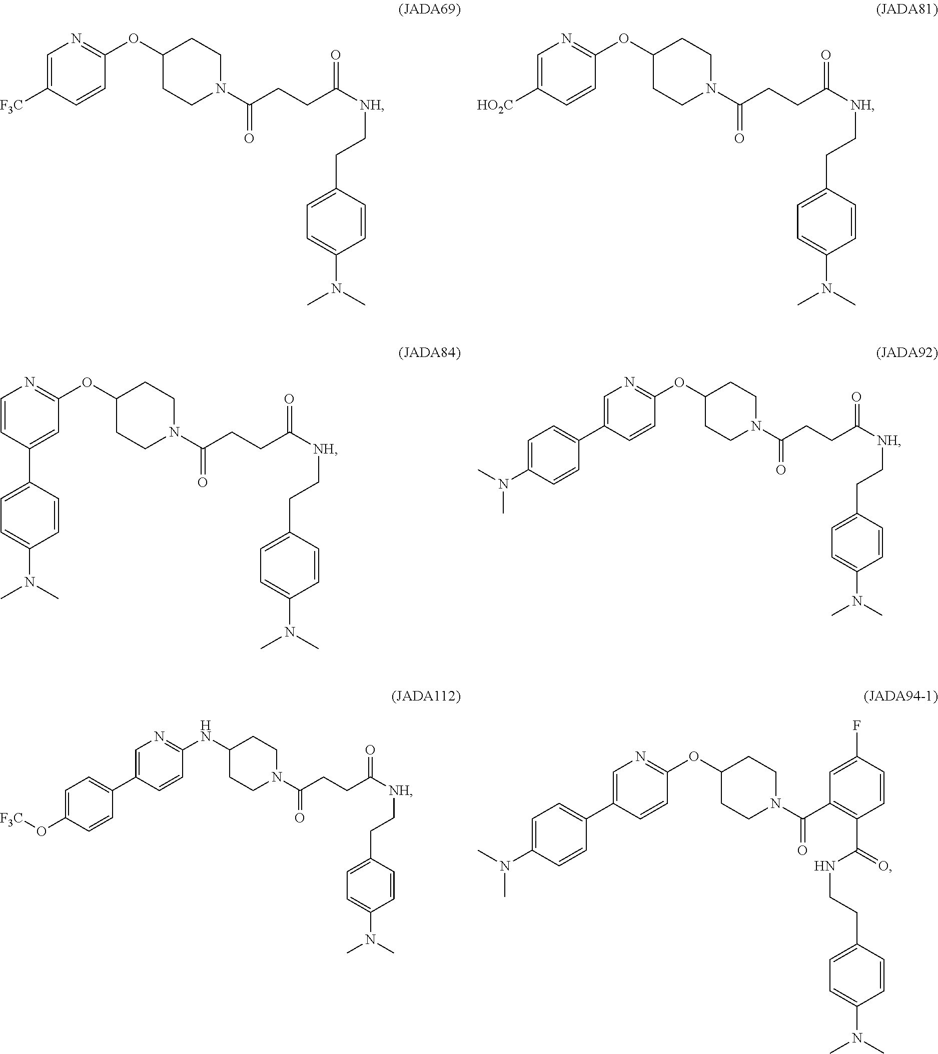 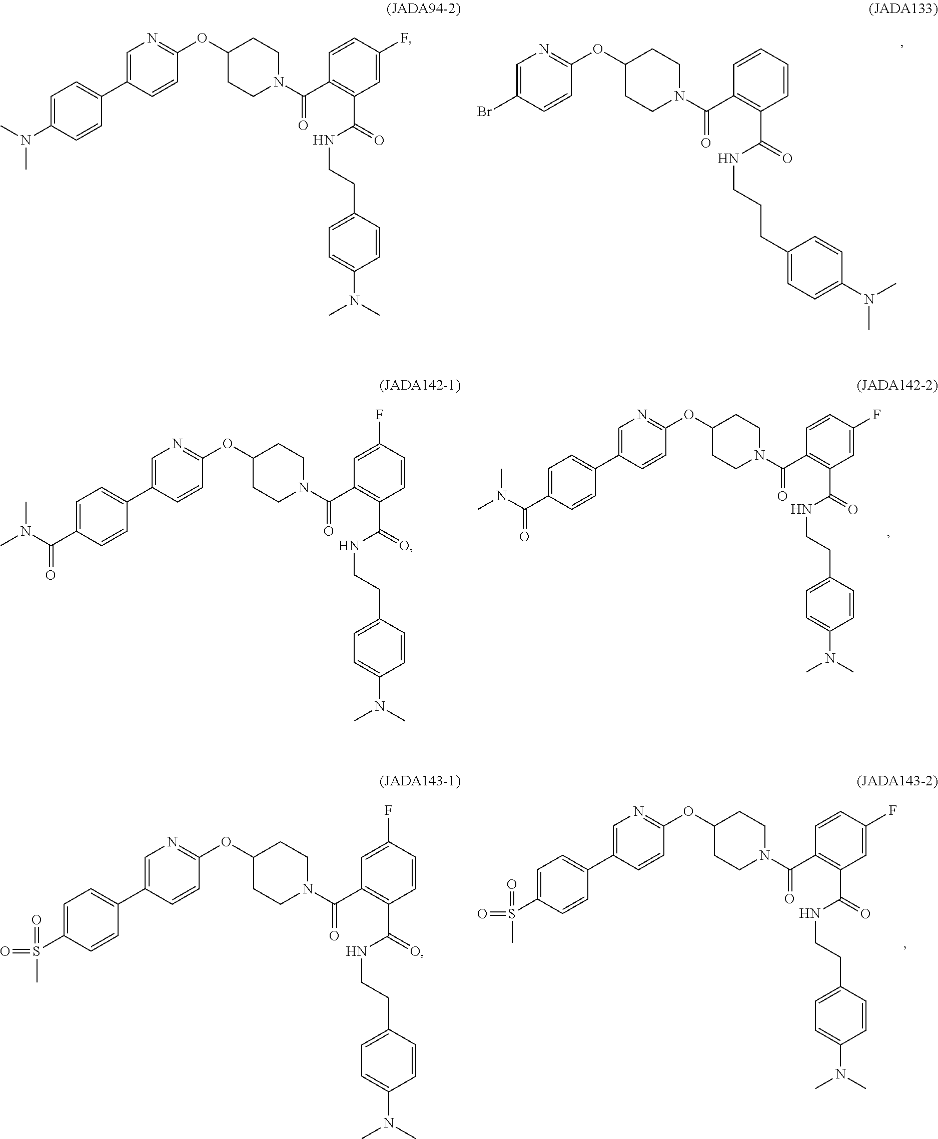 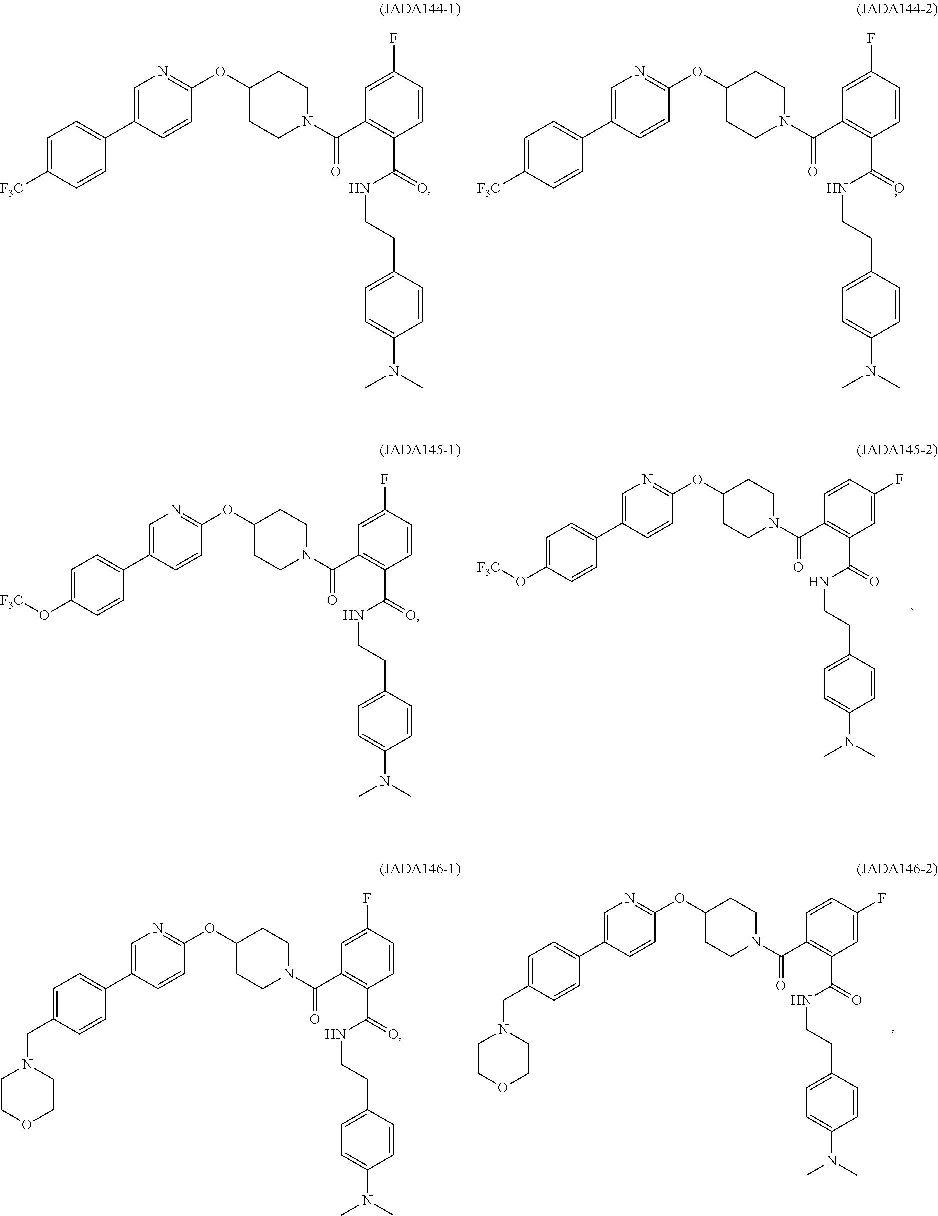 [SMILES: CN(C)c1ccc(CCNC(=O)c2ccc(F)cc2C(=O)N2CCC(Oc3ccc(-c4ccc(C(F)(F)F)cc4)cn3)CC2)cc1 ; CN(C)c1ccc(CCNC(=O)c2ccc(F)cc2C(=O)N2CCC(Oc3ccc(-c4ccc(C(F)(F)F)cc4)cn3)CC2)cc1 ; CN(C)c1ccc(CCNC(=O)c2ccc(F)cc2C(=O)N2CCC(Oc3ccc(-c4ccc(OC(F)(F)F)cc4)cn3)CC2)cc1 ; CN(C)c1ccc(CCNC(=O)c2cc(F)ccc2C(=O)N2CCC(Oc3ccc(-c4ccc(OC(F)(F)F)cc4)cn3)CC2)cc1 ; CN(C)c1ccc(CCNC(=O)c2ccc(F)cc2C(=O)N2CCC(Oc3ccc(-c4ccc(CN5CCOCC5)cc4)cn3)CC2)cc1 ; CN(C)c1ccc(CCNC(=O)c2cc(F)ccc2C(=O)N2CCC(Oc3ccc(-c4ccc(CN5CCOCC5)cc4)cn3)CC2)cc1] 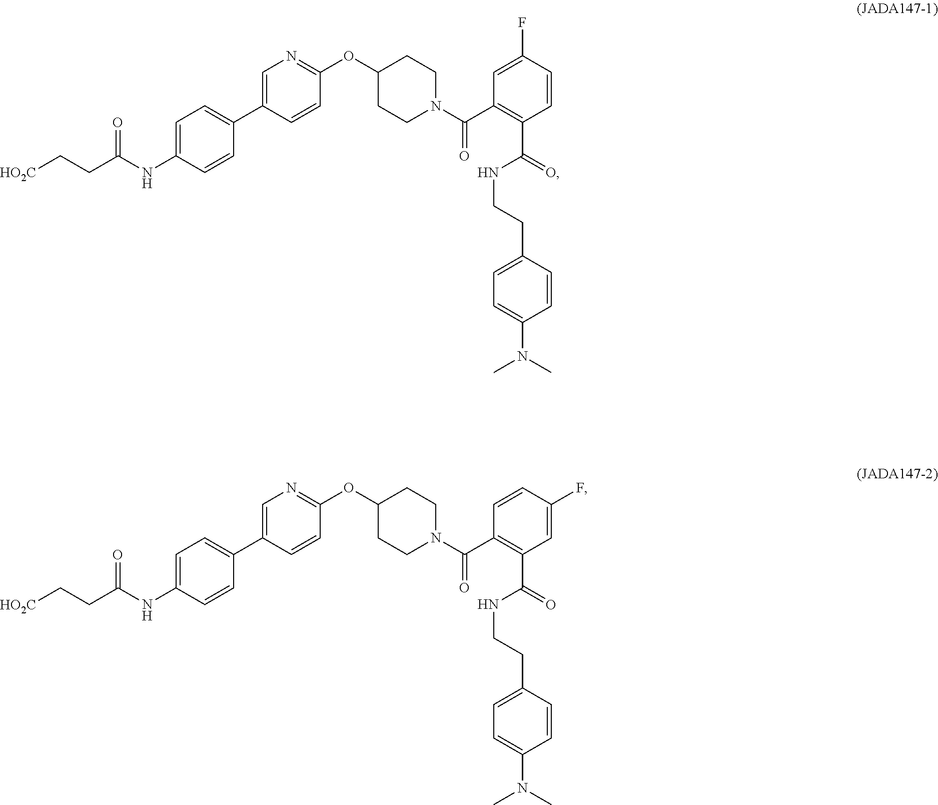 [SMILES: CN(C)c1ccc(CCNC(=O)c2ccc(F)cc2C(=O)N2CCC(Oc3ccc(-c4ccc(NC(=O)CCC(=O)O)cc4)cn3)CC2)cc1 ; CN(C)c1ccc(CCNC(=O)c2cc(F)ccc2C(=O)N2CCC(Oc3ccc(-c4ccc(NC(=O)CCC(=O)O)cc4)cn3)CC2)cc1] 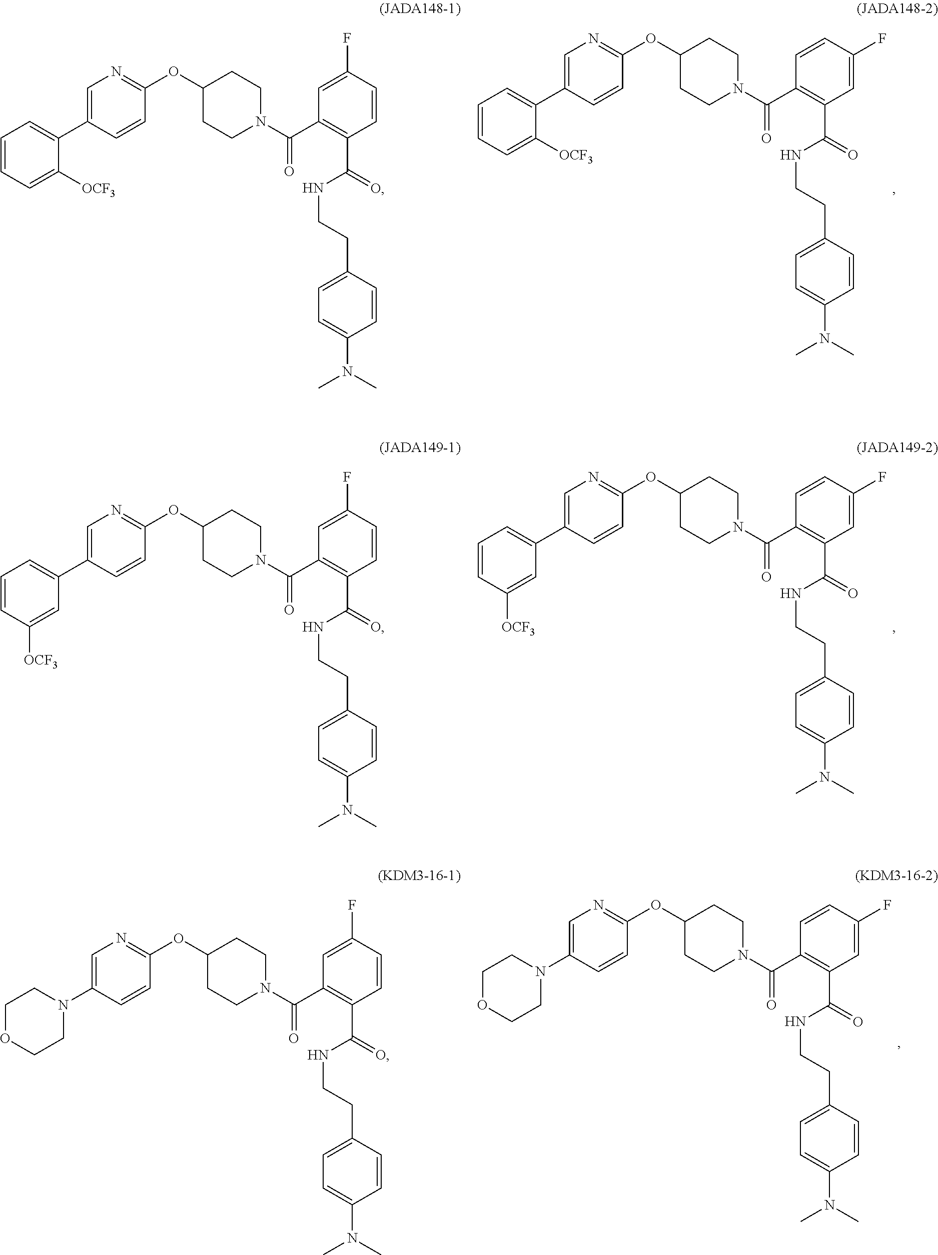 [SMILES: CN(C)c1ccc(CCNC(=O)c2ccc(F)cc2C(=O)N2CCC(Oc3ccc(-c4ccccc4OC(F)(F)F)cn3)CC2)cc1 ; CN(C)c1ccc(CCNC(=O)c2cc(F)ccc2C(=O)N2CCC(Oc3ccc(-c4ccccc4OC(F)(F)F)cn3)CC2)cc1 ; CN(C)c1ccc(CCNC(=O)c2ccc(F)cc2C(=O)N2CCC(Oc3ccc(-c4cccc(OC(F)(F)F)c4)cn3)CC2)cc1 ; CN(C)c1ccc(CCNC(=O)c2cc(F)ccc2C(=O)N2CCC(Oc3ccc(-c4cccc(OC(F)(F)F)c4)cn3)CC2)cc1 ; CN(C)c1ccc(CCNC(=O)c2ccc(F)cc2C(=O)N2CCC(Oc3ccc(N4CCOCC4)cn3)CC2)cc1 ; CN(C)c1ccc(CCNC(=O)c2cc(F)ccc2C(=O)N2CCC(Oc3ccc(N4CCOCC4)cn3)CC2)cc1] 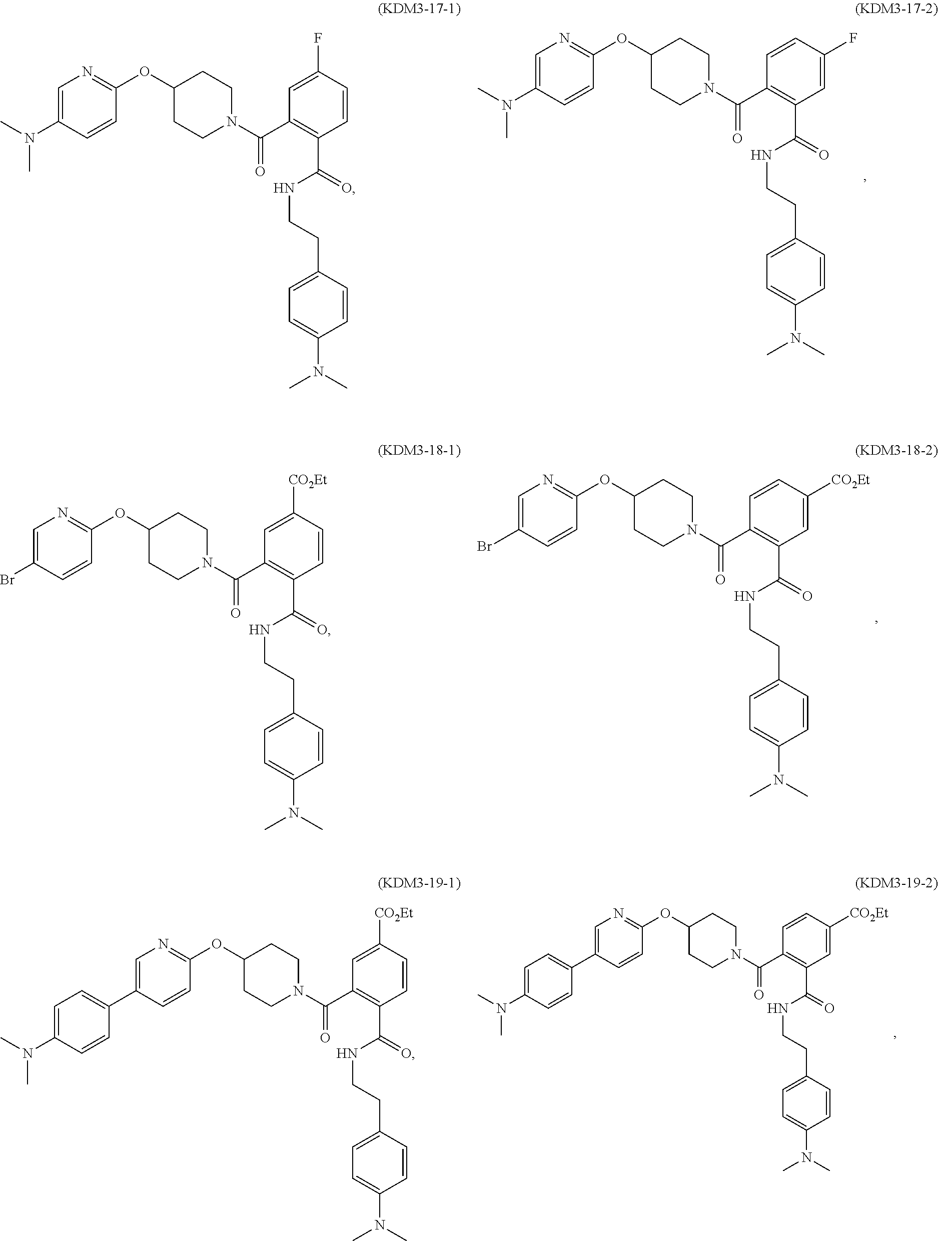 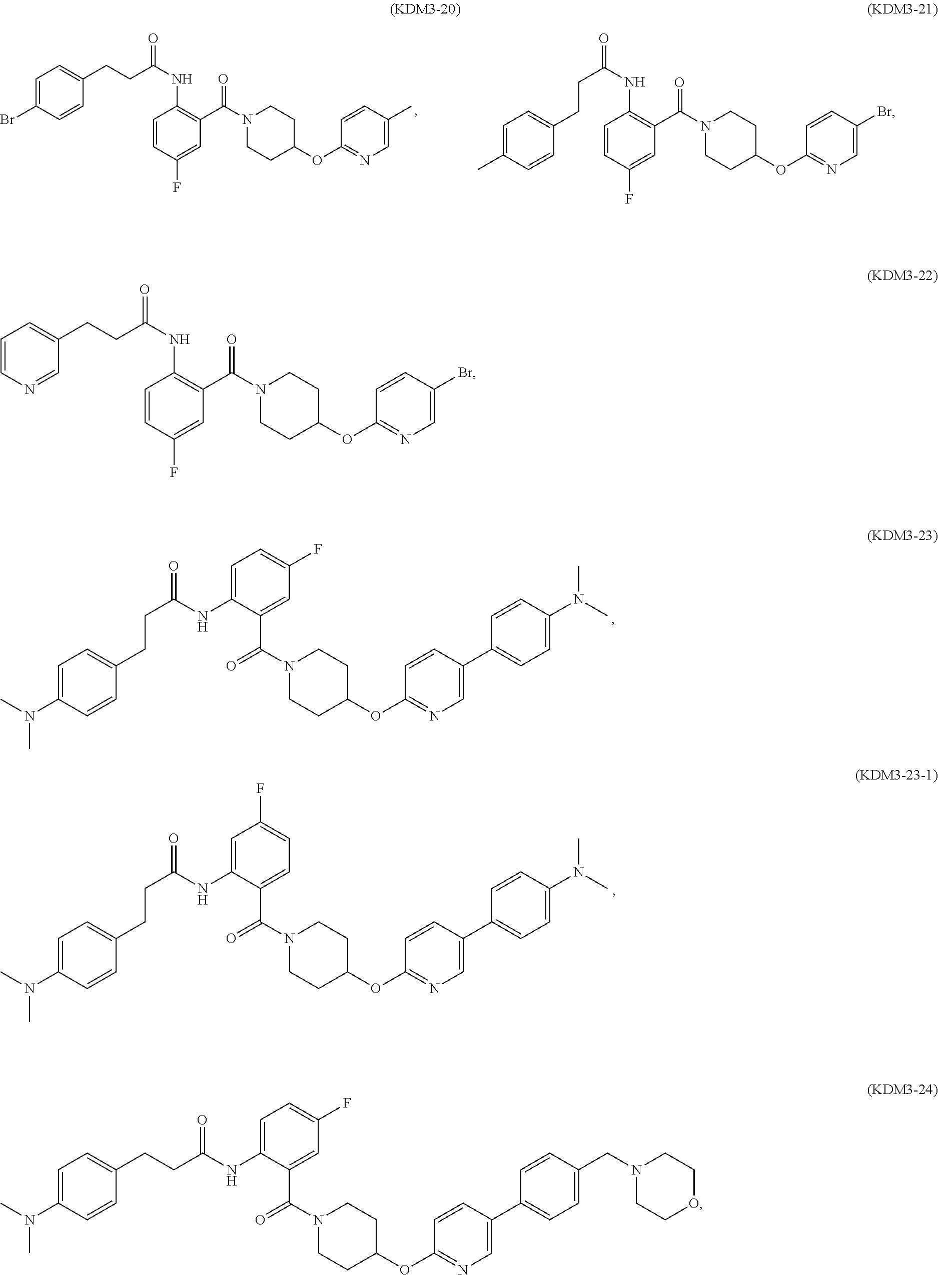 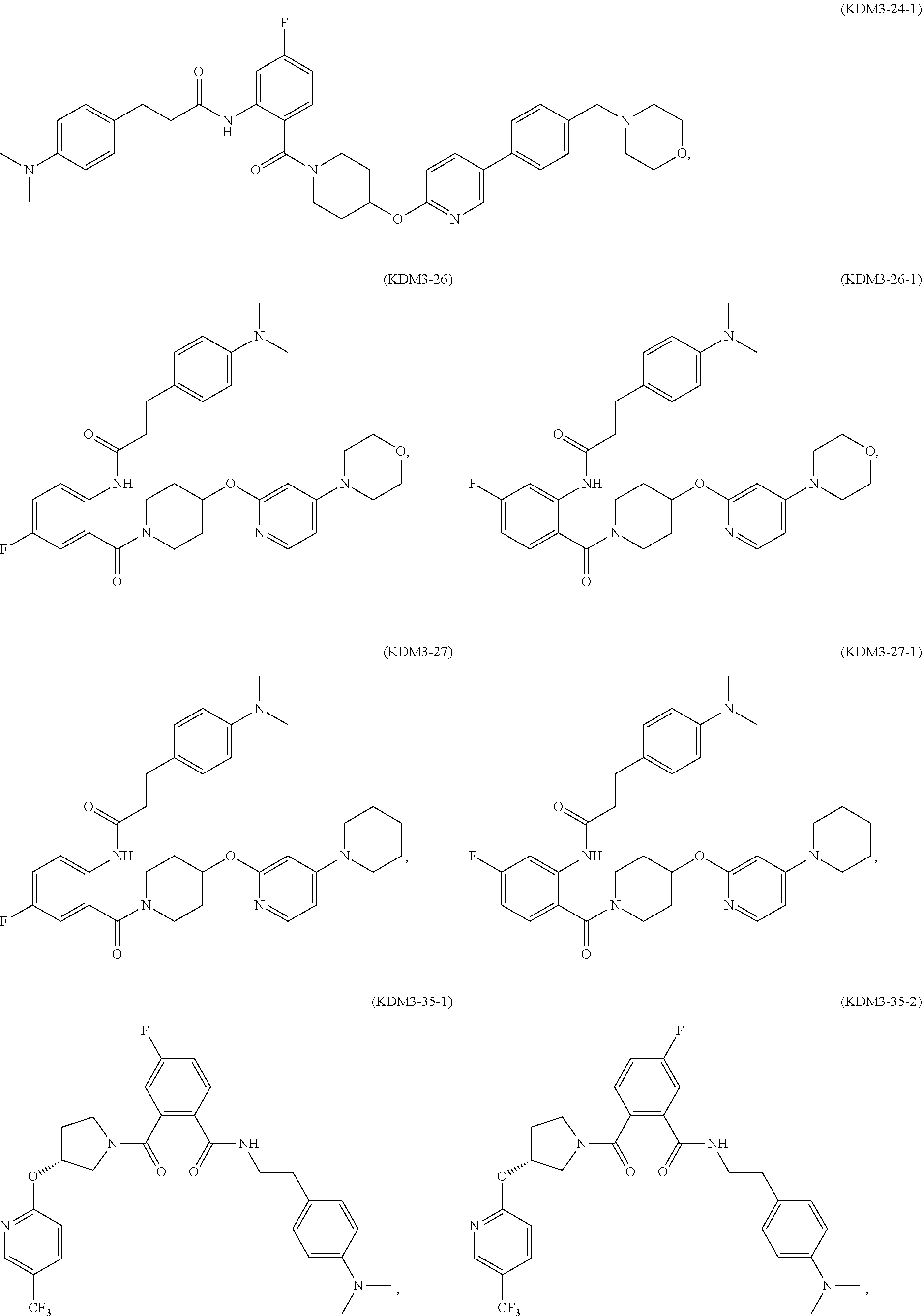 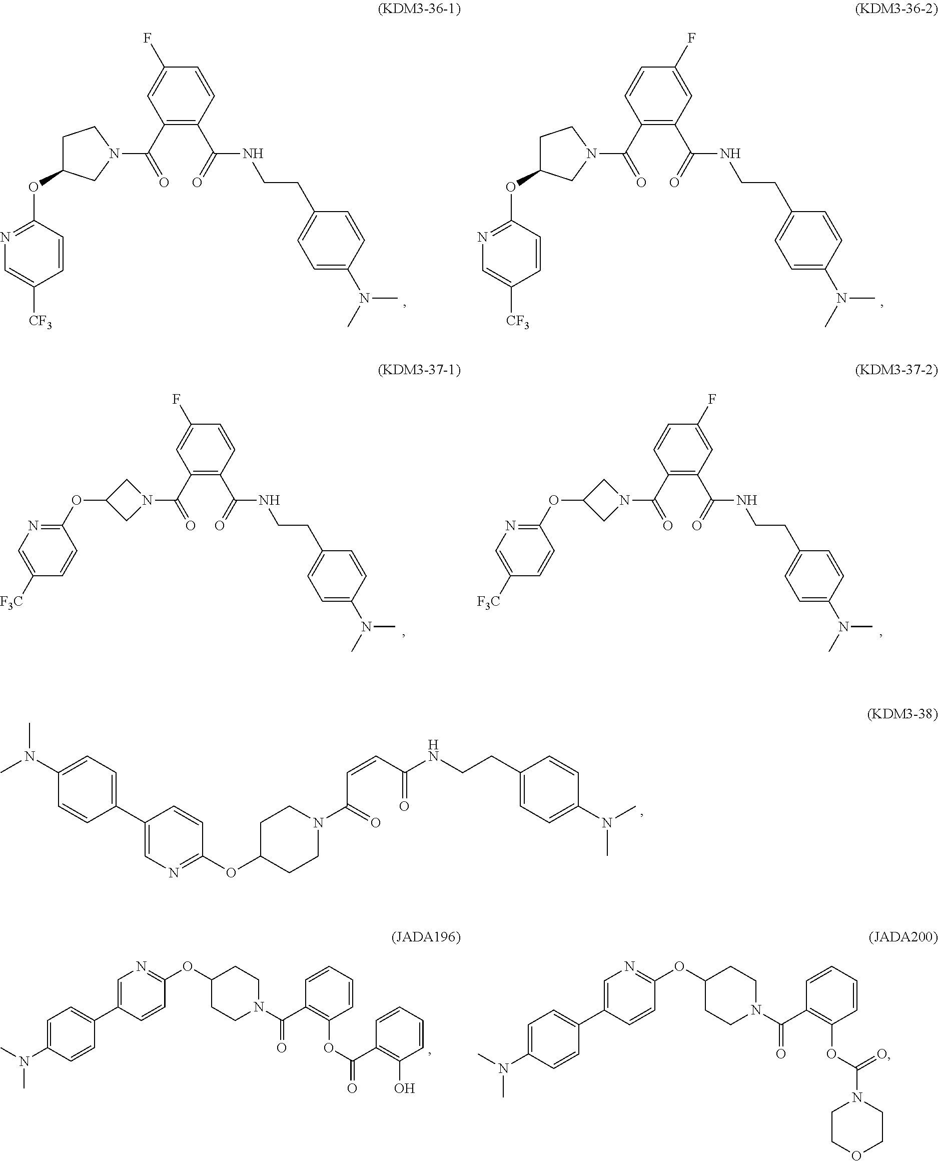 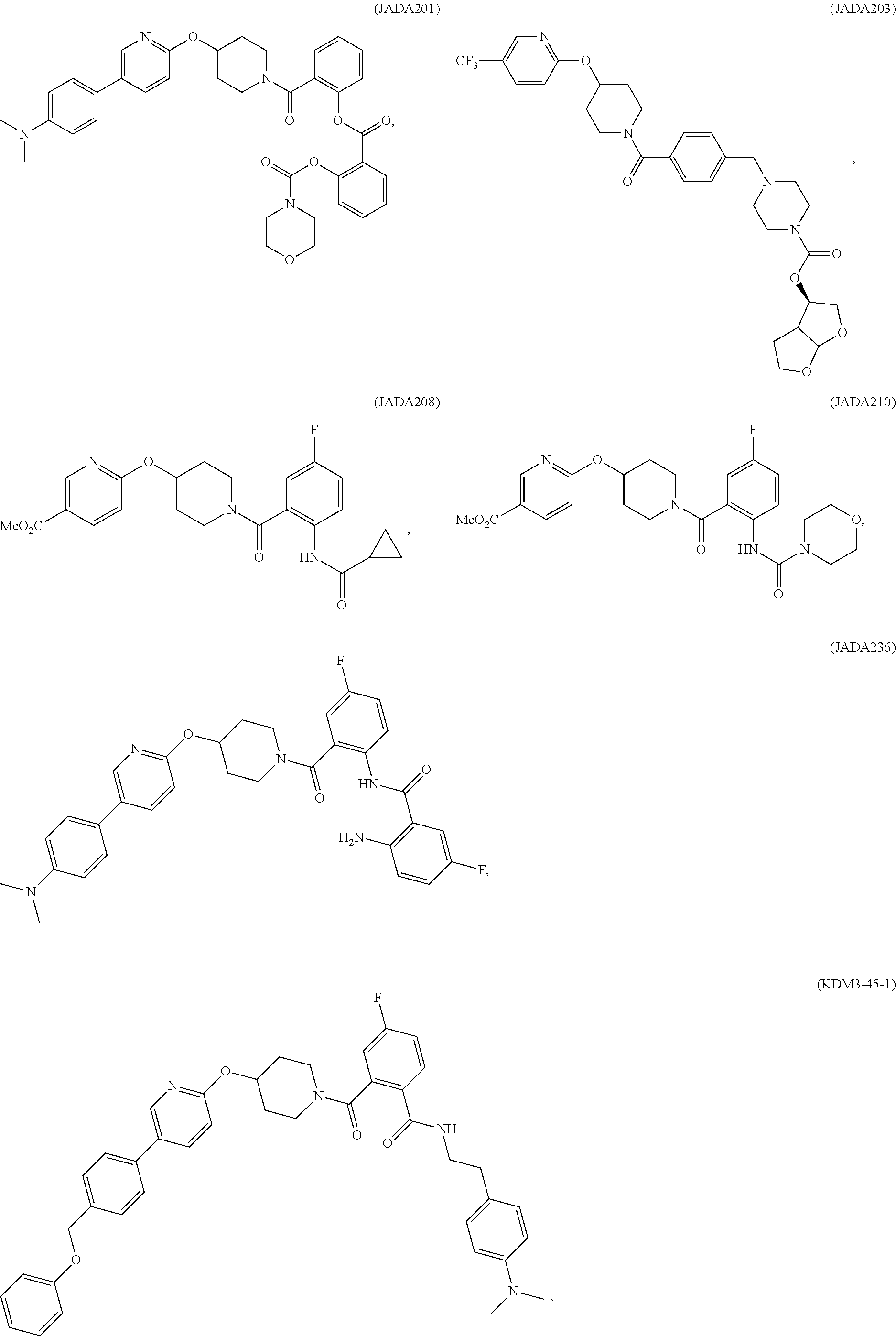 [SMILES: CN(C)c1ccc(-c2ccc(OC3CCN(C(=O)c4ccccc4OC(=O)c4ccccc4OC(=O)N4CCOCC4)CC3)nc2)cc1 ; O=C(O[C@H]1COC2OCCC21)N1CCN(Cc2ccc(C(=O)N3CCC(Oc4ccc(C(F)(F)F)cn4)CC3)cc2)CC1 ; COC(=O)c1ccc(OC2CCN(C(=O)c3cc(F)ccc3NC(=O)C3CC3)CC2)nc1 ; COC(=O)c1ccc(OC2CCN(C(=O)c3cc(F)ccc3NC(=O)N3CCOCC3)CC2)nc1 ; CN(C)c1ccc(-c2ccc(OC3CCN(C(=O)c4cc(F)ccc4NC(=O)c4cc(F)ccc4N)CC3)nc2)cc1 ; CN(C)c1ccc(CCNC(=O)c2ccc(F)cc2C(=O)N2CCC(Oc3ccc(-c4ccc(COc5ccccc5)cc4)cn3)CC2)cc1] 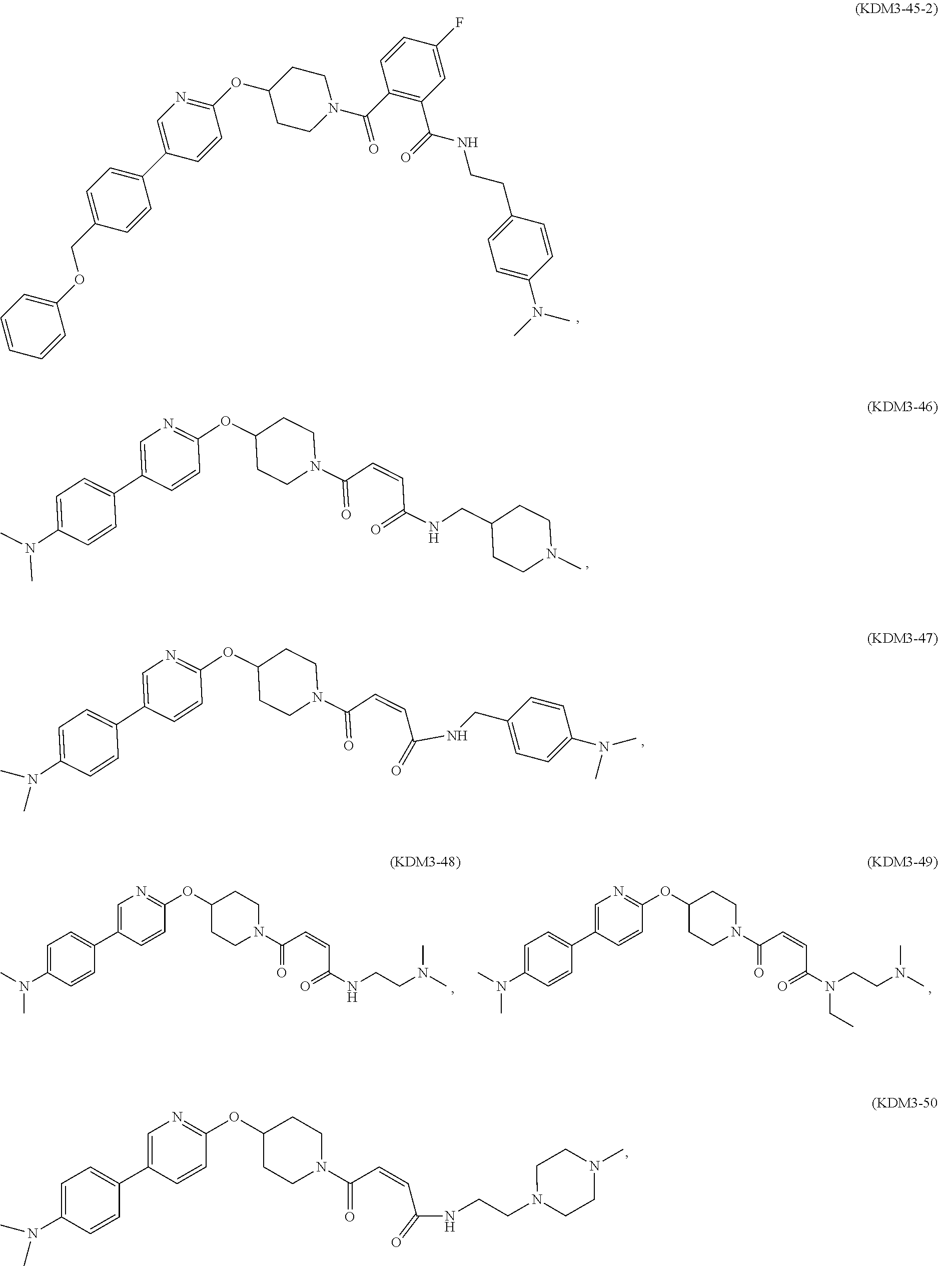 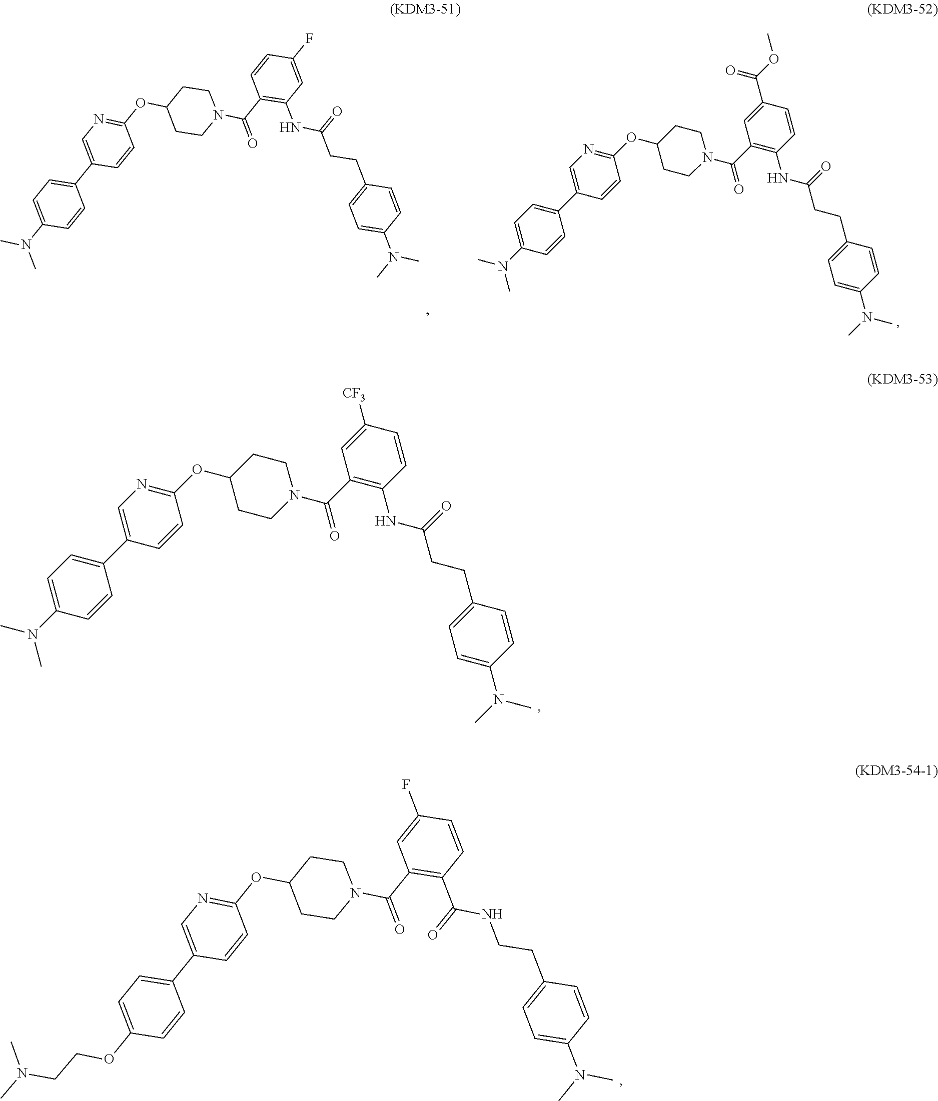 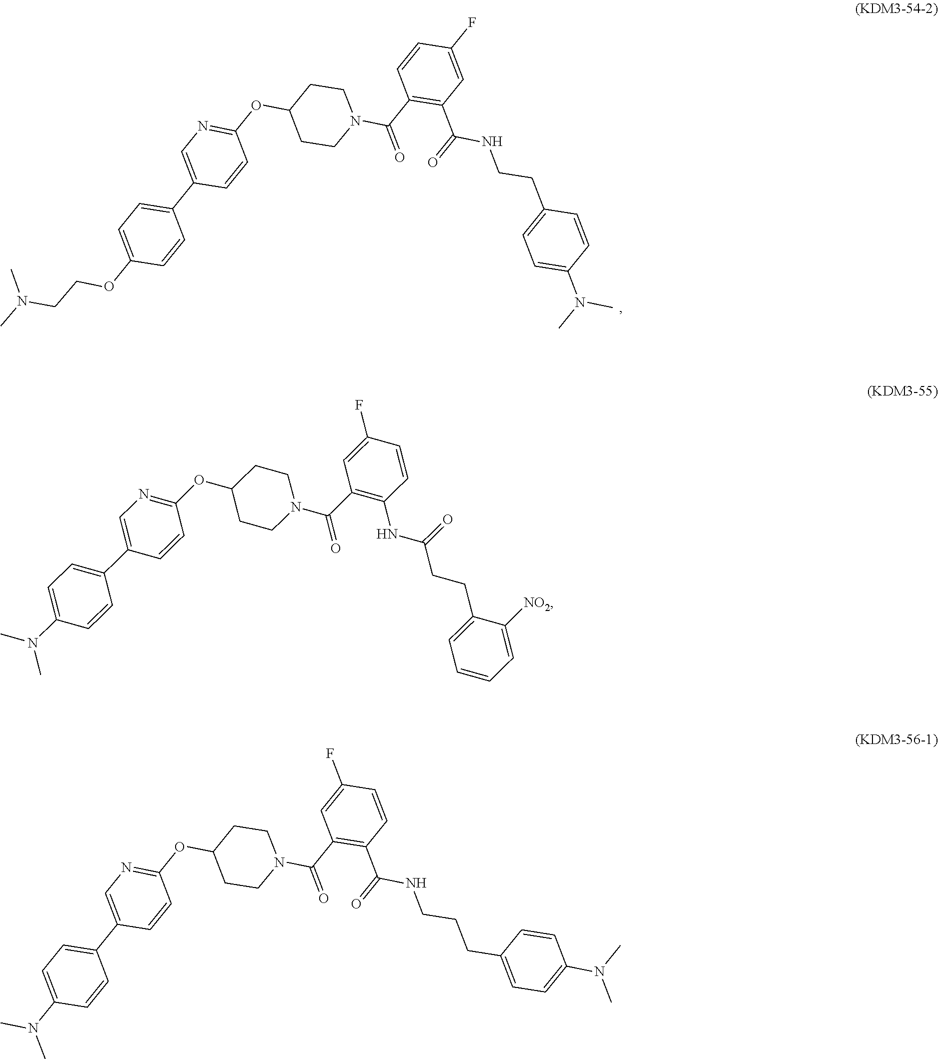 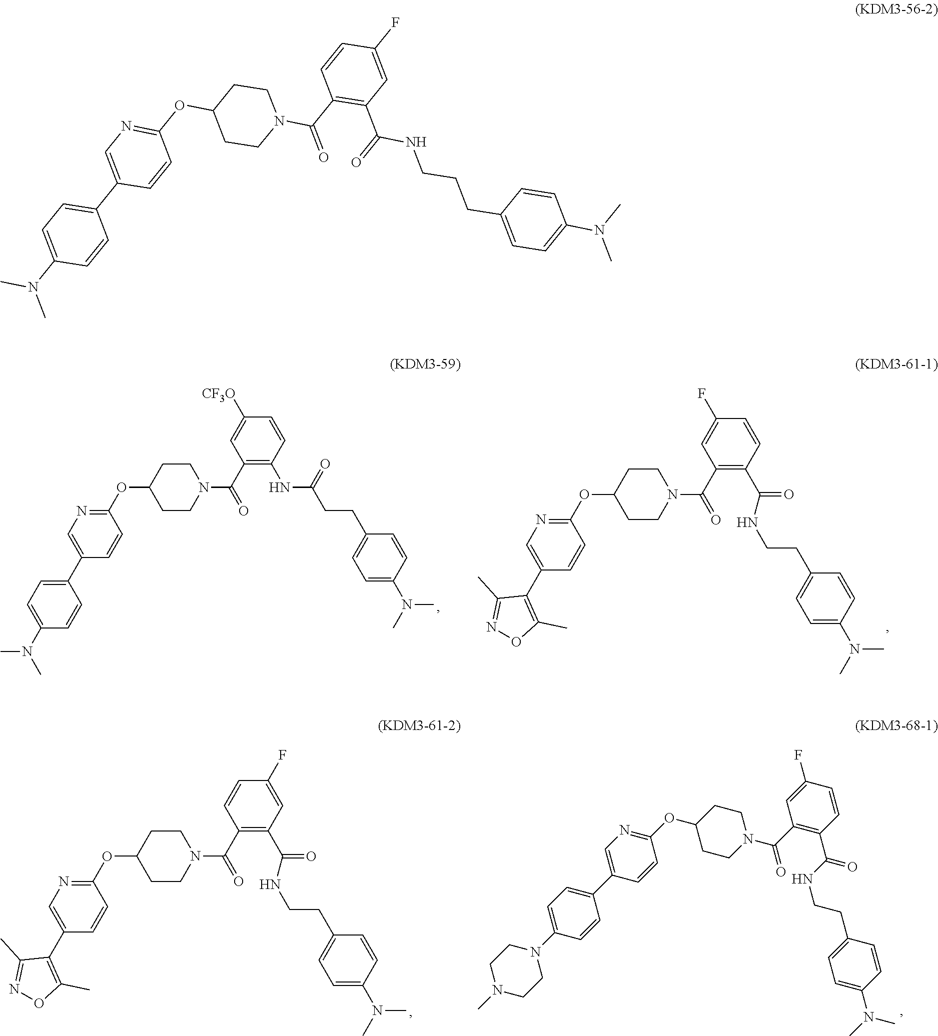 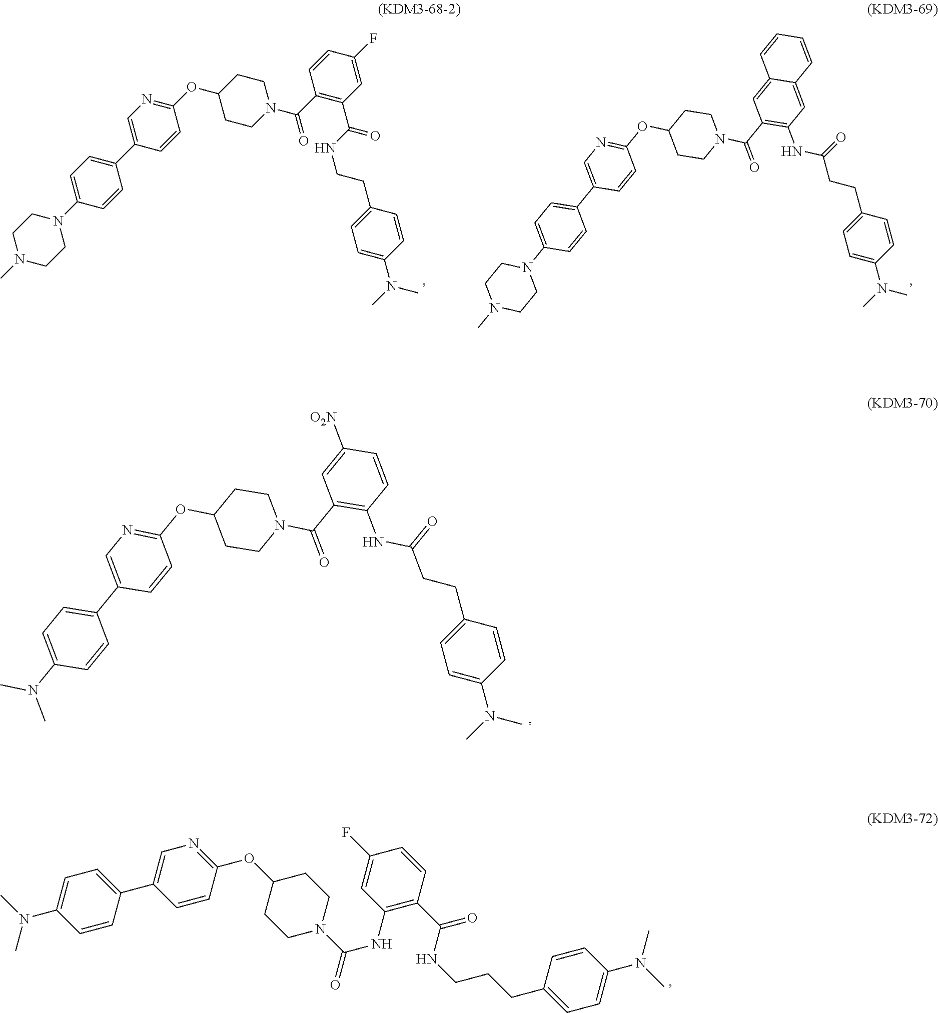 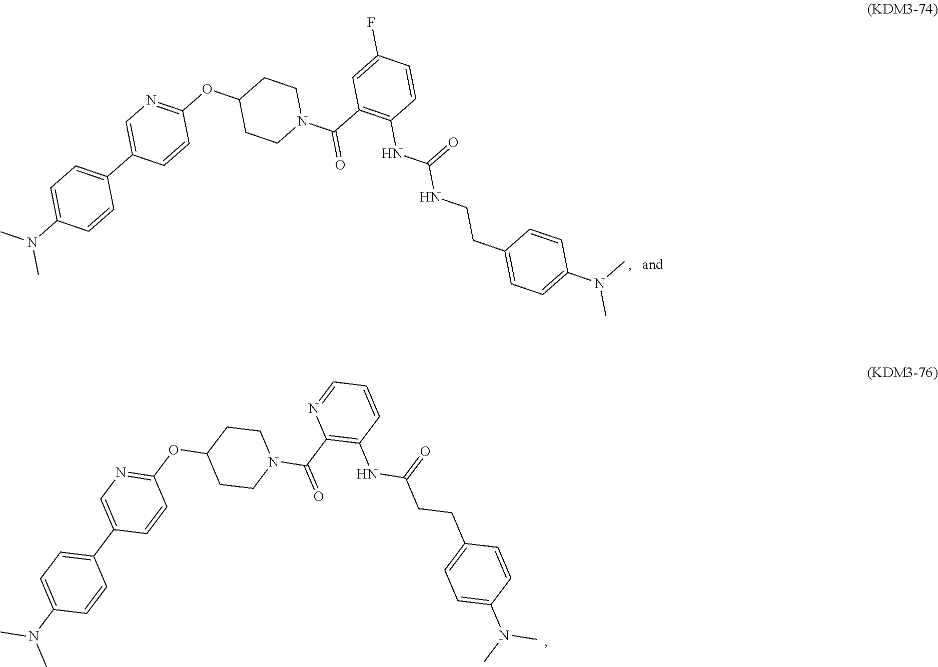
and pharmaceutically acceptable salts, hydrates, solvates, polymorphs, co-crystals, tautomers, stereoisomers, isotopically labeled derivatives, and prodrugs thereof.

131. A compound of Formula (II): 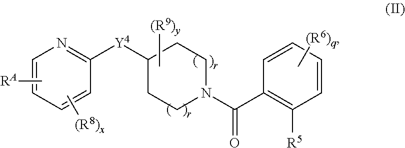
or a pharmaceutically acceptable, hydrate, solvate, polymorph, co-crystal, tautomer, stereoisomer, isotopically labeled derivative, or prodrug thereof, wherein:
each instance of R^{A} is independently optionally substituted aryl, optionally substituted heteroaryl, optionally substituted carbocyclyl, optionally substituted heterocyclyl, or —N(R^{N})₂;
each instance of R⁸ is independently hydrogen, halogen, —CN, —N₃, —NO₂, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, optionally substituted sulfonyl, optionally substituted sulfinyl, —OR^{O}, —N(R^{N})₂, or —SR^{S};
x is an integer from 0-3, inclusive;
Y⁴ is —C(R^{C})₂—, —O—, —NR^{N}—, or —S—;
each instance of R⁹ is independently hydrogen, halogen, —CN, —N₃, —NO₂, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, —OR^{O}, —N(R^{N})₂, or —SR^{S};
y is 0, or an integer from 1 to 9, inclusive;
each instance of r is independently 0 or 1;
R⁵ is hydrogen, —OH, —NO₂, —N(R^{N1})₂, or —C(═O)N(R^{N1})₂, —C(═O)OR^{O1}, or —C(═O)R^{C1};
each instance of R^{N1} is independently hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, optionally substituted sulfonyl, optionally substituted sulfinyl, or a nitrogen protecting group; or two R^{N1} bonded to the same nitrogen atom are taken together with the intervening atoms to form optionally substituted heterocyclyl or optionally substituted heteroaryl;
each instance of R⁶ is independently hydrogen, halogen, —CN, —N₃, —NO₂, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, optionally substituted sulfonyl, optionally substituted sulfinyl, —OR^{O}, —N(R^{N})₂, or —SR^{S};
q is 0, or an integer from 1 to 4, inclusive;
each instance of R^{N} is independently hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, optionally substituted sulfonyl, optionally substituted sulfinyl, or a nitrogen protecting group; or two R^{N} bonded to the same nitrogen atom are taken together with the intervening atoms to form optionally substituted heterocyclyl or optionally substituted heteroaryl;
each instance of R^{C} is independently hydrogen, halogen, —CN, —N₃, —NO₂, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, optionally substituted sulfonyl, optionally substituted sulfinyl, —OR^{O}, —N(R^{N})₂, or —SR^{S}; or two R^{C} bonded to the same carbon are taken together with the intervening atoms to form optionally substituted carbocyclyl or optionally substituted heterocyclyl;
each instance of R^{O} is independently hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, or an oxygen protecting group; and
each instance of R^{O1} is independently hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, or an oxygen protecting group;
each instance of R^{C1} is independently hydrogen, halogen, —CN, —N₃, —NO₂, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, optionally substituted sulfonyl, optionally substituted sulfinyl, —OR^{O}, —N(R^{N})₂, or —SR^{S}; or two R^{C} bonded to the same carbon are taken together with the intervening atoms to form optionally substituted carbocyclyl or optionally substituted heterocyclyl;
each instance of R^{S} is independently hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, a sulfur protecting group, or R^{S}, taken together with the sulfur atom to which it is attached forms a disulfide group substituted with hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, or optionally substituted acyl,
provided that the compound is not:

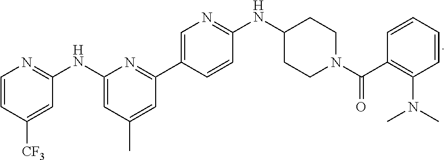 [SMILES: Cc1cc(Nc2cc(C(F)(F)F)ccn2)nc(-c2ccc(NC3CCN(C(=O)c4ccccc4N(C)C)CC3)nc2)c1]

132. The compound of claim 131, wherein:
provided that when Y⁴ is —NH—, each instance of r is 1, and R⁵ is —N(Me)₂, then R^{A} is not optionally substituted heteroaryl; or
provided that when Y⁴ is —NH—, each instance of r is 1, and R⁵ is —N(Me)₂, then R^{A} is not optionally substituted pyridyl; or
provided that when Y⁴ is —NR^{N}—, each instance of r is 1, and R⁵ is —N(R^{N1})₂, then R^{A} is not optionally substituted pyridyl; or
provided that when Y⁴ is —NH—, each instance of r is 1, and R⁵ is —N(Me)₂, then R^{A} is optionally substituted aryl; or
provided that when Y⁴ is —NR^{N}—, each instance of r is 1, and R⁵ is —N(R^{N1})₂, then R^{A} is optionally substituted aryl; or
provided that when Y⁴ is —NH—, each instance of r is 1, and R⁵ is —N(Me)₂, then R^{A} is optionally substituted phenyl; or
provided that when Y⁴ is —NR^{N}—, each instance of r is 1, and R⁵ is —N(R^{N1})₂, then R^{A} is optionally substituted phenyl.

133.-139. (canceled)

140. The compound of claim 131, wherein the compound is of Formula (II-i), (II-ii), or (II-iii): 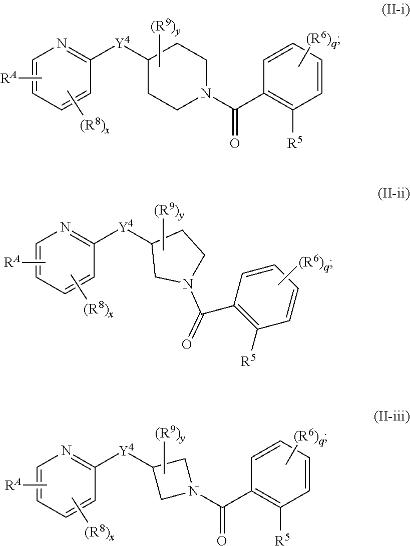
or a pharmaceutically acceptable salt, hydrate, solvate, polymorph, co-crystal, tautomer, stereoisomer, isotopically labeled derivative, or prodrug thereof.

141.-142. (canceled)

143. The compound of claim 131, wherein the compound is of Formula (II-a): 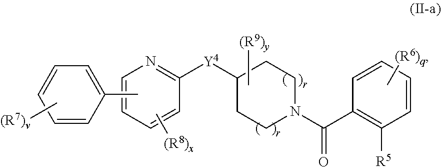
or a pharmaceutically acceptable salt, hydrate, solvate, polymorph, co-crystal, tautomer, stereoisomer, isotopically labeled derivative, or prodrug thereof, wherein
each instance of R⁷ is independently hydrogen, halogen, —CN, —N₃, —NO₂, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, optionally substituted sulfonyl, optionally substituted sulfinyl, —OR^{O}, —N(R^{N})₂, or —SR^{S}; and
v is 0, or an integer from 1 to 5, inclusive.

144. The compound of claim 131, wherein the compound is of Formula (II-a-1), (II-a-3), (II-a-4), (II-a-5), or (II-a-6): 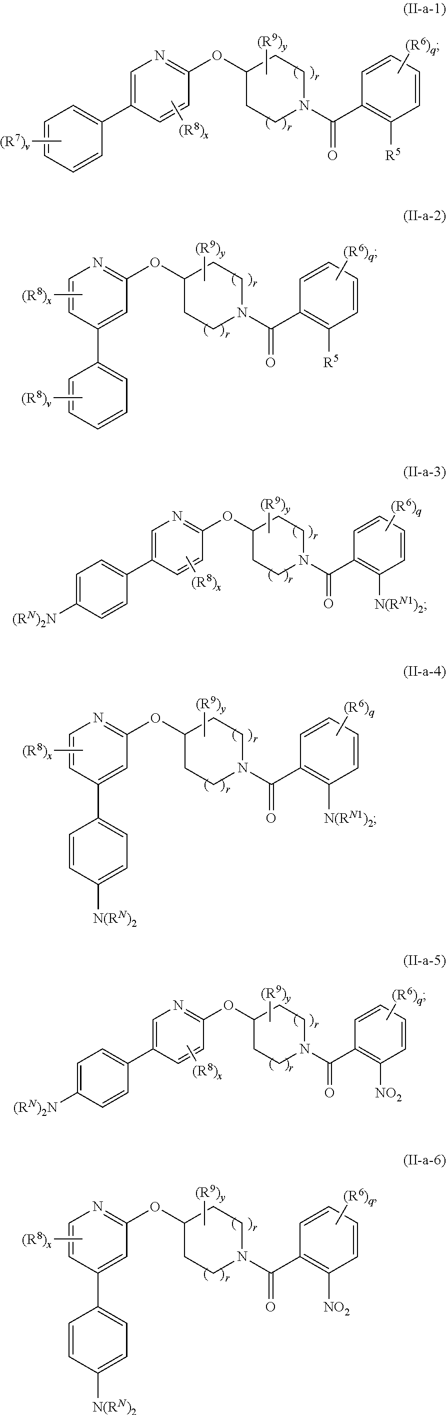
or a pharmaceutically acceptable salt, hydrate, solvate, polymorph, co-crystal, tautomer, stereoisomer, isotopically labeled derivative, or prodrug thereof.

145.-146. (canceled)

147. The compound of claim 131, wherein:
each instance of r is 1, or
one instance of r is 1, and the other instance is 0; or
each instance of r is 0; or
Y⁴ is —NR^{N}—; or
Y⁴ is —O—, —NH—, or —CH₂—; or
at least one instance of R⁹ is hydrogen; or
each instance of R⁹ is hydrogen; or
m is 0; or
at least one instance of R⁸ is optionally substituted aryl; or
at least one instance of R⁸ is optionally substituted phenyl; or
R^{A} is optionally substituted aryl; or
R^{A} is optionally substituted phenyl; or
R⁵ is —N(R^{N1})₂; or
R⁵ is —NO₂, —NH₂, —C(═O)NH₂, —C(═O)OH, R⁵ is —C(═O)OEt, or R⁵ is NHC(═O)CH₃; or
each instance of R^{N1} is hydrogen; or
at least one instance of R⁶ is optionally substituted acyl, —N(R^{N})₂, or halogen; or
at least one instance of R⁶ is —CO₂alkyl, —CO₂H, —NHC(O)Me, or —C₁; or
q is 0 or 1; or
at least one instance of R⁶ is ortho, meta, or para relative to R⁵; or
q is 1 and R⁶ is ortho, meta, or para relative to R⁵; or
at least one instance of R⁷ is —N(R^{N})₂; or
at least one instance of R⁷ is —N(Me)₂; or
v is 1;
at least one instance of R^{N} is hydrogen, acetyl, or optionally substituted C₁₋₆ alkyl;
or
at least one instance of R^{N} is unsubstituted C₁₋₃ alkyl; or
at least one instance of R^{N} is methyl; or
at least one instance of R^{O} is hydrogen, optionally substituted methyl, or optionally substituted acetyl; or
at least one instance of R^{S} is hydrogen.

148-158. (canceled)

159. The compound of claim 131, wherein at least one instance of R⁸ is of structure: 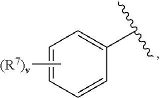
wherein:
each instance of R⁷ is independently hydrogen, halogen, —CN, —N₃, —NO₂, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, optionally substituted sulfonyl, optionally substituted sulfinyl, —OR^{O}, —N(R^{N})₂, or —SR^{S}; and
v is 0, or an integer from 1 to 5, inclusive.

160. The compound of claim 159, wherein:
at least one instance of R⁸ is of structure: 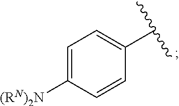
or
at least one instance of R⁸ is of structure: 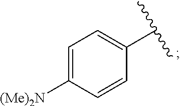
or
at least one instance of R⁸ is hydrogen; or
each instance of R⁸ is hydrogen; or
n is 0 or 1.

161.-167. (canceled)

168. The compound of claim 131, wherein R^{A} is of structure: 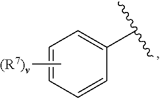
wherein:
each instance of R⁷ is independently hydrogen, halogen, —CN, —N₃, —NO₂, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, optionally substituted sulfonyl, optionally substituted sulfinyl, —OR^{O}, —N(R^{N})₂, or —SR^{S}; and
v is 0, or an integer from 1 to 5, inclusive.

169. The compound of claim 168, wherein R^{A} is of structure:

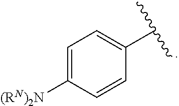

170. The compound of claim 169, wherein R^{A} is of structure:

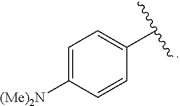

171.-205. (canceled)

206. The compound of claim 131, wherein the compound is any one of structures: 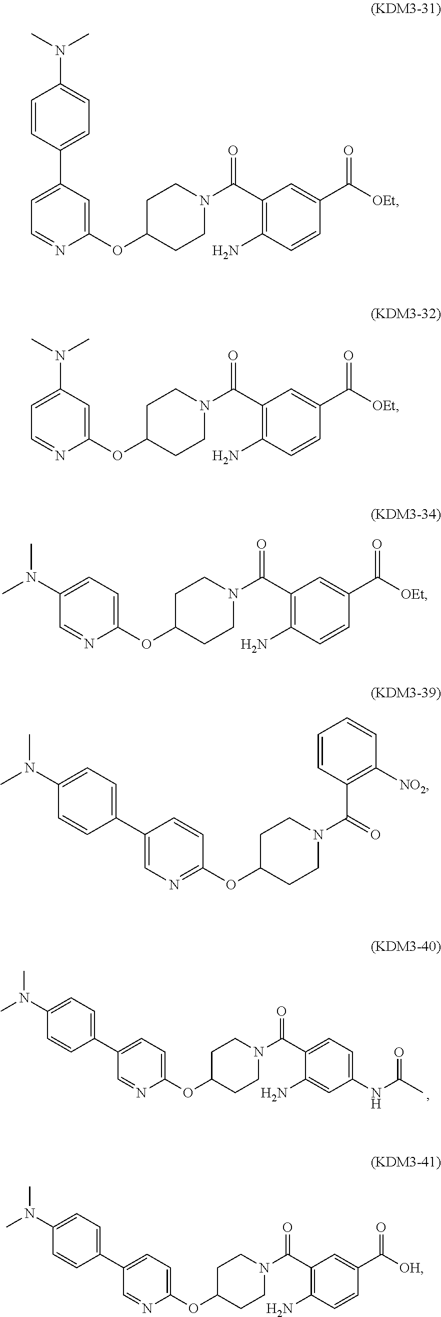 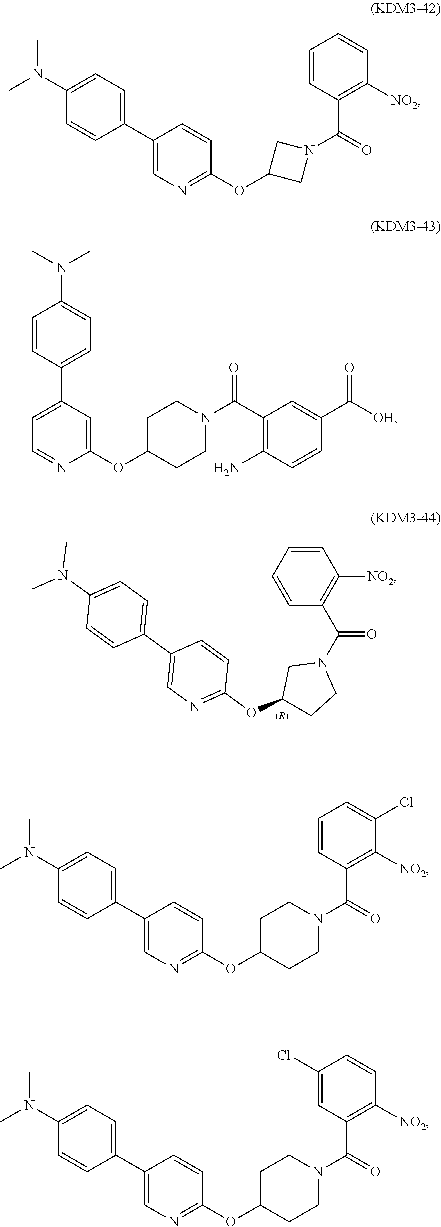 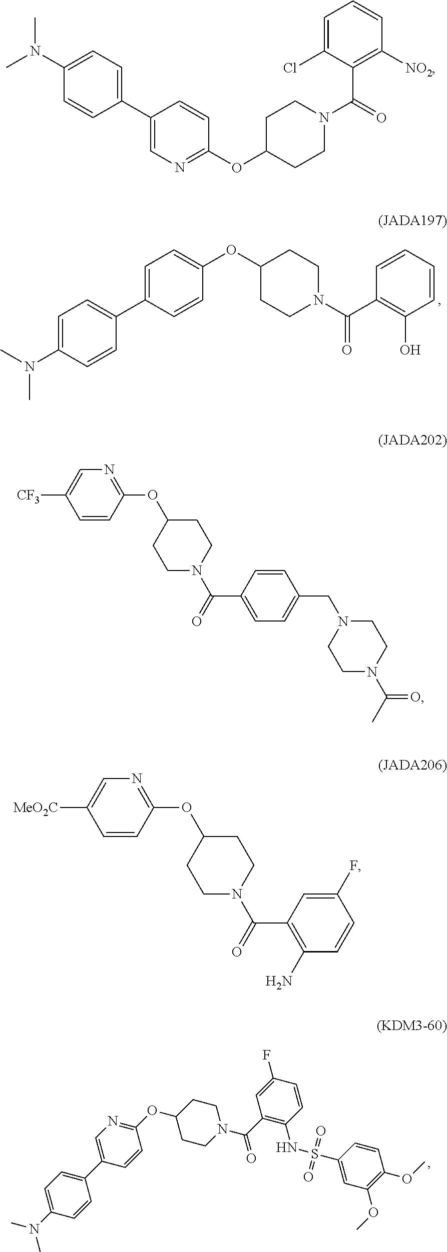 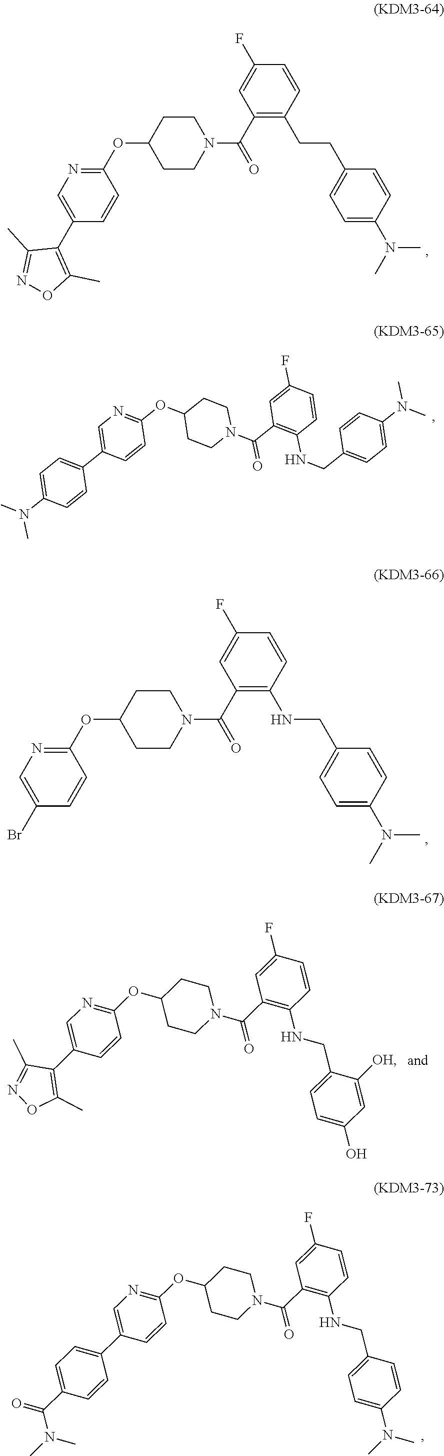
and pharmaceutically acceptable salts, hydrates, solvates, polymorphs, co-crystals, tautomers, stereoisomers, isotopically labeled derivatives, and prodrugs thereof.

207. A compound, which is represented by any one of structures: 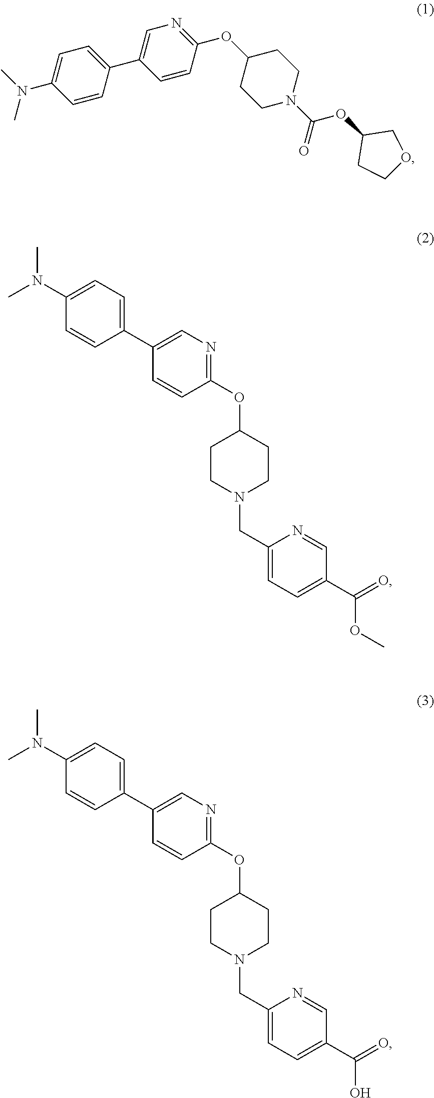 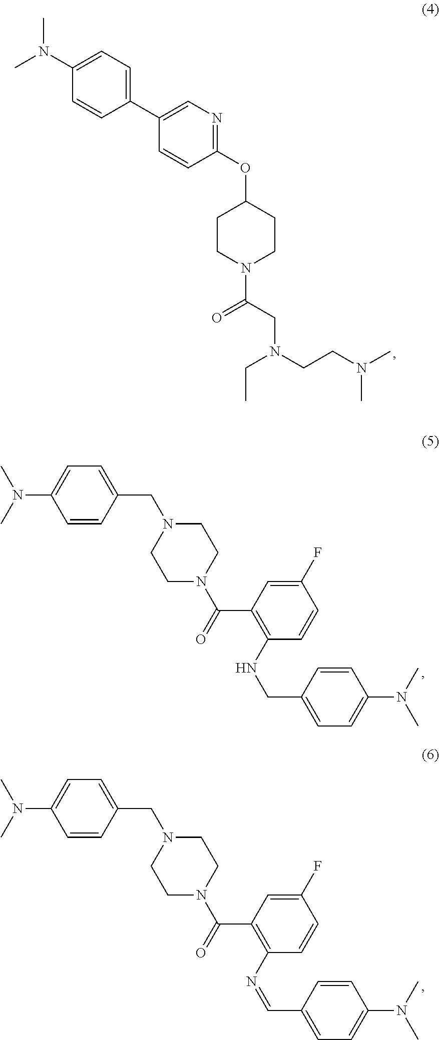 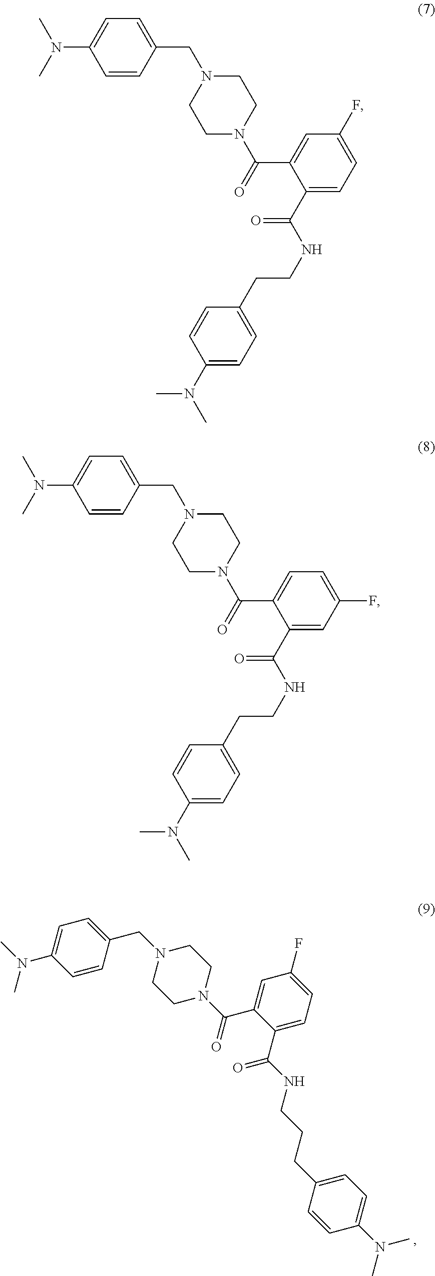 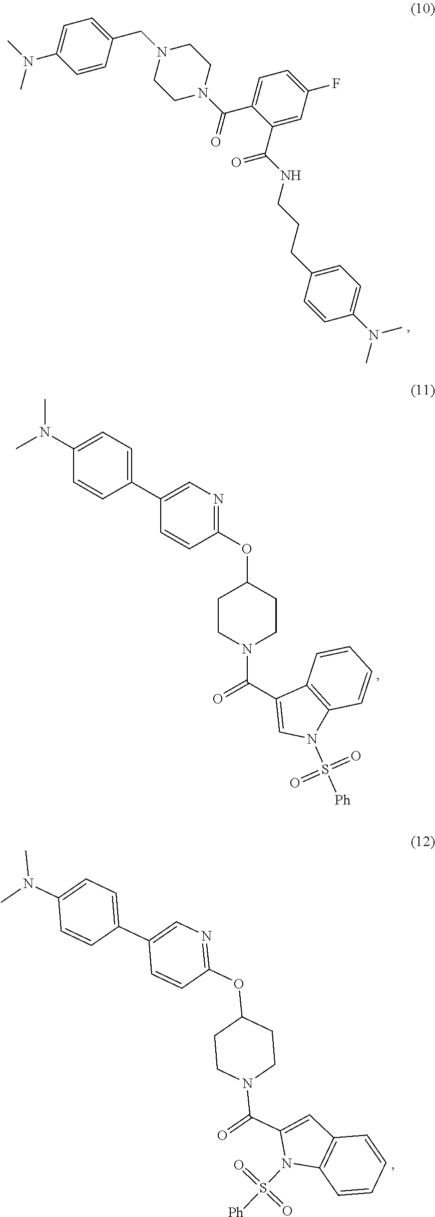 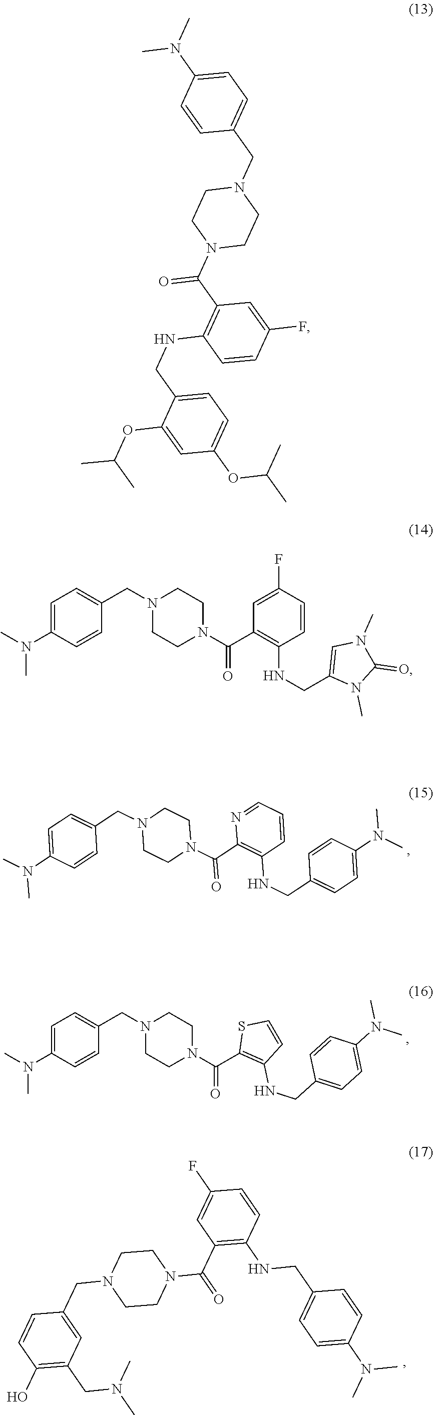 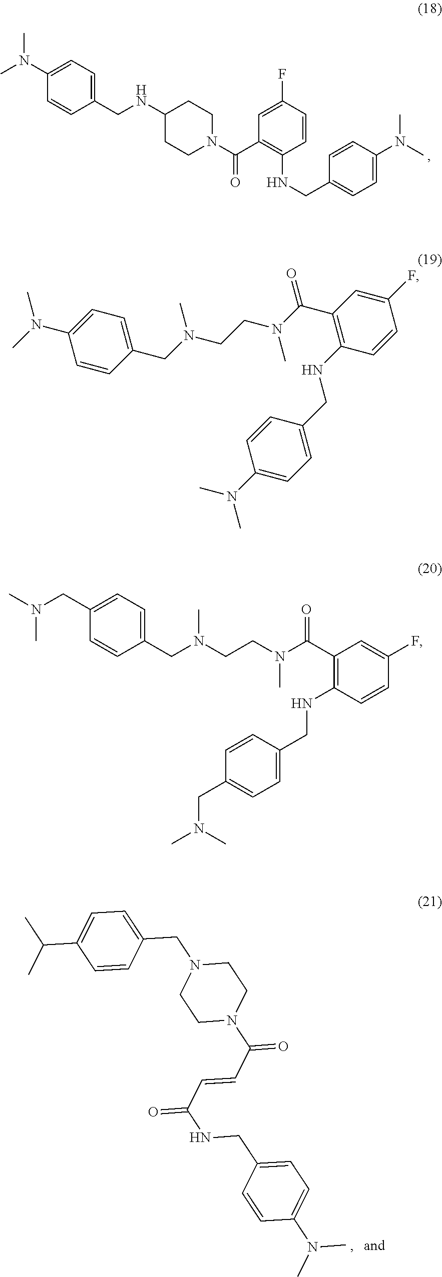 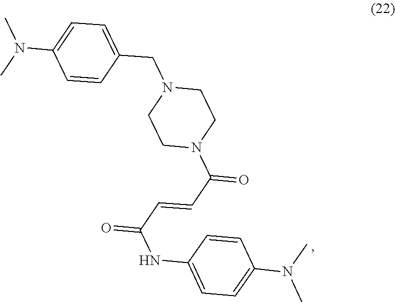
or a pharmaceutically acceptable salt, hydrate, solvate, polymorph, co-crystal, tautomer, stereoisomer, isotopically labeled derivative, and prodrug thereof.

208. A pharmaceutical composition, comprising the compound or a pharmaceutically acceptable salt, hydrate, solvate, polymorph, co-crystal, tautomer, stereoisomer, isotopically labeled derivative, or prodrug thereof of any one of claims 131, and 207, and a pharmaceutically acceptable carrier or excipient.

209. A method of treating a proliferative disease in a subject,
comprising administering to the subject a compound or a pharmaceutically acceptable salt, hydrate, solvate, polymorph, co-crystal, tautomer, stereoisomer, isotopically labeled derivative, or prodrug thereof any one of claims 1, 131, and 207, wherein the proliferative disease is cancer.

210. (canceled)

211. The method of claim 209, wherein the cancer is multiple myeloma, leukemia, lung cancer, breast cancer, liver cancer, pancreatic cancer, gastric cancer, ovarian cancer, or colon cancer.

212.-215. (canceled)

216. A method of inhibiting a histone lysine demethylase (KDM) in vivo or in vitro comprising contacting the KDM with the compound or a pharmaceutically acceptable salt, hydrate, solvate, polymorph, co-crystal, tautomer, stereoisomer, isotopically labeled derivative, or prodrug thereof, of any one of claims 1, 131, and 207, wherein the KDM is a KDM1, KDM2, KDM3, KDM4, KDM5, or KDM6.

217.-218. (canceled)

219. The method of claim 216, wherein the KDM3 is KDM3A, KDM3B, or Jumonji domain containing 1C (JMJD1C).

220.-231. (canceled)

232. The method of claim 211, wherein the leukemia is acute lymphoblastic leukemia (ALL) or acute myeloblastic leukemia (AML).
